(19) United States
(12) Patent Application Publication
(10) Pub. No.: US 2026/0362968 A1
(43) Pub. Date: Nov. 12, 2026

(54) COMPOUND AND LIGHT-EMITTING DEVICE

(71) Applicant: GUANGDONG AGLAIA OPTOELECTRONIC MATERIALS CO., LTD, Guangdong (CN)

(72) Inventors: Shaofu CHEN, Guangdong (CN); Xu WANG, Guangdong (CN); Lei DAI, Guangdong (CN); Lifei CAI, Guangdong (CN)

(73) Assignee: GUANGDONG AGLAIA OPTOELECTRONIC MATERIALS CO., LTD, Guangdong (CN)

(21) Appl. No.: 19/435,409

(22) Filed: Dec. 29, 2025

Related U.S. Application Data
(63) Continuation of application No. PCT/CN2024/087874, filed on Apr. 16, 2024.

(30) Foreign Application Priority Data
Jun. 27, 2023 (CN) 2023 1 0762332.2
Sep. 14, 2023 (CN) 2023 1 1185593.9

(51) Int. Cl. H10K 85/60; C07B 59/00; C07D 213/16; C07D 239/26; C07D 251/24; C07D 401/04; C07D 401/10; C07D 403/04; C07D 405/04; C07D 405/10; C07D 405/14; C07D 409/04; H10K 50/18

(52) U.S. Cl. CPC H10K 85/654; C07B 59/002; C07D 213/16; C07D 239/26; C07D 251/24; C07D 401/04; C07D 401/10; C07D 403/04; C07D 405/04; C07D 405/10; C07D 405/14; C07D 409/04; H10K 85/615; H10K 85/6574; H10K 50/18

(57) ABSTRACT

The present disclosure relates to a compound and a light-emitting device. The compound of the present disclosure has a structure as shown in formula (1). In the present disclosure, the compound obtained by connecting substituted alkyl fluorene to a nitrogen heterocyclic ring has the advantages of good light stability, electrical stability and heat stability, high luminous efficiency, low voltage, long lifetime, etc., and can be used in light-emitting devices. In particular, the compound is expected to be applied to the AMOLED industry as a hole blocking layer material and an electron transport layer material.

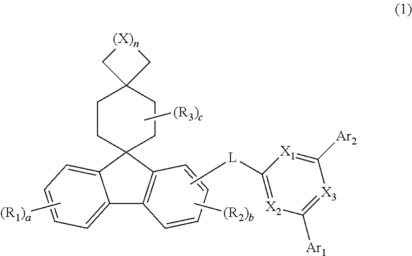

### CROSS-REFERENCE TO RELATED APPLICATIONS

This application is a bypass continuation of International Application No.: PCT/CN2024/087874 filed on Apr. 16, 2024, which claims the benefit of Chinese Patent Application No. 202310762332.2, filed on Jun. 27, 2023, and the Chinese Patent Application No. 202311185593.9, filed on Sep. 14, 2023, the entire contents of which are incorporated by reference in their entirety.

### TECHNICAL FIELD

The present disclosure relates to the technical field of organic electroluminescence, in particular, to an organic light-emitting material suitable for a light-emitting device, and more particularly, to a compound in which a substituted alkylfluorene is connected to a nitrogen heterocycle and a light-emitting device thereof.

### BACKGROUND

At present, a light-emitting device, as a new generation of display technology, has been attracting increasing attention in display and illumination technologies, with extremely broad application prospects. However, compared with market application requirements, the luminous efficiency, driving voltage, service life and other performance of a light-emitting device still needs to be further strengthened and improved, especially for OLED devices.

Generally, a basic structure of an OLED consists of thin film layers of organic functional materials with various functions sandwiched between metal iridium electrodes, resembling a sandwich structure. Driven under an electric current, holes and electrons are injected from the anode and the cathode, respectively. After moving at a certain distance, the holes and the electrons are compounded on the light-emitting layer and are released in the form of light or heat, thereby generating illumination of the OLED. However, the organic functional materials are the core compositions of the organic electroluminescent device, and their thermal stability, photochemical stability, electrochemical stability, quantum yield, film forming stability, crystallinity, color saturation, and other properties are all important factors affecting the performance of the device. Patent document 1 (CN106132937A) discloses a compound

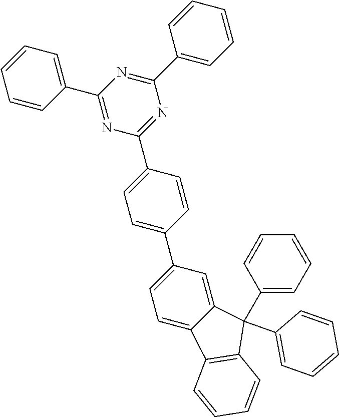 [SMILES: c1ccc(-c2nc(-c3ccccc3)nc(-c3ccc(-c4ccc5c(c4)C(c4ccccc4)(c4ccccc4)c4ccccc4-5)cc3)n2)cc1]

of which 9,9-diphenylfluorene is bonded to a triazine ring via a linker. These compounds can be used in a light-emitting device as electron injection and transport materials. Since the device has a higher voltage, its efficiency and lifetime need to be further improved. Patent document 2 (CN111433190A) discloses a compound

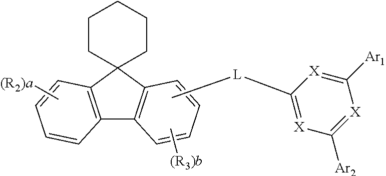

of which cyclohexylfluorene is bonded to a triazine ring via a linker. These compounds can be used in a light-emitting device as host materials and electron transport materials, however the efficiency of the device needs to be improved.

Patent document 3 (CN110615759B) discloses a compound

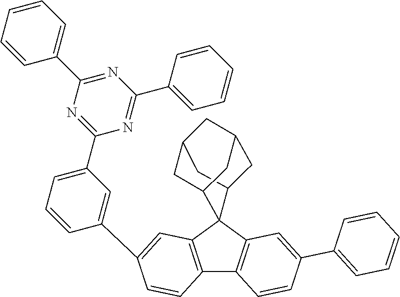 [SMILES: c1ccc(-c2ccc3c(c2)C2(c4cc(-c5cccc(-c6nc(-c7ccccc7)nc(-c7ccccc7)n6)c5)ccc4-3)C3CC4CC(C3)CC2C4)cc1]

of which adamantylfluorene is bonded to a triazine ring via a linker. These compounds are used in a light-emitting device as electron transport layer materials, however its performance such as luminous efficiency and lifetime, etc. needs to be further improved. Patent document 4 (CN114763341A) discloses a compound

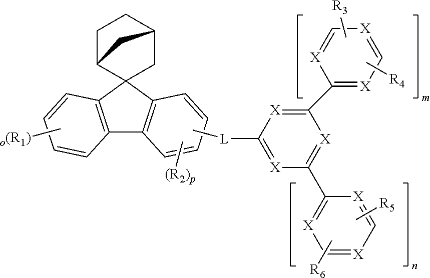

of which norbornanylfluorenyl is bonded to a nitrogen heterocyclic ring via a linker. These compounds are used in a light-emitting device as electron transport layer materials, however its performance such as material stability, luminous efficiency and lifetime, etc. needs to be further improved. Patent document 5 (KR1020210156912A) discloses a compound

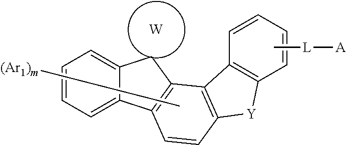

of which benzoheterocyclofluorenyl is bonded to a nitrogen heterocyclic ring via a linker. These compounds are used in a light-emitting device as electron transport layer materials, however it has strong planarity, high sublimation temperature, and poor material stability. Thus, the performance of the device such as luminous efficiency and lifetime, etc. needs to be further improved.

### SUMMARY

The present disclosure has been completed in order to solve the above-mentioned problems, and an objective of the present disclosure is to provide a high-performance light-emitting device and a novel material capable of realizing such a light-emitting device.

To achieve the aforementioned objective, the present applicant has repeatedly conducted in-depth studies and as a result, has found that a high-performance light-emitting device can be obtained by using a compound represented by the following formula (1).

One of the objectives of the present disclosure is to provide a compound of which a substituted alkylfluorene is connected to a nitrogen heterocyclic ring. This type of compound has the advantages of good film-forming properties, good optical, electrical and heat stabilities, high luminous efficiency, low voltage and long lifetime, etc., and can be used in a light-emitting device. In particular, it is expected to be applied to the AMOLED industry as a hole blocking layer material and an electron transport layer material.

To achieve the above objectives, the following technical solutions are used herein.

A compound has a structure of formula (1):

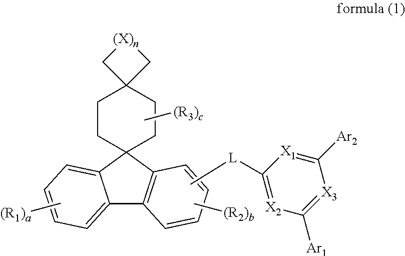

wherein X is independently selected from the group consisting of C(R₄)₂, O, S, and NR₅;
wherein n is independently selected from 0 or an integer of 1-6, when n=0, a ring is not formed through connection; when n≥1, a ring is formed through connection; and when n≥2, each X is the same or different;
wherein X₁, X₂ and X₃ are independently selected from N or CR₆, and at least one of X₁, X₂ and X₃ is N;
wherein R₁, R₂ and R₃ are the substituents on respective rings and separately independently selected from the group consisting of deuterium, halogen, cyano, nitro, C1-C40 alkyl, C1-C40 heteroalkyl, C2-C40 alkenyl, C2-C40 alkynyl, C3-C40 cycloalkyl, C3-C40 heterocycloalkyl, C6-C60 aryl, C5-C60 heteroaryl, C1-C40 alkoxy, C6-C60 aryloxy, C3-C40 alkylsilyl, C6-C60 arylsilyl, C1-C40 alkylboryl, C6-C60 arylboryl, C6-C60 arylphosphino, C6-C60 monoarylphosphino, C6-C60 diarylphosphino, and C6-C60 arylamino;
R₄, R₅ and R₆ are each independently selected from the group consisting of hydrogen, deuterium, halogen, cyano, nitro, C1-C40 alkyl, C1-C40 heteroalkyl, C2-C40 alkenyl, C2-C40 alkynyl, C3-C40 cycloalkyl, C3-C40 heterocycloalkyl, C6-C60 aryl, C5-C60 heteroaryl, C1-C40 alkoxy, C6-C60 aryloxy, C3-C40 alkylsilyl, C6-C60 arylsilyl, C1-C40 alkylboryl, C6-C60 arylboryl, C6-C60 arylphosphino, C6-C60 monoarylphosphino, C6-C60 diarylphosphino, or C6-C60 arylamino;
L is selected from a single bond, or a substituted or unsubstituted aromatic or heteroaromatic group with 6 to 30 ring atoms;
wherein, a, b, and c are each independently selected from an integer of 0-8;
wherein Ar₁ and Ar₂ are each independently selected from a substituted or unsubstituted C6-C30 aryl, or a substituted or unsubstituted C2-C30 heteroaryl;
each heteroatom in the heteroaryl, or heteroalkyl or heterocycloalkyl is independently selected from at least one heteroatom of O, S, N, Se, Si and Ge; and
the substitution refers to being substituted with deuterium, F, Cl, Br, C1-C6 alkyl, C3-C6 cycloalkyl, C1-C6 alkyl substituted amino, C6-C12 aryl, cyano, isocyano or phosphino; and the amount of substitutions of each substituent group is independently selected from one of single substitution to the maximum amount of substitutions.

In some embodiments, the compound is selected from the structure of formula (2) or formula (3):

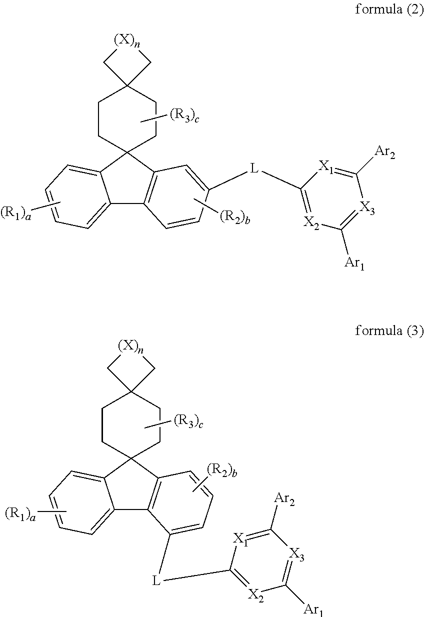

when n=0, a ring is not formed through connection and X is absent; when n≥1, a ring is formed through connection; when n≥2, X is a plurality of C(R₄)₂, or a combination of one or more C(R₄)₂ and one of O, S or NR₅.

In some embodiments,

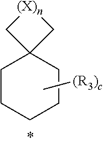

in formula (1) to formula (3) is selected from the structures of formula A-1 to formula A-8:

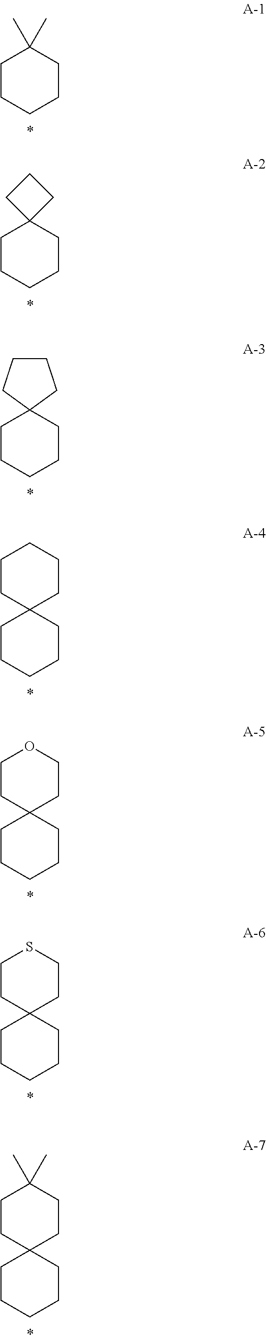

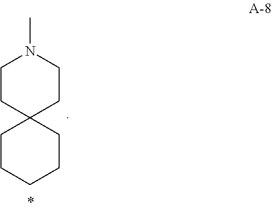

In some embodiments, wherein L is selected from a single bond and the structures of formula B-1 to formula B-18:

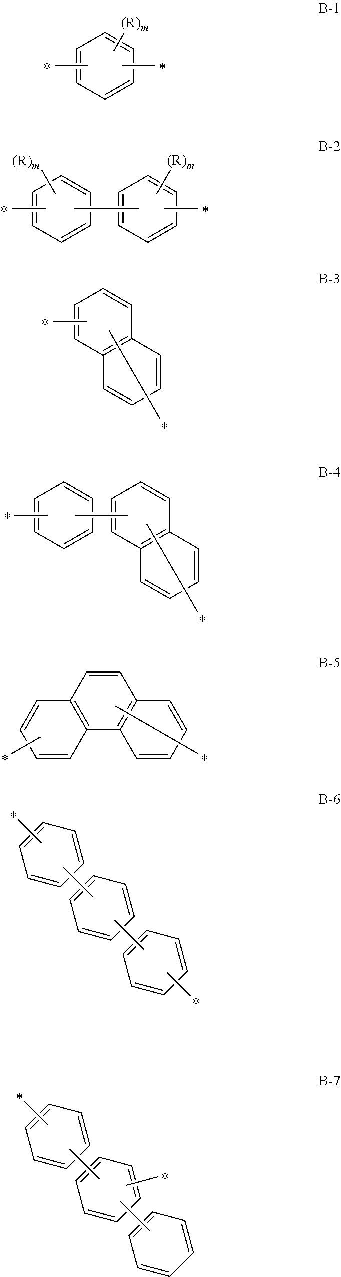

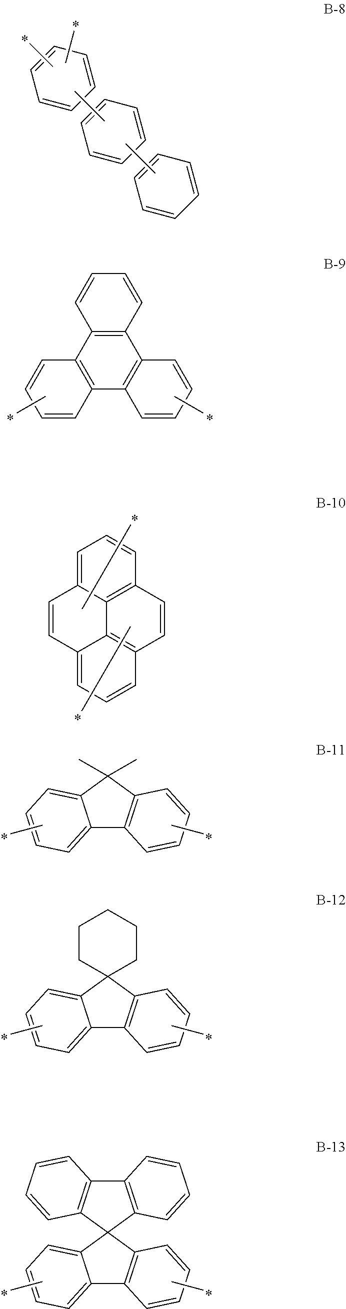

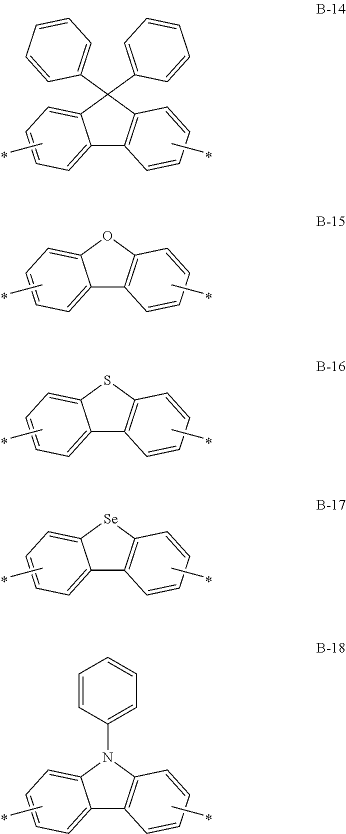

wherein R is a substituent, and each R is independently selected from the group consisting of deuterium, halogen, cyano, nitro, C1-C20 alkyl, C1-C20 heteroalkyl, C2-C20 alkenyl, C2-C20 alkynyl, C3-C20 cycloalkyl, C3-C20 heterocycloalkyl, C6-C30 aryl, C5-C30 heteroaryl, C1-C20 alkoxy, C6-C30 aryloxy, C3-C20 alkylsilyl, C6-C30 arylsilyl, C1-C20 alkylboryl, C6-C30 arylboryl, C6-C30 arylphosphino, C6-C30 monoarylphosphino, C6-C30 diarylphosphino, or C6-C30 arylamino; and
m is selected from an integer of 0 to 4.

In some embodiments, each R is independently selected from deuterium or C1-C4 alkyl.

In some embodiments, at least two of X₁ to X₃ include N;
R₁, R₂ and R₃ are each independently selected from the group consisting of deuterium, halogen, cyano, nitro, C1-C20 alkyl, C1-C20 heteroalkyl, C2-C20 alkenyl, C2-C20 alkynyl, C3-C20 cycloalkyl, C3-C20 heterocycloalkyl, C6-C30 aryl, C5-C30 heteroaryl, C1-C20 alkoxy, C6-C30 aryloxy, C3-C20 alkylsilyl, C6-C30 arylsilyl, C1-C20 alkylboryl, C6-C30 arylboryl, C6-C30 arylphosphino, C6-C30 monoarylphosphino, C6-C30 diarylphosphino, or C6-C30 arylamino;
R₆ is selected from the group consisting of hydrogen, deuterium, halogen, cyano, nitro, C1-C20 alkyl, C1-C20 heteroalkyl, C2-C20 alkenyl, C2-C20 alkynyl, C3-C20 cycloalkyl, C3-C20 heterocycloalkyl, C6-C30 aryl, C5-C30 heteroaryl, C1-C20 alkoxy, C6-C30 aryloxy, C3-C20 alkylsilyl, C6-C30 arylsilyl, C1-C20 alkylboryl, C6-C30 arylboryl, C6-C30 arylphosphino, C6-C30 monoarylphosphino, C6-C30 diarylphosphino, or C6-C30 arylamino.

Two of X₁ to X₃ are N, and the remaining one of X₁ to X₃ is CH or N; and R₁, R₂ and R₃ are each independently selected from the group consisting of deuterium, halogen, cyano, nitro, C1-C10 alkyl, C1-C10 heteroalkyl, C2-C10 alkenyl, C2-C10 alkynyl, C3-C10 cycloalkyl, C3-C10 heterocycloalkyl, C6-C20 aryl, C5-C20 heteroaryl, C1-C10 alkoxy, C6-C20 aryloxy, C3-C10 alkylsilyl, C6-C20 arylsilyl, C1-C10 alkylboryl, C6-C20 arylboryl, C6-C20 arylphosphino, C6-C20 monoarylphosphino, C6-C20 diarylphosphino, or C6-C30 arylamino.

X₁ to X₃ are each N; or X₁ and X₂ are N, and X₃ is CH; and R₁, R₂ and R₃ are each independently selected from the group consisting of deuterium, halogen, cyano, nitro, C1-C10 alkyl, C1-C10 heteroalkyl, C3-C10 cycloalkyl, C3-C10 heterocycloalkyl, C6-C12 aryl, C5-C12 heteroaryl, C1-C10 alkoxy, C6-C12 aryloxy, or C6-C12 arylamino.

In some embodiments, X₁ to X₃ are each N; and a, b, and c are each 0.

In some embodiments, Ar₁ and Ar₂ are each independently selected from the group consisting of phenyl, deuterated phenyl, methylphenyl, fluorophenyl, tert-butylphenyl, trideuterated methylphenyl, biphenyl, naphthyl, deuterated naphthyl, dibenzofuranyl, dibenzothiophenyl, 9,9-dimethylfluorenyl, 9,9-diphenylfluorenyl, spirodifluorenyl, phenanthryl, pyrenyl, chrysenyl, carbazolyl, pyridyl, pyrimidinyl, 4-cyanophenyl, 3-cyanophenyl, and benzophenanthrenyl, or a combination of at least two thereof.

As a compound in some embodiments, the structure of formula (1) contains at least one deuterium atom.

As a compound in some embodiments, the compound is selected from but not limited to the following structures:

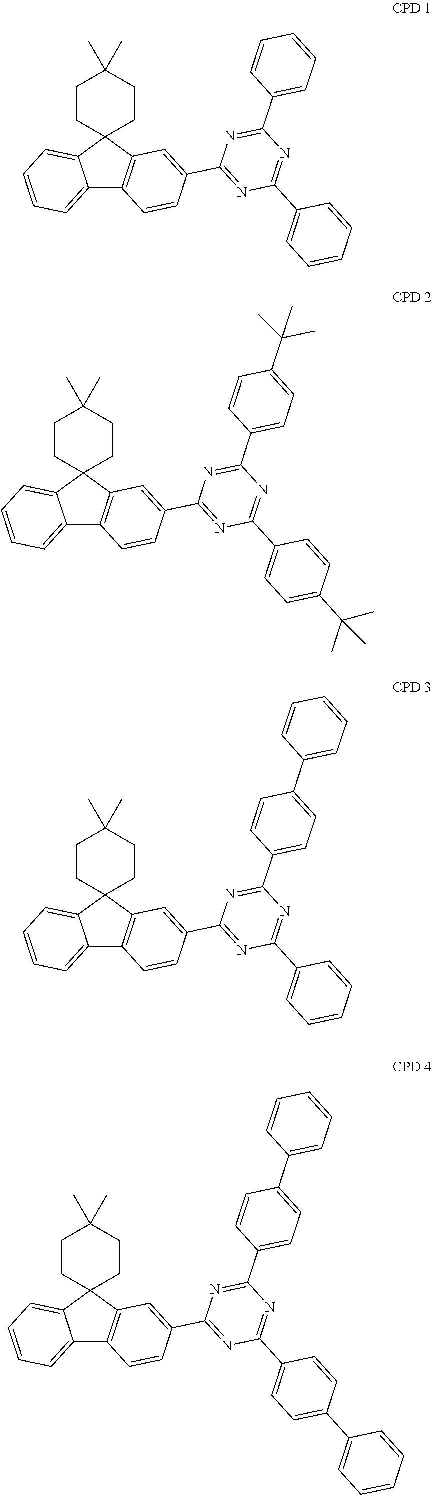

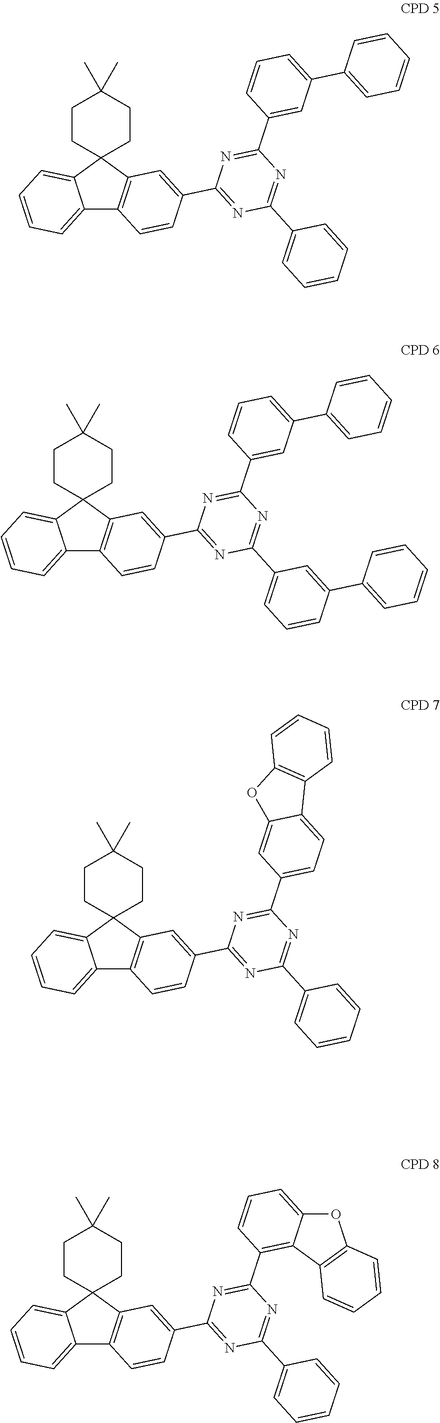 [SMILES: CC1(C)CCC2(CC1)c1ccccc1-c1ccc(-c3nc(-c4ccccc4)nc(-c4cccc(-c5ccccc5)c4)n3)cc12 ; CC1(C)CCC2(CC1)c1ccccc1-c1ccc(-c3nc(-c4cccc(-c5ccccc5)c4)nc(-c4cccc(-c5ccccc5)c4)n3)cc12 ; CC1(C)CCC2(CC1)c1ccccc1-c1ccc(-c3nc(-c4ccccc4)nc(-c4ccc5c(c4)oc4ccccc45)n3)cc12 ; CC1(C)CCC2(CC1)c1ccccc1-c1ccc(-c3nc(-c4ccccc4)nc(-c4cccc5oc6ccccc6c45)n3)cc12]

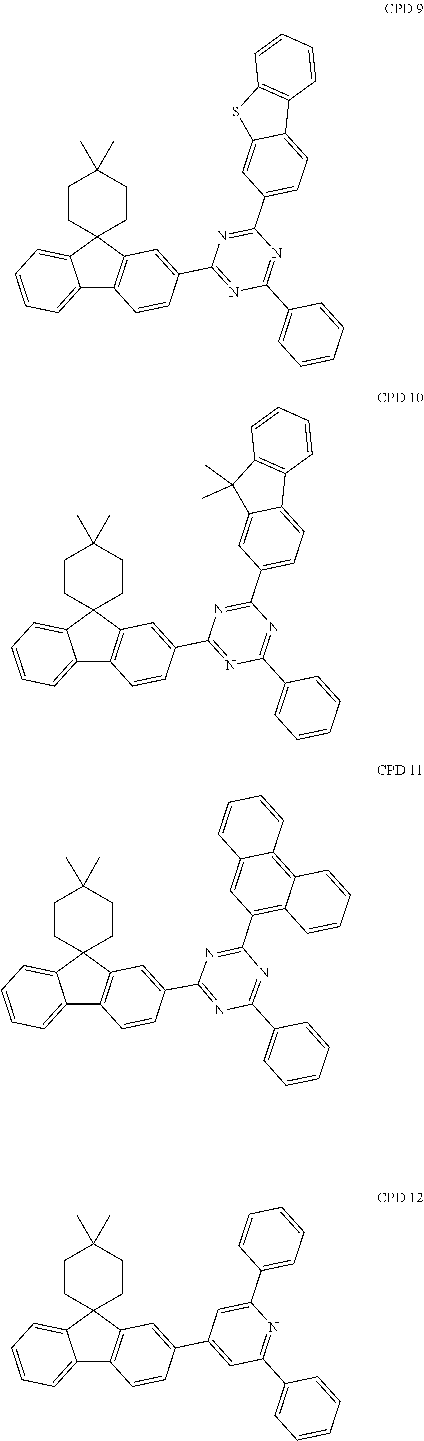 [SMILES: CC1(C)CCC2(CC1)c1ccccc1-c1ccc(-c3nc(-c4ccccc4)nc(-c4ccc5c(c4)sc4ccccc45)n3)cc12 ; CC1(C)CCC2(CC1)c1ccccc1-c1ccc(-c3nc(-c4ccccc4)nc(-c4ccc5c(c4)C(C)(C)c4ccccc4-5)n3)cc12 ; CC1(C)CCC2(CC1)c1ccccc1-c1ccc(-c3nc(-c4ccccc4)nc(-c4cc5ccccc5c5ccccc45)n3)cc12 ; CC1(C)CCC2(CC1)c1ccccc1-c1ccc(-c3cc(-c4ccccc4)nc(-c4ccccc4)c3)cc12]

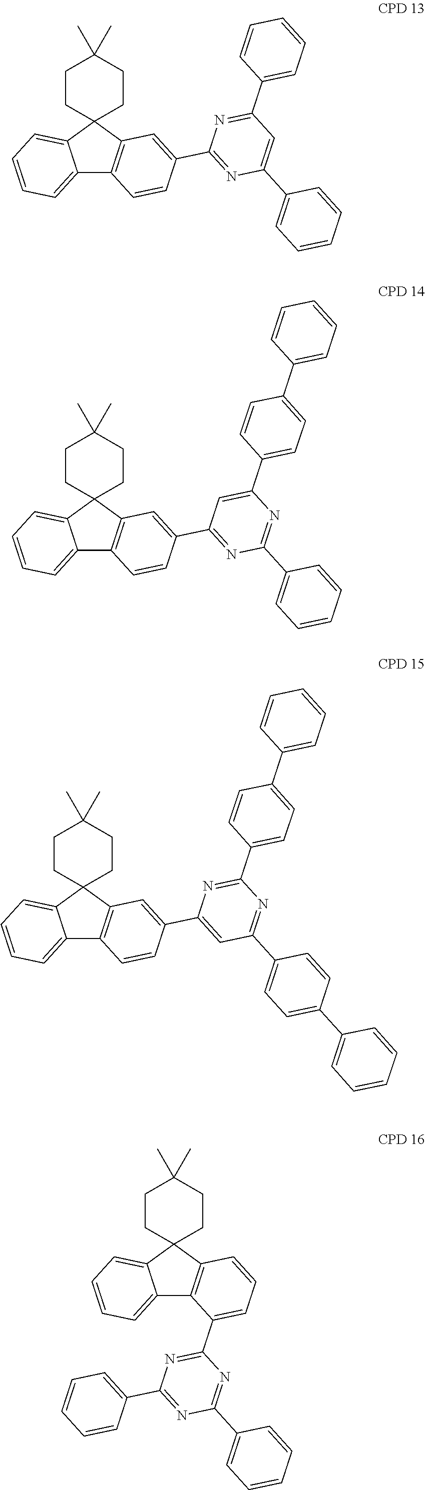 [SMILES: CC1(C)CCC2(CC1)c1ccccc1-c1ccc(-c3nc(-c4ccccc4)cc(-c4ccccc4)n3)cc12 ; CC1(C)CCC2(CC1)c1ccccc1-c1ccc(-c3cc(-c4ccc(-c5ccccc5)cc4)nc(-c4ccccc4)n3)cc12 ; CC1(C)CCC2(CC1)c1ccccc1-c1ccc(-c3cc(-c4ccc(-c5ccccc5)cc4)nc(-c4ccc(-c5ccccc5)cc4)n3)cc12 ; CC1(C)CCC2(CC1)c1ccccc1-c1c(-c3nc(-c4ccccc4)nc(-c4ccccc4)n3)cccc12]

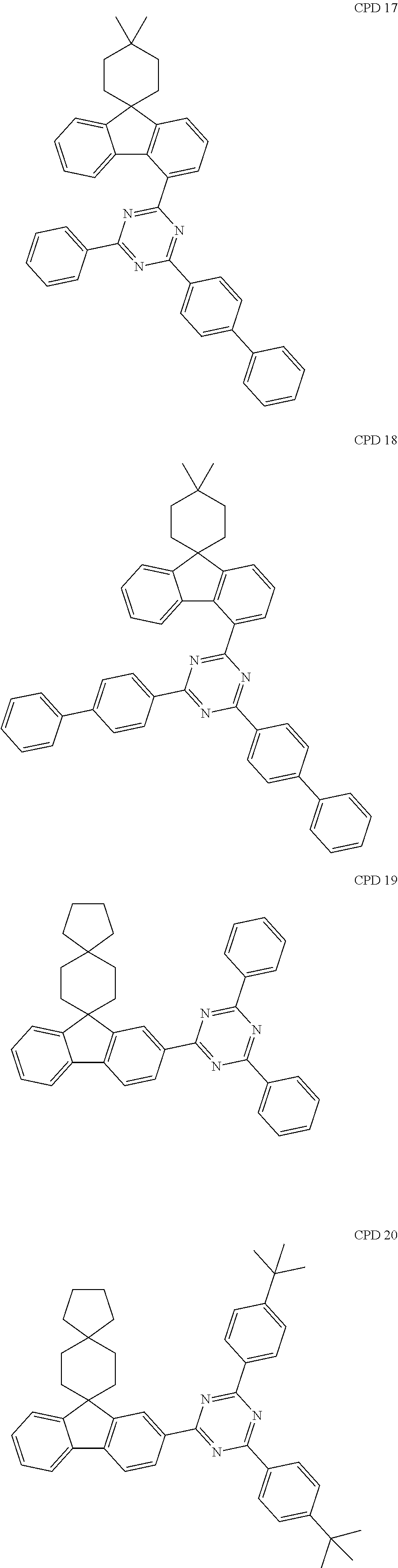 [SMILES: CC1(C)CCC2(CC1)c1ccccc1-c1c(-c3nc(-c4ccccc4)nc(-c4ccc(-c5ccccc5)cc4)n3)cccc12 ; CC1(C)CCC2(CC1)c1ccccc1-c1c(-c3nc(-c4ccc(-c5ccccc5)cc4)nc(-c4ccc(-c5ccccc5)cc4)n3)cccc12 ; c1ccc(-c2nc(-c3ccccc3)nc(-c3ccc4c(c3)C3(CCC5(CCCC5)CC3)c3ccccc3-4)n2)cc1 ; CC(C)(C)c1ccc(-c2nc(-c3ccc(C(C)(C)C)cc3)nc(-c3ccc4c(c3)C3(CCC5(CCCC5)CC3)c3ccccc3-4)n2)cc1]

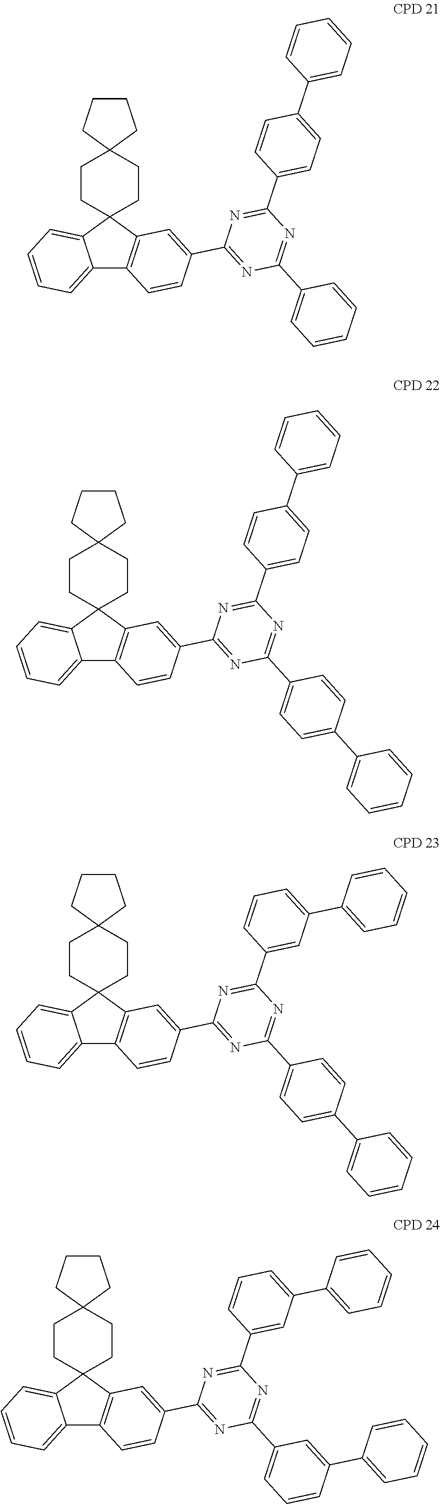 [SMILES: c1ccc(-c2ccc(-c3nc(-c4ccccc4)nc(-c4ccc5c(c4)C4(CCC6(CCCC6)CC4)c4ccccc4-5)n3)cc2)cc1 ; c1ccc(-c2ccc(-c3nc(-c4ccc(-c5ccccc5)cc4)nc(-c4ccc5c(c4)C4(CCC6(CCCC6)CC4)c4ccccc4-5)n3)cc2)cc1 ; c1ccc(-c2ccc(-c3nc(-c4cccc(-c5ccccc5)c4)nc(-c4ccc5c(c4)C4(CCC6(CCCC6)CC4)c4ccccc4-5)n3)cc2)cc1 ; c1ccc(-c2cccc(-c3nc(-c4cccc(-c5ccccc5)c4)nc(-c4ccc5c(c4)C4(CCC6(CCCC6)CC4)c4ccccc4-5)n3)c2)cc1]

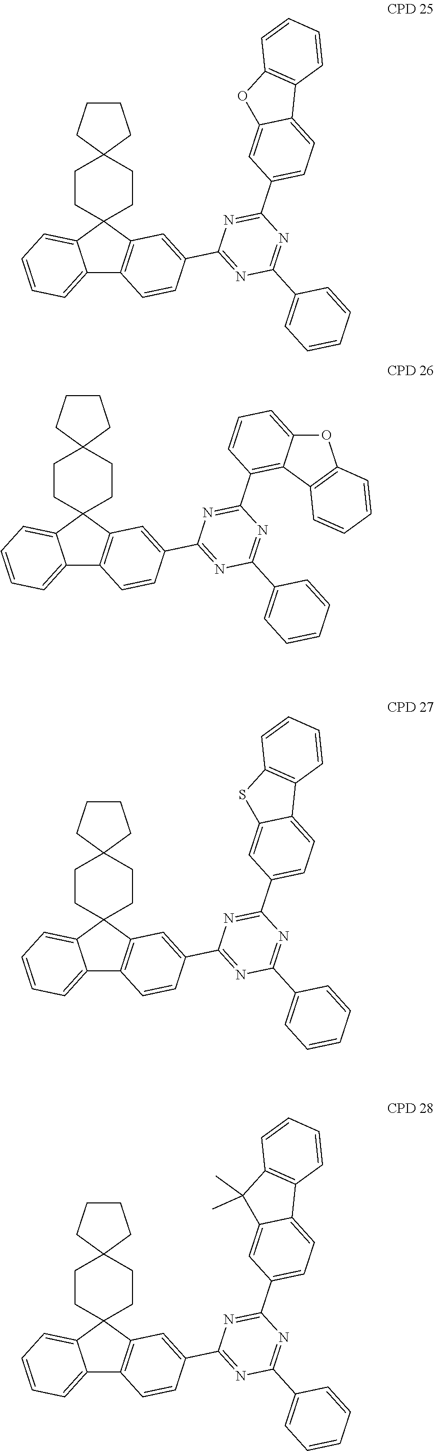 [SMILES: c1ccc(-c2nc(-c3ccc4c(c3)C3(CCC5(CCCC5)CC3)c3ccccc3-4)nc(-c3ccc4c(c3)oc3ccccc34)n2)cc1 ; c1ccc(-c2nc(-c3ccc4c(c3)C3(CCC5(CCCC5)CC3)c3ccccc3-4)nc(-c3cccc4oc5ccccc5c34)n2)cc1 ; c1ccc(-c2nc(-c3ccc4c(c3)C3(CCC5(CCCC5)CC3)c3ccccc3-4)nc(-c3ccc4c(c3)sc3ccccc34)n2)cc1 ; CC1(C)c2ccccc2-c2ccc(-c3nc(-c4ccccc4)nc(-c4ccc5c(c4)C4(CCC6(CCCC6)CC4)c4ccccc4-5)n3)cc21]

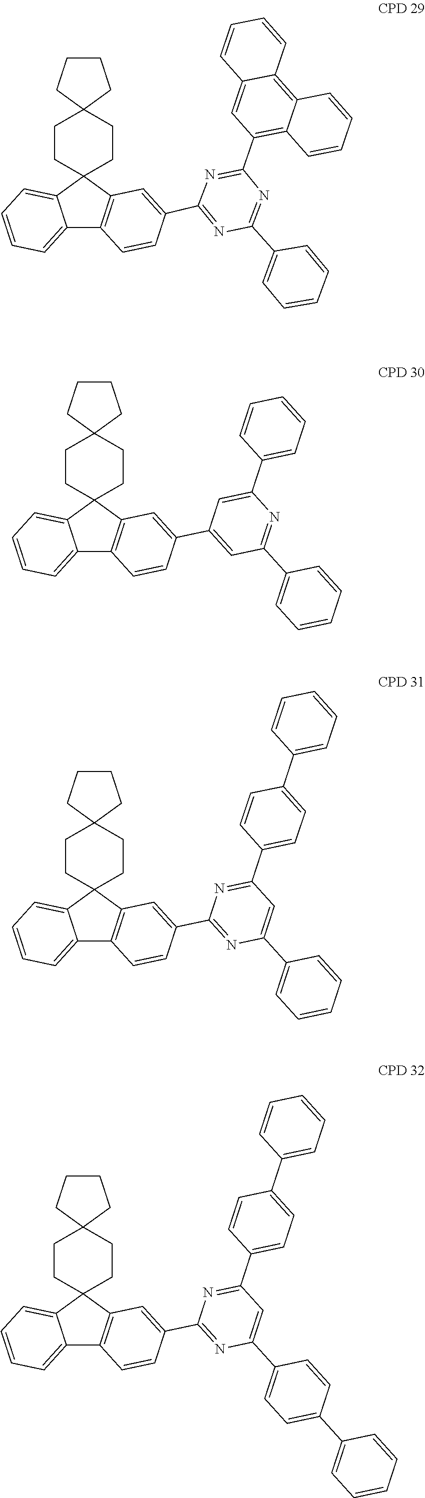 [SMILES: c1ccc(-c2nc(-c3ccc4c(c3)C3(CCC5(CCCC5)CC3)c3ccccc3-4)nc(-c3cc4ccccc4c4ccccc34)n2)cc1 ; c1ccc(-c2cc(-c3ccc4c(c3)C3(CCC5(CCCC5)CC3)c3ccccc3-4)cc(-c3ccccc3)n2)cc1 ; c1ccc(-c2ccc(-c3cc(-c4ccccc4)nc(-c4ccc5c(c4)C4(CCC6(CCCC6)CC4)c4ccccc4-5)n3)cc2)cc1 ; c1ccc(-c2ccc(-c3cc(-c4ccc(-c5ccccc5)cc4)nc(-c4ccc5c(c4)C4(CCC6(CCCC6)CC4)c4ccccc4-5)n3)cc2)cc1]

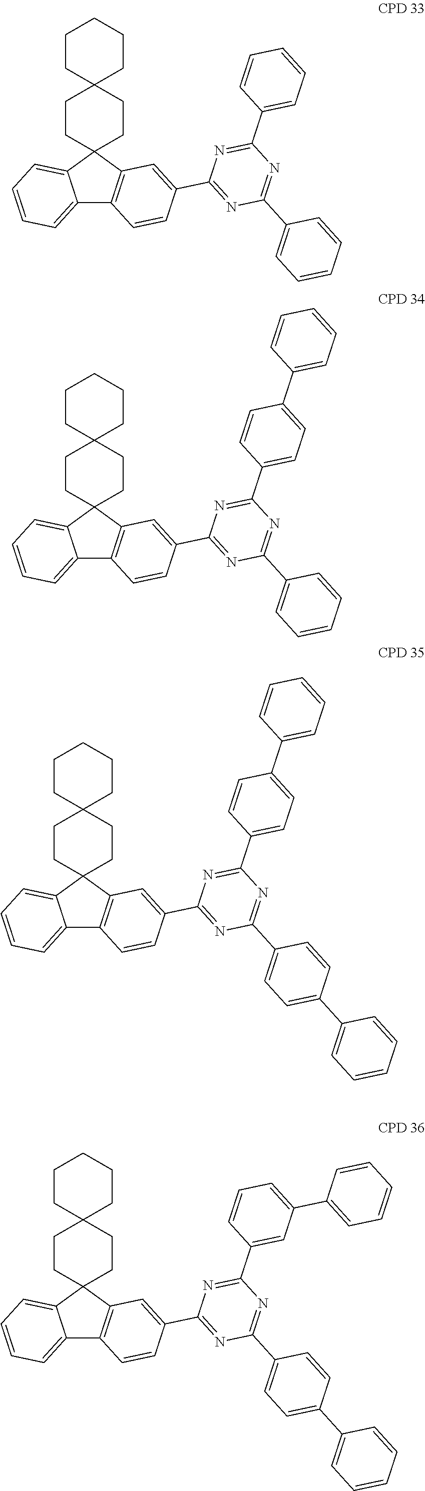

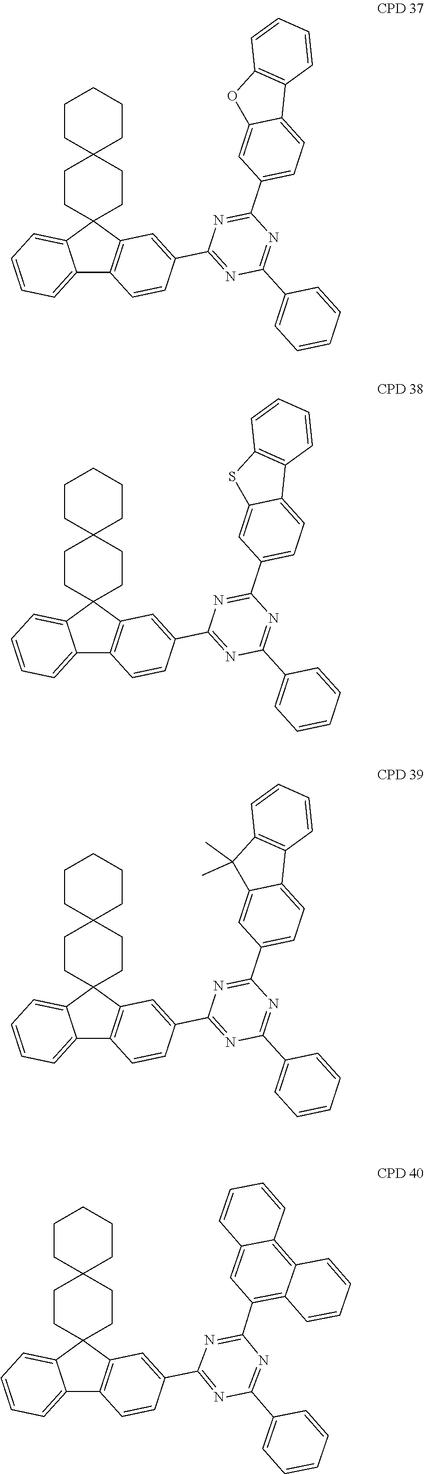 [SMILES: c1ccc(-c2nc(-c3ccc4c(c3)C3(CCC5(CCCCC5)CC3)c3ccccc3-4)nc(-c3ccc4c(c3)oc3ccccc34)n2)cc1 ; c1ccc(-c2nc(-c3ccc4c(c3)C3(CCC5(CCCCC5)CC3)c3ccccc3-4)nc(-c3ccc4c(c3)sc3ccccc34)n2)cc1 ; CC1(C)c2ccccc2-c2ccc(-c3nc(-c4ccccc4)nc(-c4ccc5c(c4)C4(CCC6(CCCCC6)CC4)c4ccccc4-5)n3)cc21 ; c1ccc(-c2nc(-c3ccc4c(c3)C3(CCC5(CCCCC5)CC3)c3ccccc3-4)nc(-c3cc4ccccc4c4ccccc34)n2)cc1]

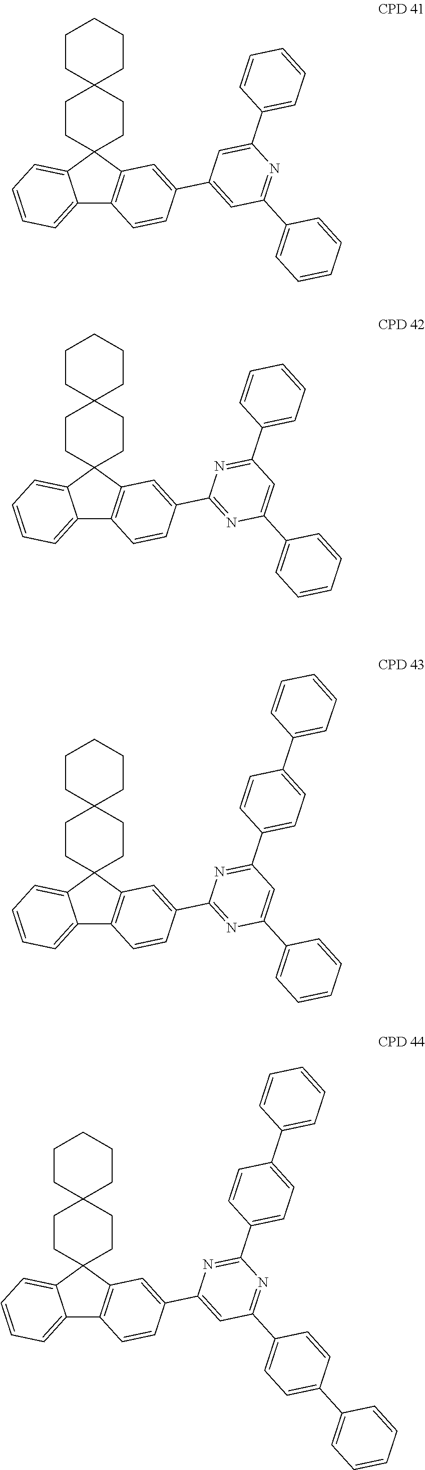 [SMILES: c1ccc(-c2cc(-c3ccc4c(c3)C3(CCC5(CCCCC5)CC3)c3ccccc3-4)cc(-c3ccccc3)n2)cc1 ; c1ccc(-c2cc(-c3ccccc3)nc(-c3ccc4c(c3)C3(CCC5(CCCCC5)CC3)c3ccccc3-4)n2)cc1 ; c1ccc(-c2ccc(-c3cc(-c4ccccc4)nc(-c4ccc5c(c4)C4(CCC6(CCCCC6)CC4)c4ccccc4-5)n3)cc2)cc1 ; c1ccc(-c2ccc(-c3cc(-c4ccc5c(c4)C4(CCC6(CCCCC6)CC4)c4ccccc4-5)nc(-c4ccc(-c5ccccc5)cc4)n3)cc2)cc1]

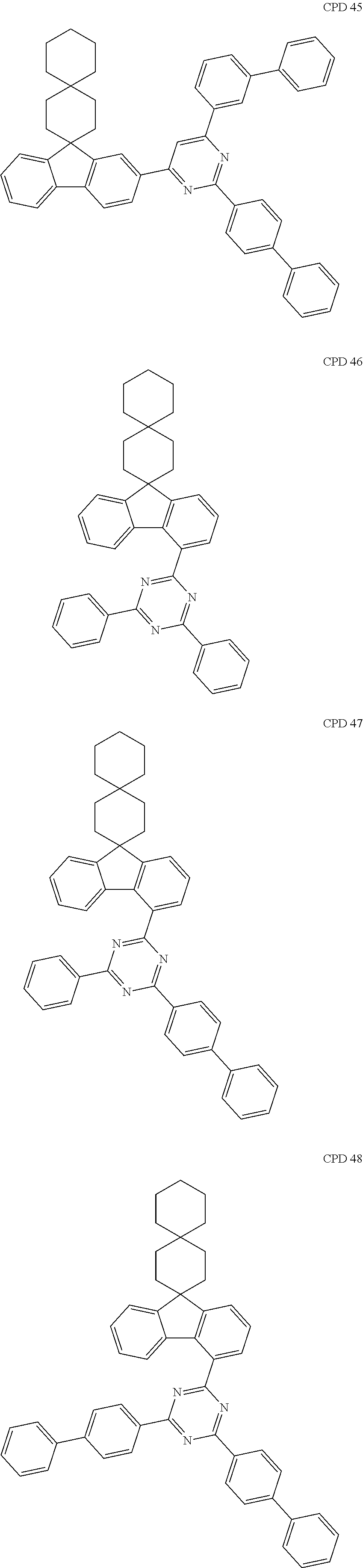 [SMILES: c1ccc(-c2ccc(-c3nc(-c4cccc(-c5ccccc5)c4)cc(-c4ccc5c(c4)C4(CCC6(CCCCC6)CC4)c4ccccc4-5)n3)cc2)cc1 ; c1ccc(-c2nc(-c3ccccc3)nc(-c3cccc4c3-c3ccccc3C43CCC4(CCCCC4)CC3)n2)cc1 ; c1ccc(-c2ccc(-c3nc(-c4ccccc4)nc(-c4cccc5c4-c4ccccc4C54CCC5(CCCCC5)CC4)n3)cc2)cc1 ; c1ccc(-c2ccc(-c3nc(-c4ccc(-c5ccccc5)cc4)nc(-c4cccc5c4-c4ccccc4C54CCC5(CCCCC5)CC4)n3)cc2)cc1]

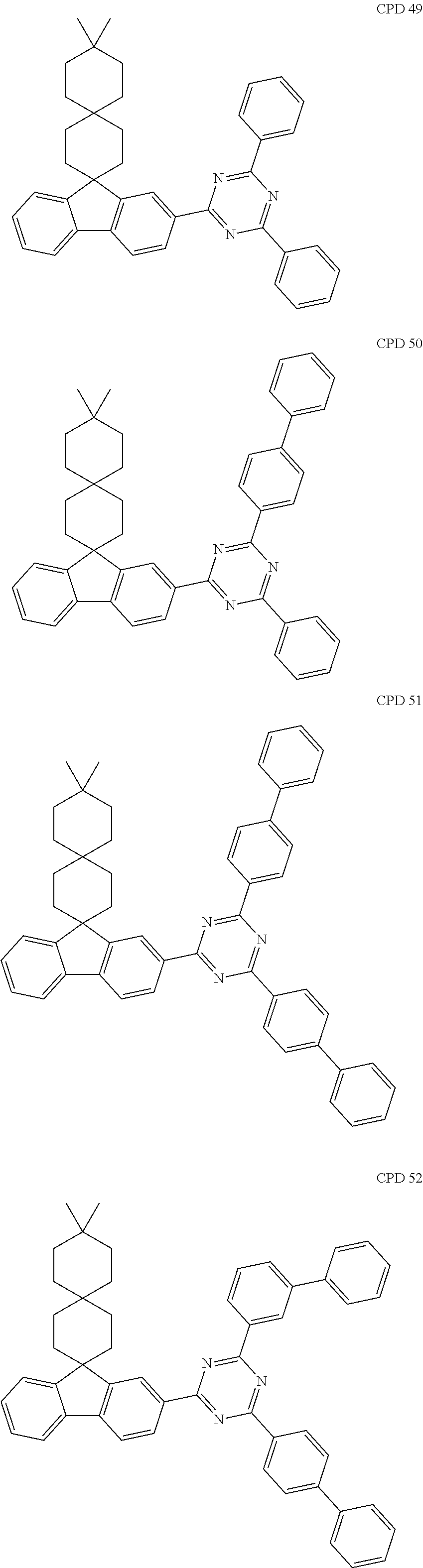

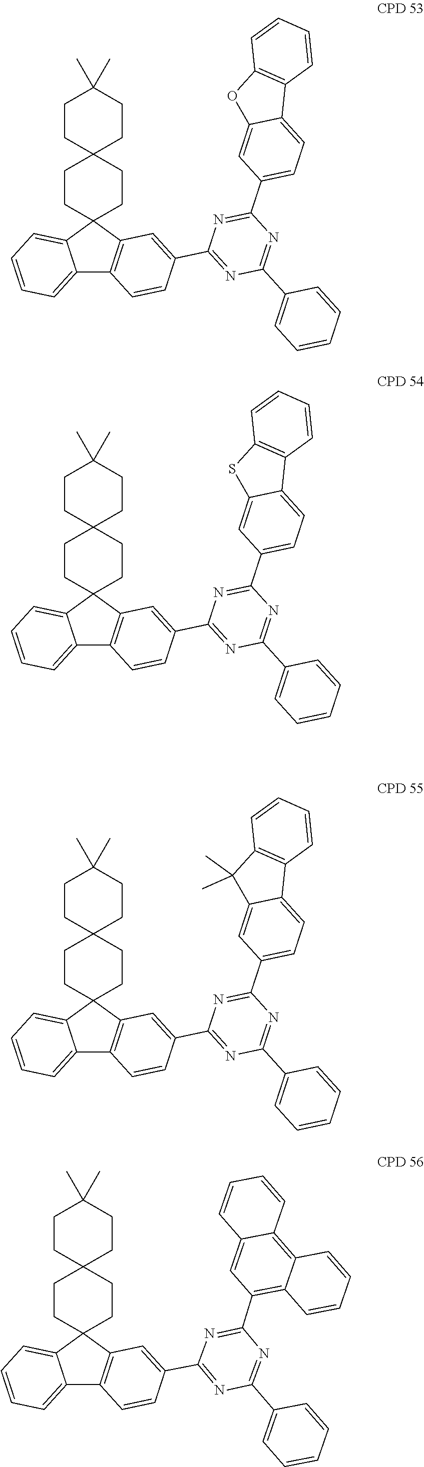

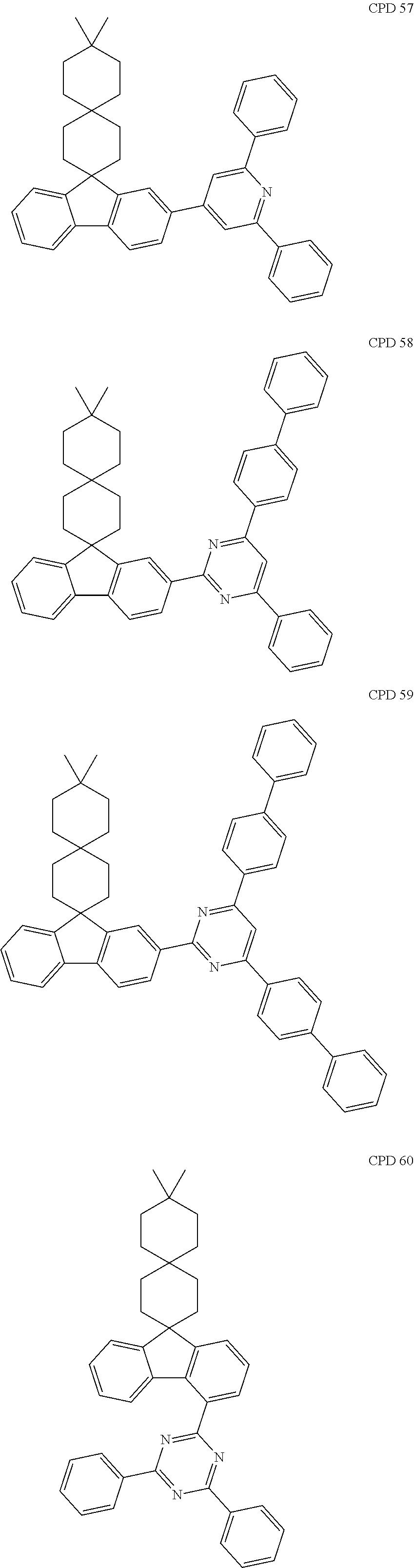 [SMILES: CC1(C)CCC2(CC1)CCC1(CC2)c2ccccc2-c2ccc(-c3cc(-c4ccccc4)nc(-c4ccccc4)c3)cc21 ; CC1(C)CCC2(CC1)CCC1(CC2)c2ccccc2-c2ccc(-c3nc(-c4ccccc4)cc(-c4ccc(-c5ccccc5)cc4)n3)cc21 ; CC1(C)CCC2(CC1)CCC1(CC2)c2ccccc2-c2ccc(-c3nc(-c4ccc(-c5ccccc5)cc4)cc(-c4ccc(-c5ccccc5)cc4)n3)cc21 ; CC1(C)CCC2(CC1)CCC1(CC2)c2ccccc2-c2c(-c3nc(-c4ccccc4)nc(-c4ccccc4)n3)cccc21]

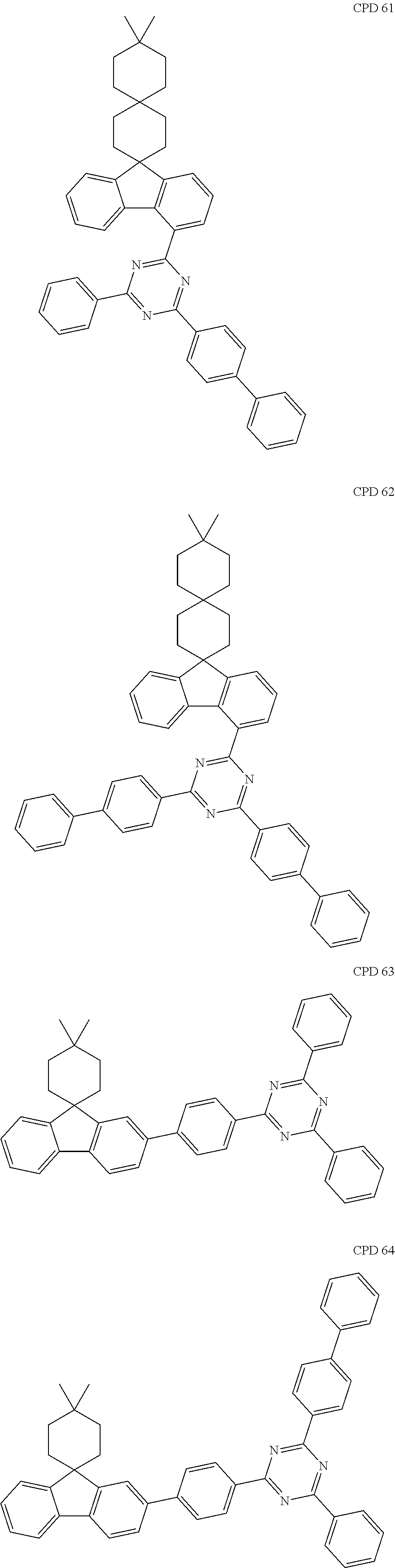 [SMILES: CC1(C)CCC2(CC1)CCC1(CC2)c2ccccc2-c2c(-c3nc(-c4ccccc4)nc(-c4ccc(-c5ccccc5)cc4)n3)cccc21 ; CC1(C)CCC2(CC1)CCC1(CC2)c2ccccc2-c2c(-c3nc(-c4ccc(-c5ccccc5)cc4)nc(-c4ccc(-c5ccccc5)cc4)n3)cccc21 ; CC1(C)CCC2(CC1)c1ccccc1-c1ccc(-c3ccc(-c4nc(-c5ccccc5)nc(-c5ccccc5)n4)cc3)cc12 ; CC1(C)CCC2(CC1)c1ccccc1-c1ccc(-c3ccc(-c4nc(-c5ccccc5)nc(-c5ccc(-c6ccccc6)cc5)n4)cc3)cc12]

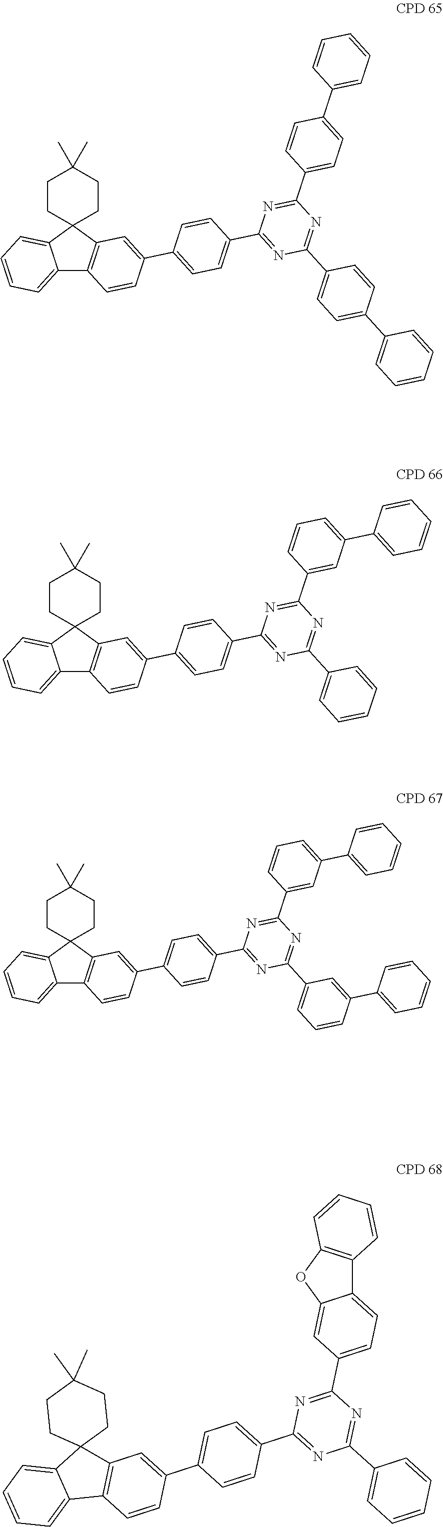 [SMILES: CC1(C)CCC2(CC1)c1ccccc1-c1ccc(-c3ccc(-c4nc(-c5ccc(-c6ccccc6)cc5)nc(-c5ccc(-c6ccccc6)cc5)n4)cc3)cc12 ; CC1(C)CCC2(CC1)c1ccccc1-c1ccc(-c3ccc(-c4nc(-c5ccccc5)nc(-c5cccc(-c6ccccc6)c5)n4)cc3)cc12 ; CC1(C)CCC2(CC1)c1ccccc1-c1ccc(-c3ccc(-c4nc(-c5cccc(-c6ccccc6)c5)nc(-c5cccc(-c6ccccc6)c5)n4)cc3)cc12 ; CC1(C)CCC2(CC1)c1ccccc1-c1ccc(-c3ccc(-c4nc(-c5ccccc5)nc(-c5ccc6c(c5)oc5ccccc56)n4)cc3)cc12]

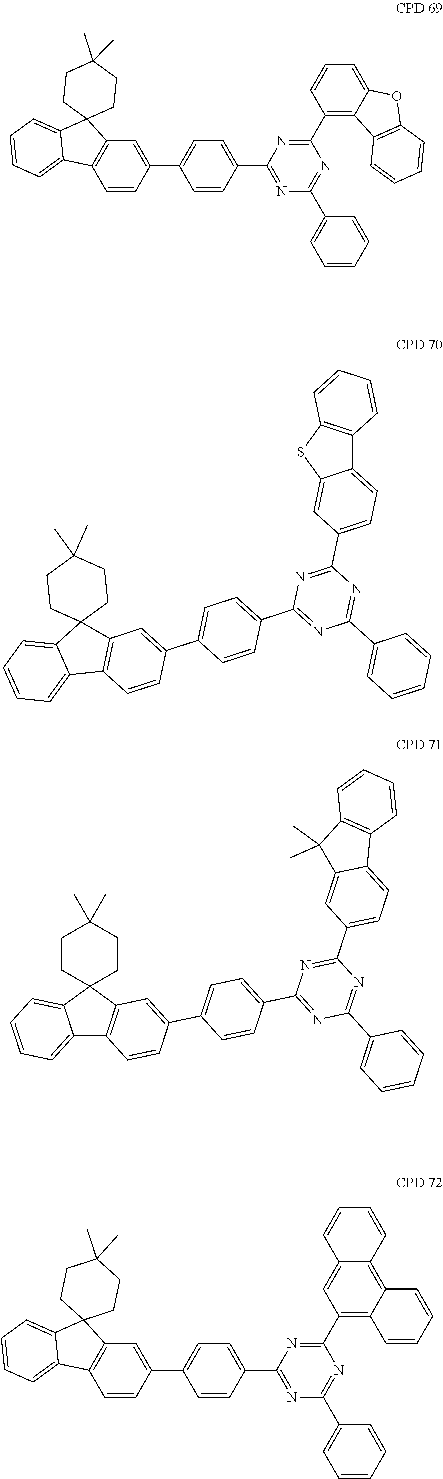 [SMILES: CC1(C)CCC2(CC1)c1ccccc1-c1ccc(-c3ccc(-c4nc(-c5ccccc5)nc(-c5cccc6oc7ccccc7c56)n4)cc3)cc12 ; CC1(C)CCC2(CC1)c1ccccc1-c1ccc(-c3ccc(-c4nc(-c5ccccc5)nc(-c5ccc6c(c5)sc5ccccc56)n4)cc3)cc12 ; CC1(C)CCC2(CC1)c1ccccc1-c1ccc(-c3ccc(-c4nc(-c5ccccc5)nc(-c5ccc6c(c5)C(C)(C)c5ccccc5-6)n4)cc3)cc12 ; CC1(C)CCC2(CC1)c1ccccc1-c1ccc(-c3ccc(-c4nc(-c5ccccc5)nc(-c5cc6ccccc6c6ccccc56)n4)cc3)cc12]

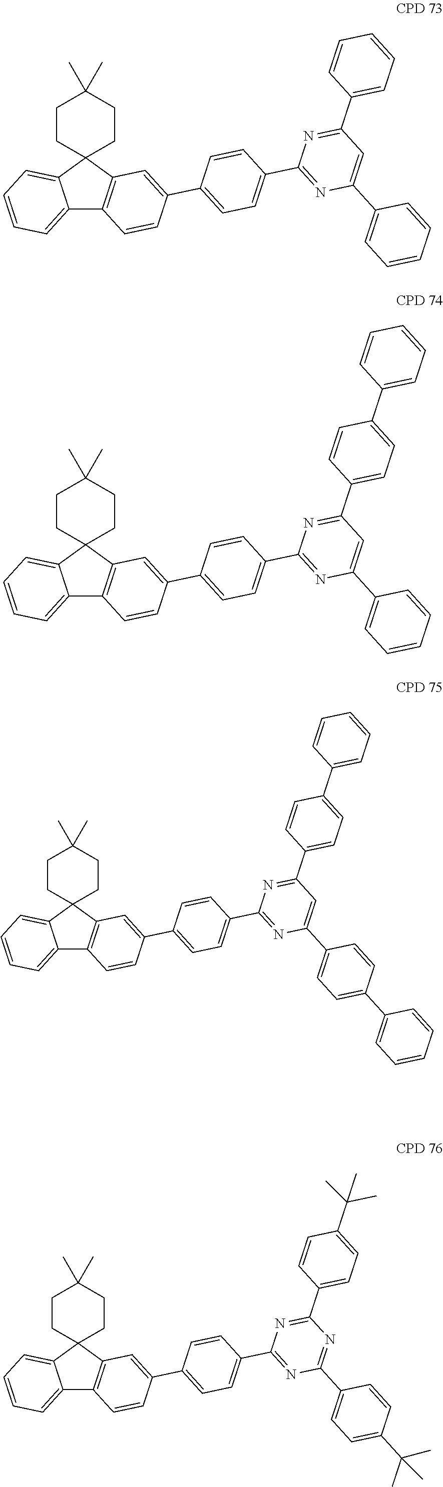 [SMILES: CC1(C)CCC2(CC1)c1ccccc1-c1ccc(-c3ccc(-c4nc(-c5ccccc5)cc(-c5ccccc5)n4)cc3)cc12 ; CC1(C)CCC2(CC1)c1ccccc1-c1ccc(-c3ccc(-c4nc(-c5ccccc5)cc(-c5ccc(-c6ccccc6)cc5)n4)cc3)cc12 ; CC1(C)CCC2(CC1)c1ccccc1-c1ccc(-c3ccc(-c4nc(-c5ccc(-c6ccccc6)cc5)cc(-c5ccc(-c6ccccc6)cc5)n4)cc3)cc12 ; CC1(C)CCC2(CC1)c1ccccc1-c1ccc(-c3ccc(-c4nc(-c5ccc(C(C)(C)C)cc5)nc(-c5ccc(C(C)(C)C)cc5)n4)cc3)cc12]

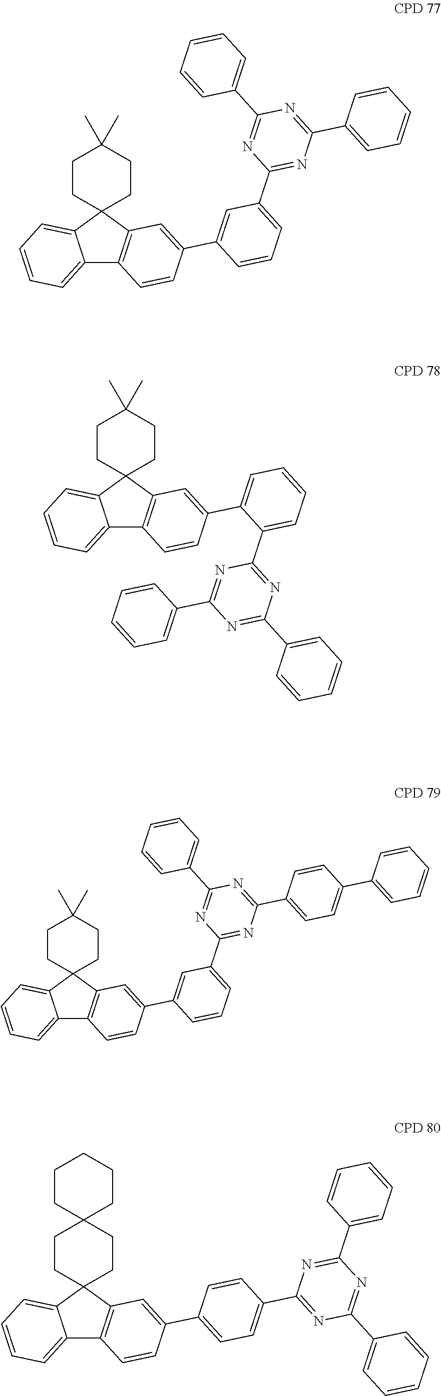 [SMILES: CC1(C)CCC2(CC1)c1ccccc1-c1ccc(-c3cccc(-c4nc(-c5ccccc5)nc(-c5ccccc5)n4)c3)cc12 ; CC1(C)CCC2(CC1)c1ccccc1-c1ccc(-c3ccccc3-c3nc(-c4ccccc4)nc(-c4ccccc4)n3)cc12 ; CC1(C)CCC2(CC1)c1ccccc1-c1ccc(-c3cccc(-c4nc(-c5ccccc5)nc(-c5ccc(-c6ccccc6)cc5)n4)c3)cc12 ; c1ccc(-c2nc(-c3ccccc3)nc(-c3ccc(-c4ccc5c(c4)C4(CCC6(CCCCC6)CC4)c4ccccc4-5)cc3)n2)cc1]

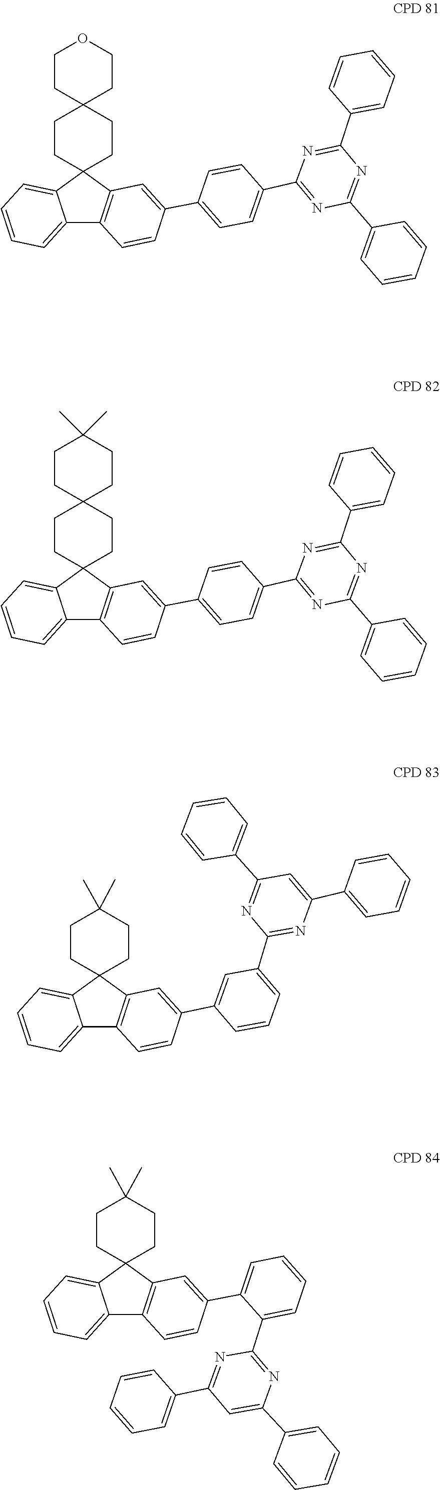 [SMILES: c1ccc(-c2nc(-c3ccccc3)nc(-c3ccc(-c4ccc5c(c4)C4(CCC6(CCOCC6)CC4)c4ccccc4-5)cc3)n2)cc1 ; CC1(C)CCC2(CC1)CCC1(CC2)c2ccccc2-c2ccc(-c3ccc(-c4nc(-c5ccccc5)nc(-c5ccccc5)n4)cc3)cc21 ; CC1(C)CCC2(CC1)c1ccccc1-c1ccc(-c3cccc(-c4nc(-c5ccccc5)cc(-c5ccccc5)n4)c3)cc12 ; CC1(C)CCC2(CC1)c1ccccc1-c1ccc(-c3ccccc3-c3nc(-c4ccccc4)cc(-c4ccccc4)n3)cc12]

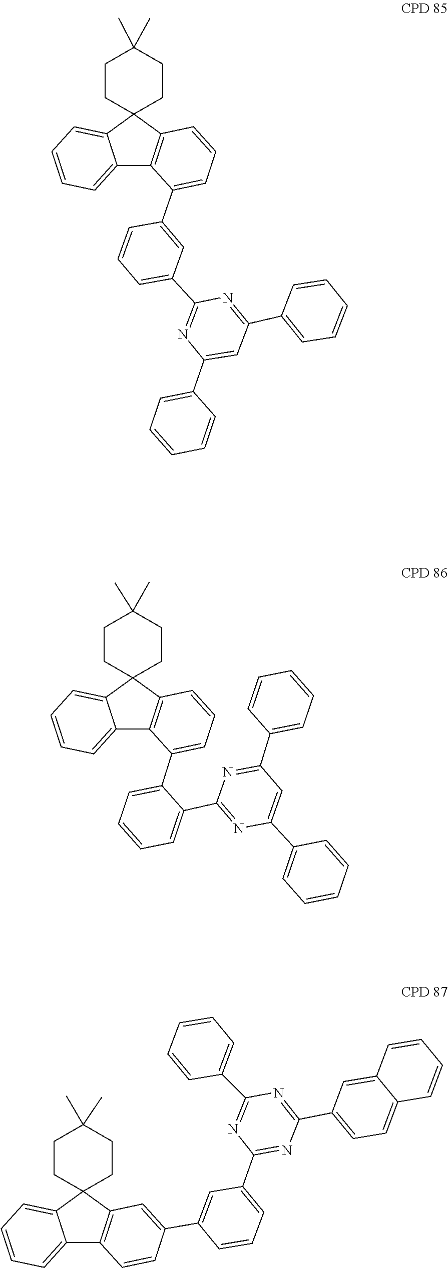 [SMILES: CC1(C)CCC2(CC1)c1ccccc1-c1c(-c3cccc(-c4nc(-c5ccccc5)cc(-c5ccccc5)n4)c3)cccc12 ; CC1(C)CCC2(CC1)c1ccccc1-c1c(-c3ccccc3-c3nc(-c4ccccc4)cc(-c4ccccc4)n3)cccc12 ; CC1(C)CCC2(CC1)c1ccccc1-c1ccc(-c3cccc(-c4nc(-c5ccccc5)nc(-c5ccc6ccccc6c5)n4)c3)cc12]

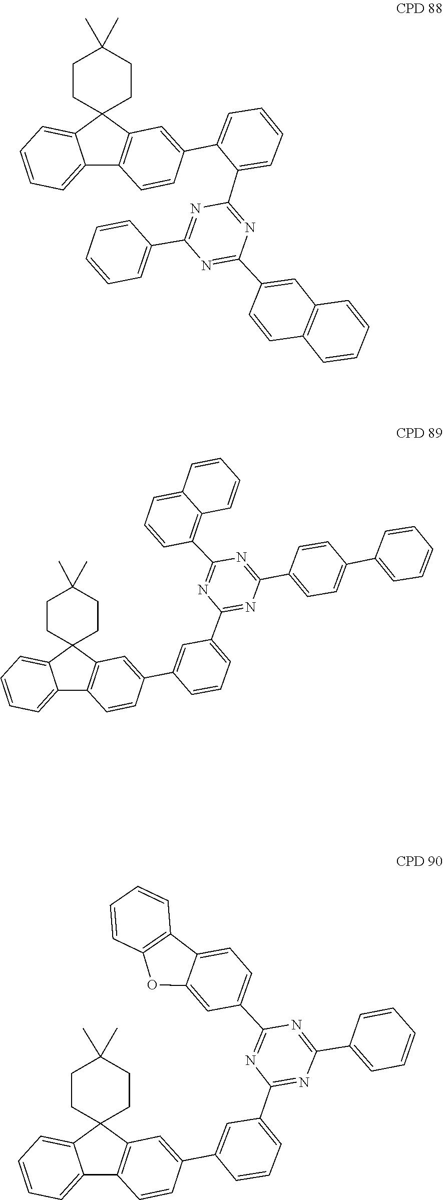 [SMILES: CC1(C)CCC2(CC1)c1ccccc1-c1ccc(-c3ccccc3-c3nc(-c4ccccc4)nc(-c4ccc5ccccc5c4)n3)cc12 ; CC1(C)CCC2(CC1)c1ccccc1-c1ccc(-c3cccc(-c4nc(-c5ccc(-c6ccccc6)cc5)nc(-c5cccc6ccccc56)n4)c3)cc12 ; CC1(C)CCC2(CC1)c1ccccc1-c1ccc(-c3cccc(-c4nc(-c5ccccc5)nc(-c5ccc6c(c5)oc5ccccc56)n4)c3)cc12]

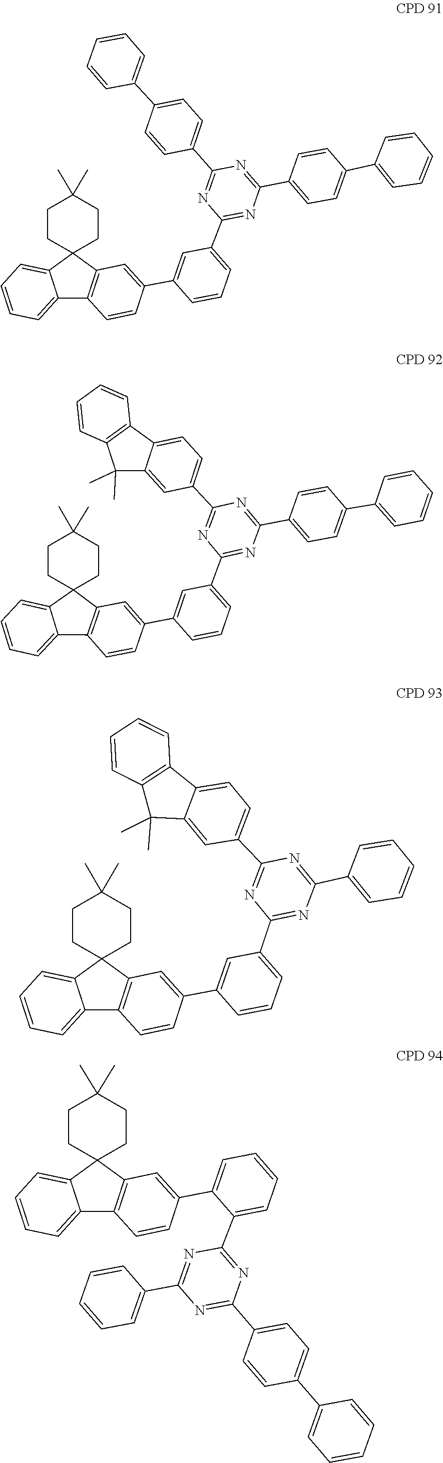

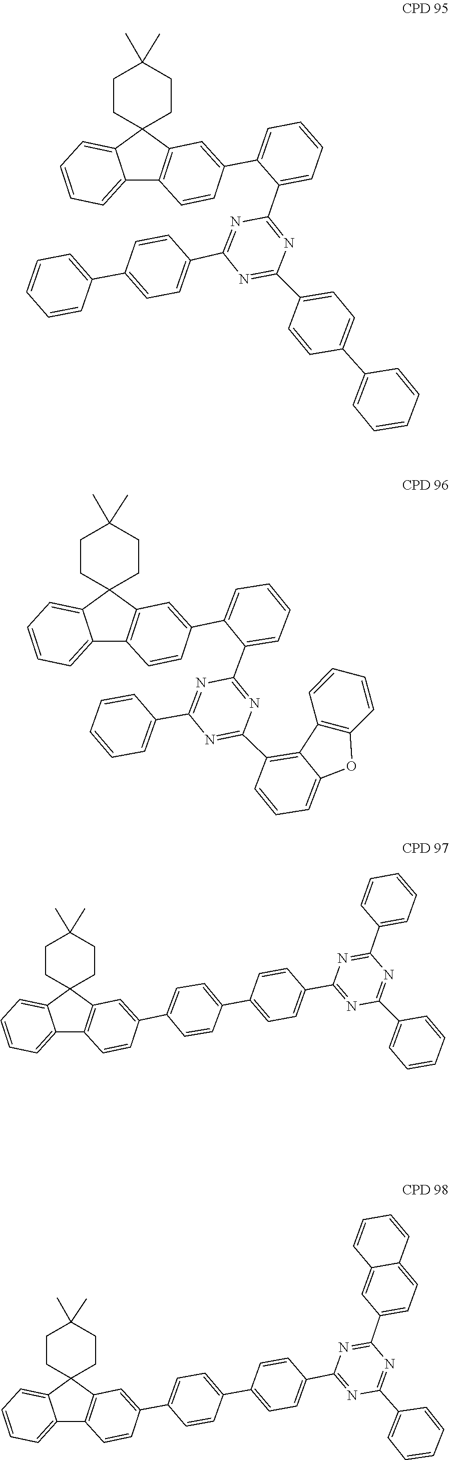 [SMILES: CC1(C)CCC2(CC1)c1ccccc1-c1ccc(-c3ccccc3-c3nc(-c4ccc(-c5ccccc5)cc4)nc(-c4ccc(-c5ccccc5)cc4)n3)cc12 ; CC1(C)CCC2(CC1)c1ccccc1-c1ccc(-c3ccccc3-c3nc(-c4ccccc4)nc(-c4cccc5oc6ccccc6c45)n3)cc12 ; CC1(C)CCC2(CC1)c1ccccc1-c1ccc(-c3ccc(-c4ccc(-c5nc(-c6ccccc6)nc(-c6ccccc6)n5)cc4)cc3)cc12 ; CC1(C)CCC2(CC1)c1ccccc1-c1ccc(-c3ccc(-c4ccc(-c5nc(-c6ccccc6)nc(-c6ccc7ccccc7c6)n5)cc4)cc3)cc12]

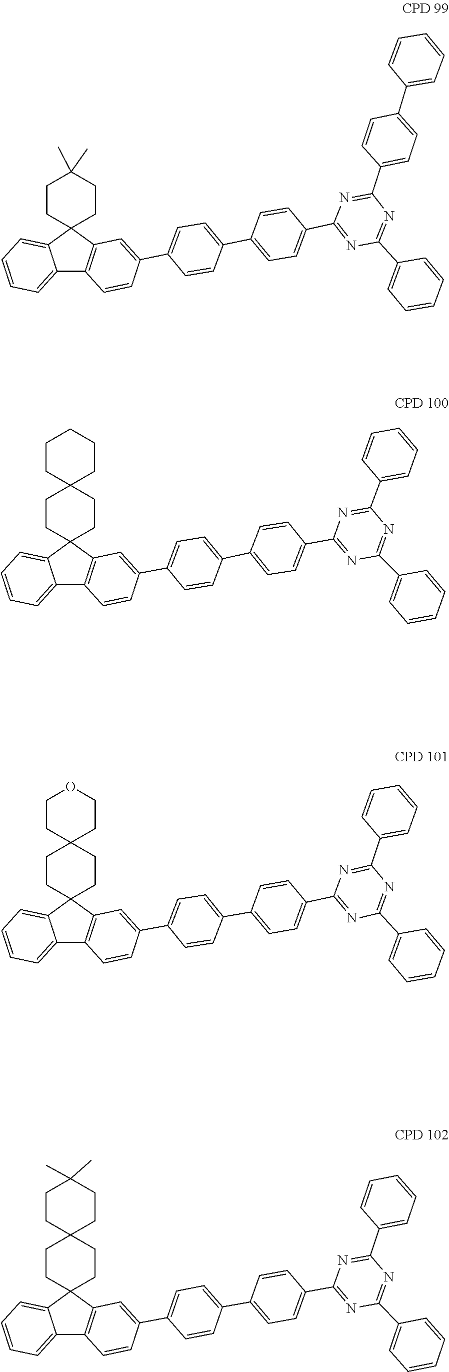 [SMILES: CC1(C)CCC2(CC1)c1ccccc1-c1ccc(-c3ccc(-c4ccc(-c5nc(-c6ccccc6)nc(-c6ccc(-c7ccccc7)cc6)n5)cc4)cc3)cc12 ; c1ccc(-c2nc(-c3ccccc3)nc(-c3ccc(-c4ccc(-c5ccc6c(c5)C5(CCC7(CCCCC7)CC5)c5ccccc5-6)cc4)cc3)n2)cc1 ; c1ccc(-c2nc(-c3ccccc3)nc(-c3ccc(-c4ccc(-c5ccc6c(c5)C5(CCC7(CCOCC7)CC5)c5ccccc5-6)cc4)cc3)n2)cc1 ; CC1(C)CCC2(CC1)CCC1(CC2)c2ccccc2-c2ccc(-c3ccc(-c4ccc(-c5nc(-c6ccccc6)nc(-c6ccccc6)n5)cc4)cc3)cc21]

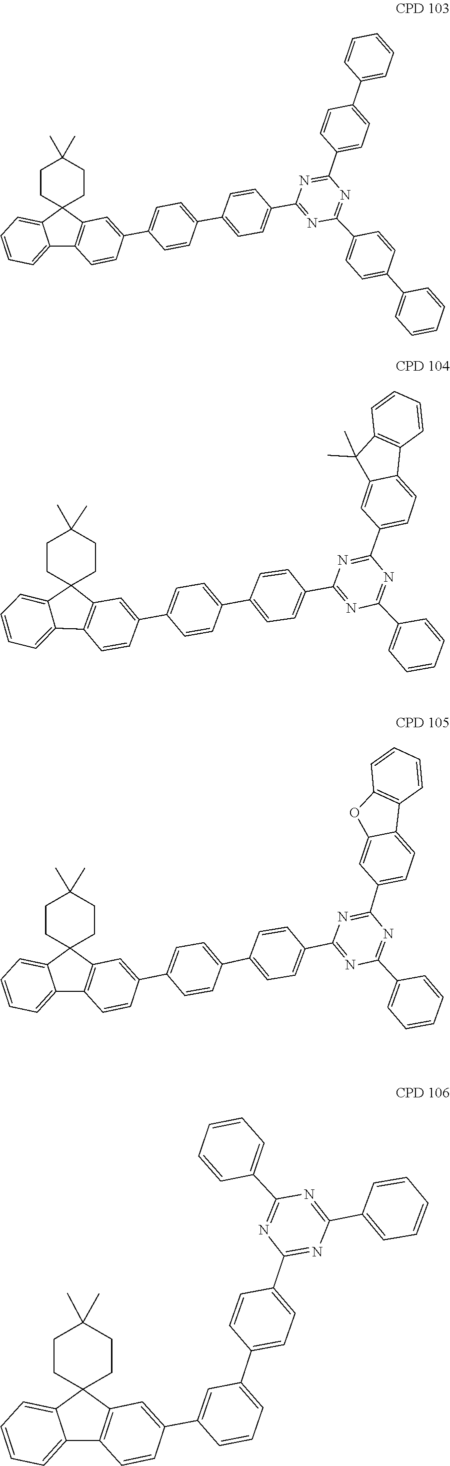 [SMILES: CC1(C)CCC2(CC1)c1ccccc1-c1ccc(-c3ccc(-c4ccc(-c5nc(-c6ccc(-c7ccccc7)cc6)nc(-c6ccc(-c7ccccc7)cc6)n5)cc4)cc3)cc12 ; CC1(C)CCC2(CC1)c1ccccc1-c1ccc(-c3ccc(-c4ccc(-c5nc(-c6ccccc6)nc(-c6ccc7c(c6)C(C)(C)c6ccccc6-7)n5)cc4)cc3)cc12 ; CC1(C)CCC2(CC1)c1ccccc1-c1ccc(-c3ccc(-c4ccc(-c5nc(-c6ccccc6)nc(-c6ccc7c(c6)oc6ccccc67)n5)cc4)cc3)cc12 ; CC1(C)CCC2(CC1)c1ccccc1-c1ccc(-c3cccc(-c4ccc(-c5nc(-c6ccccc6)nc(-c6ccccc6)n5)cc4)c3)cc12]

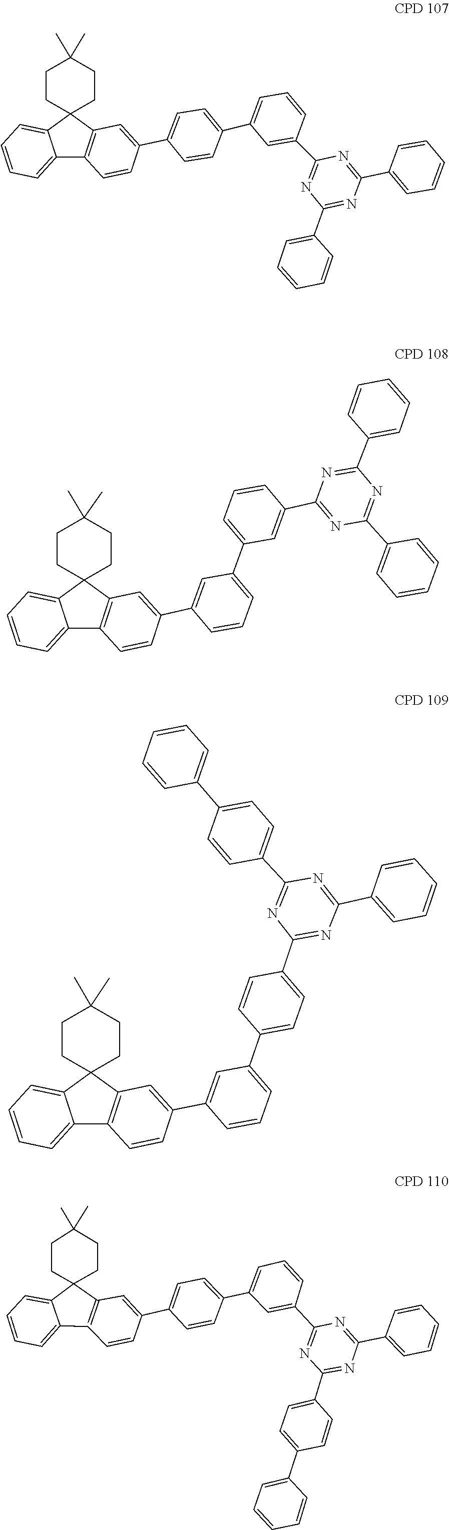 [SMILES: CC1(C)CCC2(CC1)c1ccccc1-c1ccc(-c3ccc(-c4cccc(-c5nc(-c6ccccc6)nc(-c6ccccc6)n5)c4)cc3)cc12 ; CC1(C)CCC2(CC1)c1ccccc1-c1ccc(-c3cccc(-c4cccc(-c5nc(-c6ccccc6)nc(-c6ccccc6)n5)c4)c3)cc12 ; CC1(C)CCC2(CC1)c1ccccc1-c1ccc(-c3cccc(-c4ccc(-c5nc(-c6ccccc6)nc(-c6ccc(-c7ccccc7)cc6)n5)cc4)c3)cc12 ; C=C1/C=C(c2ccc(-c3cccc(-c4nc(-c5ccccc5)nc(-c5ccc(-c6ccccc6)cc5)n4)c3)cc2)\C=C/Cc2ccccc2C12CCC(C)(C)CC2]

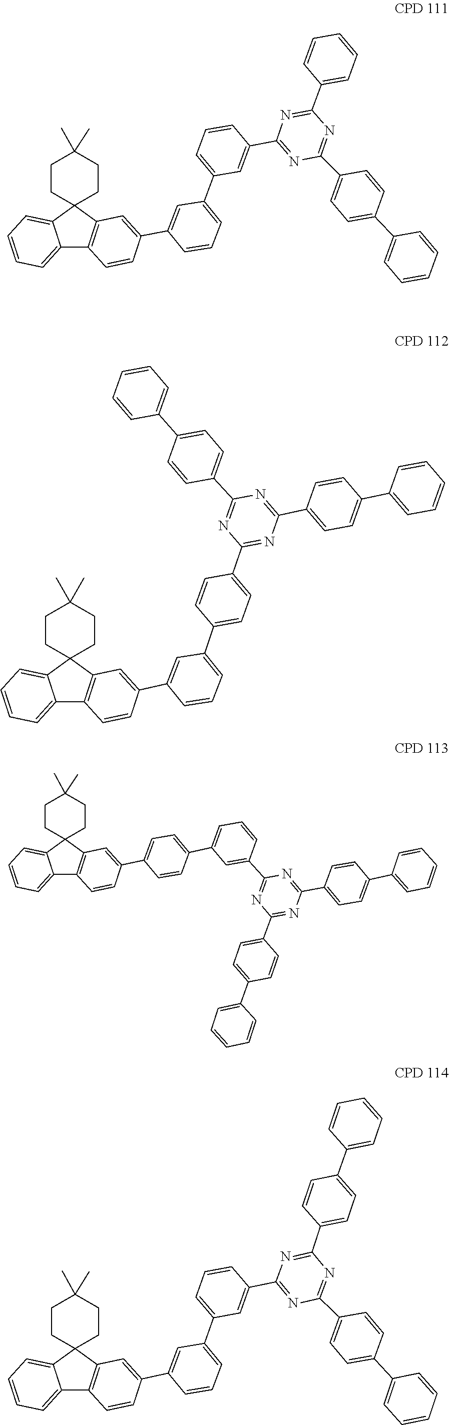 [SMILES: CC1(C)CCC2(CC1)c1ccccc1-c1ccc(-c3cccc(-c4cccc(-c5nc(-c6ccccc6)nc(-c6ccc(-c7ccccc7)cc6)n5)c4)c3)cc12 ; CC1(C)CCC2(CC1)c1ccccc1-c1ccc(-c3cccc(-c4ccc(-c5nc(-c6ccc(-c7ccccc7)cc6)nc(-c6ccc(-c7ccccc7)cc6)n5)cc4)c3)cc12 ; CC1(C)CCC2(CC1)c1ccccc1-c1ccc(-c3ccc(-c4cccc(-c5nc(-c6ccc(-c7ccccc7)cc6)nc(-c6ccc(-c7ccccc7)cc6)n5)c4)cc3)cc12 ; CC1(C)CCC2(CC1)c1ccccc1-c1ccc(-c3cccc(-c4cccc(-c5nc(-c6ccc(-c7ccccc7)cc6)nc(-c6ccc(-c7ccccc7)cc6)n5)c4)c3)cc12]

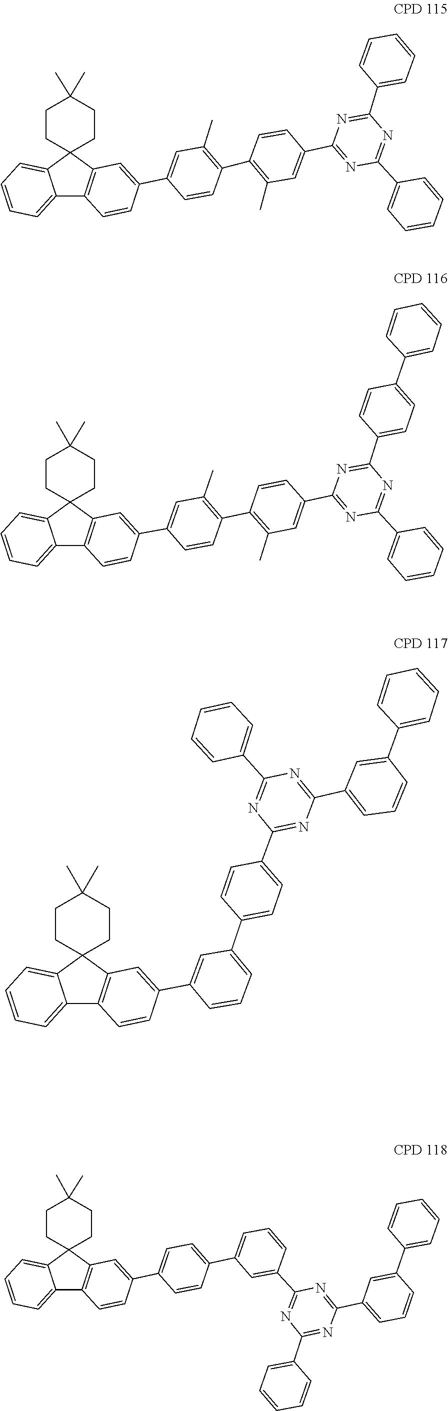 [SMILES: Cc1cc(-c2ccc3c(c2)C2(CCC(C)(C)CC2)c2ccccc2-3)ccc1-c1ccc(-c2nc(-c3ccccc3)nc(-c3ccccc3)n2)cc1C ; Cc1cc(-c2ccc3c(c2)C2(CCC(C)(C)CC2)c2ccccc2-3)ccc1-c1ccc(-c2nc(-c3ccccc3)nc(-c3ccc(-c4ccccc4)cc3)n2)cc1C ; CC1(C)CCC2(CC1)c1ccccc1-c1ccc(-c3cccc(-c4ccc(-c5nc(-c6ccccc6)nc(-c6cccc(-c7ccccc7)c6)n5)cc4)c3)cc12 ; CC1(C)CCC2(CC1)c1ccccc1-c1ccc(-c3ccc(-c4cccc(-c5nc(-c6ccccc6)nc(-c6cccc(-c7ccccc7)c6)n5)c4)cc3)cc12]

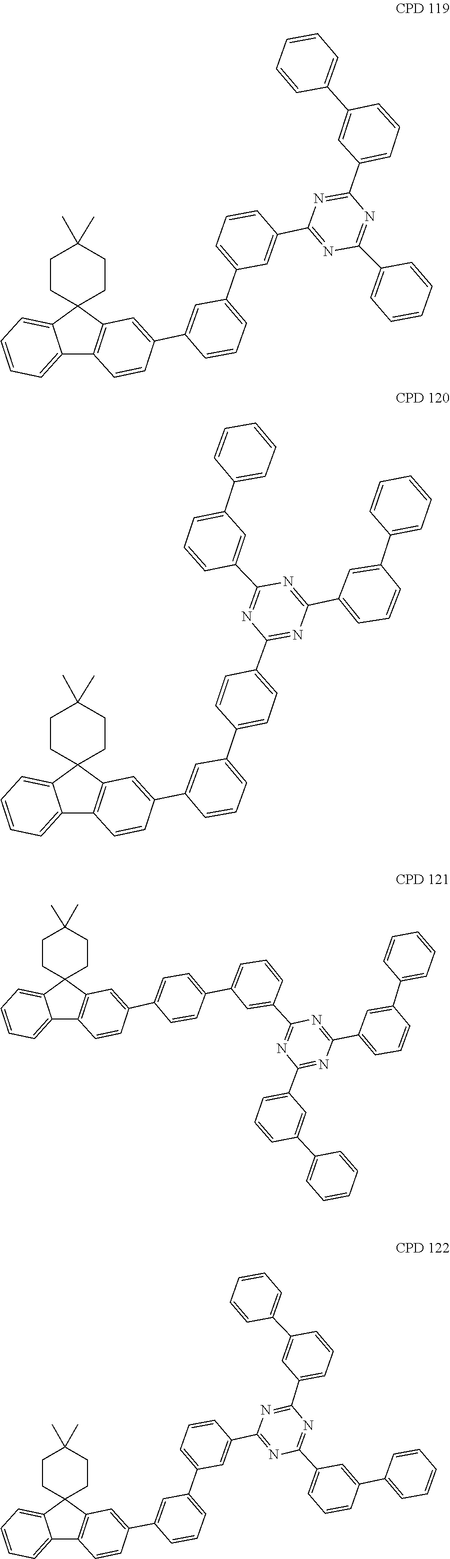 [SMILES: CC1(C)CCC2(CC1)c1ccccc1-c1ccc(-c3cccc(-c4cccc(-c5nc(-c6ccccc6)nc(-c6cccc(-c7ccccc7)c6)n5)c4)c3)cc12 ; CC1(C)CCC2(CC1)c1ccccc1-c1ccc(-c3cccc(-c4ccc(-c5nc(-c6cccc(-c7ccccc7)c6)nc(-c6cccc(-c7ccccc7)c6)n5)cc4)c3)cc12 ; CC1(C)CCC2(CC1)c1ccccc1-c1ccc(-c3ccc(-c4cccc(-c5nc(-c6cccc(-c7ccccc7)c6)nc(-c6cccc(-c7ccccc7)c6)n5)c4)cc3)cc12 ; CC1(C)CCC2(CC1)c1ccccc1-c1ccc(-c3cccc(-c4cccc(-c5nc(-c6cccc(-c7ccccc7)c6)nc(-c6cccc(-c7ccccc7)c6)n5)c4)c3)cc12]

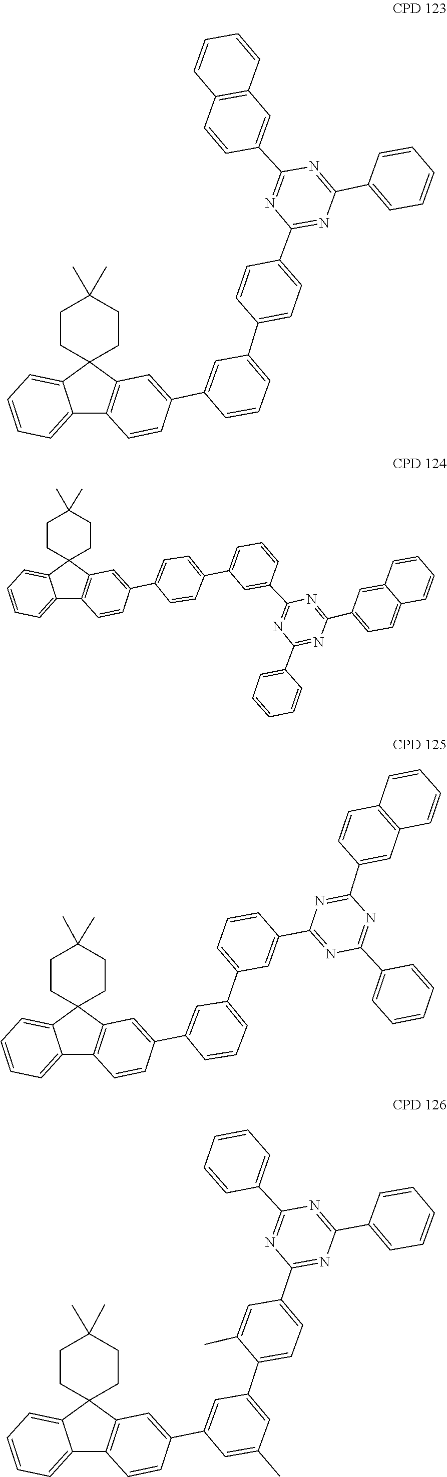 [SMILES: CC1(C)CCC2(CC1)c1ccccc1-c1ccc(-c3cccc(-c4ccc(-c5nc(-c6ccccc6)nc(-c6ccc7ccccc7c6)n5)cc4)c3)cc12 ; CC1(C)CCC2(CC1)c1ccccc1-c1ccc(-c3ccc(-c4cccc(-c5nc(-c6ccccc6)nc(-c6ccc7ccccc7c6)n5)c4)cc3)cc12 ; CC1(C)CCC2(CC1)c1ccccc1-c1ccc(-c3cccc(-c4cccc(-c5nc(-c6ccccc6)nc(-c6ccc7ccccc7c6)n5)c4)c3)cc12 ; Cc1cc(-c2ccc3c(c2)C2(CCC(C)(C)CC2)c2ccccc2-3)cc(-c2ccc(-c3nc(-c4ccccc4)nc(-c4ccccc4)n3)cc2C)c1]

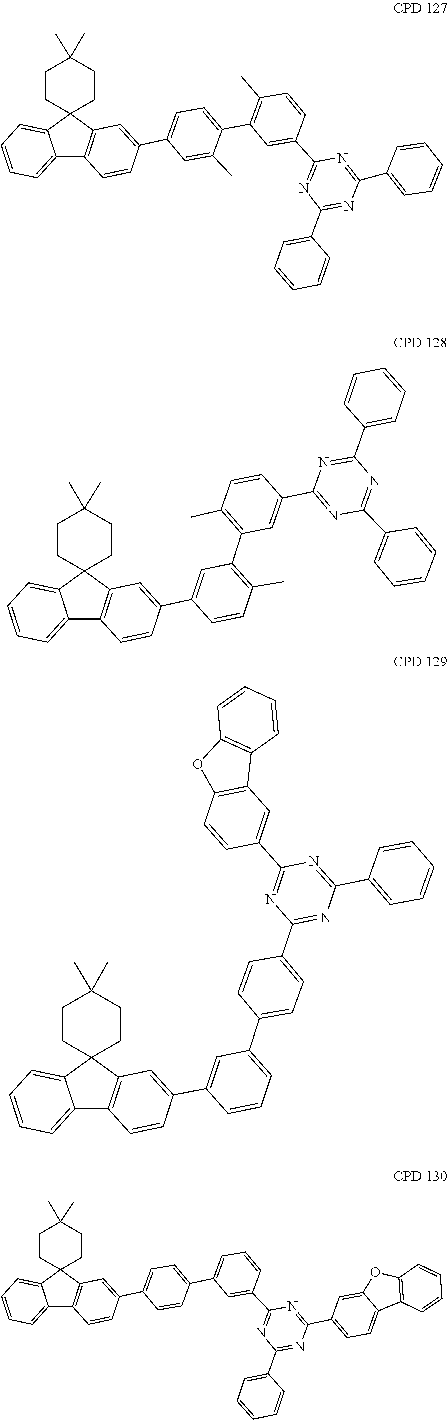 [SMILES: Cc1cc(-c2ccc3c(c2)C2(CCC(C)(C)CC2)c2ccccc2-3)ccc1-c1cc(-c2nc(-c3ccccc3)nc(-c3ccccc3)n2)ccc1C ; Cc1ccc(-c2ccc3c(c2)C2(CCC(C)(C)CC2)c2ccccc2-3)cc1-c1cc(-c2nc(-c3ccccc3)nc(-c3ccccc3)n2)ccc1C ; CC1(C)CCC2(CC1)c1ccccc1-c1ccc(-c3cccc(-c4ccc(-c5nc(-c6ccccc6)nc(-c6ccc7oc8ccccc8c7c6)n5)cc4)c3)cc12 ; CC1(C)CCC2(CC1)c1ccccc1-c1ccc(-c3ccc(-c4cccc(-c5nc(-c6ccccc6)nc(-c6ccc7c(c6)oc6ccccc67)n5)c4)cc3)cc12]

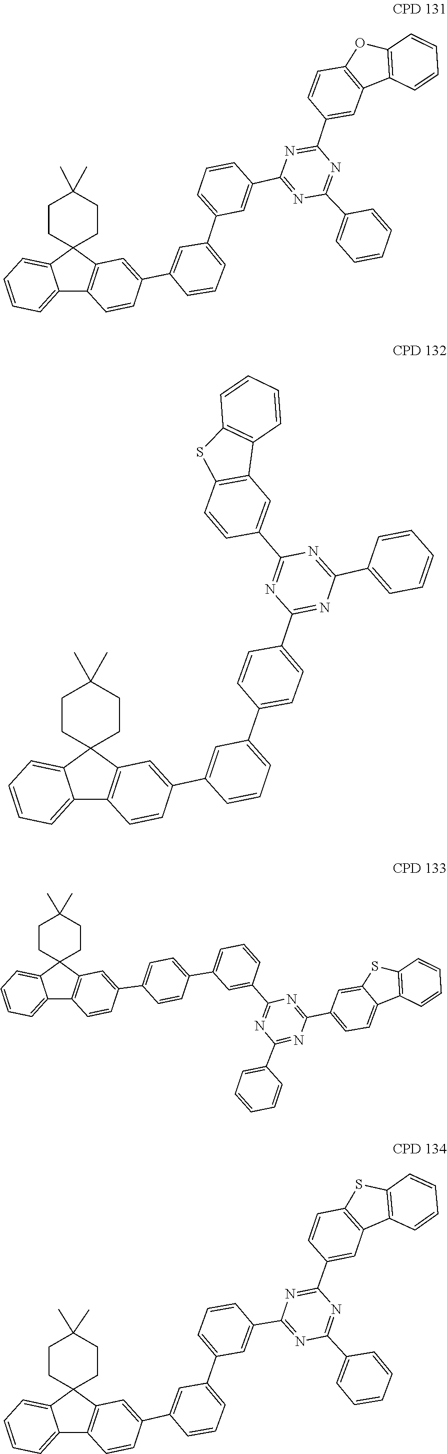 [SMILES: CC1(C)CCC2(CC1)c1ccccc1-c1ccc(-c3cccc(-c4cccc(-c5nc(-c6ccccc6)nc(-c6ccc7oc8ccccc8c7c6)n5)c4)c3)cc12 ; CC1(C)CCC2(CC1)c1ccccc1-c1ccc(-c3cccc(-c4ccc(-c5nc(-c6ccccc6)nc(-c6ccc7sc8ccccc8c7c6)n5)cc4)c3)cc12 ; CC1(C)CCC2(CC1)c1ccccc1-c1ccc(-c3ccc(-c4cccc(-c5nc(-c6ccccc6)nc(-c6ccc7c(c6)sc6ccccc67)n5)c4)cc3)cc12 ; CC1(C)CCC2(CC1)c1ccccc1-c1ccc(-c3cccc(-c4cccc(-c5nc(-c6ccccc6)nc(-c6ccc7sc8ccccc8c7c6)n5)c4)c3)cc12]

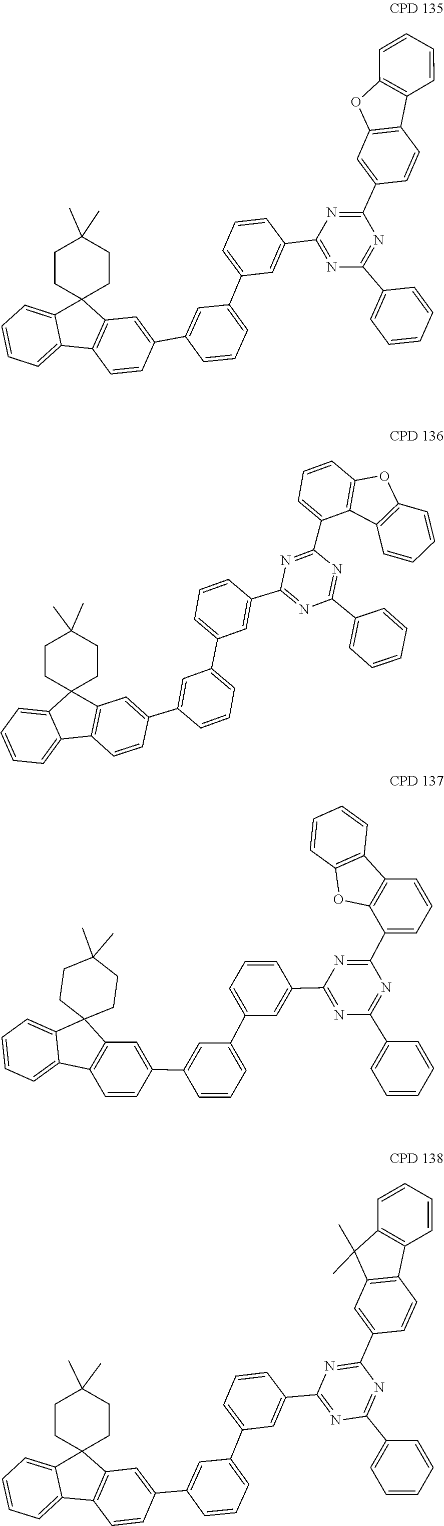 [SMILES: CC1(C)CCC2(CC1)c1ccccc1-c1ccc(-c3cccc(-c4cccc(-c5nc(-c6ccccc6)nc(-c6ccc7c(c6)oc6ccccc67)n5)c4)c3)cc12 ; CC1(C)CCC2(CC1)c1ccccc1-c1ccc(-c3cccc(-c4cccc(-c5nc(-c6ccccc6)nc(-c6cccc7oc8ccccc8c67)n5)c4)c3)cc12 ; CC1(C)CCC2(CC1)c1ccccc1-c1ccc(-c3cccc(-c4cccc(-c5nc(-c6ccccc6)nc(-c6cccc7c6oc6ccccc67)n5)c4)c3)cc12 ; CC1(C)CCC2(CC1)c1ccccc1-c1ccc(-c3cccc(-c4cccc(-c5nc(-c6ccccc6)nc(-c6ccc7c(c6)C(C)(C)c6ccccc6-7)n5)c4)c3)cc12]

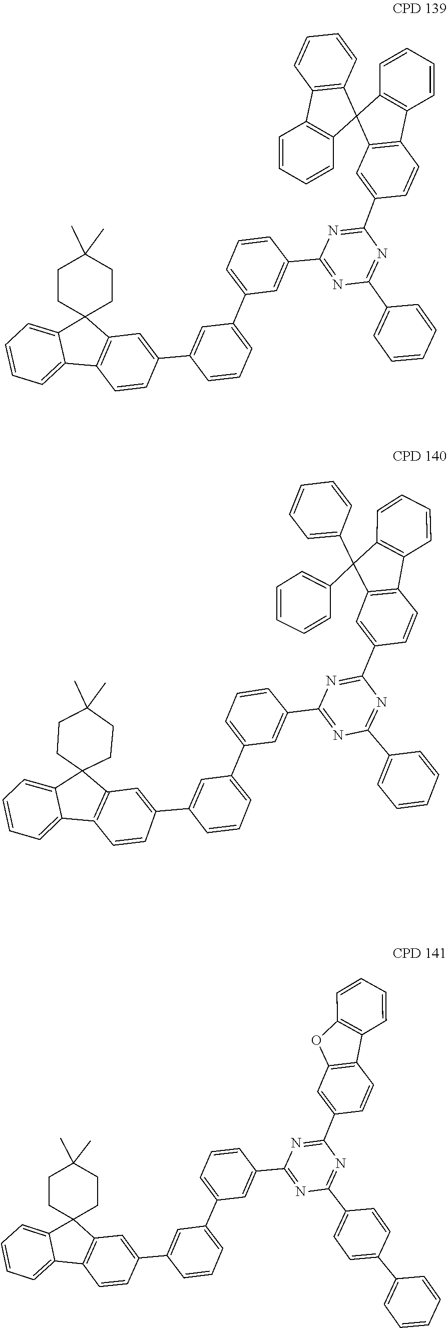 [SMILES: CC1(C)CCC2(CC1)c1ccccc1-c1ccc(-c3cccc(-c4cccc(-c5nc(-c6ccccc6)nc(-c6ccc7c(c6)C6(c8ccccc8-c8ccccc86)c6ccccc6-7)n5)c4)c3)cc12 ; C=C1/C=C(c2cccc(-c3cccc(-c4nc(-c5ccccc5)nc(-c5ccc6c(c5)C(c5ccccc5)(c5ccccc5)c5ccccc5-6)n4)c3)c2)\C=C/Cc2ccccc2C12CCC(C)(C)CC2 ; CC1(C)CCC2(CC1)c1ccccc1-c1ccc(-c3cccc(-c4cccc(-c5nc(-c6ccc(-c7ccccc7)cc6)nc(-c6ccc7c(c6)oc6ccccc67)n5)c4)c3)cc12]

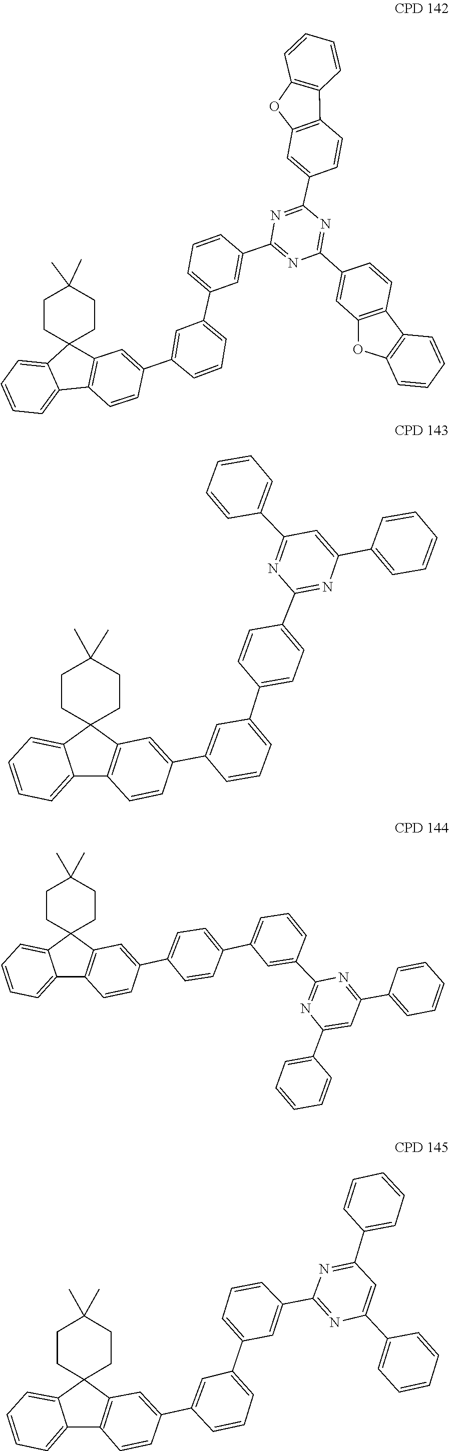 [SMILES: CC1(C)CCC2(CC1)c1ccccc1-c1ccc(-c3cccc(-c4cccc(-c5nc(-c6ccc7c(c6)oc6ccccc67)nc(-c6ccc7c(c6)oc6ccccc67)n5)c4)c3)cc12 ; CC1(C)CCC2(CC1)c1ccccc1-c1ccc(-c3cccc(-c4ccc(-c5nc(-c6ccccc6)cc(-c6ccccc6)n5)cc4)c3)cc12 ; CC1(C)CCC2(CC1)c1ccccc1-c1ccc(-c3ccc(-c4cccc(-c5nc(-c6ccccc6)cc(-c6ccccc6)n5)c4)cc3)cc12 ; C=C1/C=C(c2cccc(-c3cccc(-c4nc(-c5ccccc5)cc(-c5ccccc5)n4)c3)c2)\C=C/Cc2ccccc2C12CCC(C)(C)CC2]

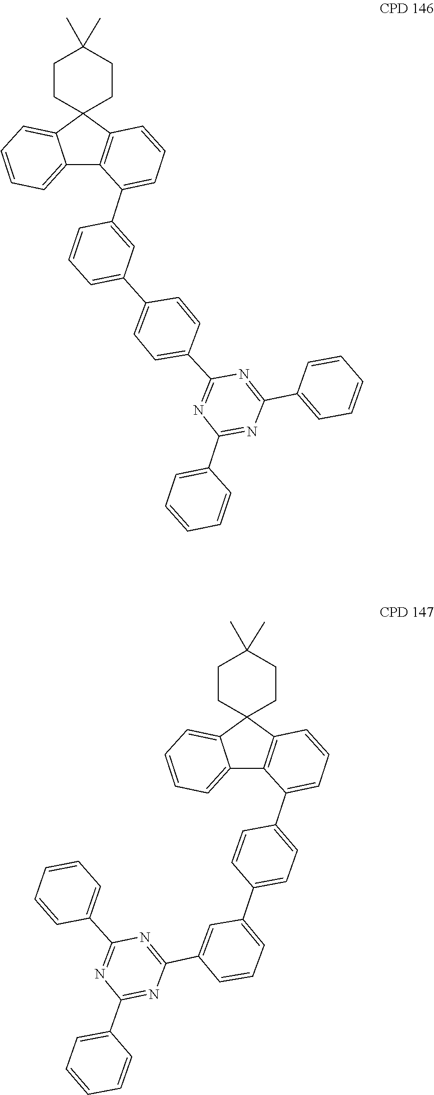 [SMILES: CC1(C)CCC2(CC1)c1ccccc1-c1c(-c3cccc(-c4ccc(-c5nc(-c6ccccc6)nc(-c6ccccc6)n5)cc4)c3)cccc12 ; CC1(C)CCC2(CC1)c1ccccc1-c1c(-c3ccc(-c4cccc(-c5nc(-c6ccccc6)nc(-c6ccccc6)n5)c4)cc3)cccc12]

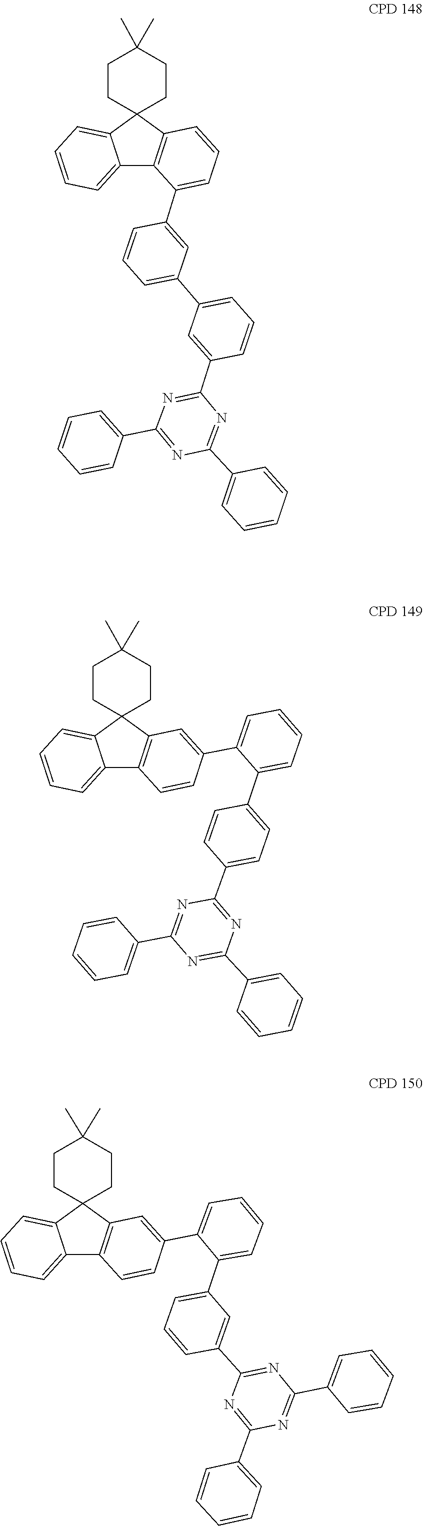 [SMILES: CC1(C)CCC2(CC1)c1ccccc1-c1c(-c3cccc(-c4cccc(-c5nc(-c6ccccc6)nc(-c6ccccc6)n5)c4)c3)cccc12 ; CC1(C)CCC2(CC1)c1ccccc1-c1ccc(-c3ccccc3-c3ccc(-c4nc(-c5ccccc5)nc(-c5ccccc5)n4)cc3)cc12 ; CC1(C)CCC2(CC1)c1ccccc1-c1ccc(-c3ccccc3-c3cccc(-c4nc(-c5ccccc5)nc(-c5ccccc5)n4)c3)cc12]

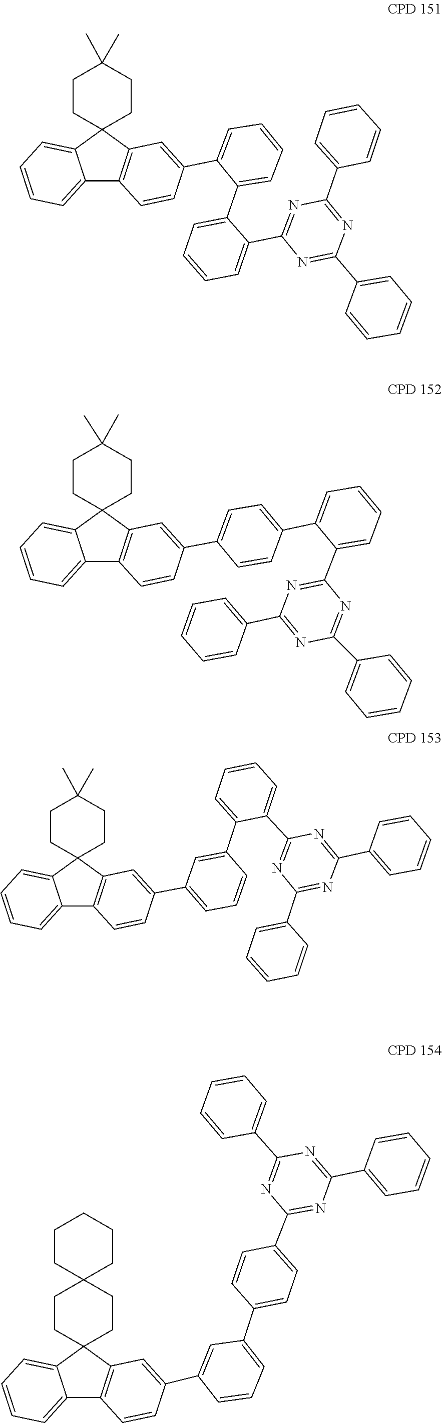 [SMILES: CC1(C)CCC2(CC1)c1ccccc1-c1ccc(-c3ccccc3-c3ccccc3-c3nc(-c4ccccc4)nc(-c4ccccc4)n3)cc12 ; CC1(C)CCC2(CC1)c1ccccc1-c1ccc(-c3ccc(-c4ccccc4-c4nc(-c5ccccc5)nc(-c5ccccc5)n4)cc3)cc12 ; CC1(C)CCC2(CC1)c1ccccc1-c1ccc(-c3cccc(-c4ccccc4-c4nc(-c5ccccc5)nc(-c5ccccc5)n4)c3)cc12 ; c1ccc(-c2nc(-c3ccccc3)nc(-c3ccc(-c4cccc(-c5ccc6c(c5)C5(CCC7(CCCCC7)CC5)c5ccccc5-6)c4)cc3)n2)cc1]

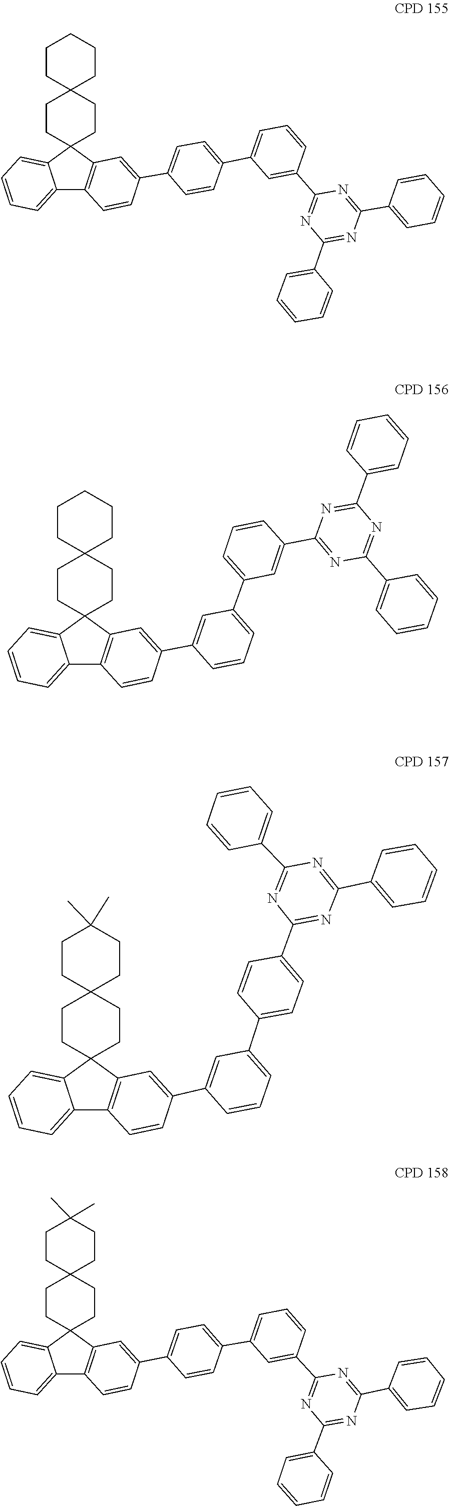 [SMILES: c1ccc(-c2nc(-c3ccccc3)nc(-c3cccc(-c4ccc(-c5ccc6c(c5)C5(CCC7(CCCCC7)CC5)c5ccccc5-6)cc4)c3)n2)cc1 ; c1ccc(-c2nc(-c3ccccc3)nc(-c3cccc(-c4cccc(-c5ccc6c(c5)C5(CCC7(CCCCC7)CC5)c5ccccc5-6)c4)c3)n2)cc1 ; CC1(C)CCC2(CC1)CCC1(CC2)c2ccccc2-c2ccc(-c3cccc(-c4ccc(-c5nc(-c6ccccc6)nc(-c6ccccc6)n5)cc4)c3)cc21 ; CC1(C)CCC2(CC1)CCC1(CC2)c2ccccc2-c2ccc(-c3ccc(-c4cccc(-c5nc(-c6ccccc6)nc(-c6ccccc6)n5)c4)cc3)cc21]

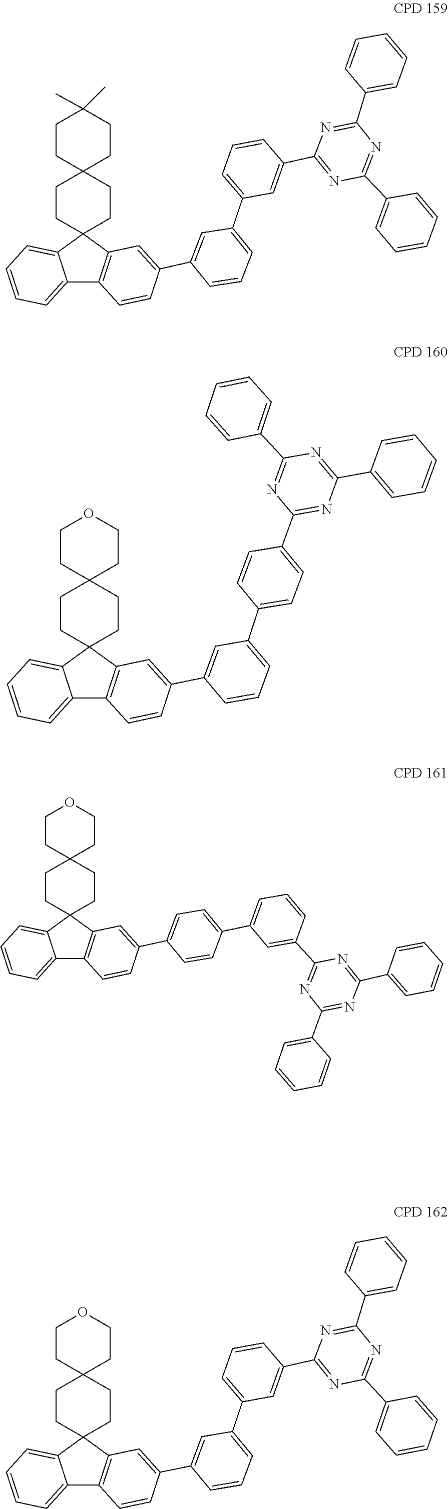 [SMILES: CC1(C)CCC2(CC1)CCC1(CC2)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3ccc(-c4cccc(-c5ccc6c(c5)C5(CCC7(CCOCC7)CC5)c5ccccc5-6)c4)cc3)n2)cc1 ; c1ccc(-c2nc(-c3ccccc3)nc(-c3cccc(-c4ccc(-c5ccc6c(c5)C5(CCC7(CCOCC7)CC5)c5ccccc5-6)cc4)c3)n2)cc1 ; c1ccc(-c2nc(-c3ccccc3)nc(-c3cccc(-c4cccc(-c5ccc6c(c5)C5(CCC7(CCOCC7)CC5)c5ccccc5-6)c4)c3)n2)cc1]

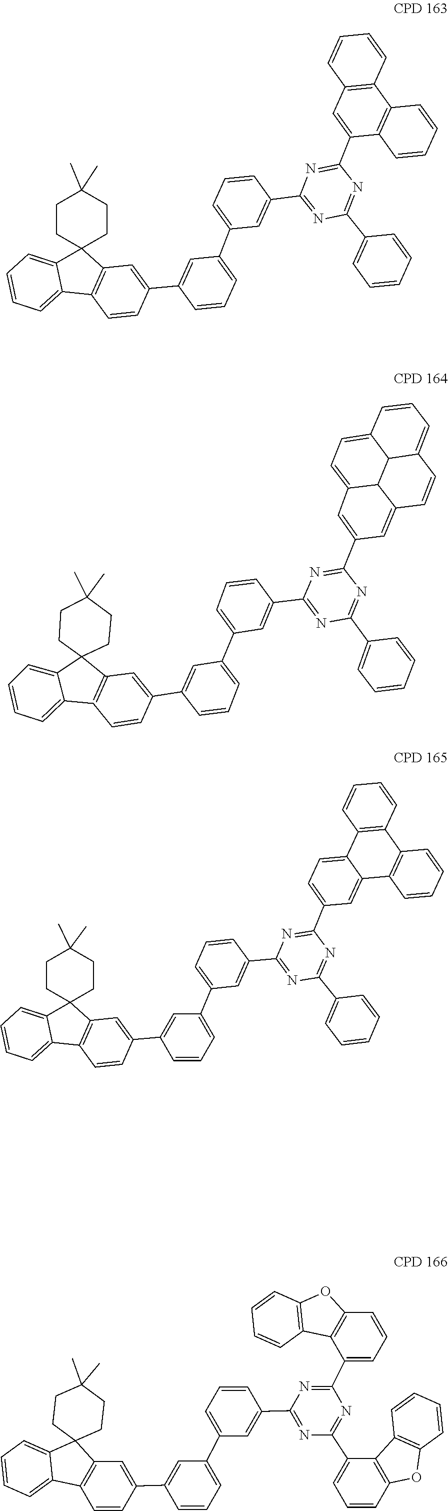 [SMILES: CC1(C)CCC2(CC1)c1ccccc1-c1ccc(-c3cccc(-c4cccc(-c5nc(-c6ccccc6)nc(-c6cc7ccccc7c7ccccc67)n5)c4)c3)cc12 ; C=C1/C=C(c2cccc(-c3cccc(-c4nc(C5=CC6=CC=C7C=CC=C8C=CC(=C5)C6C87)nc(-c5ccccc5)n4)c3)c2)\C=C/Cc2ccccc2C12CCC(C)(C)CC2 ; CC1(C)CCC2(CC1)c1ccccc1-c1ccc(-c3cccc(-c4cccc(-c5nc(-c6ccccc6)nc(-c6ccc7c8ccccc8c8ccccc8c7c6)n5)c4)c3)cc12 ; CC1(C)CCC2(CC1)c1ccccc1-c1ccc(-c3cccc(-c4cccc(-c5nc(-c6cccc7oc8ccccc8c67)nc(-c6cccc7oc8ccccc8c67)n5)c4)c3)cc12]

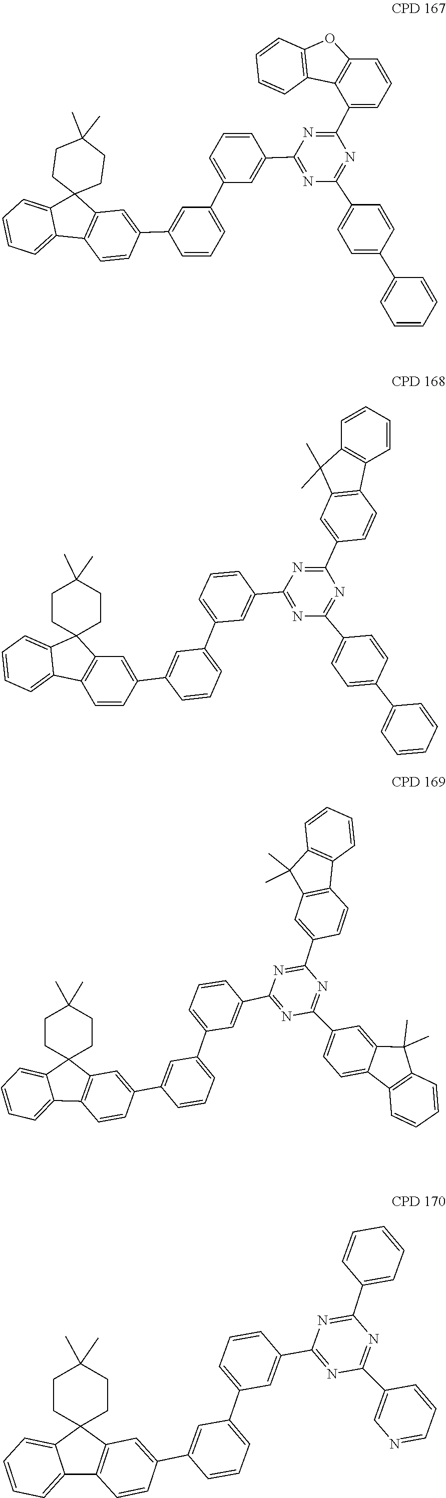 [SMILES: CC1(C)CCC2(CC1)c1ccccc1-c1ccc(-c3cccc(-c4cccc(-c5nc(-c6ccc(-c7ccccc7)cc6)nc(-c6cccc7oc8ccccc8c67)n5)c4)c3)cc12 ; CC1(C)CCC2(CC1)c1ccccc1-c1ccc(-c3cccc(-c4cccc(-c5nc(-c6ccc(-c7ccccc7)cc6)nc(-c6ccc7c(c6)C(C)(C)c6ccccc6-7)n5)c4)c3)cc12 ; CC1(C)CCC2(CC1)c1ccccc1-c1ccc(-c3cccc(-c4cccc(-c5nc(-c6ccc7c(c6)C(C)(C)c6ccccc6-7)nc(-c6ccc7c(c6)C(C)(C)c6ccccc6-7)n5)c4)c3)cc12 ; CC1(C)CCC2(CC1)c1ccccc1-c1ccc(-c3cccc(-c4cccc(-c5nc(-c6ccccc6)nc(-c6cccnc6)n5)c4)c3)cc12]

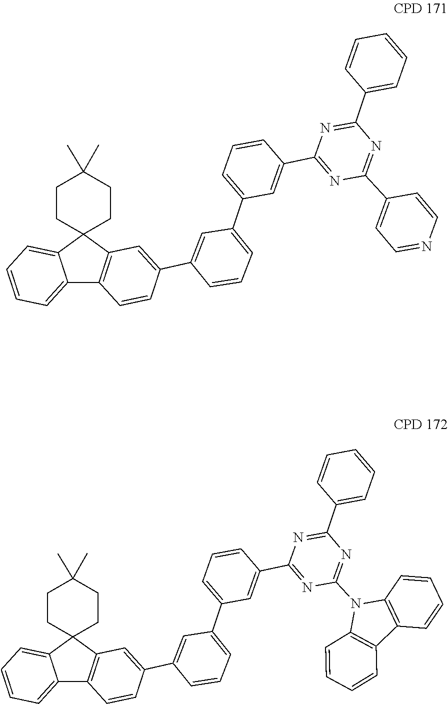 [SMILES: CC1(C)CCC2(CC1)c1ccccc1-c1ccc(-c3cccc(-c4cccc(-c5nc(-c6ccccc6)nc(-c6ccncc6)n5)c4)c3)cc12 ; CC1(C)CCC2(CC1)c1ccccc1-c1ccc(-c3cccc(-c4cccc(-c5nc(-c6ccccc6)nc(-n6c7ccccc7c7ccccc76)n5)c4)c3)cc12]

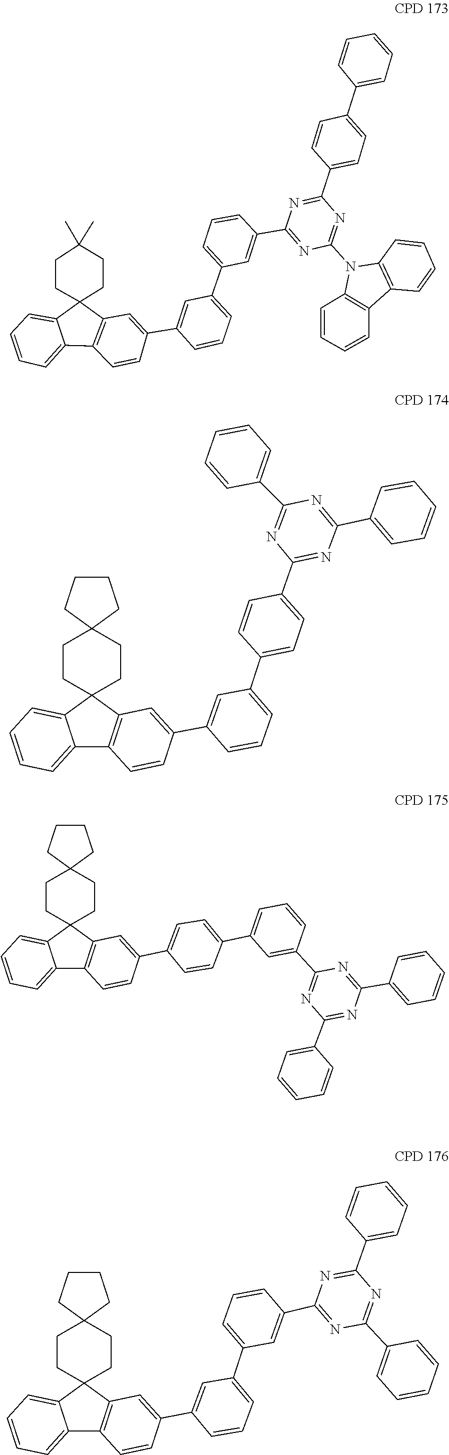 [SMILES: CC1(C)CCC2(CC1)c1ccccc1-c1ccc(-c3cccc(-c4cccc(-c5nc(-c6ccc(-c7ccccc7)cc6)nc(-n6c7ccccc7c7ccccc76)n5)c4)c3)cc12 ; c1ccc(-c2nc(-c3ccccc3)nc(-c3ccc(-c4cccc(-c5ccc6c(c5)C5(CCC7(CCCC7)CC5)c5ccccc5-6)c4)cc3)n2)cc1 ; c1ccc(-c2nc(-c3ccccc3)nc(-c3cccc(-c4ccc(-c5ccc6c(c5)C5(CCC7(CCCC7)CC5)c5ccccc5-6)cc4)c3)n2)cc1 ; c1ccc(-c2nc(-c3ccccc3)nc(-c3cccc(-c4cccc(-c5ccc6c(c5)C5(CCC7(CCCC7)CC5)c5ccccc5-6)c4)c3)n2)cc1]

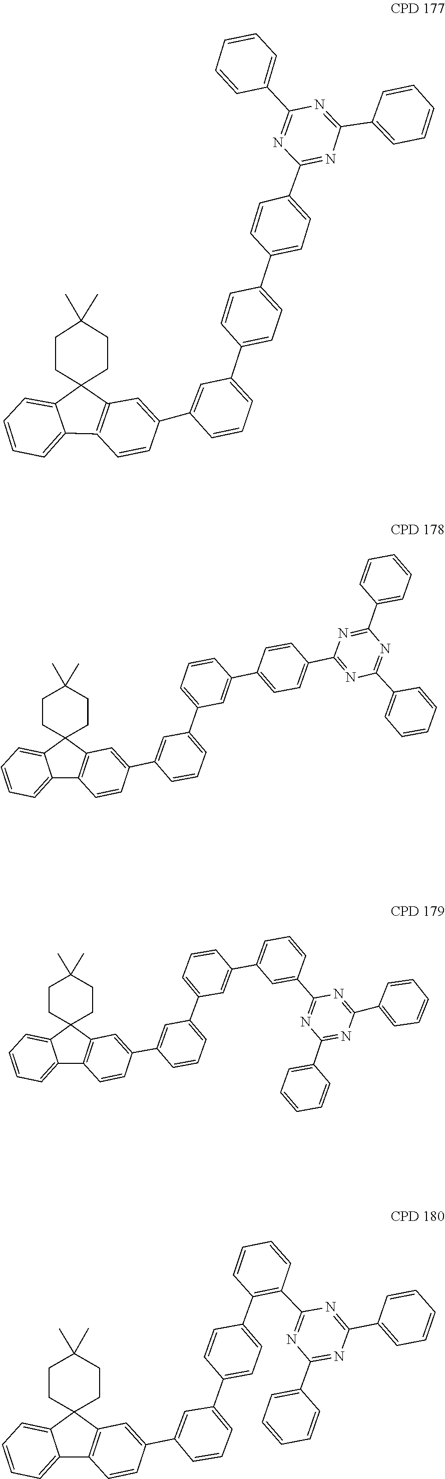 [SMILES: C=C1/C=C(c2cccc(-c3ccc(-c4ccc(-c5nc(-c6ccccc6)nc(-c6ccccc6)n5)cc4)cc3)c2)\C=C/Cc2ccccc2C12CCC(C)(C)CC2 ; CC1(C)CCC2(CC1)c1ccccc1-c1ccc(-c3cccc(-c4cccc(-c5ccc(-c6nc(-c7ccccc7)nc(-c7ccccc7)n6)cc5)c4)c3)cc12 ; CC1(C)CCC2(CC1)c1ccccc1-c1ccc(-c3cccc(-c4cccc(-c5cccc(-c6nc(-c7ccccc7)nc(-c7ccccc7)n6)c5)c4)c3)cc12 ; CC1(C)CCC2(CC1)c1ccccc1-c1ccc(-c3cccc(-c4ccc(-c5ccccc5-c5nc(-c6ccccc6)nc(-c6ccccc6)n5)cc4)c3)cc12]

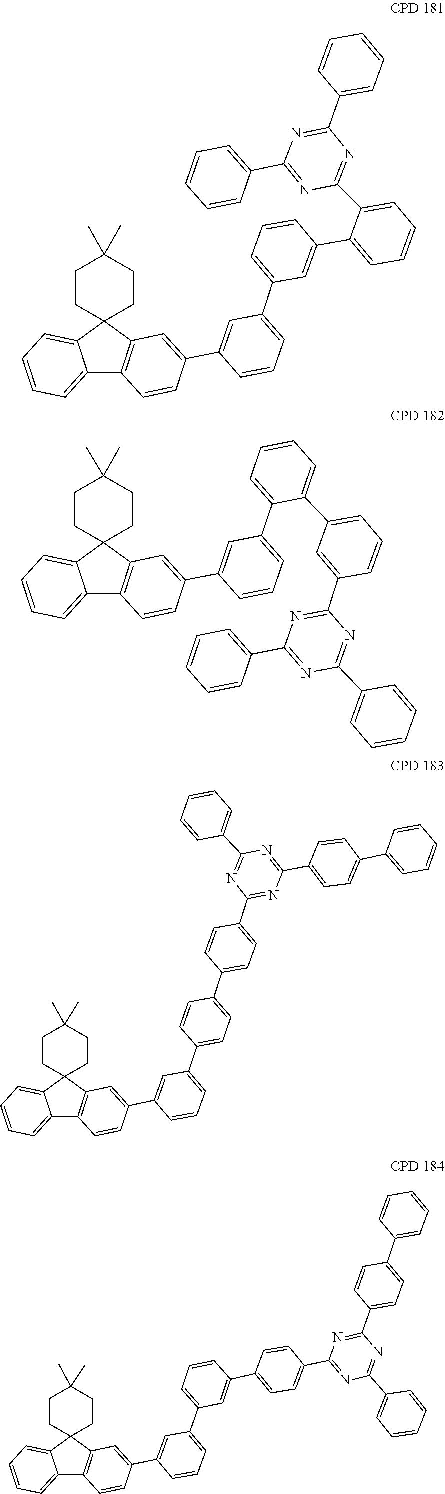 [SMILES: CC1(C)CCC2(CC1)c1ccccc1-c1ccc(-c3cccc(-c4cccc(-c5ccccc5-c5nc(-c6ccccc6)nc(-c6ccccc6)n5)c4)c3)cc12 ; CC1(C)CCC2(CC1)c1ccccc1-c1ccc(-c3cccc(-c4ccccc4-c4cccc(-c5nc(-c6ccccc6)nc(-c6ccccc6)n5)c4)c3)cc12 ; CC1(C)CCC2(CC1)c1ccccc1-c1ccc(-c3cccc(-c4ccc(-c5ccc(-c6nc(-c7ccccc7)nc(-c7ccc(-c8ccccc8)cc7)n6)cc5)cc4)c3)cc12 ; CC1(C)CCC2(CC1)c1ccccc1-c1ccc(-c3cccc(-c4cccc(-c5ccc(-c6nc(-c7ccccc7)nc(-c7ccc(-c8ccccc8)cc7)n6)cc5)c4)c3)cc12]

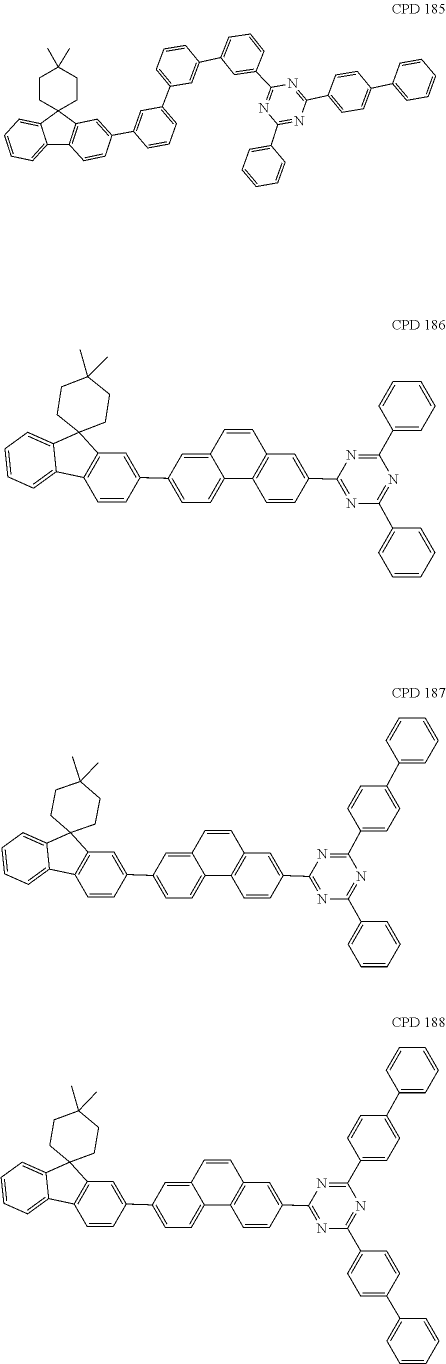 [SMILES: CC1(C)CCC2(CC1)c1ccccc1-c1ccc(-c3cccc(-c4cccc(-c5cccc(-c6nc(-c7ccccc7)nc(-c7ccc(-c8ccccc8)cc7)n6)c5)c4)c3)cc12 ; CC1(C)CCC2(CC1)c1ccccc1-c1ccc(-c3ccc4c(ccc5cc(-c6nc(-c7ccccc7)nc(-c7ccccc7)n6)ccc54)c3)cc12 ; CC1(C)CCC2(CC1)c1ccccc1-c1ccc(-c3ccc4c(ccc5cc(-c6nc(-c7ccccc7)nc(-c7ccc(-c8ccccc8)cc7)n6)ccc54)c3)cc12 ; CC1(C)CCC2(CC1)c1ccccc1-c1ccc(-c3ccc4c(ccc5cc(-c6nc(-c7ccc(-c8ccccc8)cc7)nc(-c7ccc(-c8ccccc8)cc7)n6)ccc54)c3)cc12]

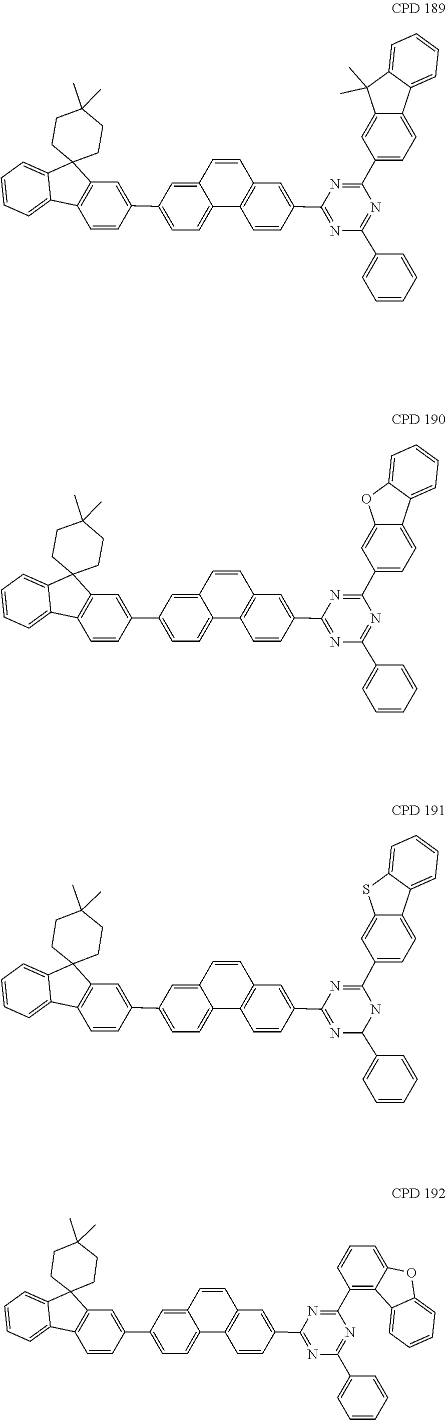

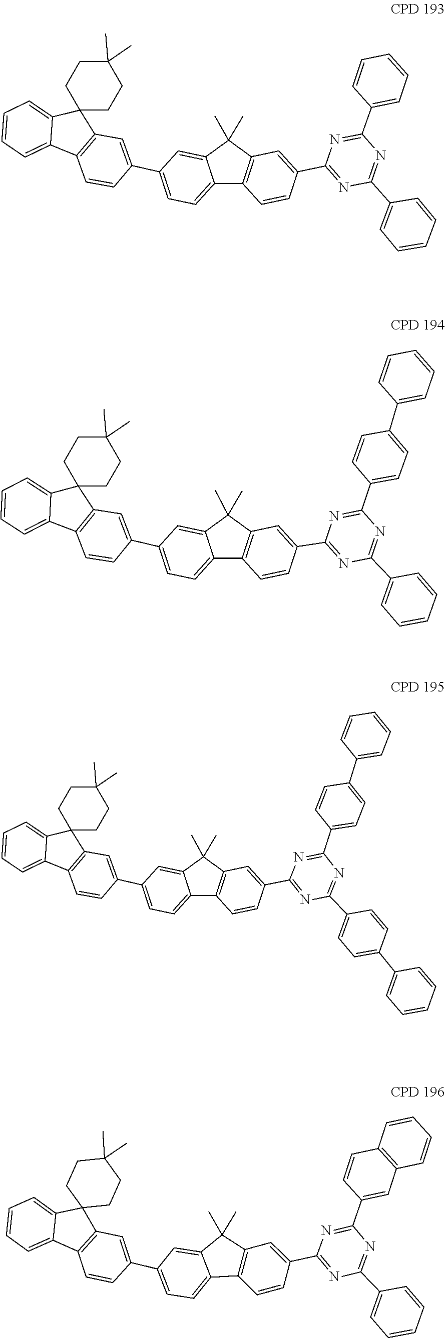 [SMILES: CC1(C)CCC2(CC1)c1ccccc1-c1ccc(-c3ccc4c(c3)C(C)(C)c3cc(-c5nc(-c6ccccc6)nc(-c6ccccc6)n5)ccc3-4)cc12 ; CC1(C)CCC2(CC1)c1ccccc1-c1ccc(-c3ccc4c(c3)C(C)(C)c3cc(-c5nc(-c6ccccc6)nc(-c6ccc(-c7ccccc7)cc6)n5)ccc3-4)cc12 ; CC1(C)CCC2(CC1)c1ccccc1-c1ccc(-c3ccc4c(c3)C(C)(C)c3cc(-c5nc(-c6ccc(-c7ccccc7)cc6)nc(-c6ccc(-c7ccccc7)cc6)n5)ccc3-4)cc12 ; CC1(C)CCC2(CC1)c1ccccc1-c1ccc(-c3ccc4c(c3)C(C)(C)c3cc(-c5nc(-c6ccccc6)nc(-c6ccc7ccccc7c6)n5)ccc3-4)cc12]

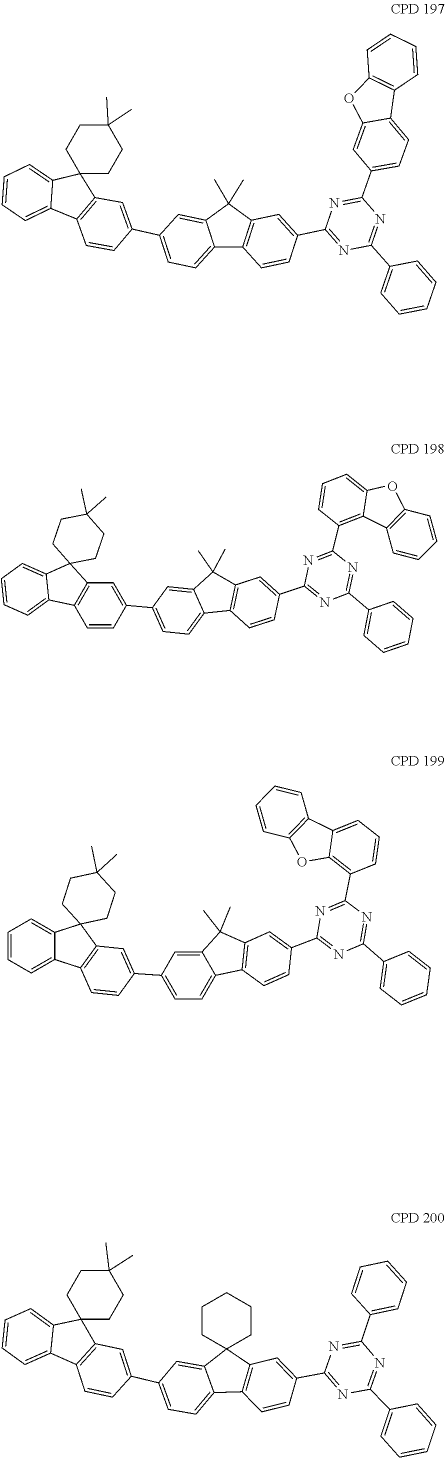 [SMILES: CC1(C)CCC2(CC1)c1ccccc1-c1ccc(-c3ccc4c(c3)C(C)(C)c3cc(-c5nc(-c6ccccc6)nc(-c6ccc7c(c6)oc6ccccc67)n5)ccc3-4)cc12 ; CC1(C)CCC2(CC1)c1ccccc1-c1ccc(-c3ccc4c(c3)C(C)(C)c3cc(-c5nc(-c6ccccc6)nc(-c6cccc7oc8ccccc8c67)n5)ccc3-4)cc12 ; CC1(C)CCC2(CC1)c1ccccc1-c1ccc(-c3ccc4c(c3)C(C)(C)c3cc(-c5nc(-c6ccccc6)nc(-c6cccc7c6oc6ccccc67)n5)ccc3-4)cc12 ; CC1(C)CCC2(CC1)c1ccccc1-c1ccc(-c3ccc4c(c3)C3(CCCCC3)c3cc(-c5nc(-c6ccccc6)nc(-c6ccccc6)n5)ccc3-4)cc12]

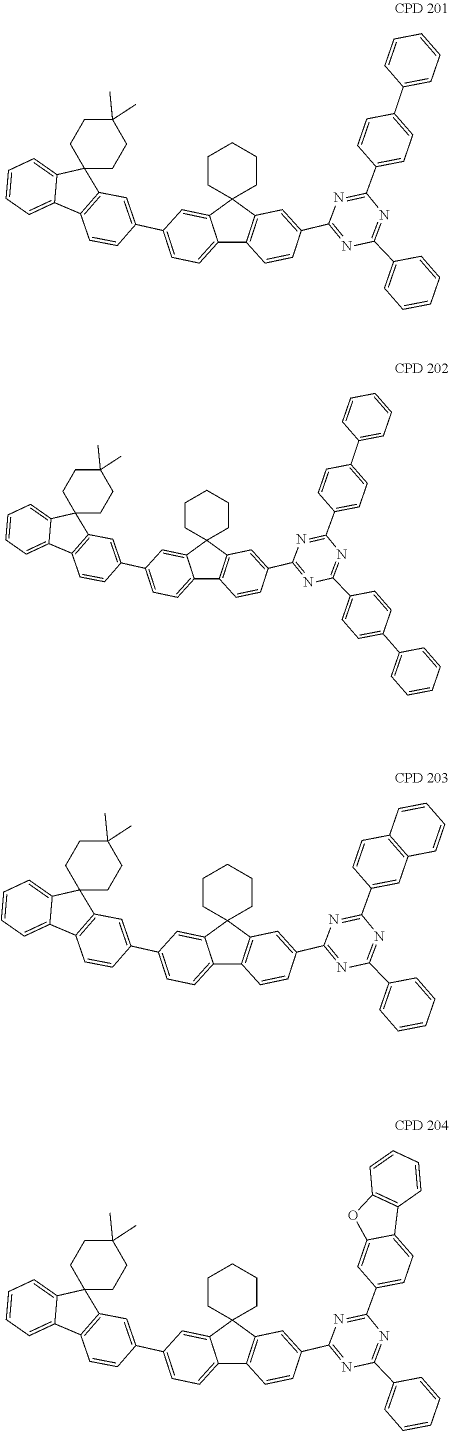 [SMILES: CC1(C)CCC2(CC1)c1ccccc1-c1ccc(-c3ccc4c(c3)C3(CCCCC3)c3cc(-c5nc(-c6ccccc6)nc(-c6ccc(-c7ccccc7)cc6)n5)ccc3-4)cc12 ; CC1(C)CCC2(CC1)c1ccccc1-c1ccc(-c3ccc4c(c3)C3(CCCCC3)c3cc(-c5nc(-c6ccc(-c7ccccc7)cc6)nc(-c6ccc(-c7ccccc7)cc6)n5)ccc3-4)cc12 ; CC1(C)CCC2(CC1)c1ccccc1-c1ccc(-c3ccc4c(c3)C3(CCCCC3)c3cc(-c5nc(-c6ccccc6)nc(-c6ccc7ccccc7c6)n5)ccc3-4)cc12 ; CC1(C)CCC2(CC1)c1ccccc1-c1ccc(-c3ccc4c(c3)C3(CCCCC3)c3cc(-c5nc(-c6ccccc6)nc(-c6ccc7c(c6)oc6ccccc67)n5)ccc3-4)cc12]

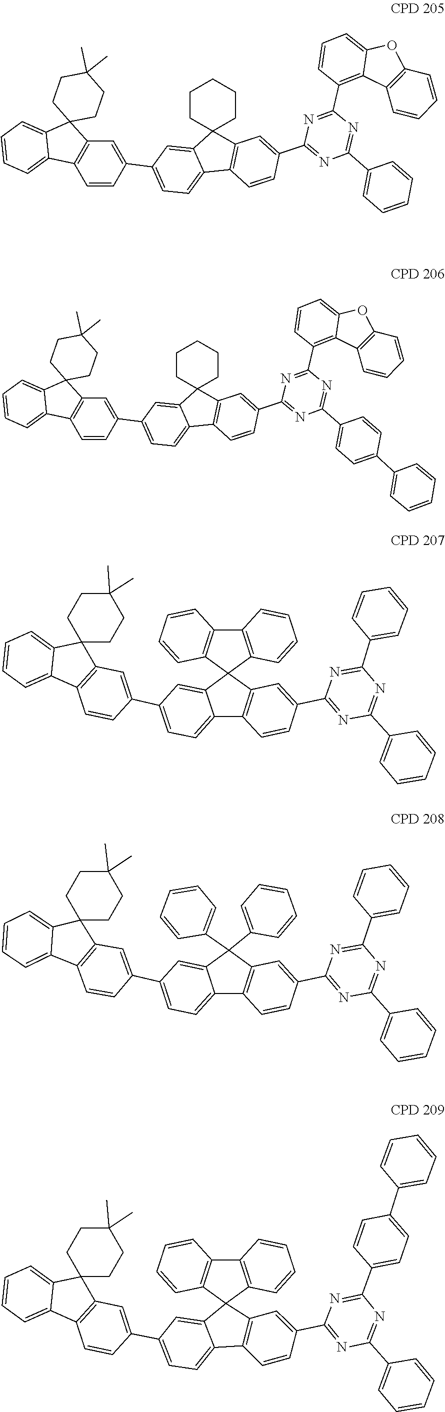

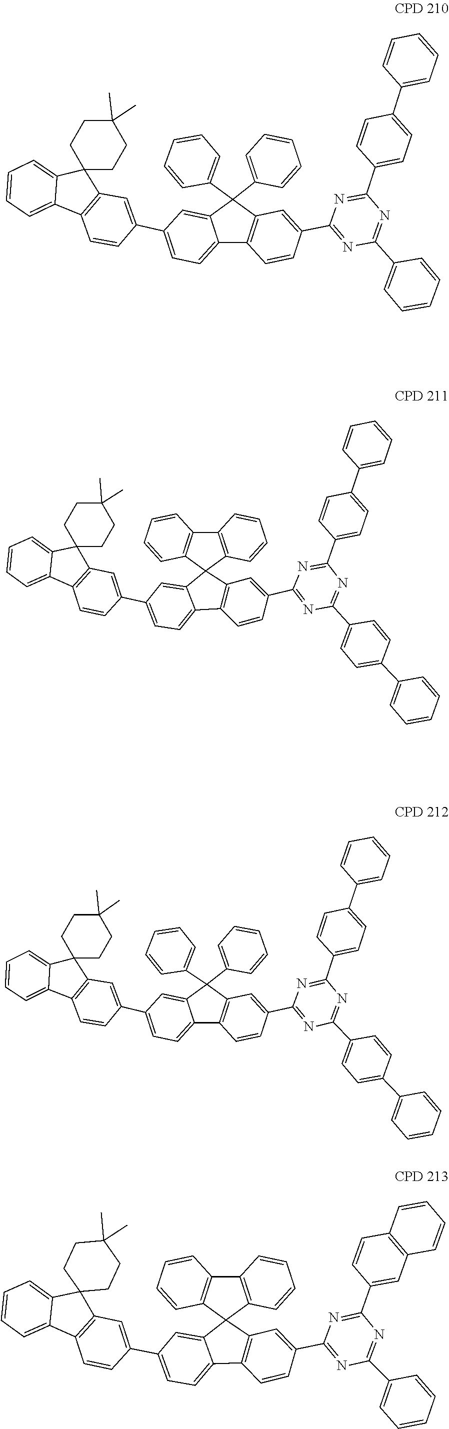

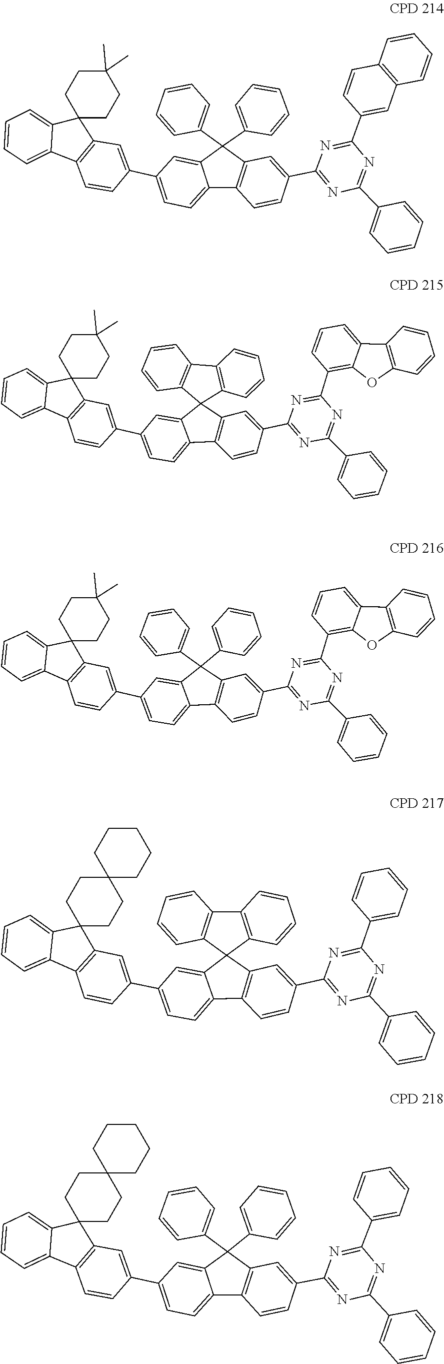

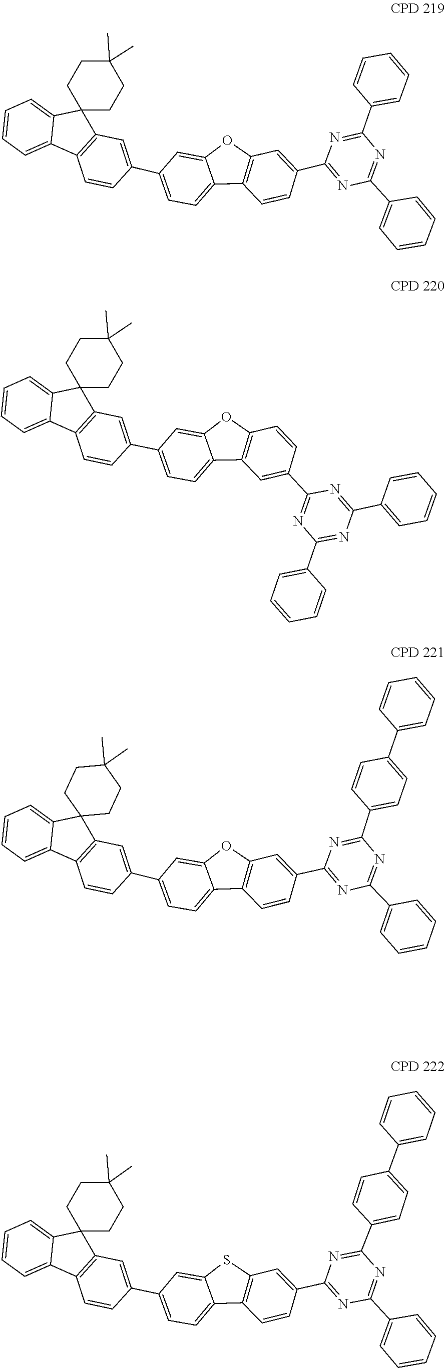 [SMILES: CC1(C)CCC2(CC1)c1ccccc1-c1ccc(-c3ccc4c(c3)oc3cc(-c5nc(-c6ccccc6)nc(-c6ccccc6)n5)ccc34)cc12 ; CC1(C)CCC2(CC1)c1ccccc1-c1ccc(-c3ccc4c(c3)oc3ccc(-c5nc(-c6ccccc6)nc(-c6ccccc6)n5)cc34)cc12 ; CC1(C)CCC2(CC1)c1ccccc1-c1ccc(-c3ccc4c(c3)oc3cc(-c5nc(-c6ccccc6)nc(-c6ccc(-c7ccccc7)cc6)n5)ccc34)cc12 ; CC1(C)CCC2(CC1)c1ccccc1-c1ccc(-c3ccc4c(c3)sc3cc(-c5nc(-c6ccccc6)nc(-c6ccc(-c7ccccc7)cc6)n5)ccc34)cc12]

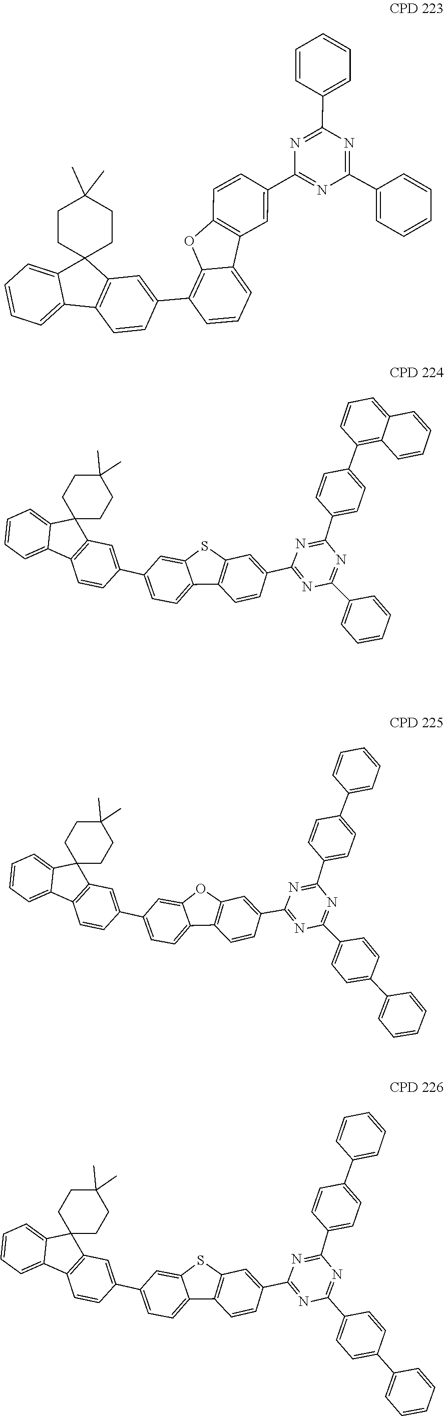 [SMILES: CC1(C)CCC2(CC1)c1ccccc1-c1ccc(-c3cccc4c3oc3ccc(-c5nc(-c6ccccc6)nc(-c6ccccc6)n5)cc34)cc12 ; CC1(C)CCC2(CC1)c1ccccc1-c1ccc(-c3ccc4c(c3)sc3cc(-c5nc(-c6ccccc6)nc(-c6ccc(-c7cccc8ccccc78)cc6)n5)ccc34)cc12 ; CC1(C)CCC2(CC1)c1ccccc1-c1ccc(-c3ccc4c(c3)oc3cc(-c5nc(-c6ccc(-c7ccccc7)cc6)nc(-c6ccc(-c7ccccc7)cc6)n5)ccc34)cc12 ; CC1(C)CCC2(CC1)c1ccccc1-c1ccc(-c3ccc4c(c3)sc3cc(-c5nc(-c6ccc(-c7ccccc7)cc6)nc(-c6ccc(-c7ccccc7)cc6)n5)ccc34)cc12]

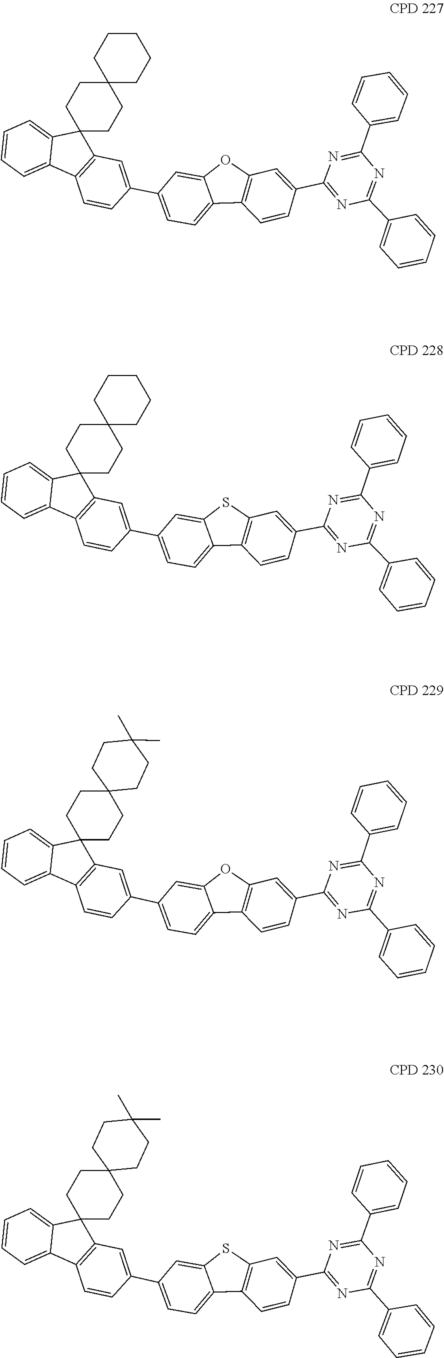 [SMILES: c1ccc(-c2nc(-c3ccccc3)nc(-c3ccc4c(c3)oc3cc(-c5ccc6c(c5)C5(CCC7(CCCCC7)CC5)c5ccccc5-6)ccc34)n2)cc1 ; c1ccc(-c2nc(-c3ccccc3)nc(-c3ccc4c(c3)sc3cc(-c5ccc6c(c5)C5(CCC7(CCCCC7)CC5)c5ccccc5-6)ccc34)n2)cc1 ; CC1(C)CCC2(CC1)CCC1(CC2)c2ccccc2-c2ccc(-c3ccc4c(c3)oc3cc(-c5nc(-c6ccccc6)nc(-c6ccccc6)n5)ccc34)cc21 ; CC1(C)CCC2(CC1)CCC1(CC2)c2ccccc2-c2ccc(-c3ccc4c(c3)sc3cc(-c5nc(-c6ccccc6)nc(-c6ccccc6)n5)ccc34)cc21]

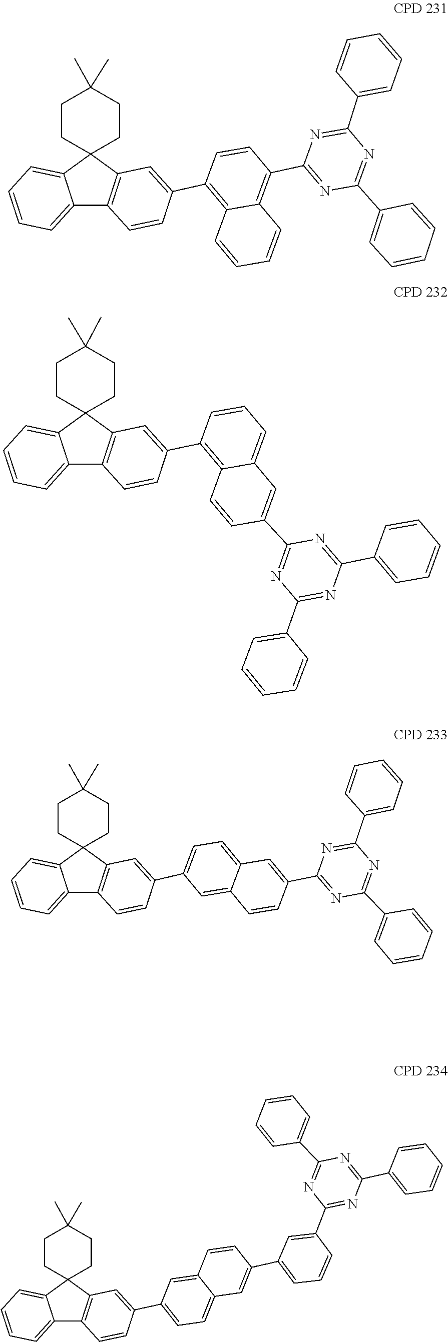 [SMILES: CC1(C)CCC2(CC1)c1ccccc1-c1ccc(-c3ccc(-c4nc(-c5ccccc5)nc(-c5ccccc5)n4)c4ccccc34)cc12 ; CC1(C)CCC2(CC1)c1ccccc1-c1ccc(-c3cccc4cc(-c5nc(-c6ccccc6)nc(-c6ccccc6)n5)ccc34)cc12 ; CC1(C)CCC2(CC1)c1ccccc1-c1ccc(-c3ccc4cc(-c5nc(-c6ccccc6)nc(-c6ccccc6)n5)ccc4c3)cc12 ; CC1(C)CCC2(CC1)c1ccccc1-c1ccc(-c3ccc4cc(-c5cccc(-c6nc(-c7ccccc7)nc(-c7ccccc7)n6)c5)ccc4c3)cc12]

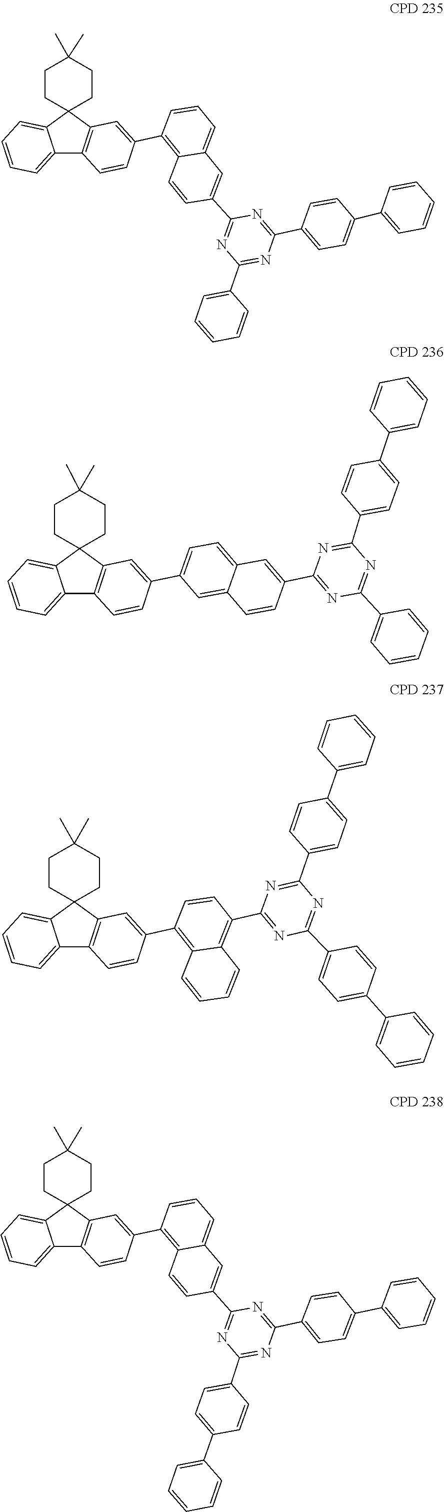 [SMILES: CC1(C)CCC2(CC1)c1ccccc1-c1ccc(-c3cccc4cc(-c5nc(-c6ccccc6)nc(-c6ccc(-c7ccccc7)cc6)n5)ccc34)cc12 ; CC1(C)CCC2(CC1)c1ccccc1-c1ccc(-c3ccc4cc(-c5nc(-c6ccccc6)nc(-c6ccc(-c7ccccc7)cc6)n5)ccc4c3)cc12 ; CC1(C)CCC2(CC1)c1ccccc1-c1ccc(-c3ccc(-c4nc(-c5ccc(-c6ccccc6)cc5)nc(-c5ccc(-c6ccccc6)cc5)n4)c4ccccc34)cc12 ; CC1(C)CCC2(CC1)c1ccccc1-c1ccc(-c3cccc4cc(-c5nc(-c6ccc(-c7ccccc7)cc6)nc(-c6ccc(-c7ccccc7)cc6)n5)ccc34)cc12]

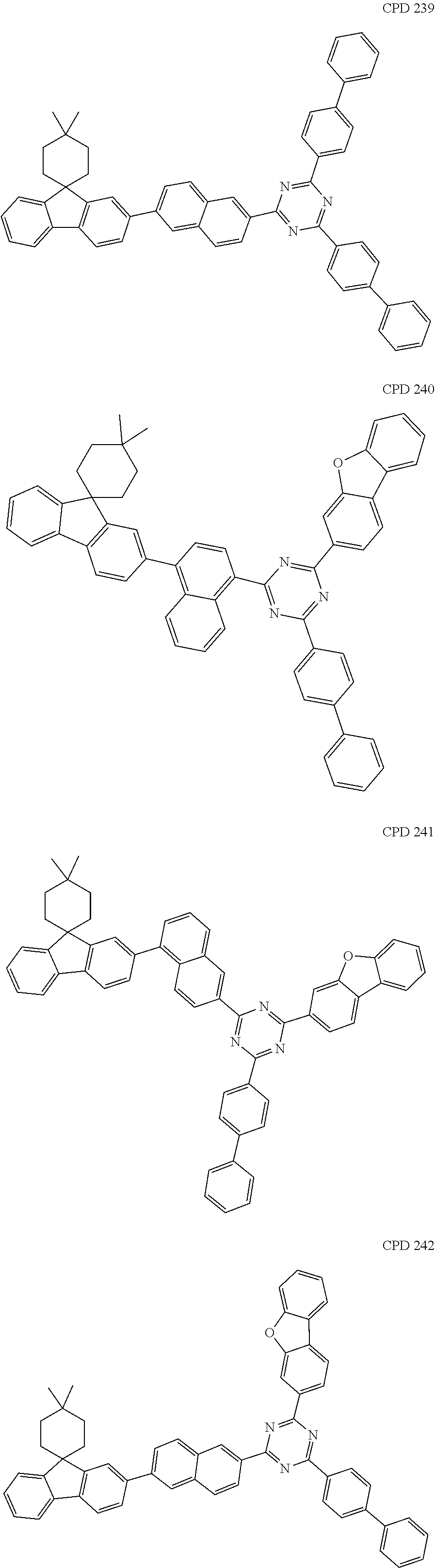 [SMILES: CC1(C)CCC2(CC1)c1ccccc1-c1ccc(-c3ccc4cc(-c5nc(-c6ccc(-c7ccccc7)cc6)nc(-c6ccc(-c7ccccc7)cc6)n5)ccc4c3)cc12 ; CC1(C)CCC2(CC1)c1ccccc1-c1ccc(-c3ccc(-c4nc(-c5ccc(-c6ccccc6)cc5)nc(-c5ccc6c(c5)oc5ccccc56)n4)c4ccccc34)cc12 ; CC1(C)CCC2(CC1)c1ccccc1-c1ccc(-c3cccc4cc(-c5nc(-c6ccc(-c7ccccc7)cc6)nc(-c6ccc7c(c6)oc6ccccc67)n5)ccc34)cc12 ; CC1(C)CCC2(CC1)c1ccccc1-c1ccc(-c3ccc4cc(-c5nc(-c6ccc(-c7ccccc7)cc6)nc(-c6ccc7c(c6)oc6ccccc67)n5)ccc4c3)cc12]

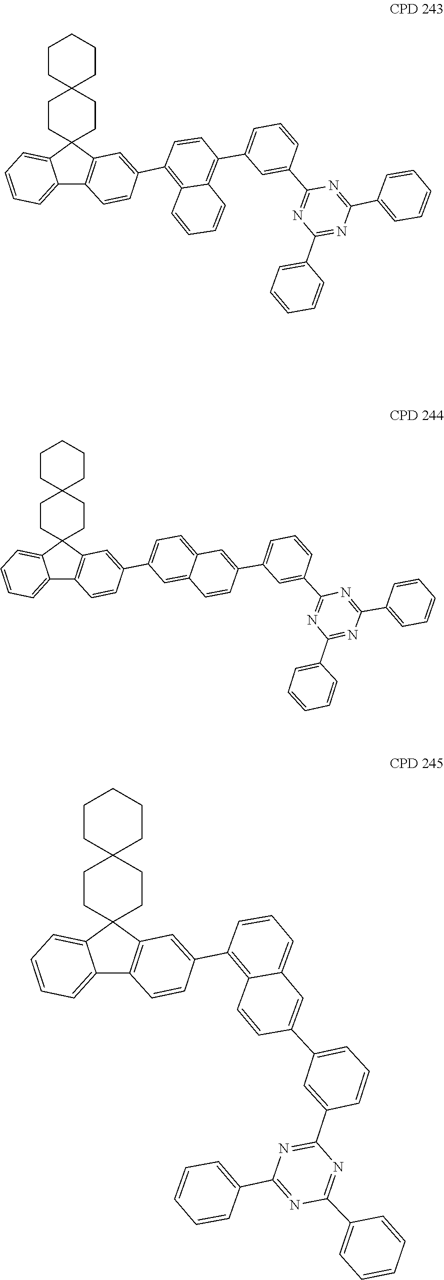 [SMILES: c1ccc(-c2nc(-c3ccccc3)nc(-c3cccc(-c4ccc(-c5ccc6c(c5)C5(CCC7(CCCCC7)CC5)c5ccccc5-6)c5ccccc45)c3)n2)cc1 ; c1ccc(-c2nc(-c3ccccc3)nc(-c3cccc(-c4ccc5cc(-c6ccc7c(c6)C6(CCC8(CCCCC8)CC6)c6ccccc6-7)ccc5c4)c3)n2)cc1 ; c1ccc(-c2nc(-c3ccccc3)nc(-c3cccc(-c4ccc5c(-c6ccc7c(c6)C6(CCC8(CCCCC8)CC6)c6ccccc6-7)cccc5c4)c3)n2)cc1]

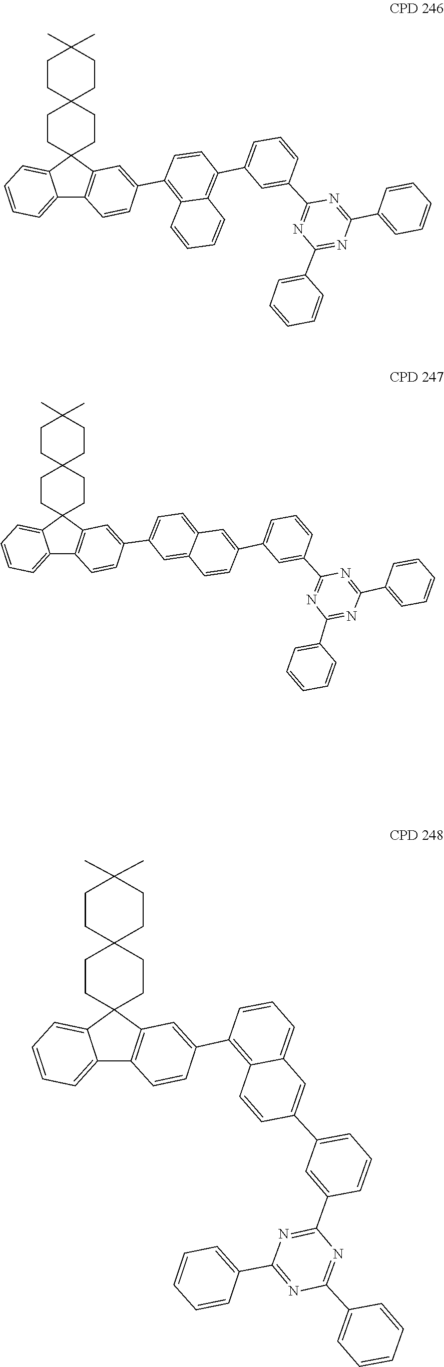 [SMILES: CC1(C)CCC2(CC1)CCC1(CC2)c2ccccc2-c2ccc(-c3ccc(-c4cccc(-c5nc(-c6ccccc6)nc(-c6ccccc6)n5)c4)c4ccccc34)cc21 ; CC1(C)CCC2(CC1)CCC1(CC2)c2ccccc2-c2ccc(-c3ccc4cc(-c5cccc(-c6nc(-c7ccccc7)nc(-c7ccccc7)n6)c5)ccc4c3)cc21 ; CC1(C)CCC2(CC1)CCC1(CC2)c2ccccc2-c2ccc(-c3cccc4cc(-c5cccc(-c6nc(-c7ccccc7)nc(-c7ccccc7)n6)c5)ccc34)cc21]

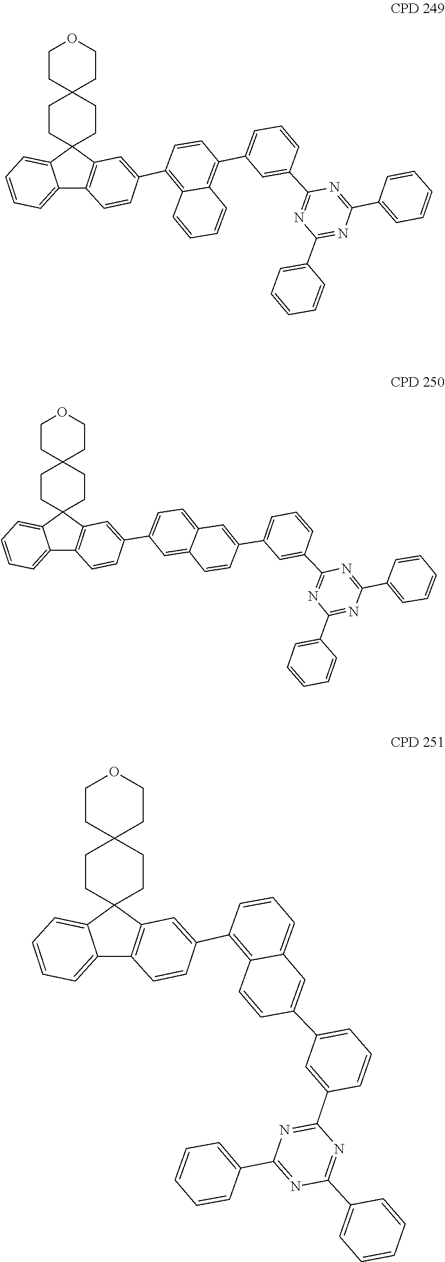 [SMILES: c1ccc(-c2nc(-c3ccccc3)nc(-c3cccc(-c4ccc(-c5ccc6c(c5)C5(CCC7(CCOCC7)CC5)c5ccccc5-6)c5ccccc45)c3)n2)cc1 ; c1ccc(-c2nc(-c3ccccc3)nc(-c3cccc(-c4ccc5cc(-c6ccc7c(c6)C6(CCC8(CCOCC8)CC6)c6ccccc6-7)ccc5c4)c3)n2)cc1 ; c1ccc(-c2nc(-c3ccccc3)nc(-c3cccc(-c4ccc5c(-c6ccc7c(c6)C6(CCC8(CCOCC8)CC6)c6ccccc6-7)cccc5c4)c3)n2)cc1]

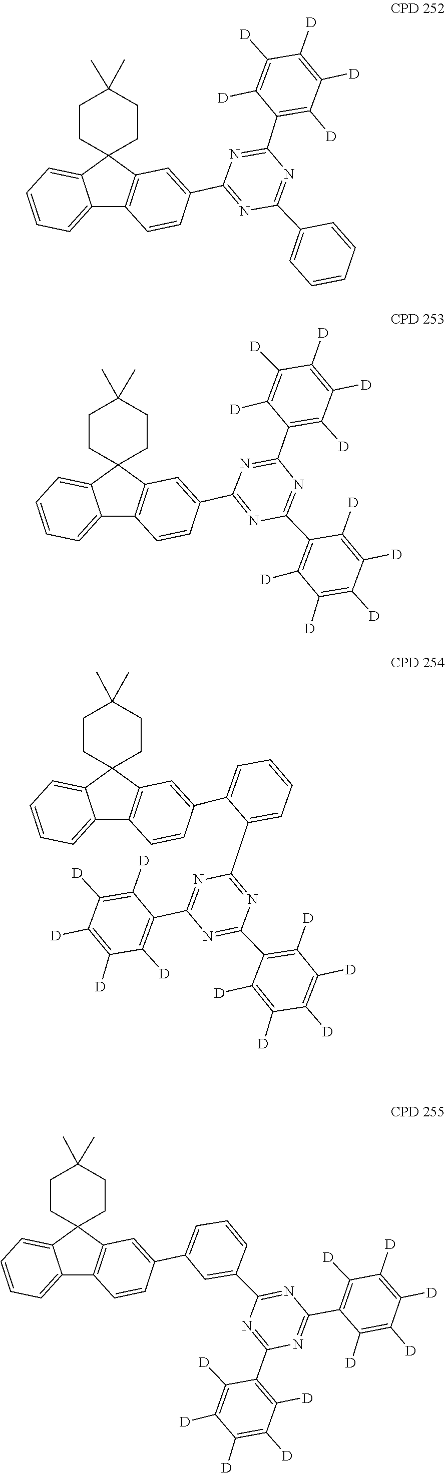 [SMILES: [2H]c1c([2H])c([2H])c(-c2nc(-c3ccccc3)nc(-c3ccc4c(c3)C3(CCC(C)(C)CC3)c3ccccc3-4)n2)c([2H])c1[2H] ; [2H]c1c([2H])c([2H])c(-c2nc(-c3ccc4c(c3)C3(CCC(C)(C)CC3)c3ccccc3-4)nc(-c3c([2H])c([2H])c([2H])c([2H])c3[2H])n2)c([2H])c1[2H] ; [2H]c1c([2H])c([2H])c(-c2nc(-c3ccccc3-c3ccc4c(c3)C3(CCC(C)(C)CC3)c3ccccc3-4)nc(-c3c([2H])c([2H])c([2H])c([2H])c3[2H])n2)c([2H])c1[2H] ; [2H]c1c([2H])c([2H])c(-c2nc(-c3cccc(-c4ccc5c(c4)C4(CCC(C)(C)CC4)c4ccccc4-5)c3)nc(-c3c([2H])c([2H])c([2H])c([2H])c3[2H])n2)c([2H])c1[2H]]

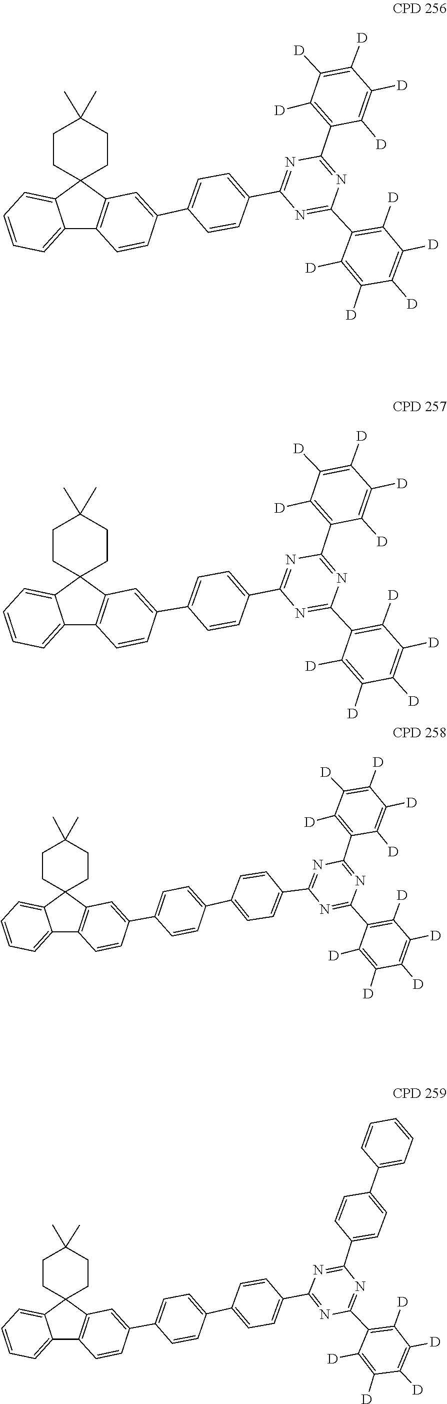

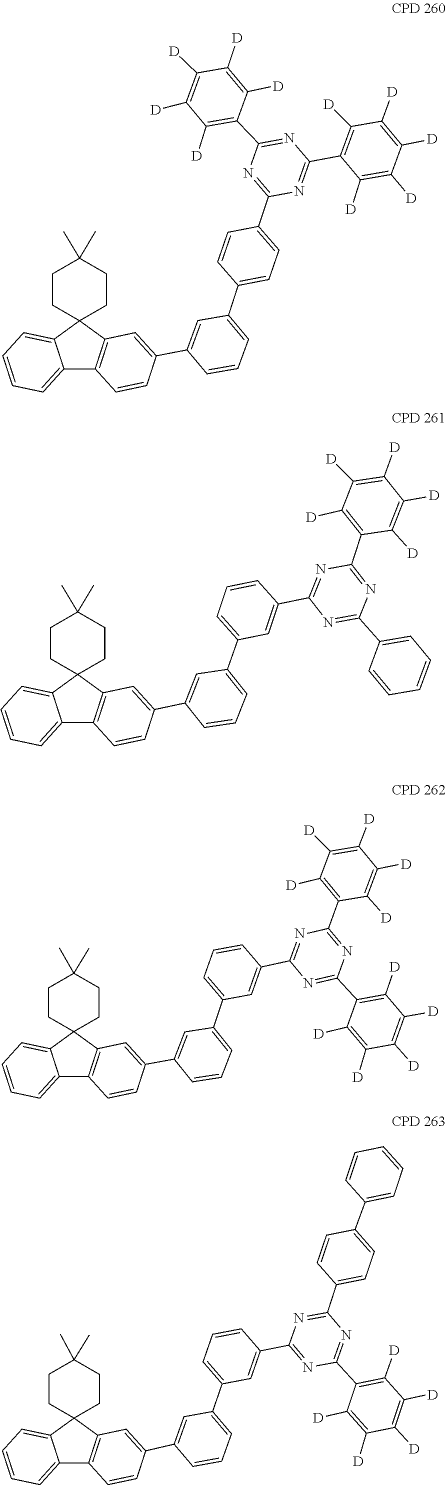

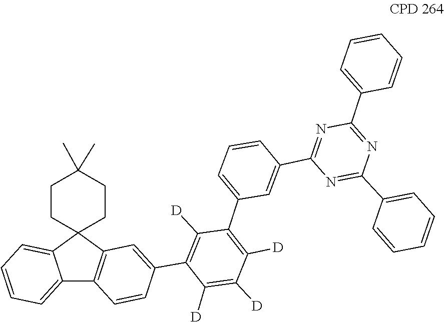 [SMILES: [2H]c1c([2H])c(-c2cccc(-c3nc(-c4ccccc4)nc(-c4ccccc4)n3)c2)c([2H])c(-c2ccc3c(c2)C2(CCC(C)(C)CC2)c2ccccc2-3)c1[2H]]

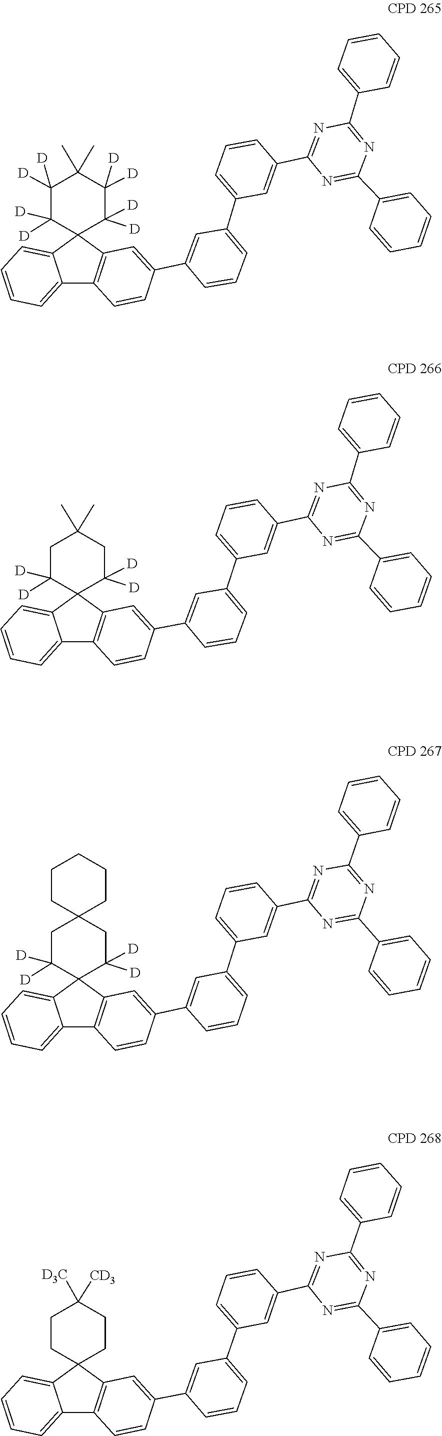 [SMILES: [2H]C1([2H])C([2H])([2H])C2(c3ccccc3-c3ccc(-c4cccc(-c5cccc(-c6nc(-c7ccccc7)nc(-c7ccccc7)n6)c5)c4)cc32)C([2H])([2H])C([2H])([2H])C1(C)C ; [2H]C1([2H])CC(C)(C)CC([2H])([2H])C12c1ccccc1-c1ccc(-c3cccc(-c4cccc(-c5nc(-c6ccccc6)nc(-c6ccccc6)n5)c4)c3)cc12 ; [2H]C1([2H])CC2(CCCCC2)CC([2H])([2H])C12c1ccccc1-c1ccc(-c3cccc(-c4cccc(-c5nc(-c6ccccc6)nc(-c6ccccc6)n5)c4)c3)cc12 ; [2H]C([2H])([2H])C1(C([2H])([2H])[2H])CCC2(CC1)c1ccccc1-c1ccc(-c3cccc(-c4cccc(-c5nc(-c6ccccc6)nc(-c6ccccc6)n5)c4)c3)cc12]

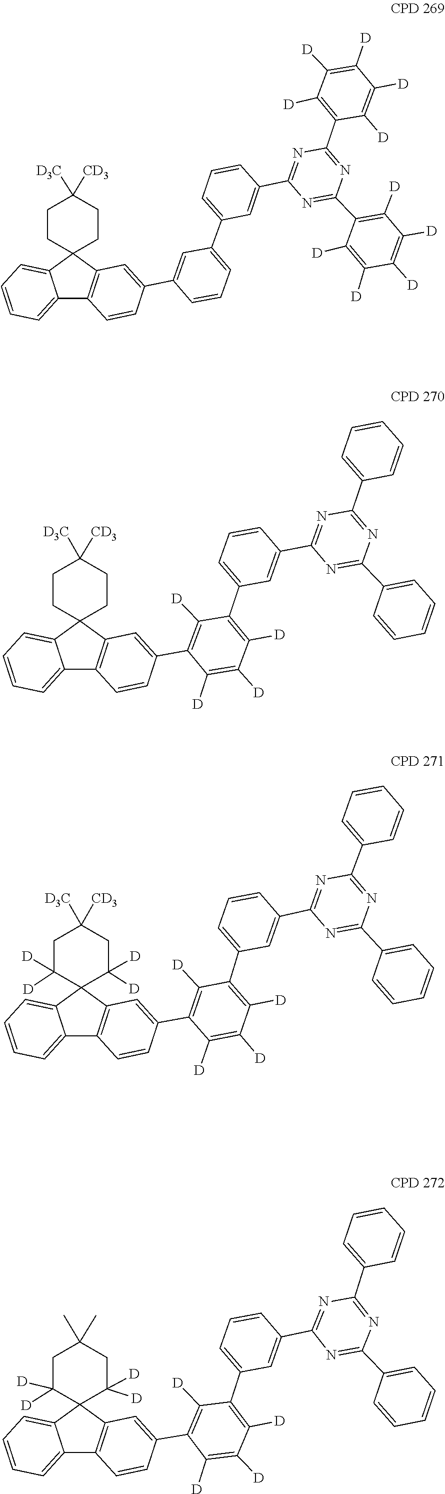

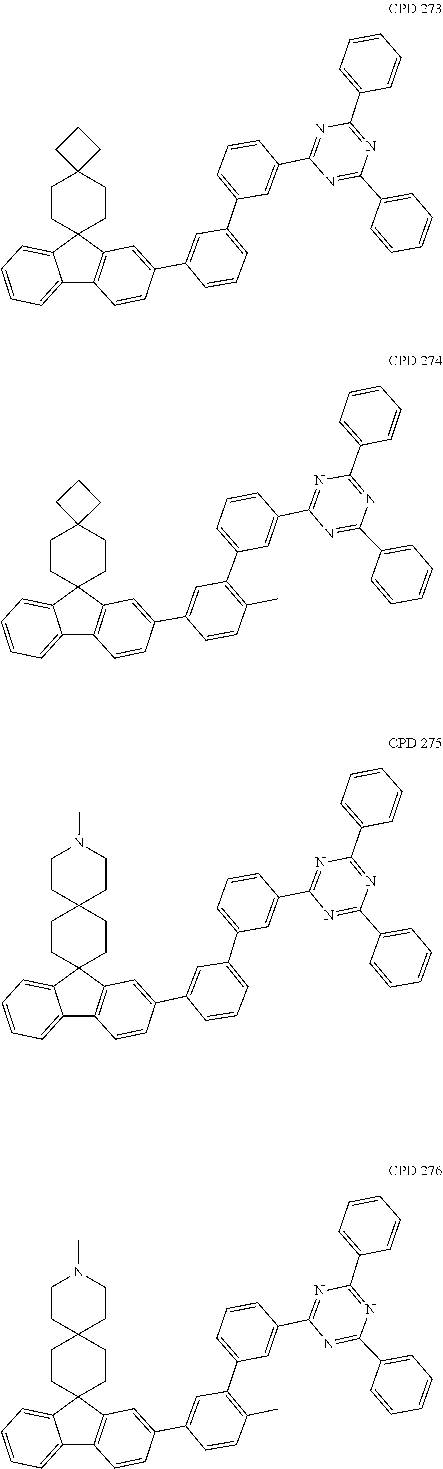 [SMILES: c1ccc(-c2nc(-c3ccccc3)nc(-c3cccc(-c4cccc(-c5ccc6c(c5)C5(CCC7(CCC7)CC5)c5ccccc5-6)c4)c3)n2)cc1 ; Cc1ccc(-c2ccc3c(c2)C2(CCC4(CCC4)CC2)c2ccccc2-3)cc1-c1cccc(-c2nc(-c3ccccc3)nc(-c3ccccc3)n2)c1 ; CN1CCC2(CC1)CCC1(CC2)c2ccccc2-c2ccc(-c3cccc(-c4cccc(-c5nc(-c6ccccc6)nc(-c6ccccc6)n5)c4)c3)cc21 ; Cc1ccc(-c2ccc3c(c2)C2(CCC4(CCN(C)CC4)CC2)c2ccccc2-3)cc1-c1cccc(-c2nc(-c3ccccc3)nc(-c3ccccc3)n2)c1]

One of the objectives of the present disclosure is also to provide a light-emitting device comprising the above compound, which includes an anode, a cathode, and one or more organic layers between the anode and the cathode, and at least one of the one or more organic layers includes the compound.

The material of the present disclosure severs as a material in at least one of alight-emitting layer, a hole blocking layer and an electron transport layer in the organic layer.

The material of the present disclosure is used in a light-emitting device as a hole blocking layer material.

The light-emitting device of the present disclosure can be an organic light-emitting device or a quantum dot light-emitting device. In some embodiments, when the light-emitting device is an organic light-emitting device, the compound can be used as a material for a light-emitting layer, a hole blocking layer, or an electron transport layer; and when the light-emitting device is a quantum dot light-emitting device, the compound severs as a material for a hole blocking layer or an electron transport layer.

In some embodiments, the light-emitting device is an organic electroluminescent device.

The material of the present disclosure has the advantages of good film-forming properties, good optical, electrical and heat stabilities, high luminous efficiency, low voltage and long lifetime, etc., and can be used in an organic light-emitting device. In particular, it is expected to be applied to the AMOLED industry as a hole blocking layer material and an electron transport layer material.

### BRIEF DESCRIPTION OF THE DRAWINGS

FIG. 1 is the ¹HNMR spectrum of the compound CPD 108; and

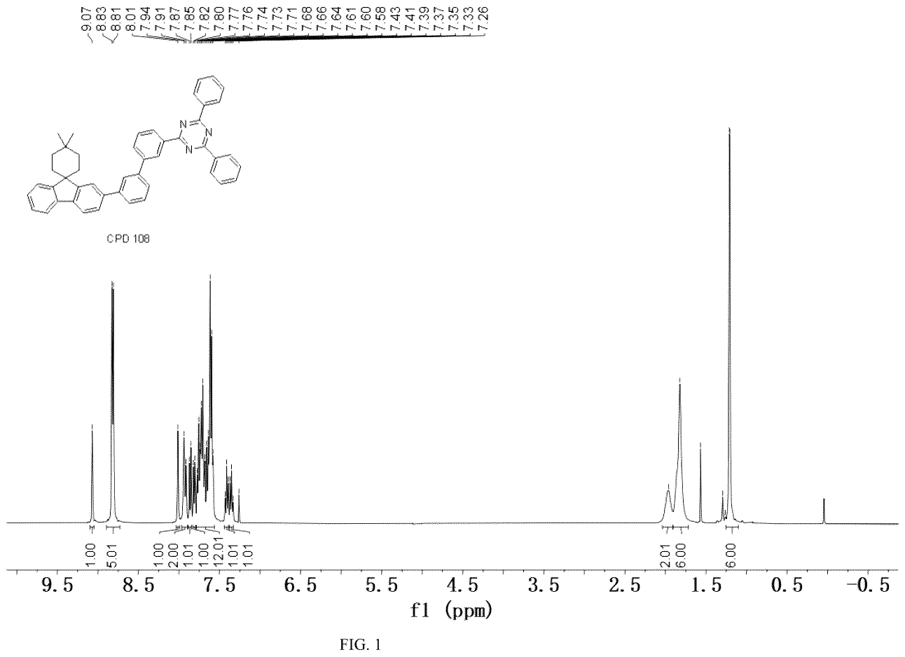

FIG. 2 is a schematic structural diagram of an organic light-emitting device according to an embodiment of the present disclosure.

### DETAILED DESCRIPTION

A compound includes a structure of formula (1):

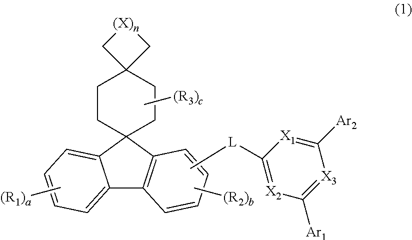

wherein X is independently selected from the group consisting of C(R₄)₂, O, S, and NR₅; wherein n is independently selected from 0 or an integer of 1-6, when n=0, a ring is not formed through connection; when n≥1, a ring is formed through connection; and when n≥2, each X is the same or different.

As an embodiment of the present disclosure, when n=0, a ring is not formed through connection and X is absent; when n≥1, a ring is formed through connection; when n≥2, X is a plurality of C(R₄)₂, or a combination of one or more C(R₄)₂ and one of O, S or NR₅;
wherein X₁, X₂ and X₃ are independently selected from N or CR₆, and at least one of X₁, X₂ and X₃ is N;
R₁, R₂ and R₃ are each independently selected from the group consisting of deuterium, halogen, cyano, nitro, C1-C40 alkyl, C1-C40 heteroalkyl, C2-C40 alkenyl, C2-C40 alkynyl, C3-C40 cycloalkyl, C3-C40 heterocycloalkyl, C6-C60 aryl, C5-C60 heteroaryl, C1-C40 alkoxy, C6-C60 aryloxy, C3-C40 alkylsilyl, C6-C60 arylsilyl, C1-C40 alkylboryl, C6-C60 arylboryl, C6-C60 monoarylphosphino, C6-C60 diarylphosphino, or C6-C60 arylamino;
R₄, R₅ and R₆ are each independently selected from the group consisting of hydrogen, deuterium, halogen, cyano, nitro, C1-C40 alkyl, C1-C40 heteroalkyl, C2-C40 alkenyl, C2-C40 alkynyl, C3-C40 cycloalkyl, C3-C40 heterocycloalkyl, C6-C60 aryl, C5-C60 heteroaryl, C1-C40 alkoxy, C6-C60 aryloxy, C3-C40 alkylsilyl, C6-C60 arylsilyl, C1-C40 alkylboryl, C6-C60 arylboryl, C6-C60 monoarylphosphino, C6-C60 diarylphosphino, or C6-C60 arylamino;
L is selected from a single bond, or a substituted or unsubstituted aromatic or heteroaromatic group with 6 to 30 ring atoms;
wherein a, b, and c are each independently selected from an integer of 0 to 8; and R₁, R₂, and R₃ are substituents on respective rings; when a, b, and c are 0, the corresponding positions on each rings are hydrogen and there is no substituent;
wherein Ar₁ and Ar₂ are each independently selected from the group consisting of a substituted or unsubstituted C6-C30 aryl, or a substituted or unsubstituted C2-C30 heteroaryl;
a heteroatom in the heteroaryl, or heteroalkyl or heterocycloalkyl is selected from at least one heteroatom of O, S, N, Se, Si and Ge; and
the substitution refers to being substituted with deuterium, F, Cl, Br, C1-C6 alkyl, C3-C6 cycloalkyl, C1-C6 alkyl substituted amino, C6-C12 aryl, cyano, isocyano or phosphino, and the amount of substitutions of each substituent group is independently selected from one of single substitution to the maximum amount of substitutions.

The embodiments are only for facilitating the understanding of the technical invention and should not be regarded as specific limitations of the present disclosure.

The raw materials and solvents, etc., involved in the synthesis of the compounds in the present disclosure were purchased from suppliers well known to those skilled in the art, such as Alfa and Acros, etc.

### Examples

### 1. Synthesis Route of Compound CPD 4

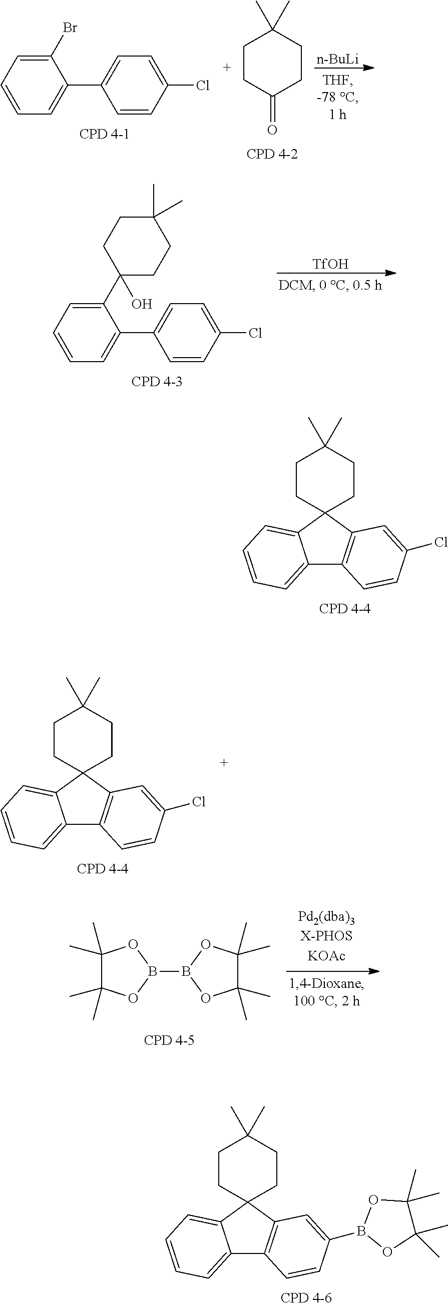

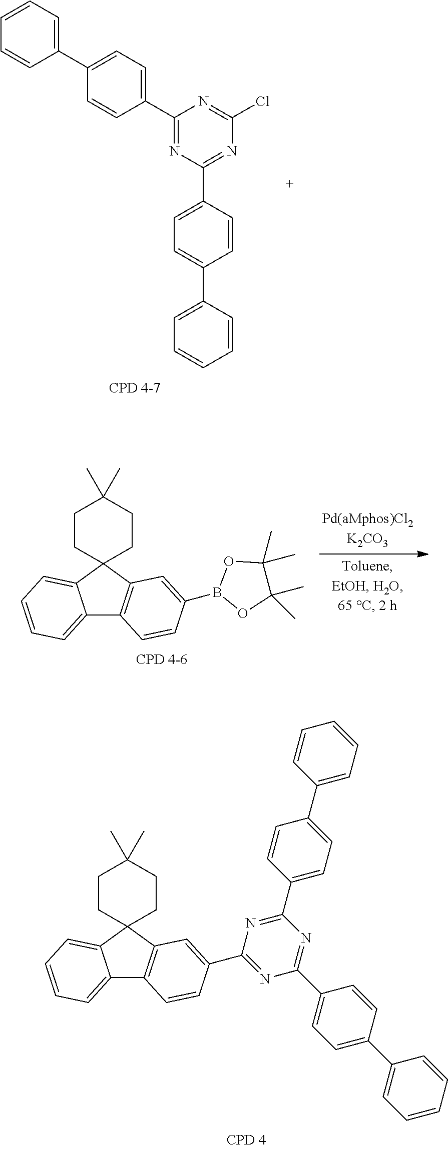 [SMILES: Clc1nc(-c2ccc(-c3ccccc3)cc2)nc(-c2ccc(-c3ccccc3)cc2)n1 ; CC1(C)CCC2(CC1)c1ccccc1-c1ccc(B3OC(C)(C)C(C)(C)O3)cc12 ; CC1(C)CCC2(CC1)c1ccccc1-c1ccc(-c3nc(-c4ccc(-c5ccccc5)cc4)nc(-c4ccc(-c5ccccc5)cc4)n3)cc12]

Synthesis of compound CPD 4-3

Compound CPD 4-1 (50.00 g, 186.88 mmol) and dry tetrahydrofuran (750 ml) were added to a 2,000 ml three-necked round-bottom flask, the system was replaced with nitrogen under vacuum for three times, next it was cooled to −78 °C, then a solution of n-butyllithium in n-hexane (121.47 ml, 242.95 mmol, a concentration of 2 mol/L) was added dropwise, while the inner temperature of the system was controlled to be no higher than −70 °C, the addition was completed over 1 hour, and the mixture was maintained at −78 °C and stirred for 1 hour. Finally, CPD 4-2 (33.02 g, 261.63 mmol) was added in one portion, and the mixture was maintained at −78 °C and stirred continuously for 1.5 hours. The reaction was monitored by TLC (ethyl acetate:n-hexane=1:30 as the developing solvent) until the raw material CPD 4-1 was consumed completely and most of CPD 4-3 was generated. Deionized water (300 ml) was added dropwise to the system to quench the reaction, then the system was raised to room temperature, the liquid separation was carried out directly, the aqueous phase was extracted once with ethyl acetate (500 ml), the organic phases were combined and concentrated under reduced pressure at 65 °C for 1 hour to obtain a light yellow solid; the sample was mixed with silica gel and dry-loaded onto a chromatography column, which was purified through silica gel column chromatography (200-300 mesh of silica gel, ethyl acetate:n-hexane=1:30 as eluent); after elution, the mixture was concentrated under reduced pressure at 65 °C for 1.5 hours to obtain CPD 4-3 as a white solid (38.51 g, purity: 99.78%, yield: 65.45%), with a mass spectrum: 315.15 (M+H).

### Synthesis of Compound CPD 4-4

CPD 4-3 (37.00 g, 117.52 mmol) and dichloromethane (370 ml) were added to a 1,000 ml three-necked round-bottom flask, next the system was cooled to 0 °C and trifluoromethanesulfonic acid (35.27 g, 235.03 mmol) was added dropwise over 15 minutes, the system was maintained at that temperature and stirred for 30 minutes. The reaction was monitored by TLC (ethyl acetate:n-hexane=1:30 as developing solvent) until the raw material CPD 4-3 was consumed completely. Deionized water (100 ml) was added dropwise to the system to quench the reaction, then the liquid separation was carried out, the sample was mixed with silica gel and dry-loaded onto a chromatography column, which was purified through silica gel column chromatography (200-300 mesh of silica gel, ethyl acetate:n-hexane=1:50 as eluent); after elution, the mixture was concentrated under reduced pressure at 65 °C for 2 hours to obtain CPD 4-4 as a white solid (29.92 g, purity: 99.91%, yield: 85.76%), with a mass spectrum: 297.12 (M+H).

### Synthesis of Compound CPD 4-6

CPD 4-4 (35.00 g, 117.91 mmol), CPD 4-5 (35.93 g, 141.49 mmol), tris(dibenzylideneacetone)dipalladium (2.16 g, 2.36 mmol), 2-dicyclohexylphosphino-2′,4′,6′-triisopropylbiphenyl (2.25 g, 4.72 mmol), potassium acetate (23.14 g, 235.82 mmol), and 1,4-dioxane (350 ml) were added to a 1,000 ml three-necked round-bottom flask, next the system was replaced with nitrogen under vacuum for three times, then the system was heated to 100 °C and reacted for 2 hours. The reaction was monitored by TLC (ethyl acetate:n-hexane=1:10 as developing agent) until the raw material CPD 4-4 was consumed completely. The reaction was cooled to 60 °C, and the solvent was removed by concentration under reduced pressure, then ethyl acetate (800 ml) was added, and the solution was washed with deionized water (300 ml×3), then liquid separation was carried out, the sample was mixed with silica gel and dry-loaded onto a chromatography column, which was purified through silica gel column chromatography (200-300 mesh of silica gel, ethyl acetate:n-hexane=1:15 as eluent); after elution, the mixture was concentrated under reduced pressure at 65 °C for 1 hours to obtain CPD 4-6 as a white solid (38.95 g, purity: 99.00%, yield: 85.06%), with a mass spectrum: 389.26 (M+H).

### Synthesis of Compound CPD 4

CPD 4-7 (20.00 g, 47.63 mmol), CPD 4-6 (19.42 g, 50.01 mmol), bis(di-tert-butyl(4-dimethylaminophenyl)phosphine)dichloropalladium (0.697 g, 0.95 mmol), potassium carbonate (13.16 g, 95.26 mmol), toluene (300 ml), ethanol (100 ml) and deionized water (100 ml) were added to a 1,000 ml three-necked round-bottom flask, and the system was replaced with nitrogen under vacuum for three times, then heated to 65 °C and reacted for 2 hours. The reaction was monitored by TLC (ethyl acetate:n-hexane=1:10 as developing agent) until the raw material CPD 4-7 was consumed completely. The reaction was reduced to room temperature, then methanol (300 ml) was added, the mixture was stirred at room temperature for 1 hour, and a large amount of solid precipitated. Toluene (450 ml) was added, and the system was heated to 100 °C for dissolution, then lowered to room temperature, and filtered through 300-400 mesh of silica gel (50 g) once, methanol (450 ml) was added to the filtrate at room temperature, the mixture was stirred at room temperature for 1 hour, suction filtered to obtain a wet product as a white solid, which was dried at 80 °C for 1 hour to obtain 25.38 g of white solid; the above solid was added into a 100 ml single-necked round bottom flask, and recrystallized once using toluene (254 ml) and methanol (127 ml), then suction filtered, the filter cake was vacuum dried at 80 °C for 5 hours to obtain CPD 4 as a white solid (22.18 g, purity: 99.96%, yield: 72.11%). 22.18 g of crude CPD 4 was purified by sublimation to obtain the sublimation-purified CPD 4 (17.96 g, purity: 99.97%, yield: 80.98%), with a mass spectrum: 646.22 (M+H). ¹H NMR (400 MHz, CDCl₃) δ 8.35-8.33 (m, 1H), 8.30-8.22 ((m, 5H), 8.13-8.11 (m, 1H), 8.07 (s, 1H), 8.04-8.00 (m, 4H), 7.92-7.88 (m, 4H), 7.82-7.79 (m, 1H), 7.76-7.73 (m, 6H), 7.69-7.61 (m, 2H), 2.61-2.58 (m, 2H), 2.36-2.30 (m, 2H), 1.91-1.88 (m, 4H), 1.20 (s, 6H).

### 2. Synthesis Route of Compound CPD 34

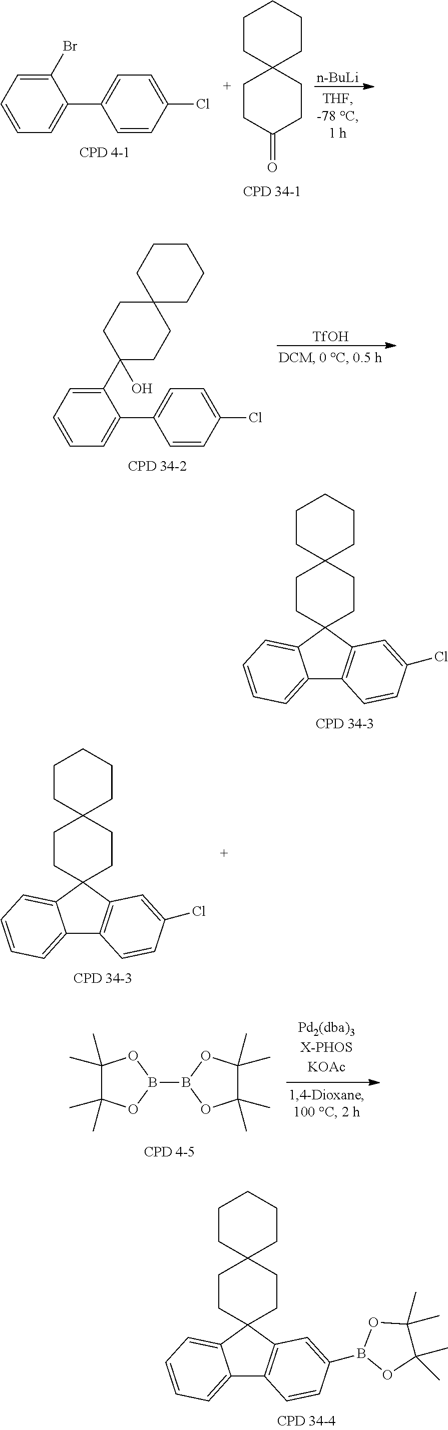

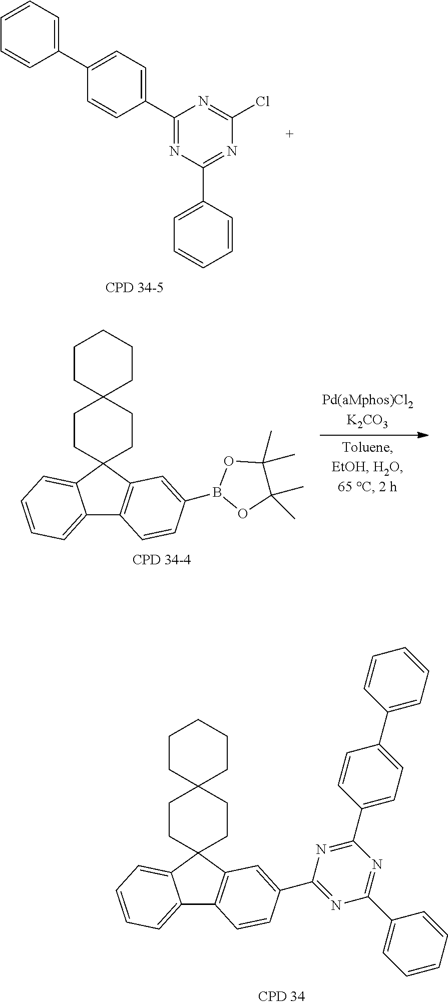 [SMILES: Clc1nc(-c2ccccc2)nc(-c2ccc(-c3ccccc3)cc2)n1 ; CC1(C)OB(c2ccc3c(c2)C2(CCC4(CCCCC4)CC2)c2ccccc2-3)OC1(C)C ; c1ccc(-c2ccc(-c3nc(-c4ccccc4)nc(-c4ccc5c(c4)C4(CCC6(CCCCC6)CC4)c4ccccc4-5)n3)cc2)cc1]

### Synthesis of Compound CPD 34-2

Referring to the synthesis and purification methods of compound CPD 4-3, it was only necessary to change the corresponding raw materials to obtain the target compound CPD 34-2 (35.12 g, purity: 99.81%, yield: 66.35%), with a mass spectrum: 355.12 (M+H).

### Synthesis of Compound CPD 34-3

Referring to the synthesis and purification methods of compound CPD 4-4, it was only necessary to change the corresponding raw materials to obtain the target compound CPD 34-3 (27.56 g, purity: 99.91%, yield: 84.36%), with a mass spectrum: 337.42 (M+H).

### Synthesis of Compound CPD 34-4

Referring to the synthesis and purification methods of compound CPD 4-6, it was only necessary to change the corresponding raw materials to obtain the target compound CPD 34-4 (29.64 g, purity: 99.87%, yield: 86.33%), with a mass spectrum: 429.02 (M+H).

### Synthesis of Compound CPD 34

Referring to the synthesis and purification methods of compound CPD 4, it was only necessary to change the corresponding raw materials to obtain the target compound CPD 34 (38.45 g, purity: 99.93%, yield: 74.12%). 38.45 g of crude CPD 34 was purified by sublimation to obtain the sublimation-purified CPD 34 (31.68 g, purity: 99.94%, yield: 82.40%), with a mass spectrum: 610.32 (M+H). ¹H NMR (400 MHz, CDCl₃) δ 8.12-8.04 (m, 2H), 8.00 (dd, J=9.6, 2.1 Hz, 1H), 7.97-7.89 (m, 3H), 7.79 (dd, J=6.7, 1.1 Hz, 1H), 7.73 (d, J=2.0 Hz, 1H), 7.72-7.66 (m, 2H), 7.60-7.55 (m, 2H), 7.55-7.44 (m, 4H), 7.44-7.37 (m, 3H), 7.36-7.27 (m, 2H), 2.38-2.33 (m, 2H), 2.13-2.08 (m, 2H), 1.71 (t, J=6.1 Hz, 4H), 1.52-1.45 (m, 4H), 1.43-1.33 (m, 6H).

### 3. Synthesis of Compound CPD 63

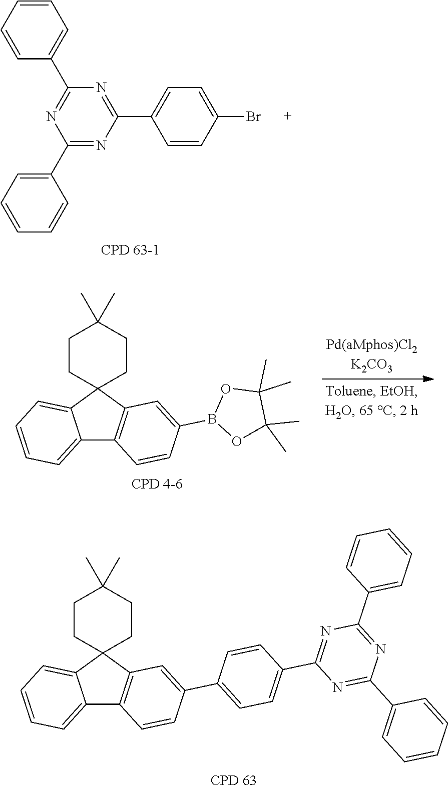 [SMILES: Brc1ccc(-c2nc(-c3ccccc3)nc(-c3ccccc3)n2)cc1 ; CC1(C)CCC2(CC1)c1ccccc1-c1ccc(B3OC(C)(C)C(C)(C)O3)cc12 ; CC1(C)CCC2(CC1)c1ccccc1-c1ccc(-c3ccc(-c4nc(-c5ccccc5)nc(-c5ccccc5)n4)cc3)cc12]

Referring to the synthesis and purification methods of compound CPD 4, it was only necessary to change the corresponding raw materials to obtain the target compound CPD 63 (32.42 g, purity: 99.94%, yield: 76.32%). 32.42 g of crude CPD 63 was purified by sublimation to obtain the sublimation-purified CPD 63 (26.02 g, purity: 99.94%, yield: 80.26%), with a mass spectrum: 570.22 (M+H). ¹H NMR (400 MHz, CDCl₃) δ 8.12-8.04 (m, 4H), 8.02 (d, J=7.7 Hz, 1H), 7.97-7.91 (m, 2H), 7.80-7.78 (m, 1H), 7.72-7.64 (m, 3H), 7.55-7.44 (m, 8H), 7.36-7.27 (m, 2H), 2.28-2.22 (m, 2H), 2.02-1.97 (m, 2H), 1.56-1.53 (m, 4H), 1.21 (s, 6H).

### 4. Synthesis of Compound CPD 83

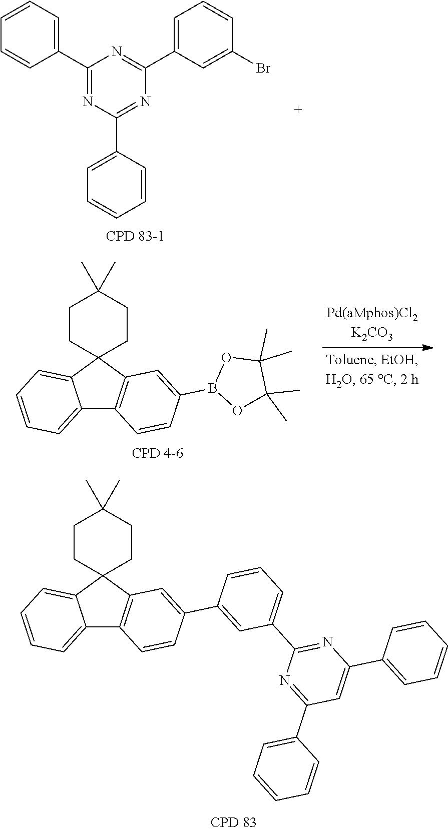

Referring to the synthesis and purification methods of compound CPD 4, it was only necessary to change the corresponding raw materials to obtain the target compound CPD 83 (28.88 g, purity: 99.95%, yield: 75.00%). 28.88 g of crude CPD 83 was purified by sublimation to obtain the sublimation-purified CPD 83 (22.80 g, purity: 99.95%, yield: 78.95%), with a mass spectrum: 570.22 (M+H). ¹H NMR (400 MHz, CDCl₃) δ 8.20 (t, J=2.1 Hz, 1H), 8.02 (d, J=7.9 Hz, 1H), 7.90-7.85 (m, 4H), 7.85-7.80 (m, 2H), 7.79-7.77 (m, 2H), 7.64-7.62 (m, 1H), 7.57-7.53 (m, 1H), 7.49-7.42 ((m, 6H), 7.36 (s, 1H), 7.36-7.27 (m, 2H), 2.28-2.22 (m, 2H), 2.02-1.97 (m, 2H), 1.56 (t, J=6.3 Hz, 4H), 1.20 (s, 6H).

### 5. Synthesis of Compound CPD 90

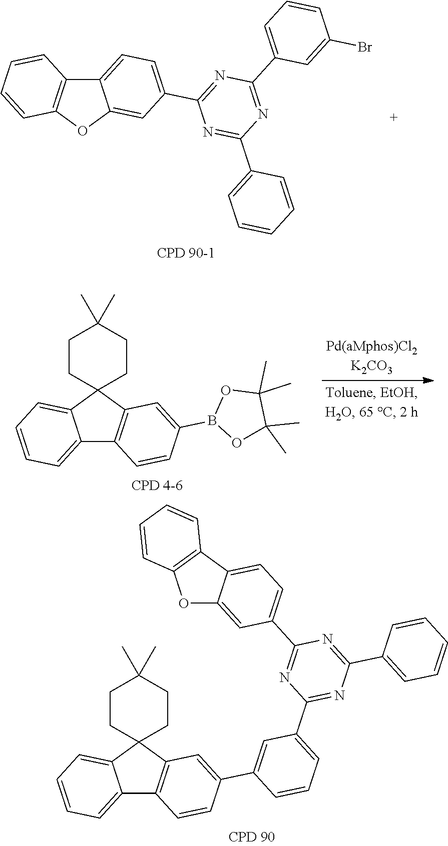

Referring to the synthesis and purification methods of compound CPD 4, it was only necessary to change the corresponding raw materials to obtain the target compound CPD 90 (29.06 g, purity: 99.93%, yield: 76.52%). 29.06 g of crude CPD 90 was purified by sublimation to obtain the sublimation-purified CPD 90 (22.56 g, purity: 99.94%, yield: 77.64%), with a mass spectrum: 660.00 (M+H). ¹H NMR (400 MHz, CDCl₃) δ 8.21 (t, J=2.1 Hz, 1H), 8.12-8.04 (m, 3H), 8.02 (d, J=7.9 Hz, 1H), 7.99-7.89 (m, 2H), 7.87-7.85 (m, 1H), 7.81-7.75 (m, 2H), 7.72 (d, J=2.2 Hz, 1H), 7.67-7.59 (m, 2H), 7.59-7.38 (m, 7H), 7.36 (s, 1H), 7.35-7.27 (m, 2H), 2.28-2.22 (m, 2H), 2.02-1.97 (m, 2H), 1.56 (t, J=6.3 Hz, 4H), 1.22 (s, 6H).

### 6. Synthesis of Compound CPD 108

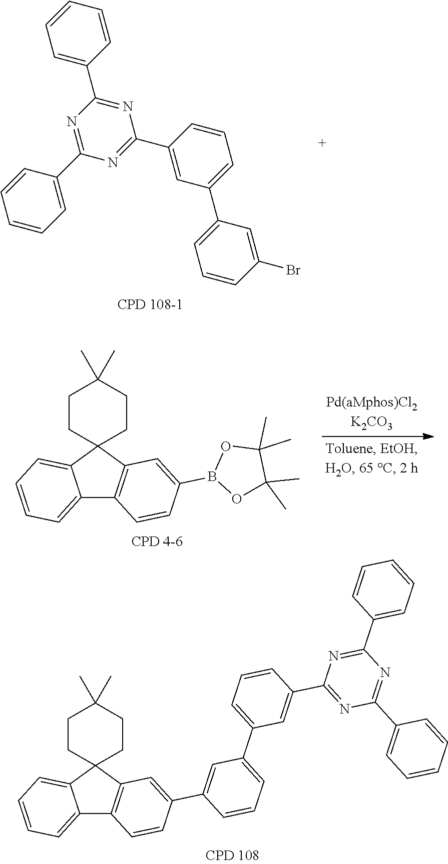 [SMILES: Brc1cccc(-c2cccc(-c3nc(-c4ccccc4)nc(-c4ccccc4)n3)c2)c1 ; CC1(C)CCC2(CC1)c1ccccc1-c1ccc(B3OC(C)(C)C(C)(C)O3)cc12 ; CC1(C)CCC2(CC1)c1ccccc1-c1ccc(-c3cccc(-c4cccc(-c5nc(-c6ccccc6)nc(-c6ccccc6)n5)c4)c3)cc12]

Referring to the synthesis and purification methods of compound CPD 4, it was only necessary to change the corresponding raw materials to obtain the target compound CPD 108 (25.11 g, purity: 99.97%, yield: 74.52%). 25.11 g of crude CPD 108 was purified by sublimation to obtain the sublimation-purified CPD 108 (18.85 g, purity: 99.97%, yield: 75.07%), with a mass spectrum: 646.21 (M+H). ¹H NMR (400 MHz, CDCl₃) δ 9.07 (s, 1H), 8.83 (d, J=6.8 Hz, 5H), 8.01 (s, 1H), 7.94-7.91 (m, 2H), 7.87-7.85 (m, 1H), 7.82-7.80 (m, 1H), 7.79-7.56 (m, 12H), 7.41 (t, J=7.3 Hz, 1H), 7.35 (t, J=7.2 Hz, 1H), 1.96 (m, 2H), 1.82 (m, 6H), 1.25 (s, 6H).

### 7. Synthesis Route of Compound CPD 119

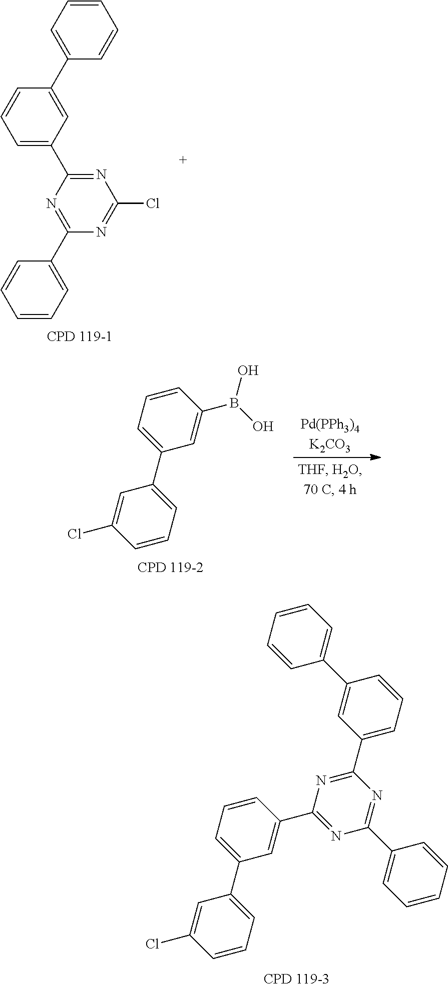 [SMILES: Clc1nc(-c2ccccc2)nc(-c2cccc(-c3ccccc3)c2)n1 ; OB(O)c1cccc(-c2cccc(Cl)c2)c1 ; Clc1cccc(-c2cccc(-c3nc(-c4ccccc4)nc(-c4cccc(-c5ccccc5)c4)n3)c2)c1]

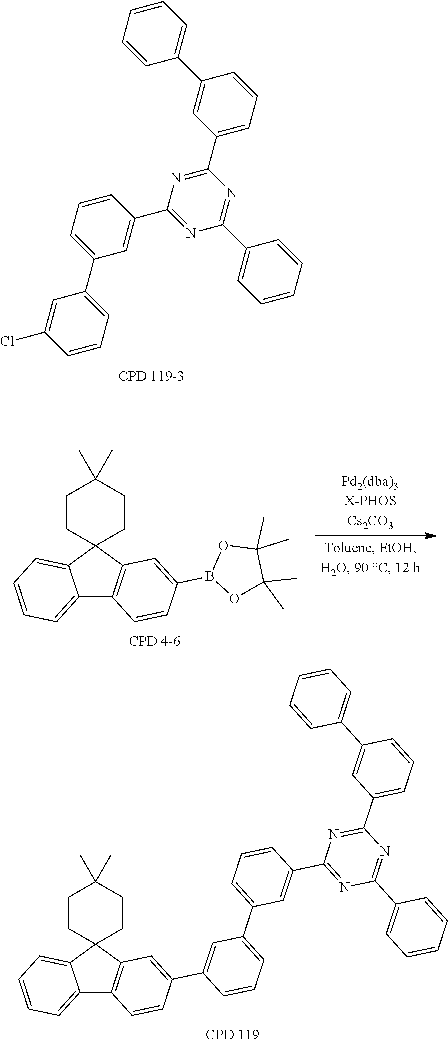 [SMILES: Clc1cccc(-c2cccc(-c3nc(-c4ccccc4)nc(-c4cccc(-c5ccccc5)c4)n3)c2)c1 ; CC1(C)CCC2(CC1)c1ccccc1-c1ccc(B3OC(C)(C)C(C)(C)O3)cc12 ; CC1(C)CCC2(CC1)c1ccccc1-c1ccc(-c3cccc(-c4cccc(-c5nc(-c6ccccc6)nc(-c6cccc(-c7ccccc7)c6)n5)c4)c3)cc12]

### Synthesis of Compound CPD 119-3

CPD 119-1 (25.00 g, 72.71 mmol), CPD 119-2 (21.97 g, 94.53 mmol), tetrakis(triphenylphosphine)palladium (1.68 g, 1.45 mmol), potassium carbonate (20.10 g, 145.42 mmol), tetrahydrofuran (375 ml), and deionized water (125 ml) were added to a 1,000 ml three-necked round-bottom flask, and the system was replaced with nitrogen under vacuum for three times, then heated to 70 °C and reacted for 4 hours. The reaction was monitored by TLC (ethyl acetate:n-hexane=1:10 as developing agent) until the raw material CPD 119-1 was consumed completely. The solvent was removed by concentration directly, then dichloromethane (500 ml) was added, and the solution was washed with deionized water (150 ml×3), then liquid separation was carried out, the sample was mixed with silica gel and dry-loaded onto a chromatography column, which was purified through silica gel column chromatography (200-300 mesh of silica gel, ethyl acetate:n-hexane=1:20 as eluent); after elution, the mixture was concentrated under reduced pressure at 65 °C for 1 hours to obtain CPD 119-3 as a white solid (29.97 g, purity: 99.32%, yield: 83.10%), with a mass spectrum: 496.20 (M+H).

### Synthesis of Compound CPD 119

CPD 119-3 (25.00 g, 50.40 mmol), CPD 4-6 (23.49 g, 60.48 mmol), tris(dibenzylideneacetone)dipalladium (0.92 g, 1.00 mmol), 2-dicyclohexylphosphino-2′,4′,6′-triisopropylbiphenyl (0.95 g, 2.00 mmol), cesium carbonate (35.56 g, 100.80 mmol), toluene (375 ml), ethanol (125 ml), and deionized water (125 ml) were added to a 2,000 ml three-necked round-bottom flask, next the system was replaced with nitrogen under vacuum for three times, then the system was heated to 90 °C and reacted for 2 hours. The reaction was monitored by TLC (ethyl acetate:n-hexane=1:10 as developing agent) until the raw material CPD 119-3 was consumed completely. The reaction was reduced to room temperature, then methanol (300 ml) was added, the mixture was stirred at room temperature for 1 hour, and a large amount of solid precipitated. Toluene (500 ml) was added, and the system was heated to 120 °C for dissolution, then lowered to room temperature, and filtered through 300-400 mesh of silica gel (60 g) once, methanol (200 ml) was added to the filtrate at room temperature, the mixture was stirred at room temperature for 1 hour, suction filtered to obtain a wet product as a white solid, which was dried at 100 °C for 1 hour to obtain 30.68 g of white solid; the above solid was added into a 1,000 ml single-necked round bottom flask, and recrystallized once using toluene (301 ml) and methanol (154 ml), then suction filtered, the filter cake was vacuum dried at 100 °C for 12 hours to obtain CPD 119 as a white solid (27.65 g, purity: 99.94%, yield: 76.00%). 27.65 g of crude CPD 119 was purified by sublimation to obtain the sublimation-purified CPD 119 (22.55 g, purity: 99.94%, yield: 81.56%), with a mass spectrum: 722.32 (M+H). ¹H NMR (400 MHz, CDCl₃) δ 8.21 (t, J=2.1 Hz, 1H), 8.12 (t, J=1.8 Hz, 1H), 8.10-8.04 (m, 2H), 8.02 (d, J=7.9 Hz, 1H), 7.90-7.82 (m, 2H), 7.81-7.75 (m, 2H), 7.71 (t, J=2.2 Hz, 1H), 7.65-7.63 (m, 1H), 7.58-7.54 (m, 7H), 7.52-7.46 (m, 4H), 7.42 (d, J=1.9 Hz, 1H), 7.41-7.36 (m, 3H), 7.36-7.27 (m, 3H), 2.28-2.22 (m, 2H), 2.04-1.98 (m, 2H), 1.58 (t, J=6.3 Hz, 4H), 1.19 (s, 6H).

### 8. Synthesis Route of Compound CPD 128

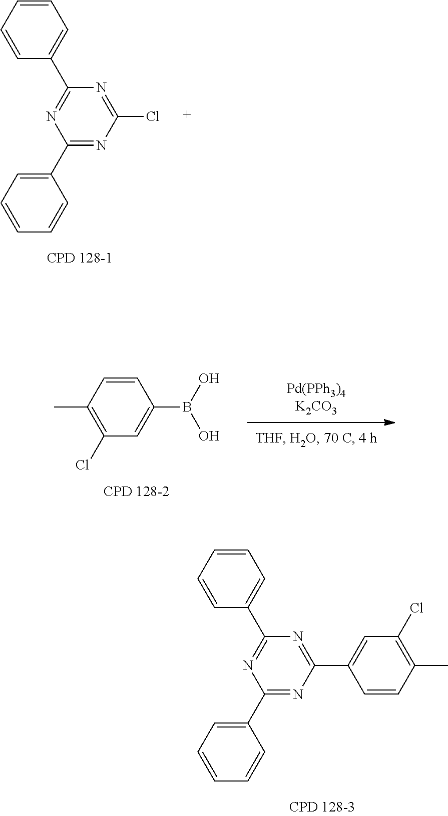 [SMILES: Clc1nc(-c2ccccc2)nc(-c2ccccc2)n1 ; Cc1ccc(B(O)O)cc1Cl ; Cc1ccc(-c2nc(-c3ccccc3)nc(-c3ccccc3)n2)cc1Cl]

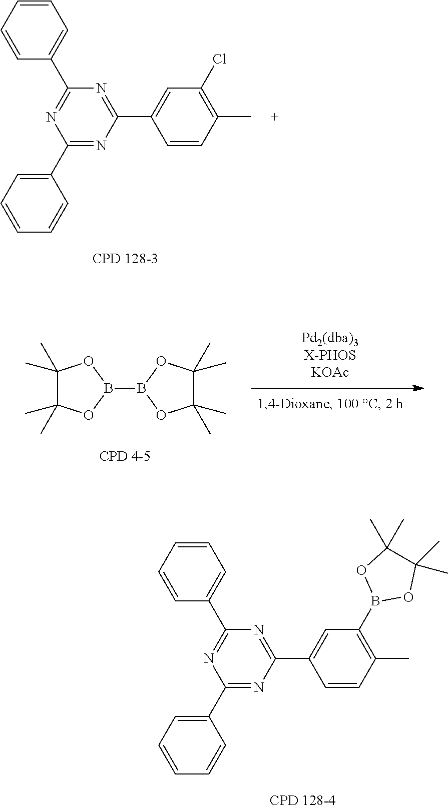

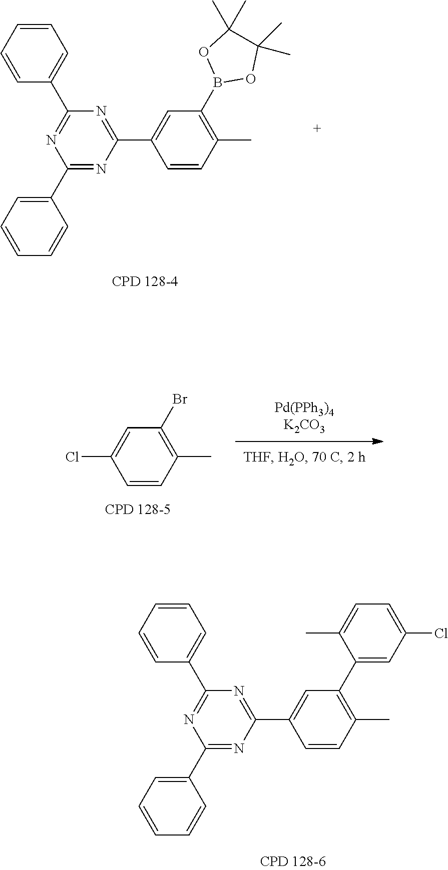

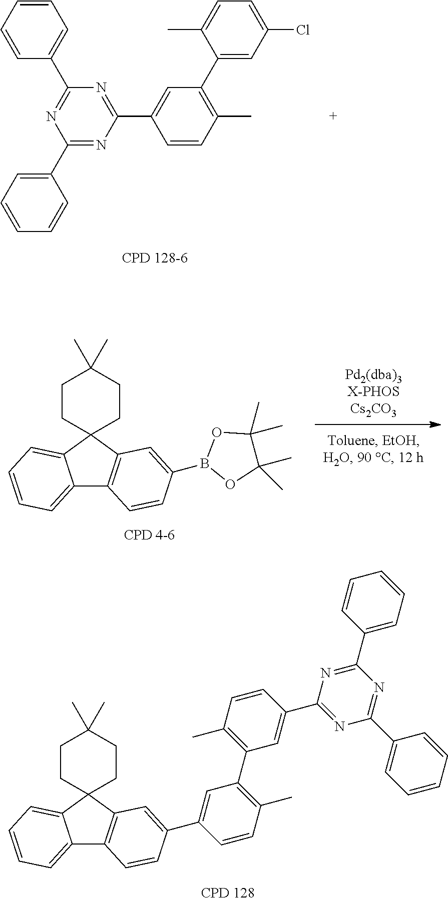 [SMILES: Cc1ccc(Cl)cc1-c1cc(-c2nc(-c3ccccc3)nc(-c3ccccc3)n2)ccc1C ; CC1(C)CCC2(CC1)c1ccccc1-c1ccc(B3OC(C)(C)C(C)(C)O3)cc12 ; Cc1ccc(-c2ccc3c(c2)C2(CCC(C)(C)CC2)c2ccccc2-3)cc1-c1cc(-c2nc(-c3ccccc3)nc(-c3ccccc3)n2)ccc1C]

### Synthesis of Compound CPD 128-3

Referring to the synthesis and purification methods of compound CPD 119-3, it was only necessary to change the corresponding raw materials to obtain the target compound CPD 128-3 (30.05 g, purity: 99.23%, yield: 82.78%), with a mass spectrum: 358.10 (M+H).

### Synthesis of Compound CPD 128-4

Referring to the synthesis and purification methods of compound CPD 4-6, it was only necessary to change the corresponding raw materials to obtain the target compound CPD 128-4 (34.66 g, purity: 98.09%, yield: 85.74%), with a mass spectrum: 450.20 (M+H).

### Synthesis of Compound CPD 128-6

Referring to the synthesis and purification methods of compound CPD 119-3, it was only necessary to change the corresponding raw materials to obtain the target compound CPD 128-6 (28.41 g, purity: 99.52%, yield: 85.09%), with a mass spectrum: 448.14 (M+H).

### Synthesis of Compound CPD 128

Referring to the synthesis and purification methods of compound CPD 119, it was only necessary to change the corresponding raw materials to obtain the target compound CPD 128 (16.01 g, purity: 99.93%, yield: 76.00%). 16.01 g of crude CPD 128 was purified by sublimation to obtain the sublimation-purified CPD 128 (12.79 g, purity: 99.95%, yield: 79.89%), with a mass spectrum: 674.36 (M+H). ¹H NMR (400 MHz, CDCl₃) δ 8.12-8.05 (m, 5H), 8.02 (d, J=7.9 Hz, 1H), 7.86-7.84 (m, 1H), 7.81-7.75 (m, 3H), 7.60-7.58 (m, 1H), 7.55-7.44 (m, 7H), 7.38-7.27 (m, 5H), 2.40 (s, 6H), 2.28-2.21 (m, 2H), 2.03-1.96 (m, 2H), 1.58 (t, J=6.3 Hz, 4H), 1.19 (s, 6H).

### 9. Synthesis Route of Compound CPD 136

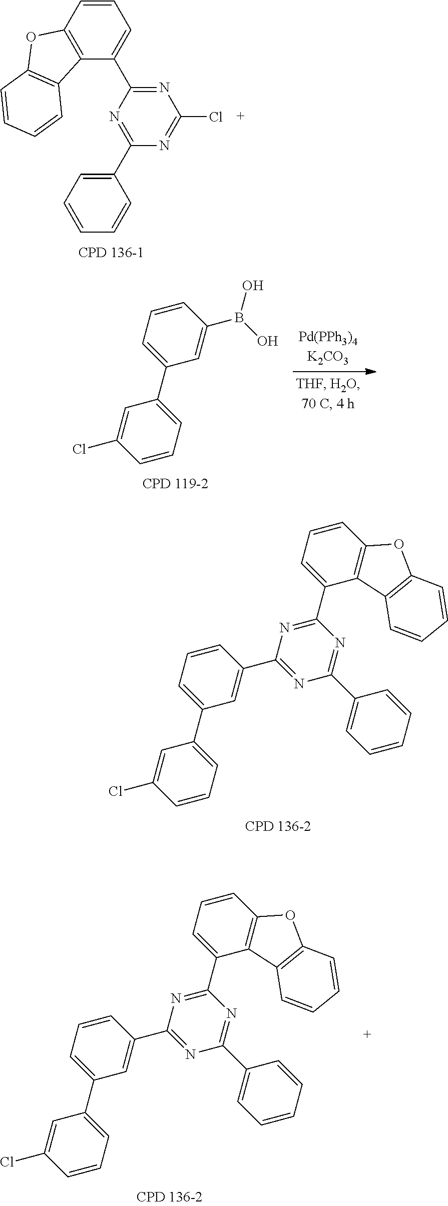 [SMILES: Clc1nc(-c2ccccc2)nc(-c2cccc3oc4ccccc4c23)n1 ; OB(O)c1cccc(-c2cccc(Cl)c2)c1 ; Clc1cccc(-c2cccc(-c3nc(-c4ccccc4)nc(-c4cccc5oc6ccccc6c45)n3)c2)c1 ; Clc1cccc(-c2cccc(-c3nc(-c4ccccc4)nc(-c4cccc5oc6ccccc6c45)n3)c2)c1]

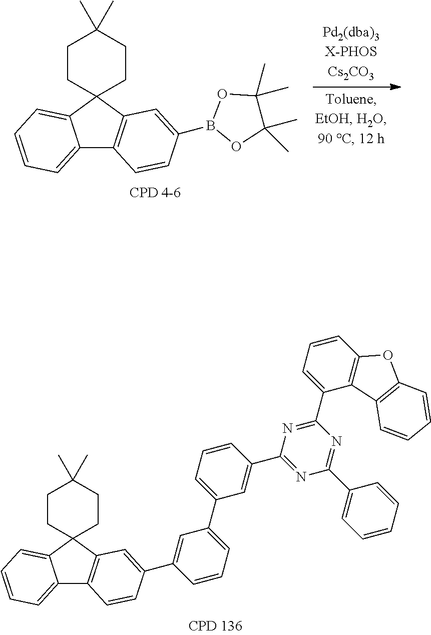 [SMILES: CC1(C)CCC2(CC1)c1ccccc1-c1ccc(B3OC(C)(C)C(C)(C)O3)cc12 ; CC1(C)CCC2(CC1)c1ccccc1-c1ccc(-c3cccc(-c4cccc(-c5nc(-c6ccccc6)nc(-c6cccc7oc8ccccc8c67)n5)c4)c3)cc12]

### Synthesis of Compound CPD 136-2

Referring to the synthesis and purification methods of compound CPD 119-3, it was only necessary to change the corresponding raw materials to obtain the target compound CPD 136-2 (45.61 g, purity: 99.46%, yield: 80.00%), with a mass spectrum: 510.13 (M+H).

### Synthesis of Compound CPD 136

Referring to the synthesis and purification methods of compound CPD 119, it was only necessary to change the corresponding raw materials to obtain the target compound CPD 136 (27.13 g, purity: 99.95%, yield: 75.19%). 27.13 g of crude CPD 136 was purified by sublimation to obtain the sublimation-purified CPD 136 (21.28 g, purity: 99.95%, yield: 78.44%), with a mass spectrum: 736.42 (M+H). ¹H NMR (400 MHz, CDCl₃) δ 8.21 (t, J=2.1 Hz, 1H), 8.12-7.99 (m, 4H), 7.90-7.83 (m, 2H), 7.81-7.75 (m, 2H), 7.71 (t, J=2.2 Hz, 1H), 7.65-7.59 (m, 2H), 7.58-7.53 (m, 4H), 7.53-7.46 (m, 5H), 7.45-7.41 (m, 2H), 7.38-7.23 (m, 4H), 2.26-2.21 (m, 2H), 2.04-1.97 (m, 2H), 1.58 (t, J=6.3 Hz, 4H), 1.20 (s, 6H).

### 10. Synthesis Route of Compound CPD 160

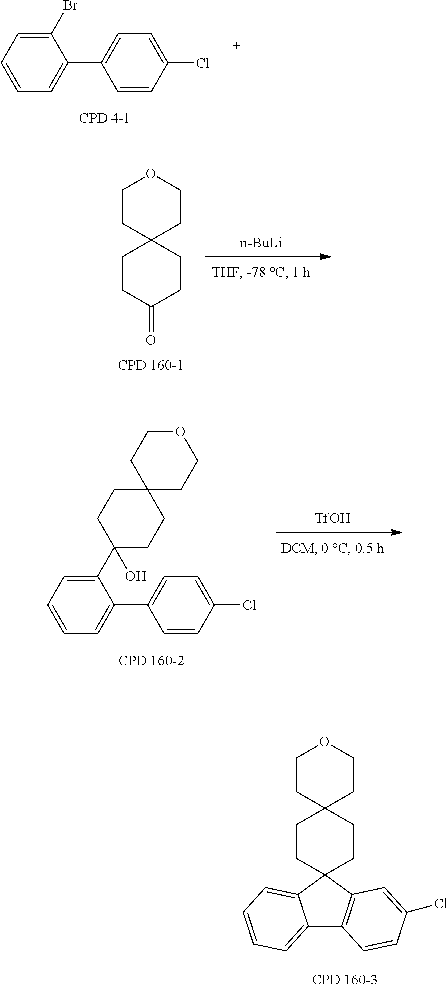

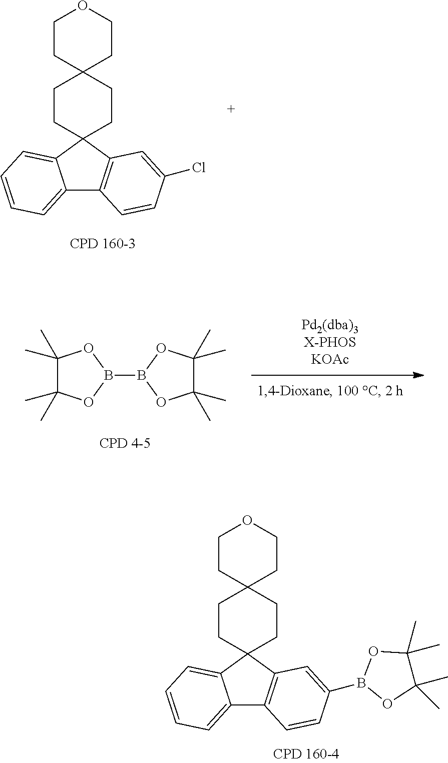

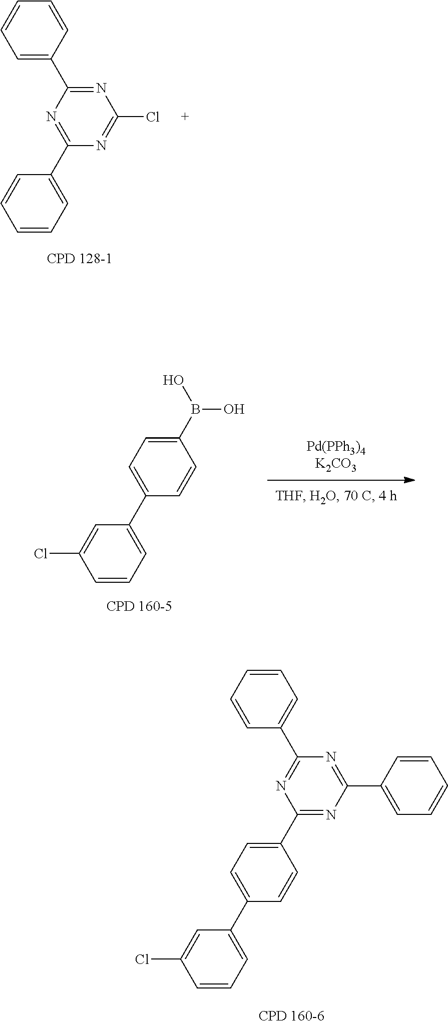 [SMILES: Clc1nc(-c2ccccc2)nc(-c2ccccc2)n1 ; OB(O)c1ccc(-c2cccc(Cl)c2)cc1 ; Clc1cccc(-c2ccc(-c3nc(-c4ccccc4)nc(-c4ccccc4)n3)cc2)c1]

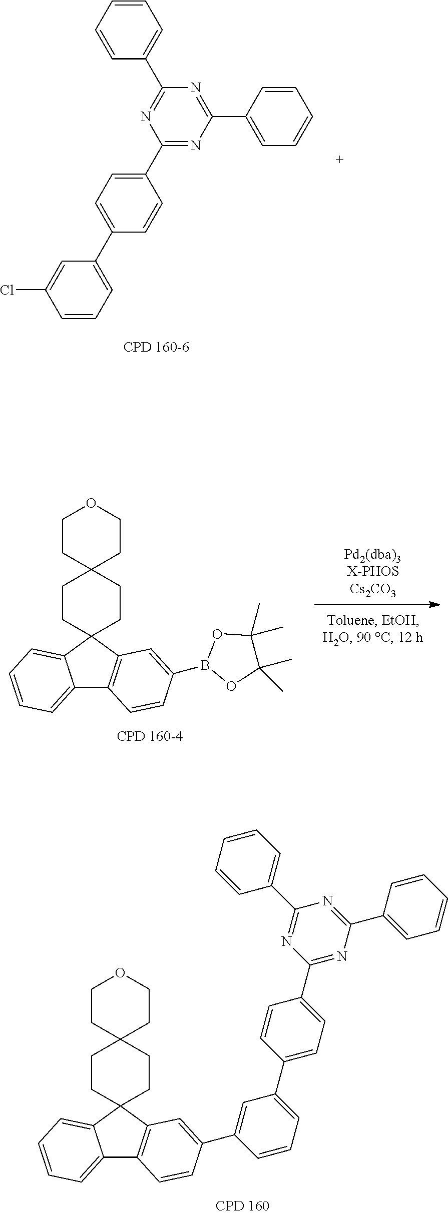 [SMILES: Clc1cccc(-c2ccc(-c3nc(-c4ccccc4)nc(-c4ccccc4)n3)cc2)c1 ; CC1(C)OB(c2ccc3c(c2)C2(CCC4(CCOCC4)CC2)c2ccccc2-3)OC1(C)C ; c1ccc(-c2nc(-c3ccccc3)nc(-c3ccc(-c4cccc(-c5ccc6c(c5)C5(CCC7(CCOCC7)CC5)c5ccccc5-6)c4)cc3)n2)cc1]

### Synthesis of Compound CPD 160-2

Referring to the synthesis and purification methods of compound CPD 4-3, it was only necessary to change the corresponding raw materials to obtain the target compound CPD 160-2 (32.56 g, purity: 99.51%, yield: 66.85%), with a mass spectrum: 357.02 (M+H).

### Synthesis of Compound CPD 160-3

Referring to the synthesis and purification methods of compound CPD 4-4, it was only necessary to change the corresponding raw materials to obtain the target compound CPD 160-3 (28.02 g, purity: 99.90%, yield: 83.65%), with a mass spectrum: 339.14 (M+H).

### Synthesis of Compound CPD 160-4

Referring to the synthesis and purification methods of compound CPD 4-6, it was only necessary to change the corresponding raw materials to obtain the target compound CPD 160-4 (30.67 g. purity: 99.90%, yield: 86.23%), with a mass spectrum: 431.22 (M+H).

### Synthesis of Compound CPD 160-6

Referring to the synthesis and purification methods of compound CPD 119-3, it was only necessary to change the corresponding raw materials to obtain the target compound CPD 160-6 (28.06 g, purity: 99.42%, yield: 82.00%), with a mass spectrum: 420.32 (M+H).

### Synthesis of Compound CPD 160

Referring to the synthesis and purification methods of compound CPD 119, it was only necessary to change the corresponding raw materials to obtain the target compound CPD 160 (22.96 g, purity: 99.96%, yield: 76.48%). 22.96 g of crude CPD 160 was purified by sublimation to obtain the sublimation-purified CPD 160 (18.25 g, purity: 99.96%, yield: 79.49%), with a mass spectrum: 688.32 (M+H). ¹H NMR (400 MHz, CDCl₃) δ 8.12-8.04 (m, 4H), 7.97-7.91 (m, 2H), 7.81-7.75 (m, 2H), 7.72-7.64 (m, 3H), 7.60-7.44 (m, 10H), 7.38-7.28 (m, 4H), 3.53-3.50 (m, 4H), 2.39-2.34 (m, 2H), 2.14-2.08 (m, 2H), 1.69 (t, J=6.2 Hz, 4H), 1.50-1.44 (m, 4H).

### 11. Synthesis Route of Compound CPD 179

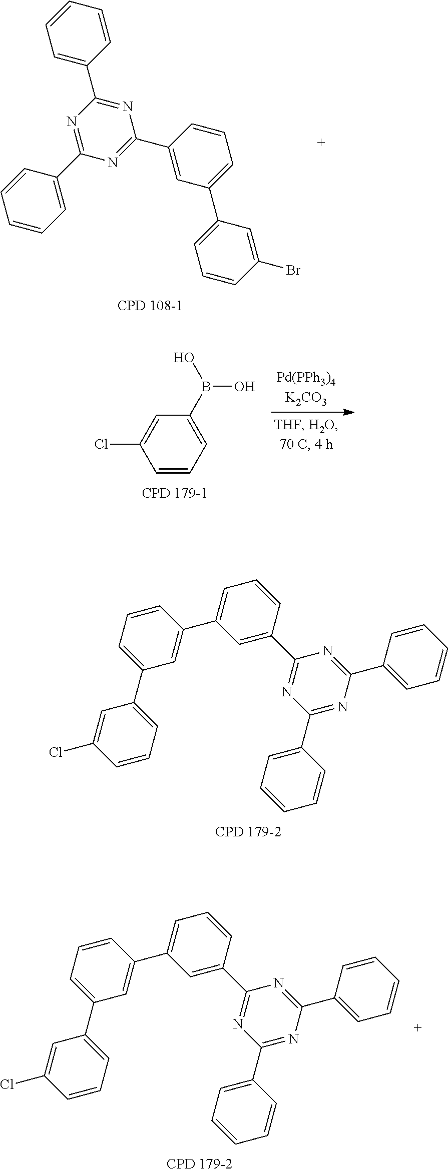 [SMILES: Brc1cccc(-c2cccc(-c3nc(-c4ccccc4)nc(-c4ccccc4)n3)c2)c1 ; OB(O)c1cccc(Cl)c1 ; Clc1cccc(-c2cccc(-c3cccc(-c4nc(-c5ccccc5)nc(-c5ccccc5)n4)c3)c2)c1 ; Clc1cccc(-c2cccc(-c3cccc(-c4nc(-c5ccccc5)nc(-c5ccccc5)n4)c3)c2)c1]

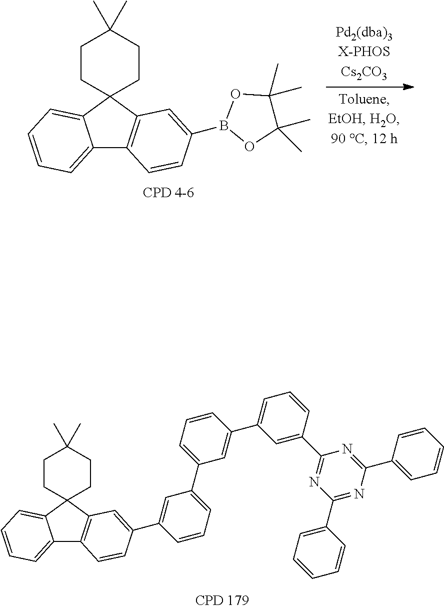 [SMILES: CC1(C)CCC2(CC1)c1ccccc1-c1ccc(B3OC(C)(C)C(C)(C)O3)cc12 ; CC1(C)CCC2(CC1)c1ccccc1-c1ccc(-c3cccc(-c4cccc(-c5cccc(-c6nc(-c7ccccc7)nc(-c7ccccc7)n6)c5)c4)c3)cc12]

### Synthesis of Compound CPD 179-2

Referring to the synthesis and purification methods of compound CPD 119-3, it was only necessary to change the corresponding raw materials to obtain the target compound CPD 179-2 (25.33 g, purity: 99.89%, yield: 82.96%), with a mass spectrum: 496.16 (M+H).

### Synthesis of Compound CPD 179

Referring to the synthesis and purification methods of compound CPD 119, it was only necessary to change the corresponding raw materials to obtain the target compound CPD 179 (24.63 g, purity: 99.94%, yield: 73.56%). 24.63 g of crude CPD 179 was purified by sublimation to obtain the sublimation-purified CPD 179 (20.82 g, purity: 99.96%, yield: 84.54%), with a mass spectrum: 722.30 (M+H). ¹H NMR (400 MHz, CDCl₃) δ 8.21 (t, J=2.1 Hz, 1H), 8.12-8.04 (m, 4H), 8.02 (d, J=7.9 Hz, 1H), 7.87-7.85 (m, 1H), 7.81-7.75 (m, 2H), 7.71 (t, J=2.2 Hz, 2H), 7.65-7.62 (m, 1H), 7.60-7.55 (m, 5H), 7.53-7.45 (m, 8H), 7.36 (s, 1H), 7.36-7.28 (m, 3H), 2.29-2.21 (m, 2H), 2.01-1.96 (m, 2H), 1.57 (t, J=6.3 Hz, 4H), 1.20 (s, 6H).

### 12. Synthesis Route of Compound CPD 223

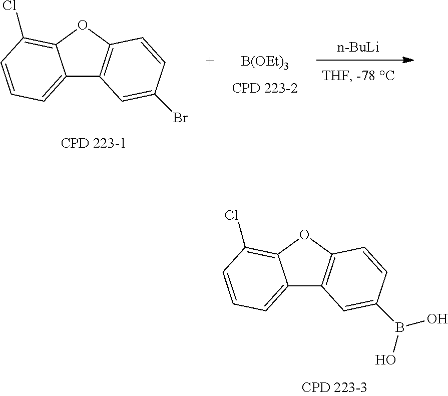

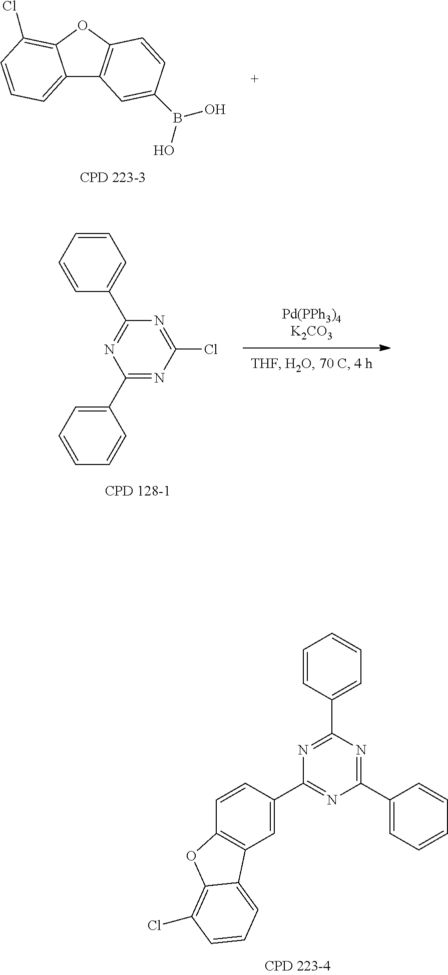 [SMILES: OB(O)c1ccc2oc3c(Cl)cccc3c2c1 ; Clc1nc(-c2ccccc2)nc(-c2ccccc2)n1 ; Clc1cccc2c1oc1ccc(-c3nc(-c4ccccc4)nc(-c4ccccc4)n3)cc12]

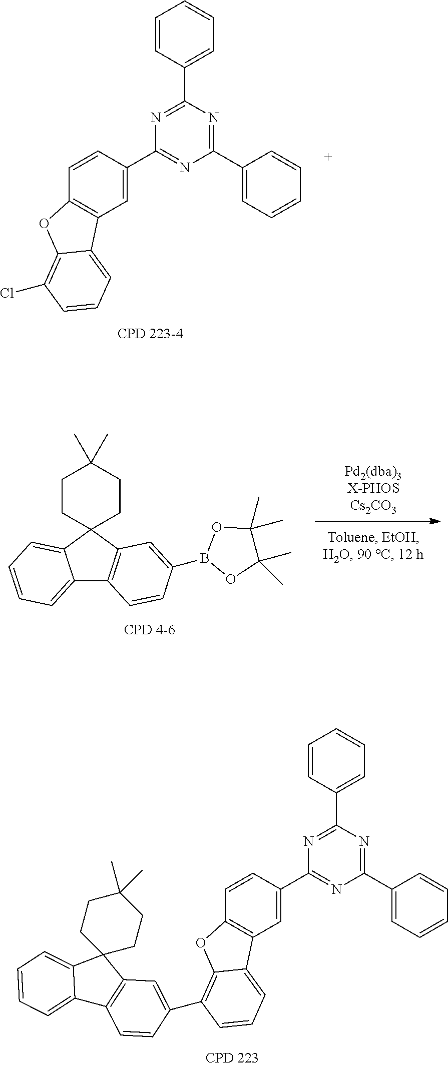 [SMILES: Clc1cccc2c1oc1ccc(-c3nc(-c4ccccc4)nc(-c4ccccc4)n3)cc12 ; CC1(C)CCC2(CC1)c1ccccc1-c1ccc(B3OC(C)(C)C(C)(C)O3)cc12 ; CC1(C)CCC2(CC1)c1ccccc1-c1ccc(-c3cccc4c3oc3ccc(-c5nc(-c6ccccc6)nc(-c6ccccc6)n5)cc34)cc12]

### Synthesis of Compound CPD 223-3

Compound CPD 223-1 (50.00 g, 177.60 mmol) and dry tetrahydrofuran (750 ml) were added to a 2,000 ml three-necked round-bottom flask, the system was replaced with nitrogen under vacuum for three times, next it was cooled to −78 °C, then a solution of n-butyllithium in n-hexane (97.68 ml, 195.36 mmol, a concentration of 2 mol/L) was added dropwise, while the inner temperature of the system was controlled to be no higher than −70 °C, the addition was completed over 1 hour, and the mixture was maintained at −78 °C and stirred for 1 hour. CPD 223-2 (38.89 g, 266.40 mmol) was slowly added dropwise over 10 minutes, and the mixture was naturally warmed up and stirred for 1 hour. The reaction was monitored by TLC (ethyl acetate:n-hexane=1:10 as the developing solvent) until the raw material CPD 223-1 was consumed completely and most of the CPD 223-3 was generated. Hydrochloric acid aqueous solution (2 mol/L, 100 ml) was added dropwise to the system to quench the reaction, and a large amount of solid precipitated, which was stirred at room temperature for 1 hour and suction filtered to obtain a white solid, the solid was beaten with n-hexane (100 ml) at room temperature for 1 hour, then suction filtered, the filter cake was vacuum dried at 65 °C for 3 hours to obtain CPD 223-3 as a white solid (36.84 g, purity: 98.06%, yield: 84.16%), with a mass spectrum: 247.04 (M+H).

### Synthesis of Compound CPD 223-4

Referring to the synthesis and purification methods of compound CPD 119-3, it was only necessary to change the corresponding raw materials to obtain the target compound CPD 223-4 (27.80 g, purity: 99.54%, yield: 78.96%), with a mass spectrum: 434.26 (M+H).

### Synthesis of Compound CPD 223

Referring to the synthesis and purification methods of compound CPD 119, it was only necessary to change the corresponding raw materials to obtain the target compound CPD 223 (23.30 g, purity: 99.96%, yield: 76.60%). 23.30 g of crude CPD 223 was purified by sublimation to obtain the sublimation-purified CPD 223 (18.64 g, purity: 99.96%, yield: 80.00%), with a mass spectrum: 660.20 (M+H). ¹H NMR (400 MHz, CDCl3) δ 8.17 (d, J=2.7 Hz, 1H), 8.12-8.03 (m, 6H), 7.92-7.85 (m, 2H), 7.81-7.74 (m, 2H), 7.68 (dd, J=7.8, 2.1 Hz, 1H), 7.58-7.44 (m, 8H), 7.38-7.28 (m, 3H), 2.28-2.21 (m, 2H), 2.00-1.96 (m, 2H), 1.56 (t, J=6.3 Hz, 4H), 1.21 (s, 6H).

### 13. Synthesis Route of Compound CPD 255

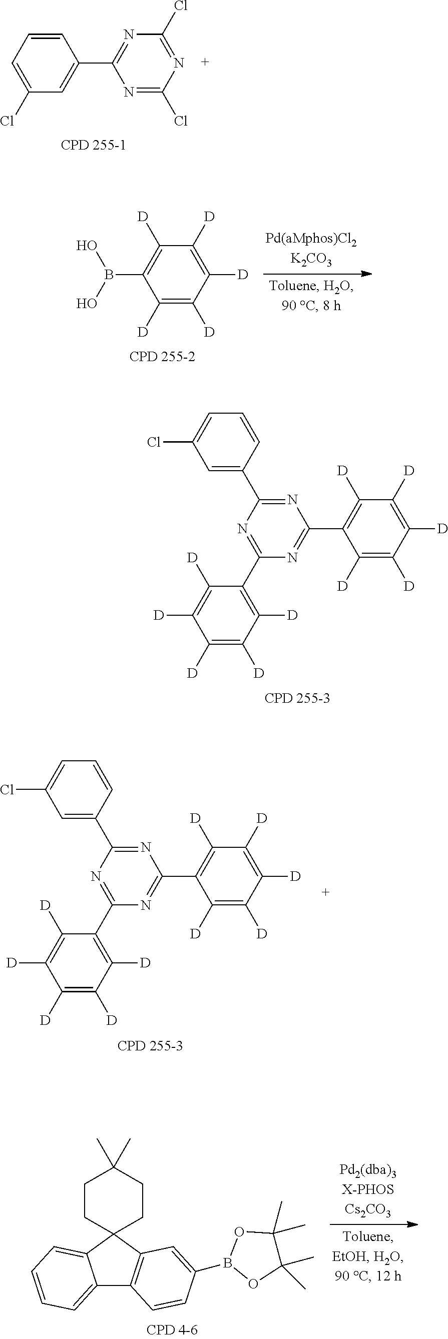 [SMILES: Clc1cccc(-c2nc(Cl)nc(Cl)n2)c1 ; [2H]c1c([2H])c([2H])c(B(O)O)c([2H])c1[2H] ; [2H]c1c([2H])c([2H])c(-c2nc(-c3cccc(Cl)c3)nc(-c3c([2H])c([2H])c([2H])c([2H])c3[2H])n2)c([2H])c1[2H] ; [2H]c1c([2H])c([2H])c(-c2nc(-c3cccc(Cl)c3)nc(-c3c([2H])c([2H])c([2H])c([2H])c3[2H])n2)c([2H])c1[2H] ; CC1(C)CCC2(CC1)c1ccccc1-c1ccc(B3OC(C)(C)C(C)(C)O3)cc12]

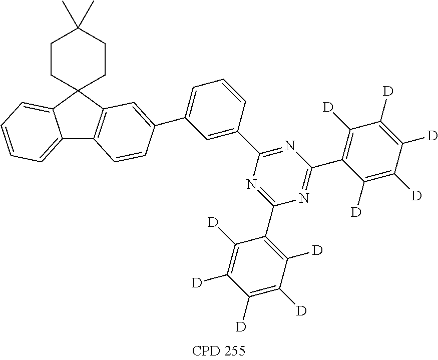 [SMILES: [2H]c1c([2H])c([2H])c(-c2nc(-c3cccc(-c4ccc5c(c4)C4(CCC(C)(C)CC4)c4ccccc4-5)c3)nc(-c3c([2H])c([2H])c([2H])c([2H])c3[2H])n2)c([2H])c1[2H]]

### Synthesis of Compound CPD 255-3

Referring to the synthesis and purification methods of compound CPD 4, it was only necessary to change the corresponding raw materials, the reaction solvents were toluene and deionized water, and the reaction was reacted at 90 °C for 8 hours to obtain the target compound CPD 255-3 (18.24 g. purity: 99.67%, yield: 67.15%), with a mass spectrum: 354.12 (M+H).

### Synthesis of Compound CPD 255

Referring to the synthesis and purification methods of compound CPD 119, it was only necessary to change the corresponding raw materials to obtain the target compound CPD 255 (18.31 g, purity: 99.94%, yield: 74.52%). 18.31 g of crude CPD 255 was purified by sublimation to obtain the sublimation-purified CPD 255 (14.85 g, purity: 99.96%, yield: 81.11%), with a mass spectrum: 580.34 (M+H). ¹H NMR (400 MHz, CDCl₃) δ 8.21 (t, J=2.1 Hz, 1H), 8.02 (d, J=7.8 Hz, 1H), 7.87-7.85 (m, 1H), 7.81-7.75 (m, 2H), 7.65-7.63 (m, 1H), 7.57-7.53 (m, 1H), 7.49-7.46 (m, 1H), 7.38-7.27 (m, 3H), 2.27-2.21 (m, 2H), 2.00-1.95 (m, 2H), 1.54 (t, J=6.3 Hz, 4H), 1.21 (s, 6H).

### 14. Synthesis Route of Compound CPD 262

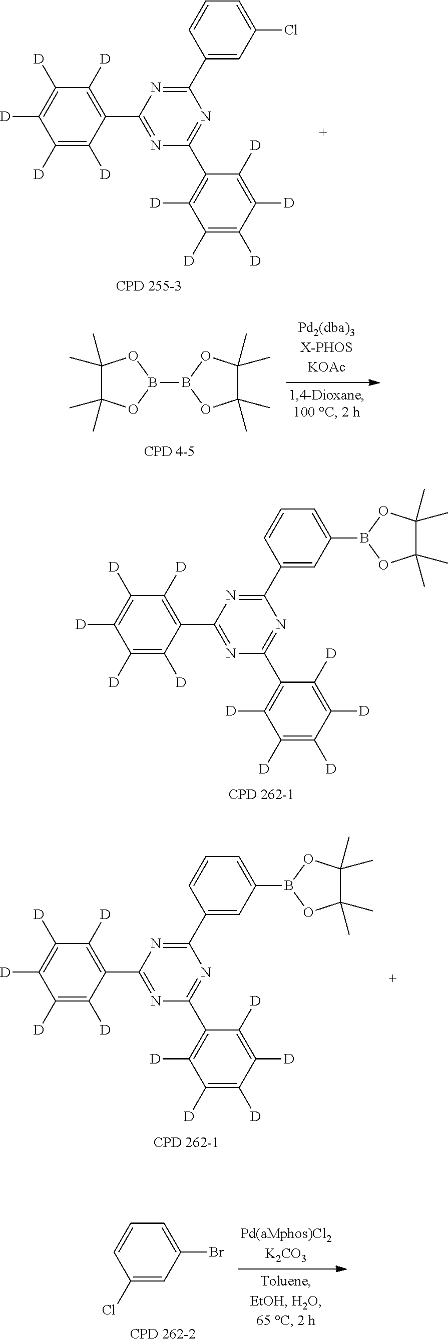

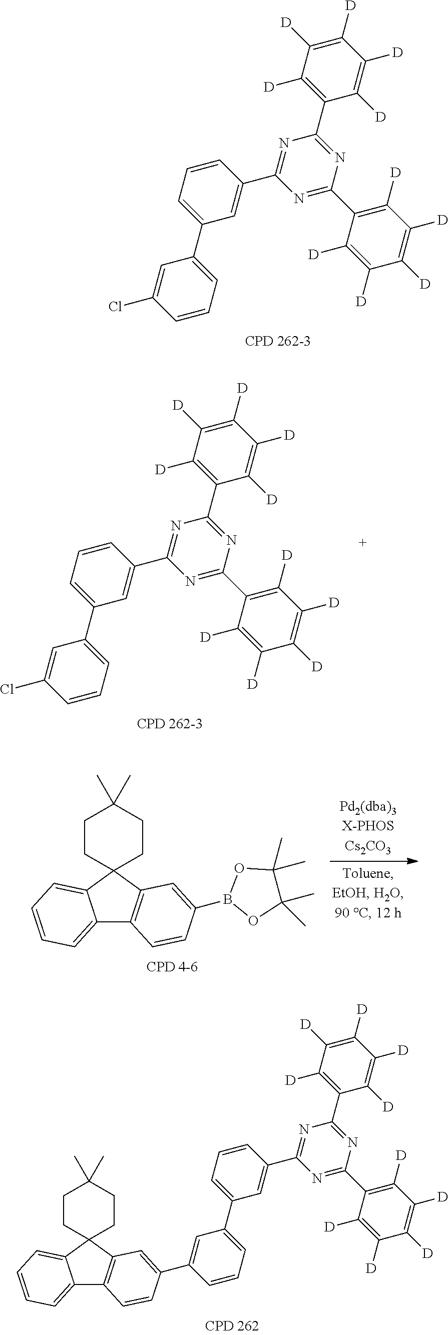 [SMILES: [2H]c1c([2H])c([2H])c(-c2nc(-c3cccc(-c4cccc(Cl)c4)c3)nc(-c3c([2H])c([2H])c([2H])c([2H])c3[2H])n2)c([2H])c1[2H] ; [2H]c1c([2H])c([2H])c(-c2nc(-c3cccc(-c4cccc(Cl)c4)c3)nc(-c3c([2H])c([2H])c([2H])c([2H])c3[2H])n2)c([2H])c1[2H] ; CC1(C)CCC2(CC1)c1ccccc1-c1ccc(B3OC(C)(C)C(C)(C)O3)cc12 ; [2H]c1c([2H])c([2H])c(-c2nc(-c3cccc(-c4cccc(-c5ccc6c(c5)C5(CCC(C)(C)CC5)c5ccccc5-6)c4)c3)nc(-c3c([2H])c([2H])c([2H])c([2H])c3[2H])n2)c([2H])c1[2H]]

### Synthesis of Compound CPD 262-1

Referring to the synthesis and purification methods of compound CPD 4-6, it was only necessary to change the corresponding raw materials to obtain the target compound CPD 262-1 (20.33 g, purity: 99.75%, yield: 67.15%), with a mass spectrum: 446.22 (M+H).

### Synthesis of Compound CPD 262-3

Referring to the synthesis and purification methods of compound CPD 4, it was only necessary to change the corresponding raw materials to obtain the target compound CPD 262-3 (13.56 g, purity: 99.89%, yield: 78.06%), with a mass spectrum: 430.14 (M+H).

### Synthesis of Compound CPD 262

Referring to the synthesis and purification methods of compound CPD 119, it was only necessary to change the corresponding raw materials to obtain the target compound CPD 262 (14.36 g, purity: 99.96%, yield: 78.47%). 14.36 g of crude CPD 262 was purified by sublimation to obtain the sublimation-purified CPD 262 (11.01 g, purity: 99.96%, yield: 76.68%), with a mass spectrum: 656.34 (M+H). ¹H NMR (400 MHz, CDCl₃) δ 8.21 (t, J=2.1 Hz, 1H), 8.02 (d, J=7.8 Hz, 1H), 7.8-7.77 (m, 3H), 7.71 (t, J=2.2 Hz, 1H), 7.65-7.63 (m, 1H), 7.60-7.52 (m, 3H), 7.52-7.43 (m, 2H), 7.36-7.27 (m, 3H), 2.26-2.21 (m, 2H), 2.00-1.96 (m, 2H), 1.57 (t, J=6.3 Hz, 4H), 1.19 (s, 6H).

### 15. Synthesis Route of Compound CPD 266

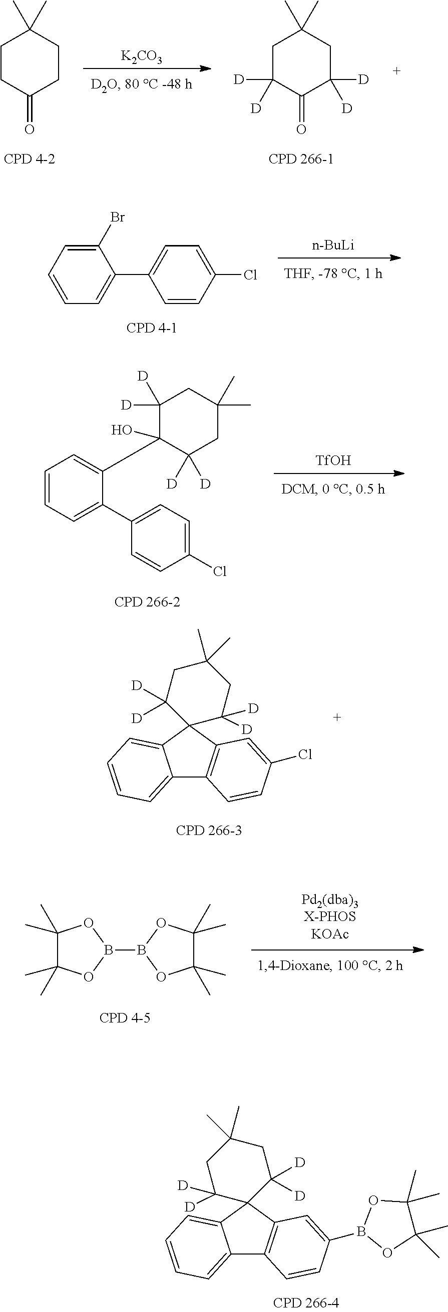

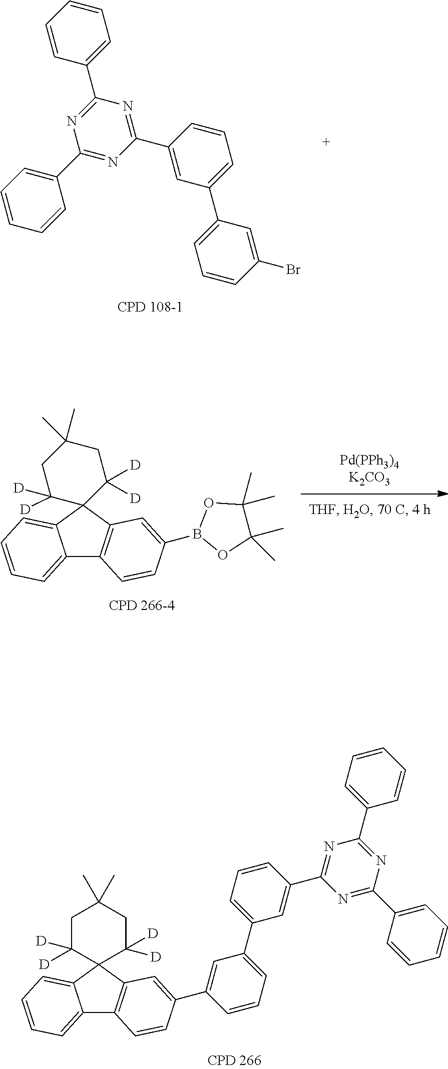 [SMILES: Brc1cccc(-c2cccc(-c3nc(-c4ccccc4)nc(-c4ccccc4)n3)c2)c1 ; [2H]C1([2H])CC(C)(C)CC([2H])([2H])C12c1ccccc1-c1ccc(B3OC(C)(C)C(C)(C)O3)cc12 ; [2H]C1([2H])CC(C)(C)CC([2H])([2H])C12c1ccccc1-c1ccc(-c3cccc(-c4cccc(-c5nc(-c6ccccc6)nc(-c6ccccc6)n5)c4)c3)cc12]

### Synthesis of Compound CPD 266-1

CPD 4-2 (50.00 g, 396.20 mmol), anhydrous potassium carbonate (54.76 g, 396.20 mmol), and deuteroxide (500 g) were added into a 1,000 ml three-necked round bottom flask, the system was replaced with nitrogen under vacuum for three times, then sealed for reaction, next the system was heated to 80 °C and reacted for 48 hours, the reaction was monitored using GC-MS and H NMR Spectroscopy until the raw material CPD 4-2 was consumed completely. The system was cooled to room temperature, and the liquid separation was carried out directly, dichloromethane (200 ml) was added to the organic phase, suction filtered with 300-400 mesh of silica gel loaded on chromatography column (60 g), the filtrate was combined and concentrated under reduced pressure at 50 °C for 3 hours to obtain CPD 266-1 as a colorless liquid (20.33 g, purity: 99.90%, deuterated ratio for four D was 99%, yield: 80.74%), with a mass spectrum: 131.12 ((M+H).

### Synthesis of Compound CPD 266-2

Referring to the synthesis and purification methods of compound CPD 4-3, it was only necessary to change the corresponding raw materials to obtain the target compound CPD 266-2 (39.61 g, purity: 99.77%, yield: 64.71%), with a mass spectrum: 319.16 (M+H).

### Synthesis of Compound CPD 266-3

Referring to the synthesis and purification methods of compound CPD 4-4, it was only necessary to change the corresponding raw materials to obtain the target compound CPD 266-3 (28.42 g, purity: 99.90%, yield: 86.06%), with a mass spectrum: 301.02 (M+H).

### Synthesis of Compound CPD 266-4

Referring to the synthesis and purification methods of compound CPD 4-6, it was only necessary to change the corresponding raw materials to obtain the target compound CPD 266-4 (26.52 g, purity: 99.01%, yield: 81.33%), with a mass spectrum: 393.22 (M+H).

### Synthesis of Compound CPD 266

Referring to the synthesis and purification methods of compound CPD 119-3, it was only necessary to change the corresponding raw materials to obtain the target compound CPD 266 (24.50 g, purity: 99.97%, yield: 76.12%). 24.50 g of crude CPD 266 was purified by sublimation to obtain the sublimation-purified CPD 266 (19.28 g, purity: 99.97%, yield: 78.70%), with a mass spectrum: 650.24 (M+H). ¹H NMR (400 MHz, CDCl₃) δ 8.21 (t, J=2.1 Hz, 1H), 8.16 (d, J=7.7 Hz, 1H), 8.12-8.04 (m, 4H), 8.00-7.95 (m, 1H), 7.86-7.75 (m, 3H), 7.7-7.64 (m, 2H), 7.60-7.44 (m, 12H), 7.35-7.29 (m, 1H), 2.22-2.12 (m, 4H), 1.22 (s, 6H).

### 16. Synthesis Route of Compound CPD 274

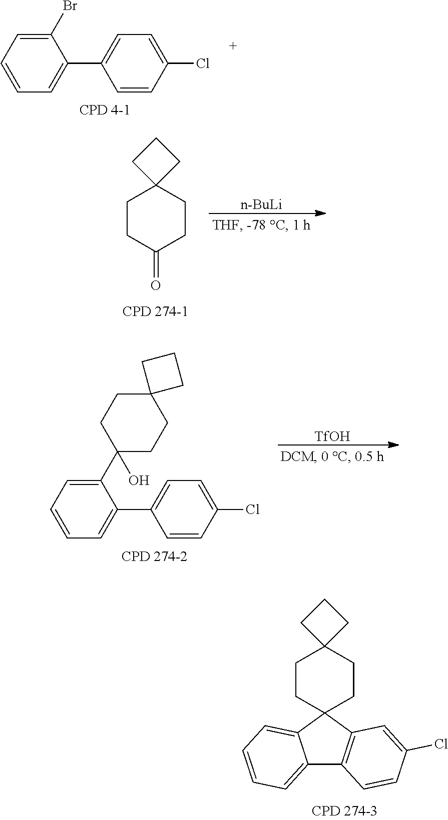

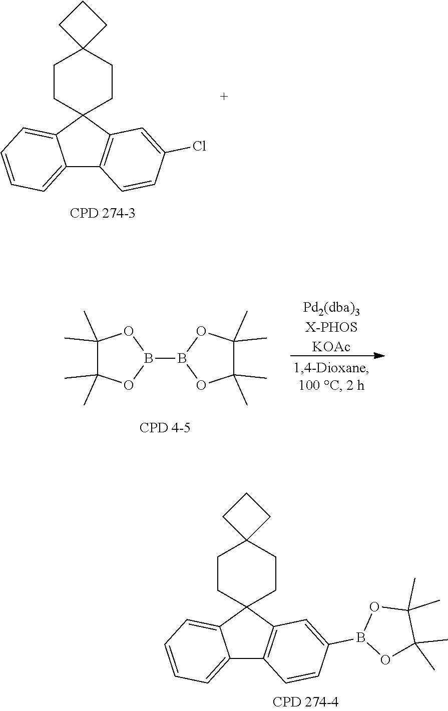

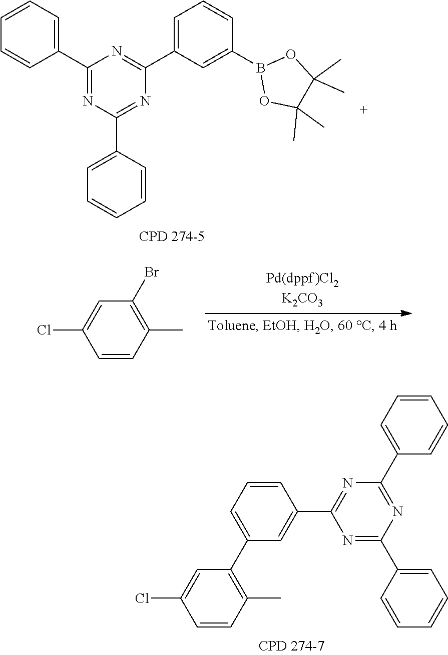

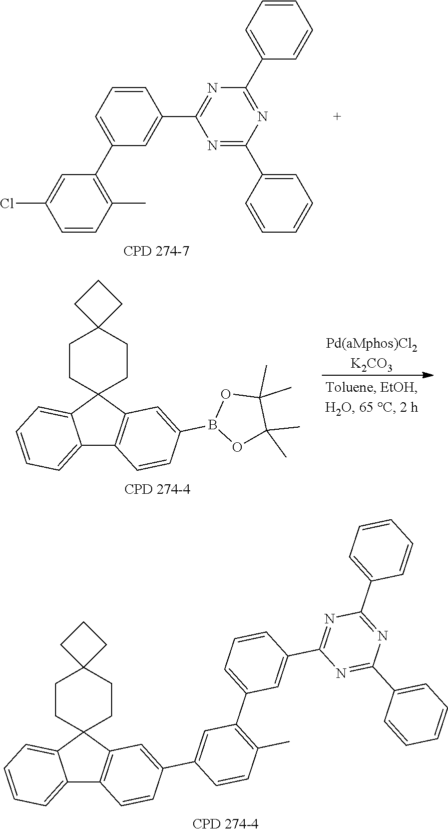 [SMILES: Cc1ccc(Cl)cc1-c1cccc(-c2nc(-c3ccccc3)nc(-c3ccccc3)n2)c1 ; CC1(C)OB(c2ccc3c(c2)C2(CCC4(CCC4)CC2)c2ccccc2-3)OC1(C)C ; "Pd(aMphos)Cl2K2CO3" ; Cc1ccc(-c2ccc3c(c2)C2(CCC4(CCC4)CC2)c2ccccc2-3)cc1-c1cccc(-c2nc(-c3ccccc3)nc(-c3ccccc3)n2)c1]

### Synthesis of Compound CPD 274-2

Referring to the synthesis and purification methods of compound CPD 4-3, it was only necessary to change the corresponding raw materials to obtain the target compound CPD 274-2 (36.41 g, purity: 99.52%, yield: 62.22%), with a mass spectrum: 327.16 (M+H).

### Synthesis of Compound CPD 274-3

Referring to the synthesis and purification methods of compound CPD 4-4, it was only necessary to change the corresponding raw materials to obtain the target compound CPD 274-3 (27.41 g, purity: 99.89%, yield: 83.45%), with a mass spectrum: 309.12 (M+H).

### Synthesis of Compound CPD 274-4

Referring to the synthesis and purification methods of compound CPD 4-6, it was only necessary to change the corresponding raw materials to obtain the target compound CPD 274-4 (32.41 g, purity: 98.89%, yield: 86.56%), with a mass spectrum: 401.26 (M+H).

### Synthesis of Compound CPD 274-7

CPD 274-5 (25.00 g, 57.43 mmol), CPD 274-6 (12.98 g, 63.17 mmol), 1,1-bis(diphenylphosphino)ferrocenedichloropalladium (0.841 g, 1.15 mmol), potassium carbonate (15.87 g, 114.86 mmol), toluene (375 ml), ethanol (125 ml) and deionized water (125 ml) were added to a 1,000 ml three-necked round-bottom flask, and the system was replaced with nitrogen under vacuum for three times, then heated to 60 °C and reacted for 4 hours. The reaction was monitored by TLC (ethyl acetate:n-hexane=1:10 as developing agent) until the raw material CPD 274-5 was consumed completely. The reaction was reduced to room temperature, then methanol (300 ml) was added, the mixture was stirred at room temperature for 1 hour, and a large amount of solid precipitated. Toluene (600 ml) was added, and the system was heated to 100 °C for dissolution, then lowered to room temperature, and filtered through 300-400 mesh of silica gel (60 g) once, methanol (450 ml) was added to the filtrate at room temperature, the mixture was stirred at room temperature for 1 hour, suction filtered to obtain a wet product as a white solid, which was dried at 80 °C for 1 hour to obtain 21.63 g of white solid; the above solid was added into a 500 ml single-necked round bottom flask, and recrystallized once using tetrahydrofuran (130 ml) and methanol (130 ml), then suction filtered, the filter cake was vacuum dried at 80 °C for 8 hours to obtain CPD 274-7 as a white solid (18.72 g, purity: 99.51%, yield: 75.12%), with a mass spectrum: 433.14 (M+H).

### Synthesis of Compound CPD 274

Referring to the synthesis and purification methods of compound CPD 4, it was only necessary to change the corresponding raw materials to obtain the target compound CPD 274 (15.69 g, purity: 99.95%, yield: 75.41%). 15.69 g of crude CPD 274 was purified by sublimation to obtain the sublimation-purified CPD 274 (12.55 g, purity: 99.95%, yield: 79.99%), with a mass spectrum: 672.20 (M+H). ¹H NMR (400 MHz, CDCl₃) δ 8.18 (t, J=2.2 Hz, 1H), 8.11-8.04 (m, 4H), 8.02 (d, J=7.9 Hz, 1H), 7.93 (d, J=2.4 Hz, 1H), 7.87-7.85 (m, 1H), 7.80-7.77 (m, 2H), 7.60-7.55 (m, 2H), 7.55-7.43 (m, 8H), 7.38-7.32 (m, 2H), 7.32-7.27 (m, 2H), 2.41 (d, J=1.0 Hz, 3H), 2.35-2.31 (m, 2H), 2.10-2.05 (m, 2H), 1.61 (t, J=5.4 Hz, 4H), 1.53-1.51 (m, 6H).

### 17. Synthesis Route of Compound CPD 276

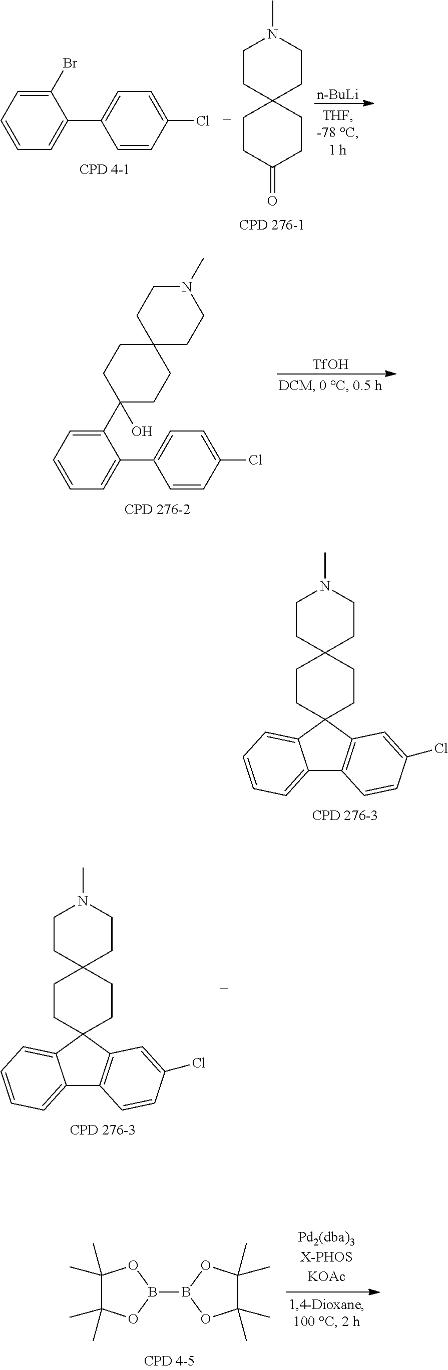

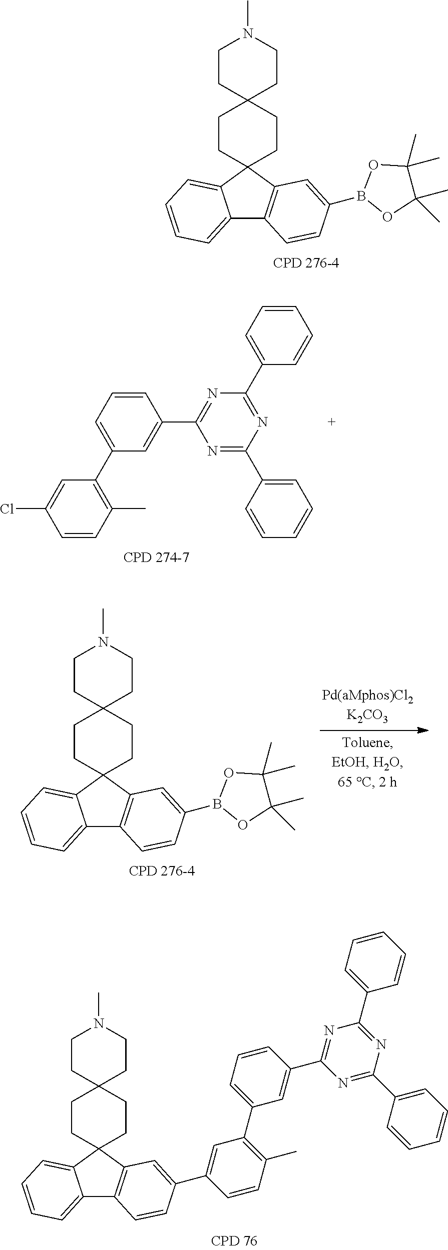 [SMILES: CN1CCC2(CC1)CCC1(CC2)c2ccccc2-c2ccc(B3OC(C)(C)C(C)(C)O3)cc21 ; Cc1ccc(Cl)cc1-c1cccc(-c2nc(-c3ccccc3)nc(-c3ccccc3)n2)c1 ; CN1CCC2(CC1)CCC1(CC2)c2ccccc2-c2ccc(B3OC(C)(C)C(C)(C)O3)cc21 ; Cc1ccc(-c2ccc3c(c2)C2(CCC4(CCN(C)CC4)CC2)c2ccccc2-3)cc1-c1cccc(-c2nc(-c3ccccc3)nc(-c3ccccc3)n2)c1]

### Synthesis of Compound CPD 276-2

Referring to the synthesis and purification methods of compound CPD 4-3, it was only necessary to change the corresponding raw materials to obtain the target compound CPD 276-2 (32.0 g, purity: 99.63%, yield: 63.52%), with a mass spectrum: 370.12 (M+H).

### Synthesis of Compound CPD 276-3

Referring to the synthesis and purification methods of compound CPD 4-4, it was only necessary to change the corresponding raw materials to obtain the target compound CPD 276-3 (25.14 g. purity: 99.68%, yield: 82.63%), with a mass spectrum: 352.18 (M+H).

### Synthesis of Compound CPD 276-4

Referring to the synthesis and purification methods of compound CPD 4-6, it was only necessary to change the corresponding raw materials to obtain the target compound CPD 276-4 (26.84 g, purity: 98.53%, yield: 83.59%), with a mass spectrum: 444.26 (M+H).

### Synthesis of Compound CPD 276

Referring to the synthesis and purification methods of compound CPD 4, it was only necessary to change the corresponding raw materials to obtain the target compound CPD 276 (16.87 g, purity: 99.94%, yield: 73.52%). 16.87 g of crude CPD 276 was purified by sublimation to obtain the sublimation-purified CPD 276 (13.26 g, purity: 99.95%, yield: 78.61%), with a mass spectrum: 715.32 (M+H). ¹H NMR (400 MHz, CDCl₃) δ 8.18 (t, J=2.1 Hz, 1H), 8.12-8.04 (m, 4H), 8.02 (d, J=7.9 Hz, 1H), 7.93 (d, J=2.4 Hz, 1H), 7.87-7.85 (m, 1H), 7.81-7.75 (m, 2H), 7.62-7.41 (m, 10H), 7.37-7.29 (m, 4H), 2.54-2.49 (m, 4H), 2.41 (s, 3H), 2.39-2.34 (m, 2H), 2.33 (s, 3H), 2.14-2.08 (m, 2H), 1.78 (t, J=6.2 Hz, 4H), 1.57-1.52 (m, 4H).

### Application Example: Fabrication of Organic Electroluminescent Device

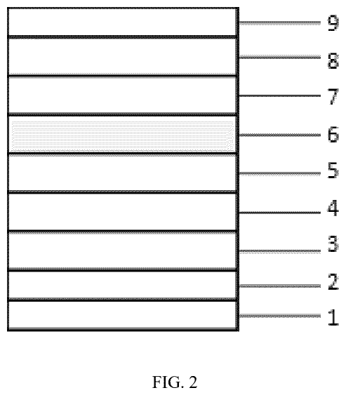

As shown in FIG. 2, the organic electroluminescent device included a glass substrate 1, an anode 2, a hole injection layer 3, a first hole transport layer 4 (HTL1), a second hole transport layer 5 (HTL2), a light-emitting layer 6, a hole blocking layer 7 (HBL), an electron transport layer 8 (ETL), and a cathode 9 arranged in stack.

The method for preparing the organic electroluminescent device included:

A 50 mm×50 mm×1.0 mm of glass substrate 1 with ITO (100 nm)transparent electrodes (anode 2 and cathode 9) was ultrasonically cleaned in ethanol for 10 minutes, then oven-dried at 150 °C, and then plasma treated with N₂ for 30 minutes. The washed glass substrate was mounted on a substrate holder of a vacuum evaporation device. First, the compound HATCN was evaporated on the surface of the side with transparent electrode wires in a manner of covering the transparent electrode to form a thin film of a film thickness of 5 nm (hole injection layer 3). Next, a layer of HTM1 was evaporated to form a thin film of a film thickness of 60 nm as HTL1 (a first hole transport layer 4), then, a layer of HTM2 was evaporated on the HTM1 thin film to form a thin film of a film thickness of 10 nm as HTL2 (a second hole transport layer 5). Then, the host material and the guest material were evaporated on the HTM2 film layer using a co-evaporation mode (the weight ratio of the host material to the doping material was 98%: 2%), forming a light-emitting layer 6 with a film thickness of 25 nm. A hole blocking layer 7 (HBL) with a film thickness of 5 nm and an electron transport layer 8 with a film thickness of 350 nm were sequentially formed on the light-emitting layer by evaporation, wherein the material of the hole blocking layer was the compounds of the present disclosure or the comparative compounds, and the material of the electron transport layer was the electron transport layer material ETL or the compounds of the present disclosure: LiQ with a weight ratio of 1:1. Then, Mg/Ag (100 nm, 1:9) was evaporated as the cathode material using the co-evaporation mode.

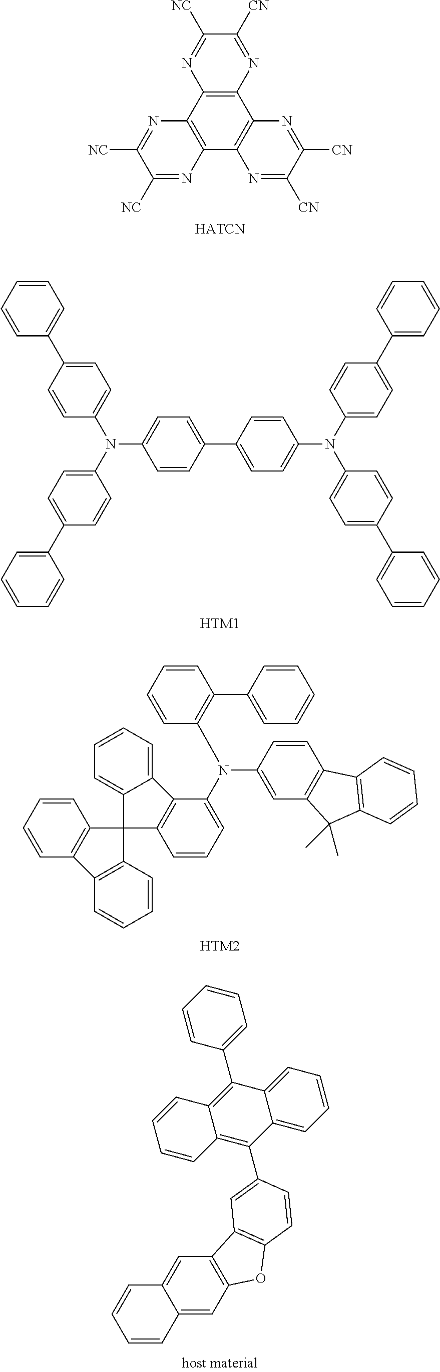

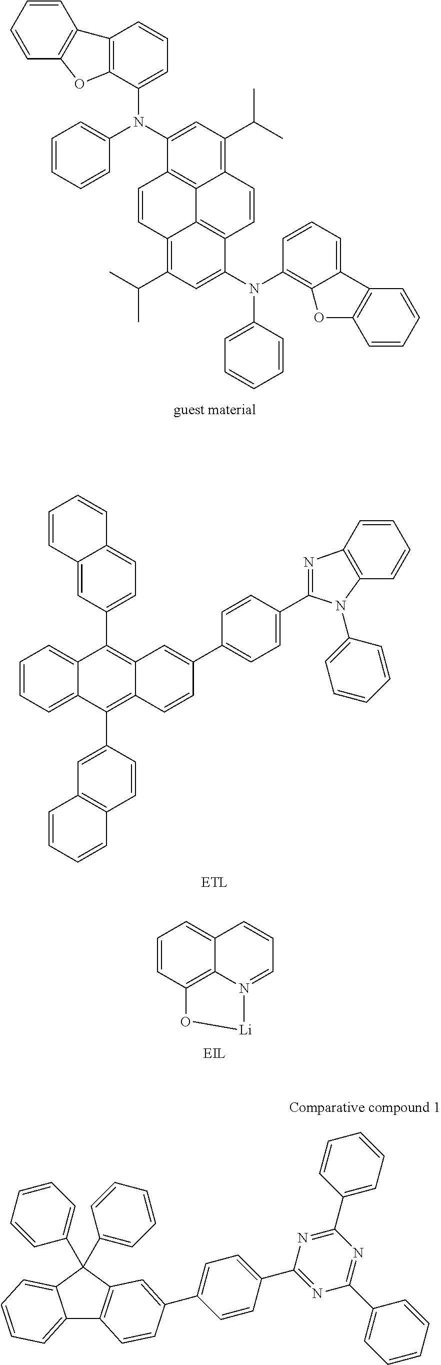

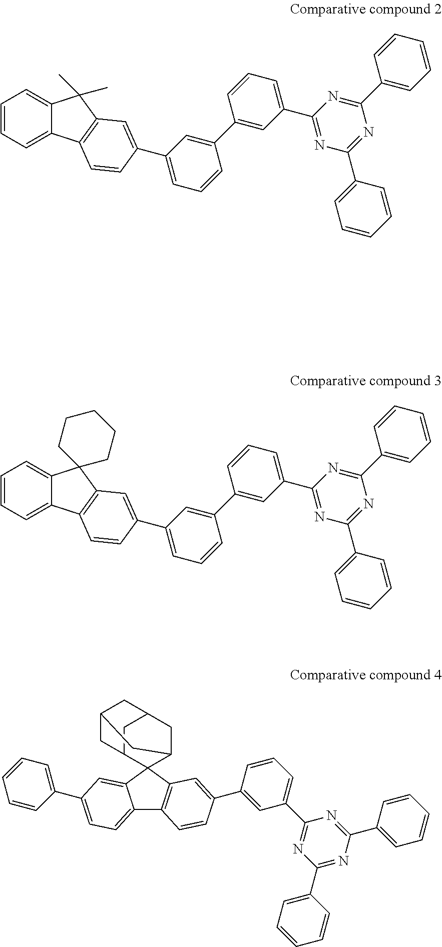 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-c4cccc(-c5ccc6c(c5)C5(CCCCC5)c5ccccc5-6)c4)c3)n2)cc1 ; c1ccc(-c2ccc3c(c2)C2(c4cc(-c5cccc(-c6nc(-c7ccccc7)nc(-c7ccccc7)n6)c5)ccc4-3)C3CC4CC(C3)CC2C4)cc1]

### Evaluation:

The above devices were subjected to the device performance tests. In the Examples and Comparative examples, a constant current power supply (Keithley 2400) was used to flow through the light-emitting element at a fixed current density, and a spectroradiometer (CS 2000) was used to measure the luminescent spectrum. At the same time, the voltage value, efficiency and the time when the test luminance reached 95% of the initial luminance (LT95) of the device were measured. The results are shown in Table 1:

TABLE 1

| | Compound | Compound | Starting | Current | |
|---|---|---|---|---|---|
| | (Hole Blocking | (Electron | Voltage | Efficiency | LT95(h) |
| | Layer) | Transport Layer) | (v) | (cd/a) | @1,000 nits |
| Example 1 | CPD 4 | ETL:LiQ | 3.66 | 8.21 | 157 |
| Example 2 | CPD 34 | ETL:LiQ | 3.67 | 8.33 | 169 |
| Example 3 | CPD 63 | ETL:LiQ | 3.71 | 8.51 | 175 |
| Example 4 | CPD 83 | ETL:LiQ | 3.75 | 8.11 | 152 |
| Example 5 | CPD 90 | ETL:LiQ | 3.78 | 8.13 | 179 |
| Example 6 | CPD 108 | ETL:LiQ | 3.69 | 8.41 | 188 |
| Example 7 | CPD 119 | ETL:LiQ | 3.73 | 8.37 | 177 |
| Example 8 | CPD 128 | ETL:LiQ | 3.72 | 8.69 | 180 |
| Example 9 | CPD 136 | ETL:LiQ | 3.75 | 8.42 | 154 |
| Example 10 | CPD 160 | ETL:LiQ | 3.76 | 8.52 | 163 |
| Example 11 | CPD 179 | ETL:LiQ | 3.73 | 8.28 | 158 |
| Example 12 | CPD 223 | ETL:LiQ | 3.65 | 8.46 | 163 |
| Example 13 | CPD 255 | ETL:LiQ | 3.69 | 8.32 | 192 |
| Example 14 | CPD 262 | ETL:LiQ | 3.70 | 8.52 | 213 |
| Example 15 | CPD 266 | ETL:LiQ | 3.68 | 8.37 | 203 |
| Example 16 | CPD 274 | ETL:LiQ | 3.71 | 8.43 | 171 |
| Example 17 | CPD 276 | ETL:LiQ | 3.64 | 8.46 | 162 |
| Comparative | Comparative | ETL:LiQ | 3.83 | 7.43 | 95 |
| example 1 | compound 1 | | | | |
| Comparative | Comparative | ETL:LiQ | 3.92 | 8.01 | 122 |
| example 2 | compound 2 | | | | |
| Comparative | Comparative | ETL:LiQ | 3.85 | 8.08 | 119 |
| example 3 | compound 3 | | | | |
| Comparative | Comparative | ETL:LiQ | 3.96 | 7.66 | 105 |
| example 4 | compound 4 | | | | |
| Example 18 | Comparative | CPD 4:LiQ | 3.73 | 8.36 | 153 |
| | compound 1 | | | | |
| Example 19 | Comparative | CPD 34:LiQ | 3.76 | 8.61 | 141 |
| | compound 2 | | | | |
| Example 20 | CPD 108 | CPD 4:LiQ | 3.72 | 8.52 | 167 |
| Example 21 | CPD 128 | CPD 34:LiQ | 3.75 | 8.78 | 163 |
| Comparative | Comparative | Comparative | 3.96 | 7.33 | 91 |
| example 5 | compound 1 | compound 1:LiQ | | | |
| Comparative | Comparative | Comparative | 3.93 | 7.98 | 112 |
| example 6 | compound 2 | compound 2:LiQ | | | |

From the data comparison in the above table, it can be seen that the use of the compounds of the present disclosure in an organic electroluminescent device as a hole blocking layer material and an electron transport layer material shows more superior performance in terms of driving voltage, luminous efficiency and lifetime of device compared to the comparative compounds.

The above results show that the compounds of the present disclosure have the advantages of good optical, electrical and heat stabilities, high luminous efficiency, low voltage and long lifetime, etc., and can be used in an organic light-emitting device. In particular, the compounds of the present disclosure have the potential to be applied to the AMOLED industry as a hole blocking layer material and an electron transport layer material.

1. A compound having a structure of formula (1): 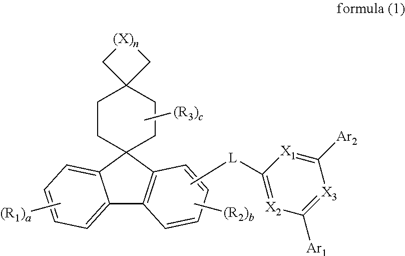
wherein each X is independently selected from the group consisting of C(R₄)₂, O, S, and NR₅;
wherein n is 0 or an integer of 1-6, when n=0, a ring is not formed through connection; when n≥1, a ring is formed through connection; and when n≥2, each X is the same or different;
wherein X₁, X₂ and X₃ are independently selected from N or CR₆, and at least one of X₁, X₂ and X₃ is N;
R₁, R₂ and R₃ are each independently selected from the group consisting of deuterium, halogen, cyano, nitro, C1-C40 alkyl, C1-C40 heteroalkyl, C2-C40 alkenyl, C2-C40 alkynyl, C3-C40 cycloalkyl, C3-C40 heterocycloalkyl, C6-C60 aryl, C5-C60 heteroaryl, C1-C40 alkoxy, C6-C60 aryloxy, C3-C40 alkylsilyl, C6-C60 arylsilyl, C1-C40 alkylboryl, C6-C60 arylboryl, C6-C60 monoarylphosphino, C6-C60 diarylphosphino, and C6-C60 arylamino;
R₄, R₅ and R₆ are substituents on respective rings and separately independently selected from the group consisting of hydrogen, deuterium, halogen, cyano, nitro, C1-C40 alkyl, C1-C40 heteroalkyl, C2-C40 alkenyl, C2-C40 alkynyl, C3-C40 cycloalkyl, C3-C40 heterocycloalkyl, C6-C60 aryl, C5-C60 heteroaryl, C1-C40 alkoxy, C6-C60 aryloxy, C3-C40 alkylsilyl, C6-C60 arylsilyl, C1-C40 alkylboryl, C6-C60 arylboryl, C6-C60 monoarylphosphino, C6-C60 diarylphosphino, and C6-C60 arylamino;
L is a single bond, a substituted or unsubstituted aromatic group with 6 to 30 ring atoms, or a substituted or unsubstituted heteroaromatic group with 6 to 30 ring atoms;
wherein a, b, and c are each independently selected from an integer of 0-8,
wherein Ar₁ and Ar₂ are each independently selected from a substituted or unsubstituted C6-C30 aryl, or a substituted or unsubstituted C2-C30 heteroaryl;
a heteroatom in the heteroaryl, heteroalkyl or heterocycloalkyl is selected from at least one heteroatom of O, S, N, Se, Si or Ge; and
the substitution refers to being substituted with deuterium, F, Cl, Br, C1-C6 alkyl, C3-C6 cycloalkyl, C1-C6 alkyl substituted amino, C6-C12 aryl, cyano, isocyano or phosphino; and the amount of substitutions of each substituent group is independently selected from one of single substitution to the maximum amount of substitutions.

2. The compound according to claim 1, having a structure of formula (2) or formula (3):

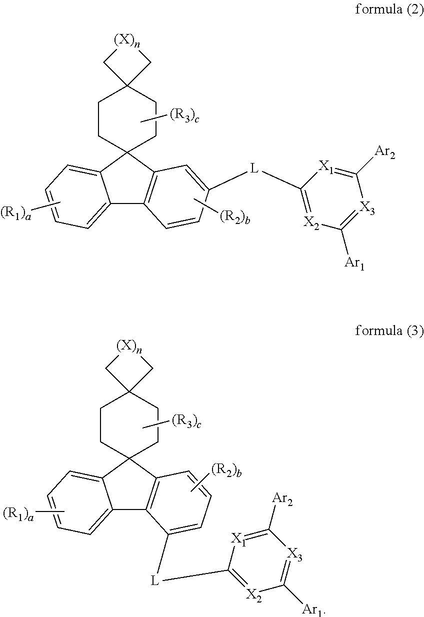

3. The compound according to claim 2, wherein when n=0, a ring is not formed through connection and X is absent; when n≥1, a ring is formed through connection; when n≥2, X is a plurality of C(R₄)₂, or a combination of one or more C(R₄)₂ and one of O, S and NR₅.

4. The compound according to claim 2, wherein 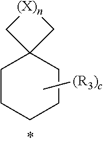
in the formula (1) to formula (3) is selected from one of the structures of formula A-1 to formula A-8:

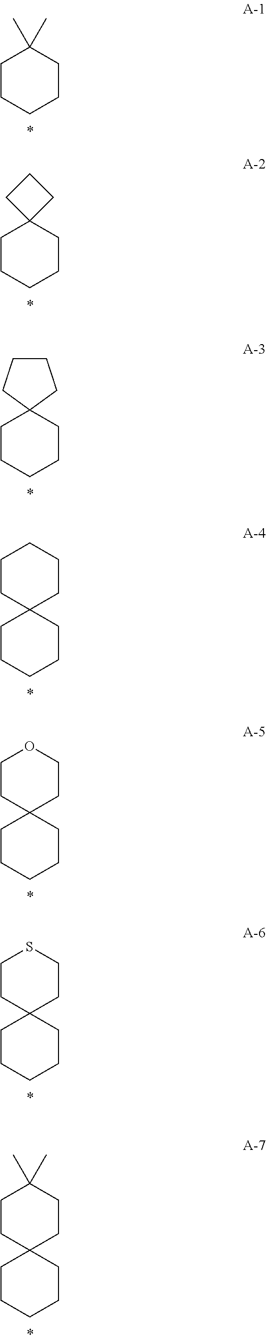

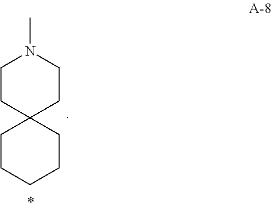

5. The compound according to claim 1, wherein L in the formula (1) is selected from a single bond or one of the structures of formula B-1 to formula B-18: 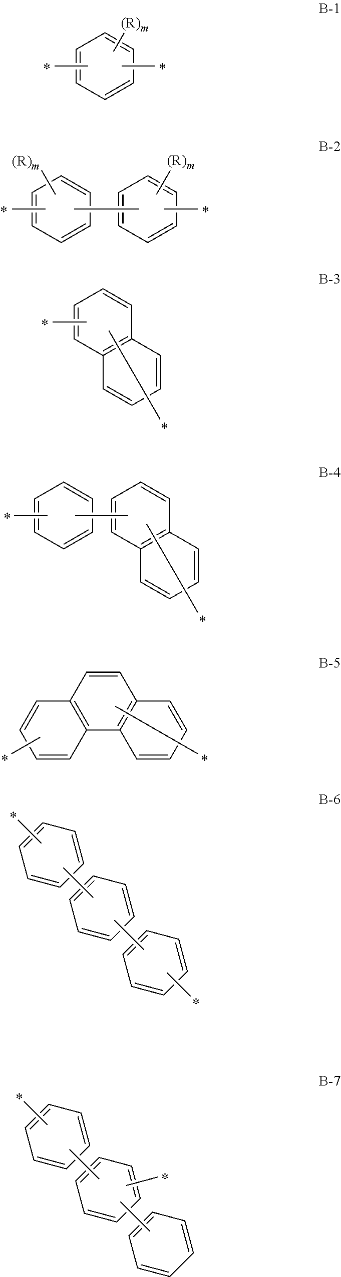 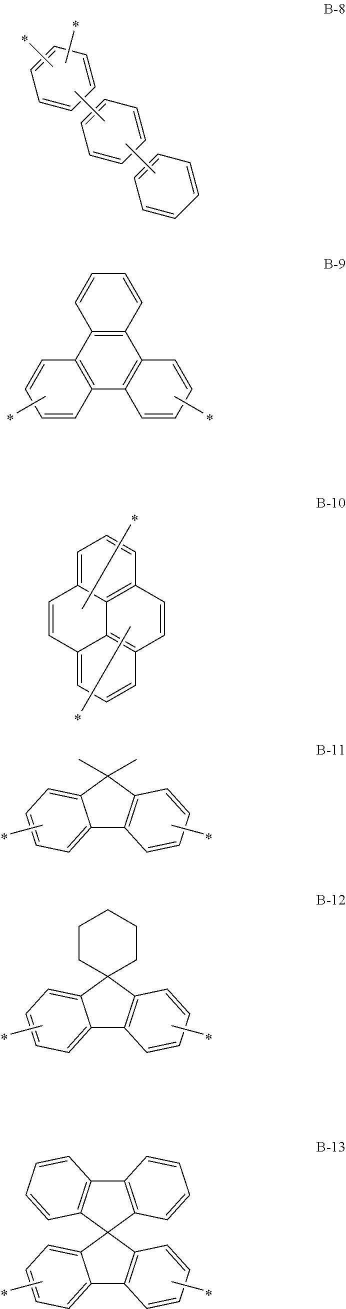 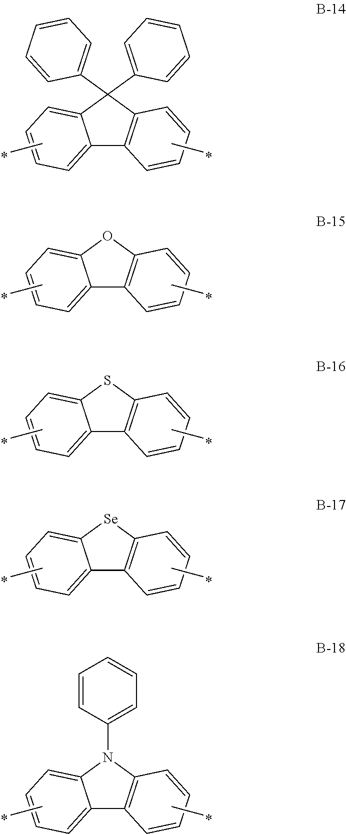
wherein each R is independently selected from the group consisting of deuterium, halogen, cyano, nitro, C1-C20 alkyl, C1-C20 heteroalkyl, C2-C20 alkenyl, C2-C20 alkynyl, C3-C20 cycloalkyl, C3-C20 heterocycloalkyl, C6-C30 aryl, C5-C30 heteroaryl, C1-C20 alkoxy, C6-C30 aryloxy, C3-C20 alkylsilyl, C6-C30 arylsilyl, C1-C20 alkylboryl, C6-C30 arylboryl, C6-C30 monoarylphosphino, C6-C30 diarylphosphino, and C6-C30 arylamino; and m is selected from an integer of 0-4.

6. The compound according to claim 5, wherein each R is independently selected from deuterium or C1-C4 alkyl.

7. The compound according to claim 4, wherein at least two of X₁ to X₃ comprise N, and R₁, R₂ and R₃ are each independently selected from the group consisting of deuterium, halogen, cyano, nitro, C1-C20 alkyl, C1-C20 heteroalkyl, C2-C20 alkenyl, C2-C20 alkynyl, C3-C20 cycloalkyl, C3-C20 heterocycloalkyl, C6-C30 aryl, C5-C30 heteroaryl, C1-C20 alkoxy, C6-C30 aryloxy, C3-C20 alkylsilyl, C6-C30 arylsilyl, C1-C20 alkylboryl, C6-C30 arylboryl, C6-C30 monoarylphosphino, C6-C30 diarylphosphino, and C6-C30 arylamino; and
R₆ is selected from the group consisting of hydrogen, deuterium, halogen, cyano, nitro, C1-C20 alkyl, C1-C20 heteroalkyl, C2-C20 alkenyl, C2-C20 alkynyl, C3-C20 cycloalkyl, C3-C20 heterocycloalkyl, C6-C30 aryl, C5-C30 heteroaryl, C1-C20 alkoxy, C6-C30 aryloxy, C3-C20 alkylsilyl, C6-C30 arylsilyl, C1-C20 alkylboryl, C6-C30 arylboryl, C6-C30 monoarylphosphino, C6-C30 diarylphosphino, and C6-C30 arylamino.

8. The compound according to claim 7, wherein two of X₁ to X₃ are N, and the remaining one of X₁ to X₃ is CH or N.

9. The compound according to claim 7, wherein X₁ to X₃ are each N.

10. The compound according to claim 9, wherein X₁ to X₃ are each N; and a, b, and c are each 0.

11. The compound according to claim 1, wherein Ar₁ and Ar₂ are each independently selected from the group consisting of phenyl, deuterated phenyl, methylphenyl, fluorophenyl, tert-butylphenyl, trideuterated methylphenyl, biphenyl, naphthyl, deuterated naphthyl, dibenzofuranyl, dibenzothiophenyl, 9,9-dimethylfluorenyl, 9,9-diphenylfluorenyl, spirodifluorenyl, phenanthryl, pyrenyl, chrysenyl, carbazolyl, pyridyl, pyrimidinyl, 4-cyanophenyl, 3-cyanophenyl, and benzophenanthrenyl, or a combination of at least two thereof.

12. The compound according to claim 1, wherein the compound of formula (1) comprises at least one deuterium atom.

13. The compound according to claim 1, having one of the following structures:

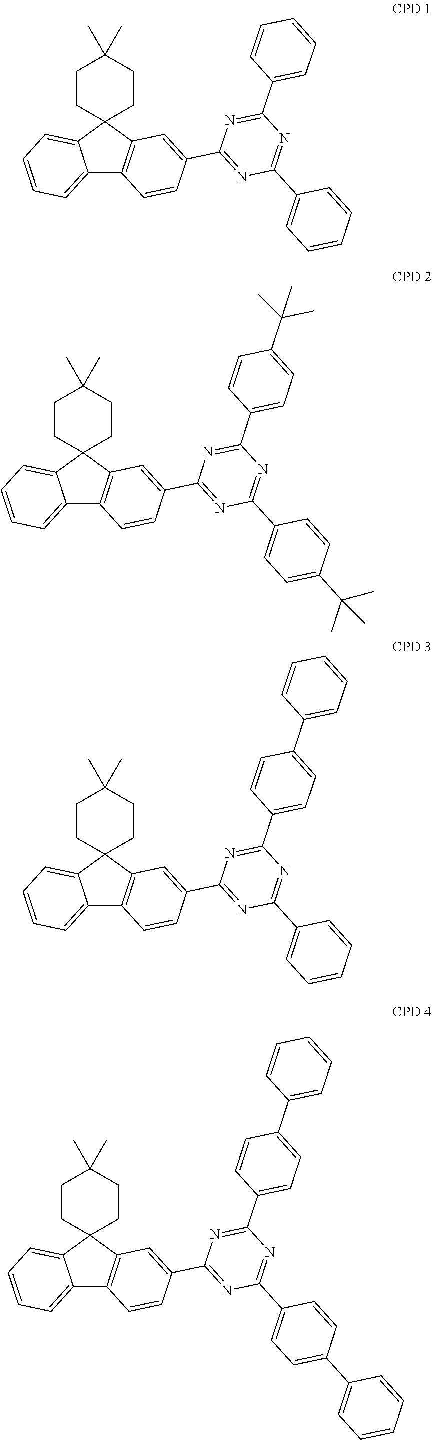

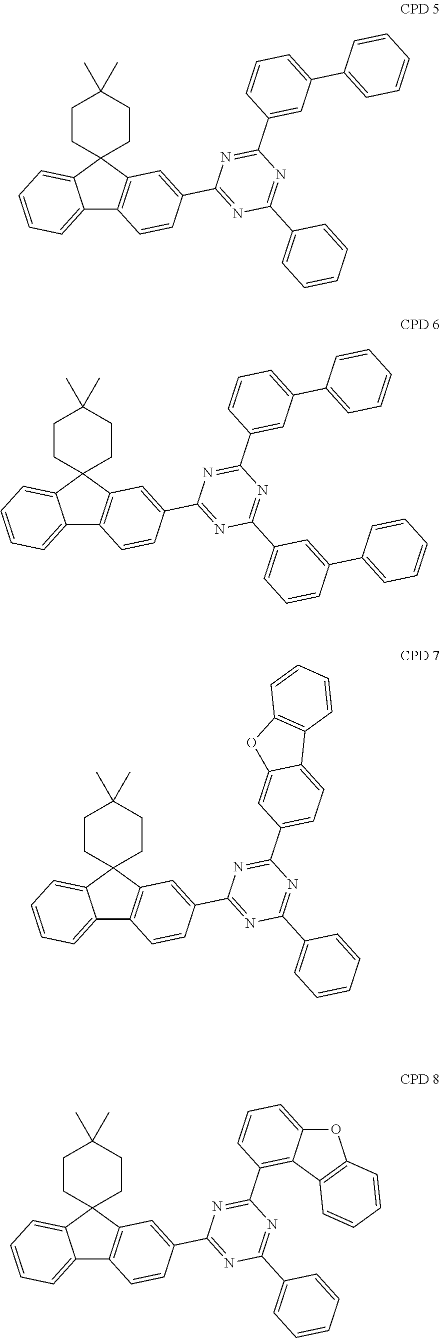 [SMILES: CC1(C)CCC2(CC1)c1ccccc1-c1ccc(-c3nc(-c4ccccc4)nc(-c4cccc(-c5ccccc5)c4)n3)cc12 ; CC1(C)CCC2(CC1)c1ccccc1-c1ccc(-c3nc(-c4cccc(-c5ccccc5)c4)nc(-c4cccc(-c5ccccc5)c4)n3)cc12 ; CC1(C)CCC2(CC1)c1ccccc1-c1ccc(-c3nc(-c4ccccc4)nc(-c4ccc5c(c4)oc4ccccc45)n3)cc12 ; CC1(C)CCC2(CC1)c1ccccc1-c1ccc(-c3nc(-c4ccccc4)nc(-c4cccc5oc6ccccc6c45)n3)cc12]

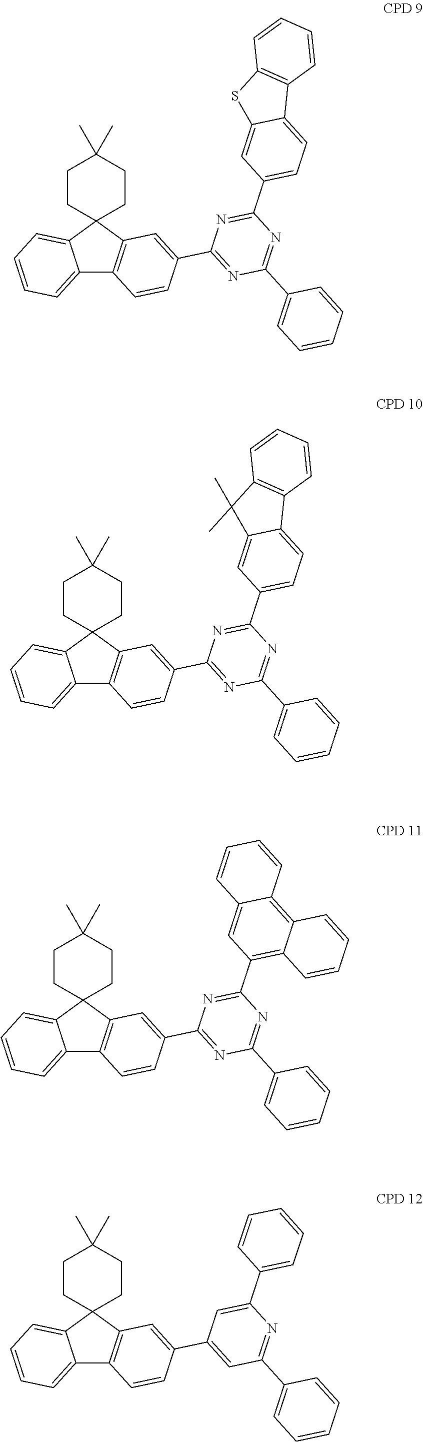 [SMILES: CC1(C)CCC2(CC1)c1ccccc1-c1ccc(-c3nc(-c4ccccc4)nc(-c4ccc5c(c4)sc4ccccc45)n3)cc12 ; CC1(C)CCC2(CC1)c1ccccc1-c1ccc(-c3nc(-c4ccccc4)nc(-c4ccc5c(c4)C(C)(C)c4ccccc4-5)n3)cc12 ; CC1(C)CCC2(CC1)c1ccccc1-c1ccc(-c3nc(-c4ccccc4)nc(-c4cc5ccccc5c5ccccc45)n3)cc12 ; CC1(C)CCC2(CC1)c1ccccc1-c1ccc(-c3cc(-c4ccccc4)nc(-c4ccccc4)c3)cc12]

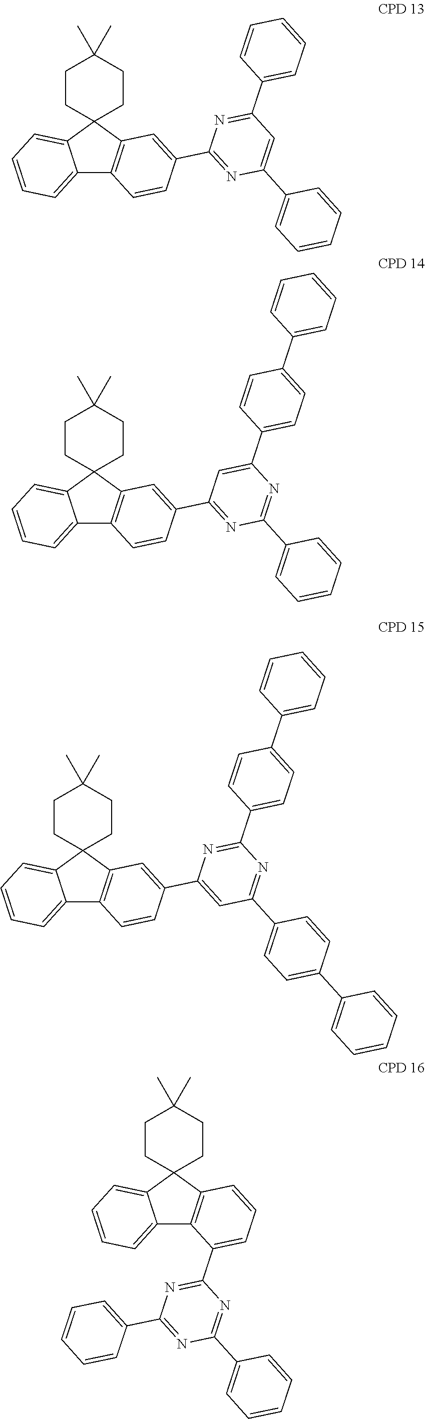

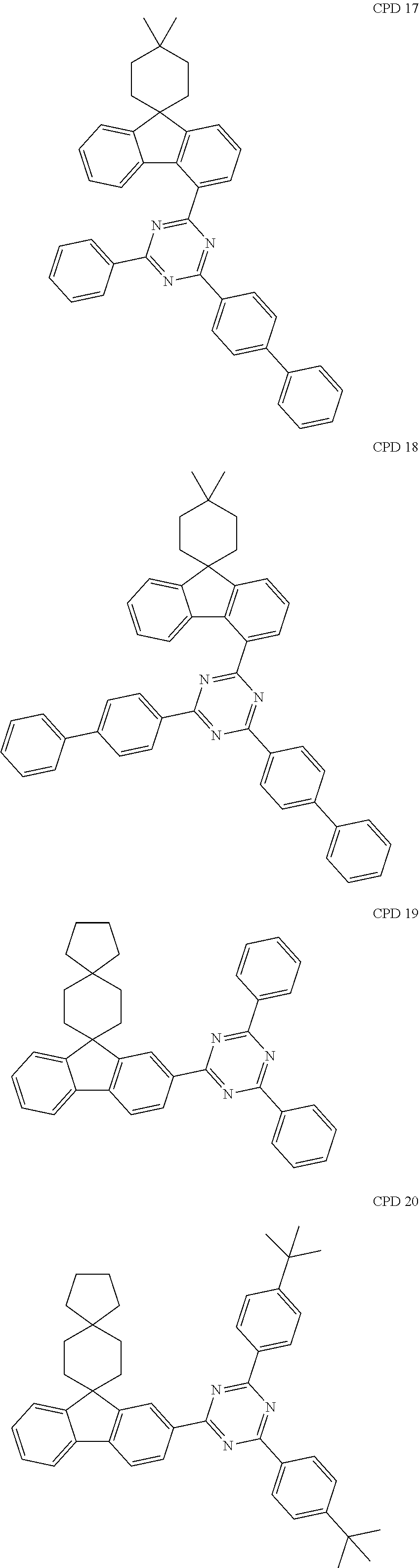 [SMILES: CC1(C)CCC2(CC1)c1ccccc1-c1c(-c3nc(-c4ccccc4)nc(-c4ccc(-c5ccccc5)cc4)n3)cccc12 ; CC1(C)CCC2(CC1)c1ccccc1-c1c(-c3nc(-c4ccc(-c5ccccc5)cc4)nc(-c4ccc(-c5ccccc5)cc4)n3)cccc12 ; c1ccc(-c2nc(-c3ccccc3)nc(-c3ccc4c(c3)C3(CCC5(CCCC5)CC3)c3ccccc3-4)n2)cc1 ; CC(C)(C)c1ccc(-c2nc(-c3ccc(C(C)(C)C)cc3)nc(-c3ccc4c(c3)C3(CCC5(CCCC5)CC3)c3ccccc3-4)n2)cc1]

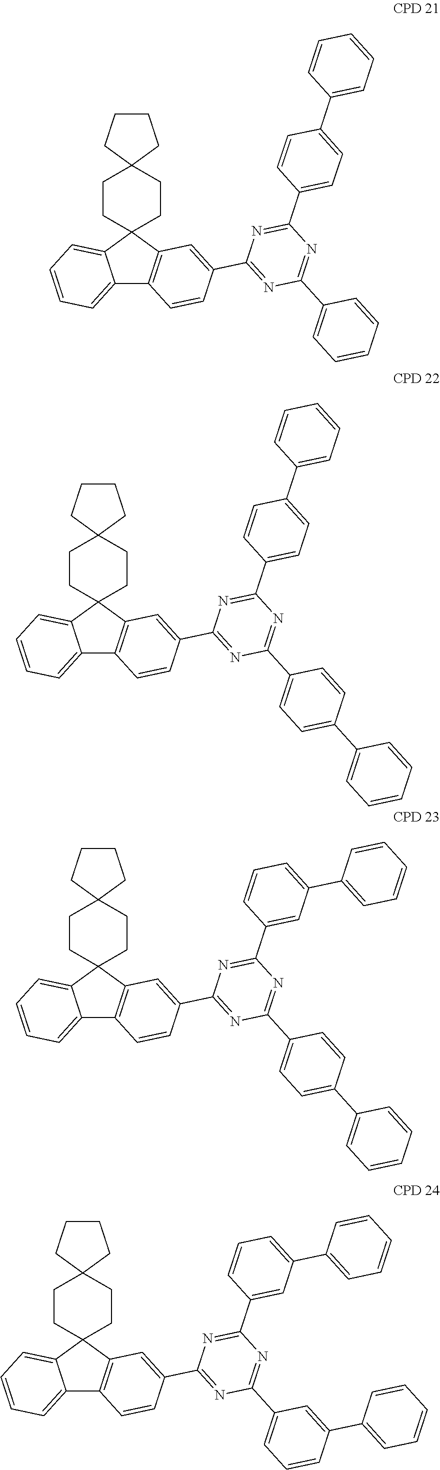

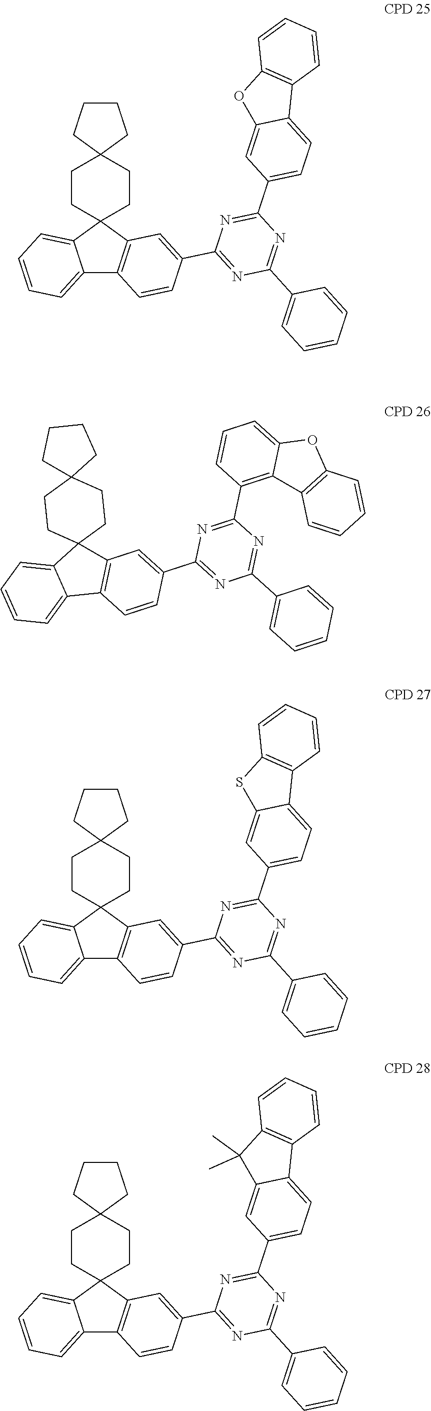

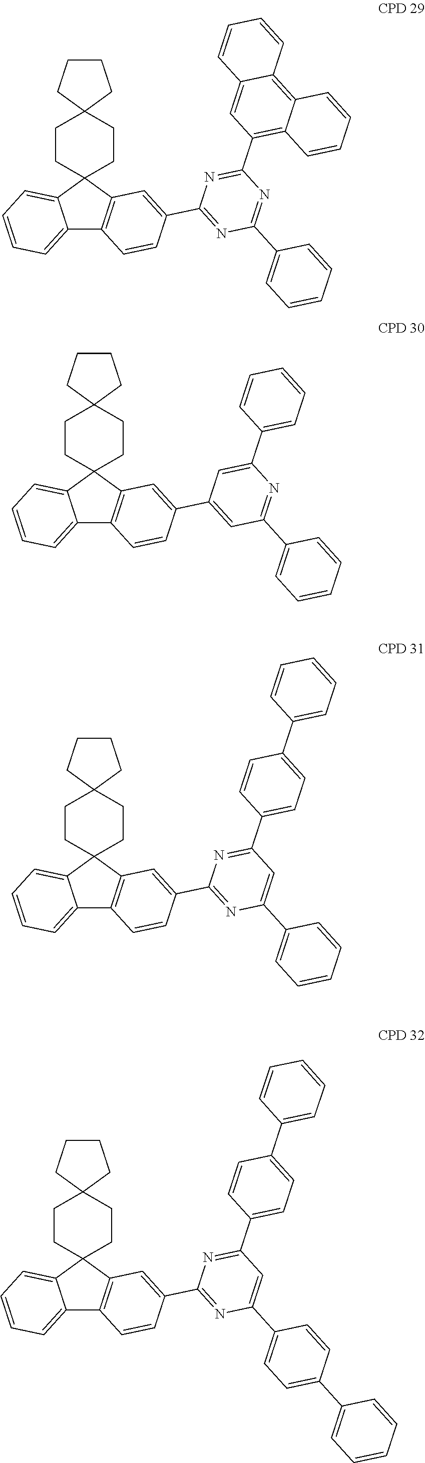 [SMILES: c1ccc(-c2nc(-c3ccc4c(c3)C3(CCC5(CCCC5)CC3)c3ccccc3-4)nc(-c3cc4ccccc4c4ccccc34)n2)cc1 ; c1ccc(-c2cc(-c3ccc4c(c3)C3(CCC5(CCCC5)CC3)c3ccccc3-4)cc(-c3ccccc3)n2)cc1 ; c1ccc(-c2ccc(-c3cc(-c4ccccc4)nc(-c4ccc5c(c4)C4(CCC6(CCCC6)CC4)c4ccccc4-5)n3)cc2)cc1 ; c1ccc(-c2ccc(-c3cc(-c4ccc(-c5ccccc5)cc4)nc(-c4ccc5c(c4)C4(CCC6(CCCC6)CC4)c4ccccc4-5)n3)cc2)cc1]

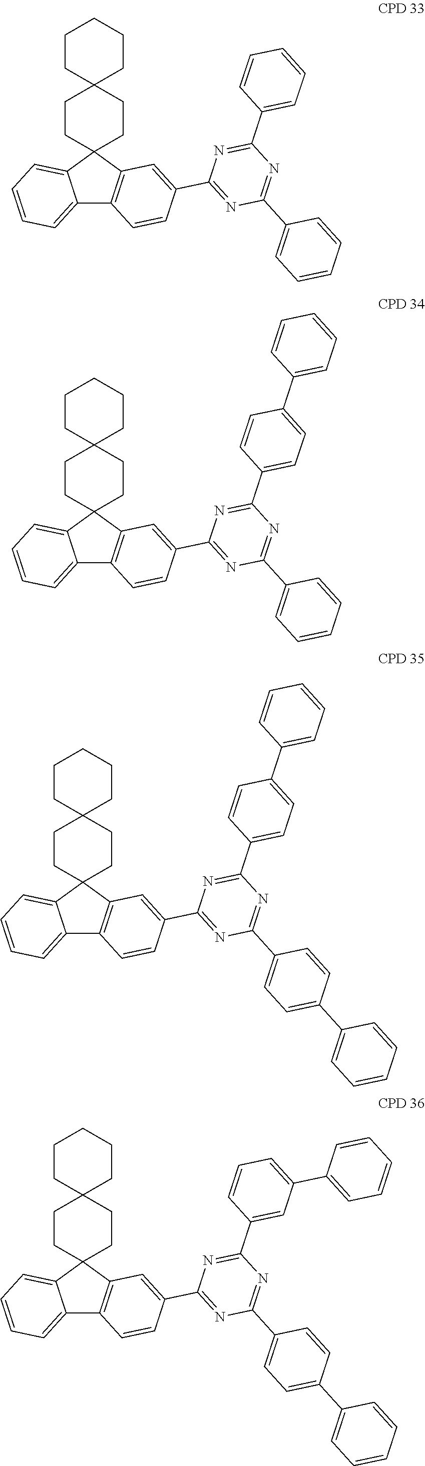

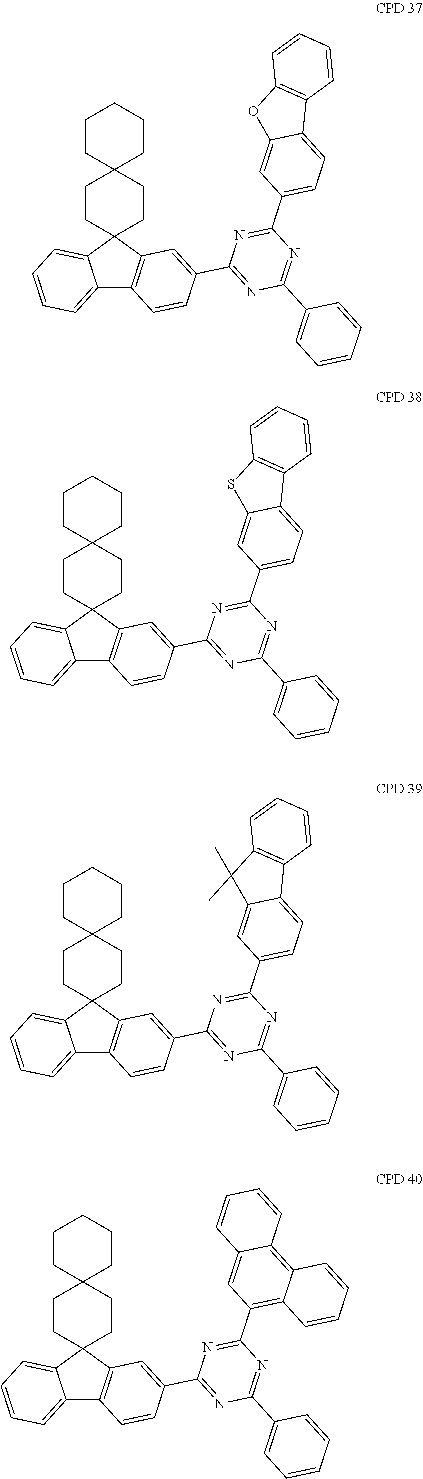 [SMILES: c1ccc(-c2nc(-c3ccc4c(c3)C3(CCC5(CCCCC5)CC3)c3ccccc3-4)nc(-c3ccc4c(c3)oc3ccccc34)n2)cc1 ; c1ccc(-c2nc(-c3ccc4c(c3)C3(CCC5(CCCCC5)CC3)c3ccccc3-4)nc(-c3ccc4c(c3)sc3ccccc34)n2)cc1 ; CC1(C)c2ccccc2-c2ccc(-c3nc(-c4ccccc4)nc(-c4ccc5c(c4)C4(CCC6(CCCCC6)CC4)c4ccccc4-5)n3)cc21 ; c1ccc(-c2nc(-c3ccc4c(c3)C3(CCC5(CCCCC5)CC3)c3ccccc3-4)nc(-c3cc4ccccc4c4ccccc34)n2)cc1]

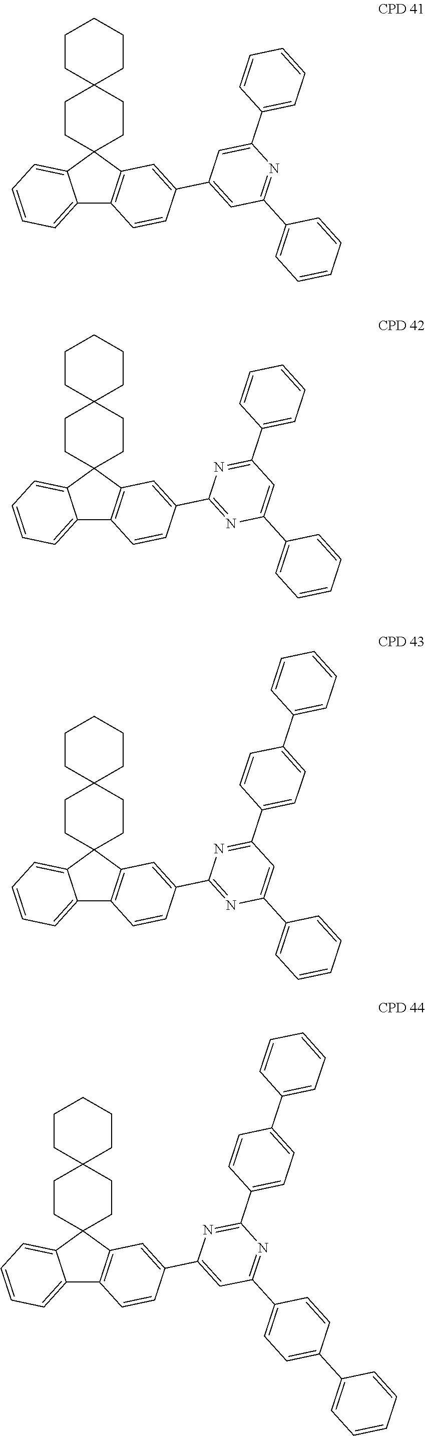 [SMILES: c1ccc(-c2cc(-c3ccc4c(c3)C3(CCC5(CCCCC5)CC3)c3ccccc3-4)cc(-c3ccccc3)n2)cc1 ; c1ccc(-c2cc(-c3ccccc3)nc(-c3ccc4c(c3)C3(CCC5(CCCCC5)CC3)c3ccccc3-4)n2)cc1 ; c1ccc(-c2ccc(-c3cc(-c4ccccc4)nc(-c4ccc5c(c4)C4(CCC6(CCCCC6)CC4)c4ccccc4-5)n3)cc2)cc1 ; c1ccc(-c2ccc(-c3cc(-c4ccc5c(c4)C4(CCC6(CCCCC6)CC4)c4ccccc4-5)nc(-c4ccc(-c5ccccc5)cc4)n3)cc2)cc1]

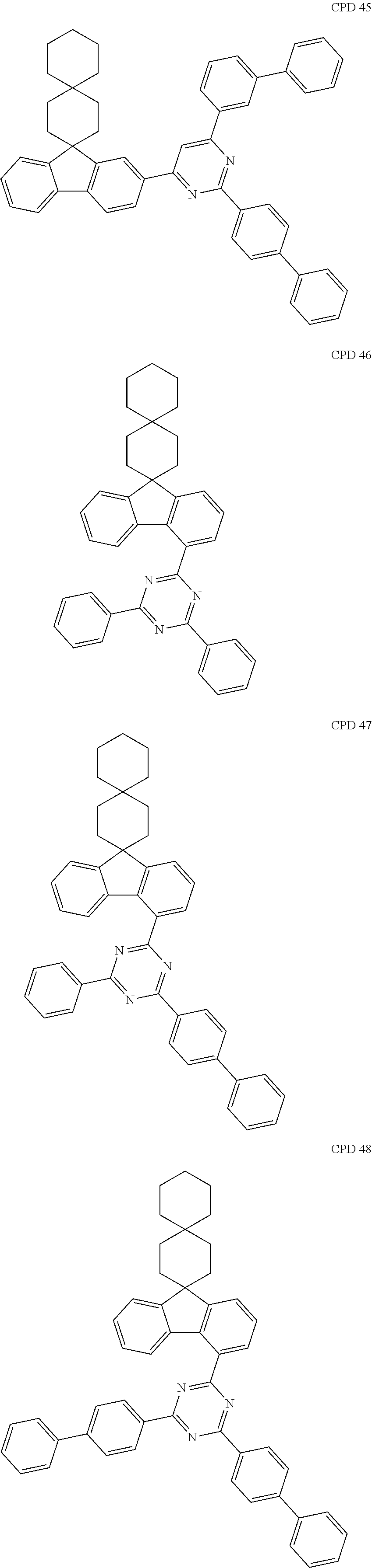 [SMILES: c1ccc(-c2ccc(-c3nc(-c4cccc(-c5ccccc5)c4)cc(-c4ccc5c(c4)C4(CCC6(CCCCC6)CC4)c4ccccc4-5)n3)cc2)cc1 ; c1ccc(-c2nc(-c3ccccc3)nc(-c3cccc4c3-c3ccccc3C43CCC4(CCCCC4)CC3)n2)cc1 ; c1ccc(-c2ccc(-c3nc(-c4ccccc4)nc(-c4cccc5c4-c4ccccc4C54CCC5(CCCCC5)CC4)n3)cc2)cc1 ; c1ccc(-c2ccc(-c3nc(-c4ccc(-c5ccccc5)cc4)nc(-c4cccc5c4-c4ccccc4C54CCC5(CCCCC5)CC4)n3)cc2)cc1]

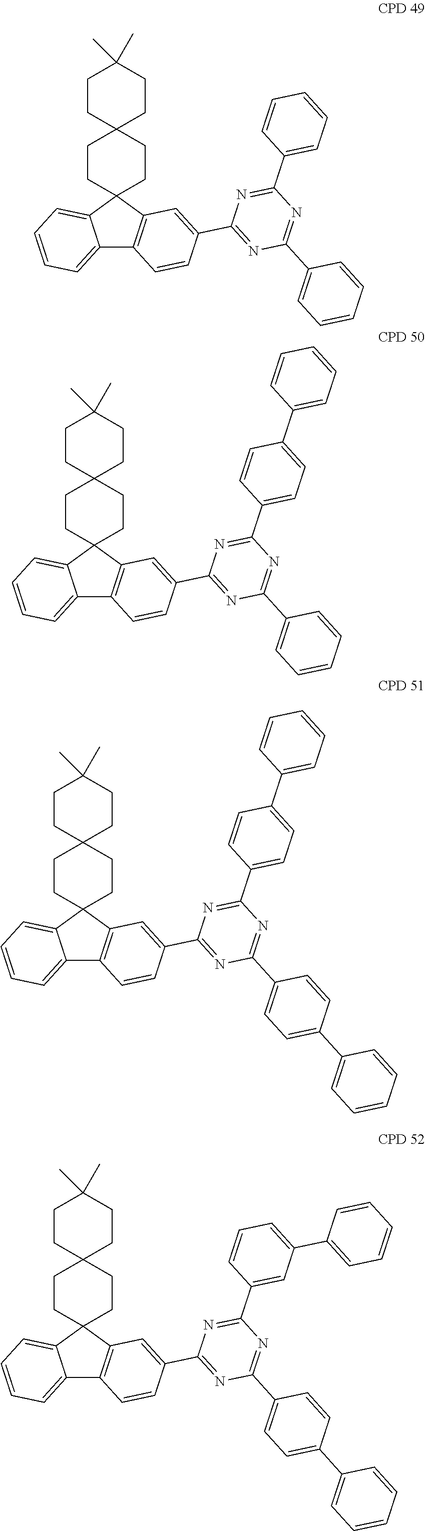

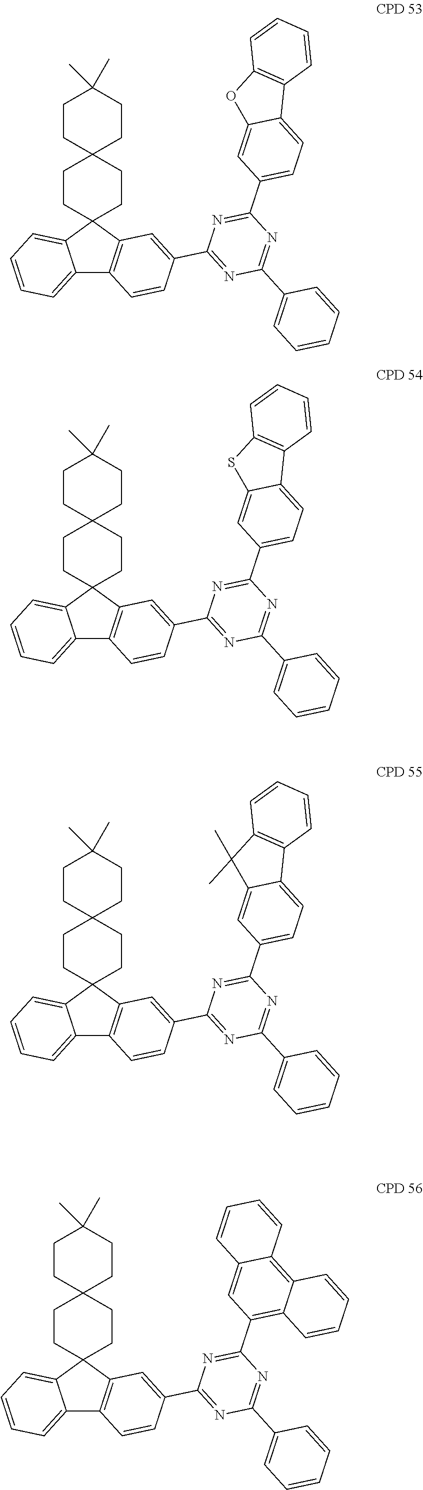

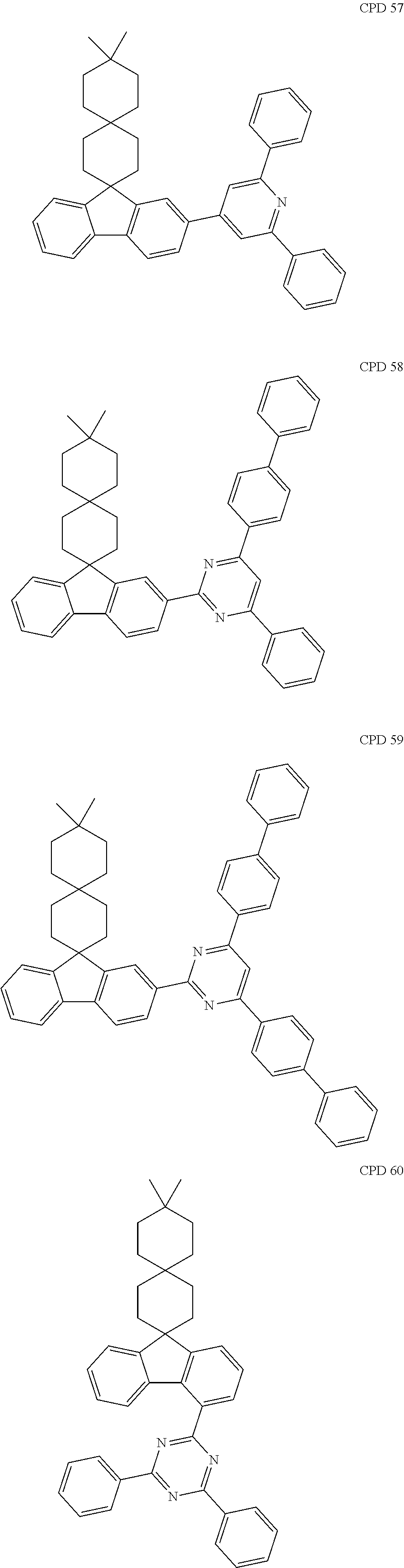 [SMILES: CC1(C)CCC2(CC1)CCC1(CC2)c2ccccc2-c2ccc(-c3cc(-c4ccccc4)nc(-c4ccccc4)c3)cc21 ; CC1(C)CCC2(CC1)CCC1(CC2)c2ccccc2-c2ccc(-c3nc(-c4ccccc4)cc(-c4ccc(-c5ccccc5)cc4)n3)cc21 ; CC1(C)CCC2(CC1)CCC1(CC2)c2ccccc2-c2ccc(-c3nc(-c4ccc(-c5ccccc5)cc4)cc(-c4ccc(-c5ccccc5)cc4)n3)cc21 ; CC1(C)CCC2(CC1)CCC1(CC2)c2ccccc2-c2c(-c3nc(-c4ccccc4)nc(-c4ccccc4)n3)cccc21]

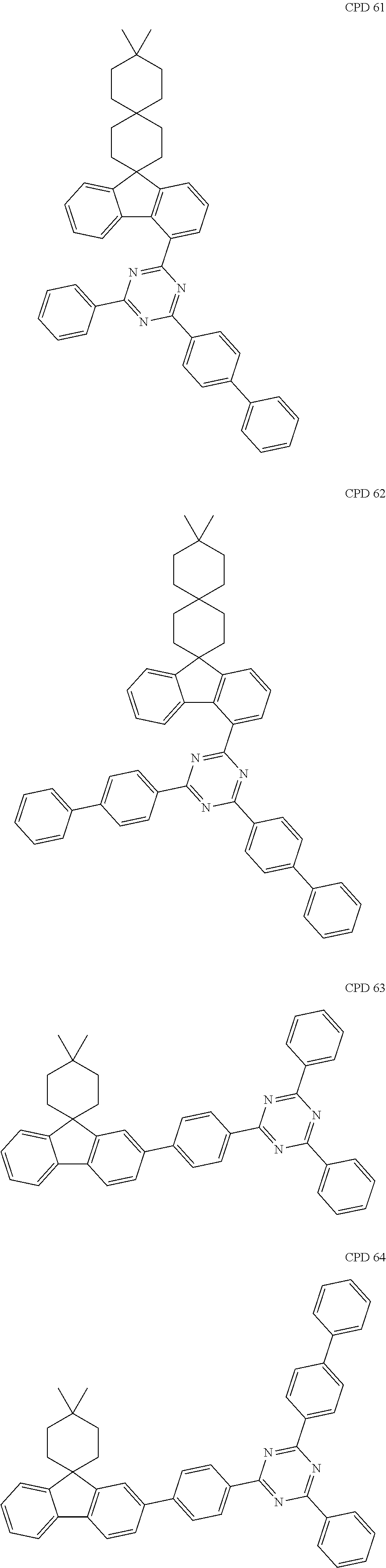 [SMILES: CC1(C)CCC2(CC1)CCC1(CC2)c2ccccc2-c2c(-c3nc(-c4ccccc4)nc(-c4ccc(-c5ccccc5)cc4)n3)cccc21 ; CC1(C)CCC2(CC1)CCC1(CC2)c2ccccc2-c2c(-c3nc(-c4ccc(-c5ccccc5)cc4)nc(-c4ccc(-c5ccccc5)cc4)n3)cccc21 ; CC1(C)CCC2(CC1)c1ccccc1-c1ccc(-c3ccc(-c4nc(-c5ccccc5)nc(-c5ccccc5)n4)cc3)cc12 ; CC1(C)CCC2(CC1)c1ccccc1-c1ccc(-c3ccc(-c4nc(-c5ccccc5)nc(-c5ccc(-c6ccccc6)cc5)n4)cc3)cc12]

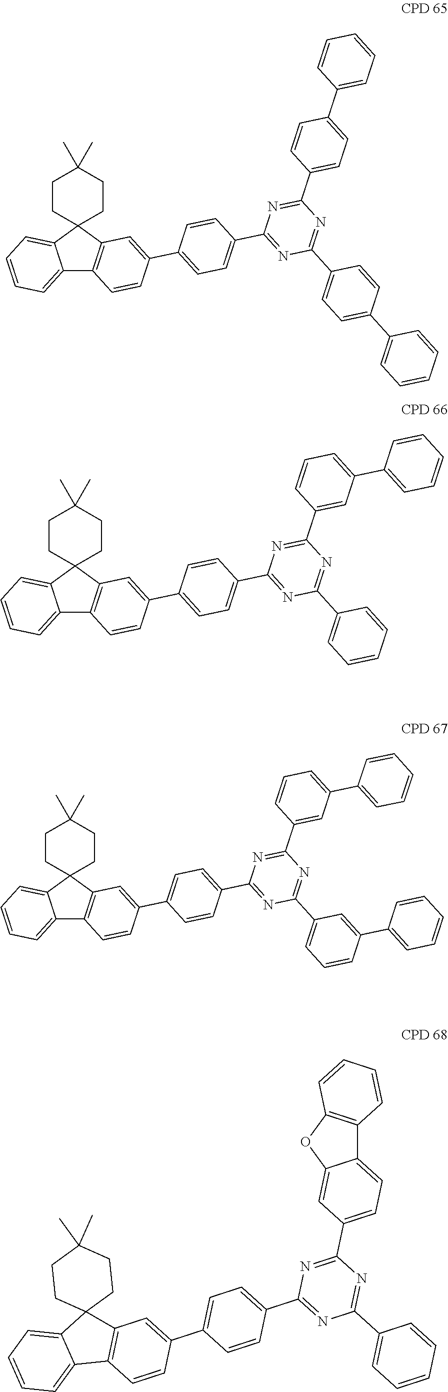 [SMILES: CC1(C)CCC2(CC1)c1ccccc1-c1ccc(-c3ccc(-c4nc(-c5ccc(-c6ccccc6)cc5)nc(-c5ccc(-c6ccccc6)cc5)n4)cc3)cc12 ; CC1(C)CCC2(CC1)c1ccccc1-c1ccc(-c3ccc(-c4nc(-c5ccccc5)nc(-c5cccc(-c6ccccc6)c5)n4)cc3)cc12 ; CC1(C)CCC2(CC1)c1ccccc1-c1ccc(-c3ccc(-c4nc(-c5cccc(-c6ccccc6)c5)nc(-c5cccc(-c6ccccc6)c5)n4)cc3)cc12 ; CC1(C)CCC2(CC1)c1ccccc1-c1ccc(-c3ccc(-c4nc(-c5ccccc5)nc(-c5ccc6c(c5)oc5ccccc56)n4)cc3)cc12]

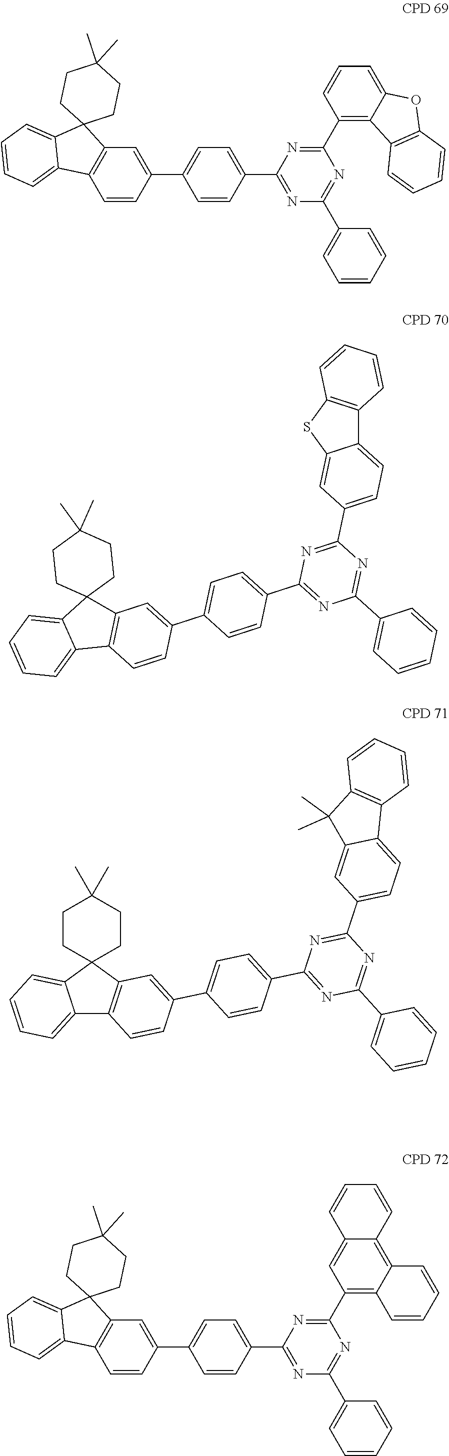 [SMILES: CC1(C)CCC2(CC1)c1ccccc1-c1ccc(-c3ccc(-c4nc(-c5ccccc5)nc(-c5cccc6oc7ccccc7c56)n4)cc3)cc12 ; CC1(C)CCC2(CC1)c1ccccc1-c1ccc(-c3ccc(-c4nc(-c5ccccc5)nc(-c5ccc6c(c5)sc5ccccc56)n4)cc3)cc12 ; CC1(C)CCC2(CC1)c1ccccc1-c1ccc(-c3ccc(-c4nc(-c5ccccc5)nc(-c5ccc6c(c5)C(C)(C)c5ccccc5-6)n4)cc3)cc12 ; CC1(C)CCC2(CC1)c1ccccc1-c1ccc(-c3ccc(-c4nc(-c5ccccc5)nc(-c5cc6ccccc6c6ccccc56)n4)cc3)cc12]

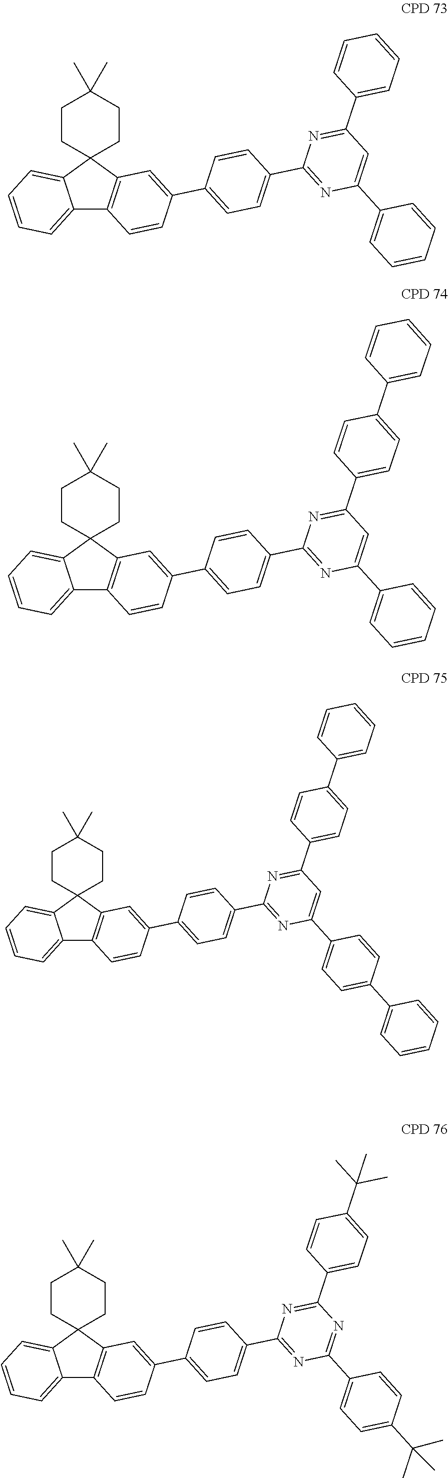 [SMILES: CC1(C)CCC2(CC1)c1ccccc1-c1ccc(-c3ccc(-c4nc(-c5ccccc5)cc(-c5ccccc5)n4)cc3)cc12 ; CC1(C)CCC2(CC1)c1ccccc1-c1ccc(-c3ccc(-c4nc(-c5ccccc5)cc(-c5ccc(-c6ccccc6)cc5)n4)cc3)cc12 ; CC1(C)CCC2(CC1)c1ccccc1-c1ccc(-c3ccc(-c4nc(-c5ccc(-c6ccccc6)cc5)cc(-c5ccc(-c6ccccc6)cc5)n4)cc3)cc12 ; CC1(C)CCC2(CC1)c1ccccc1-c1ccc(-c3ccc(-c4nc(-c5ccc(C(C)(C)C)cc5)nc(-c5ccc(C(C)(C)C)cc5)n4)cc3)cc12]

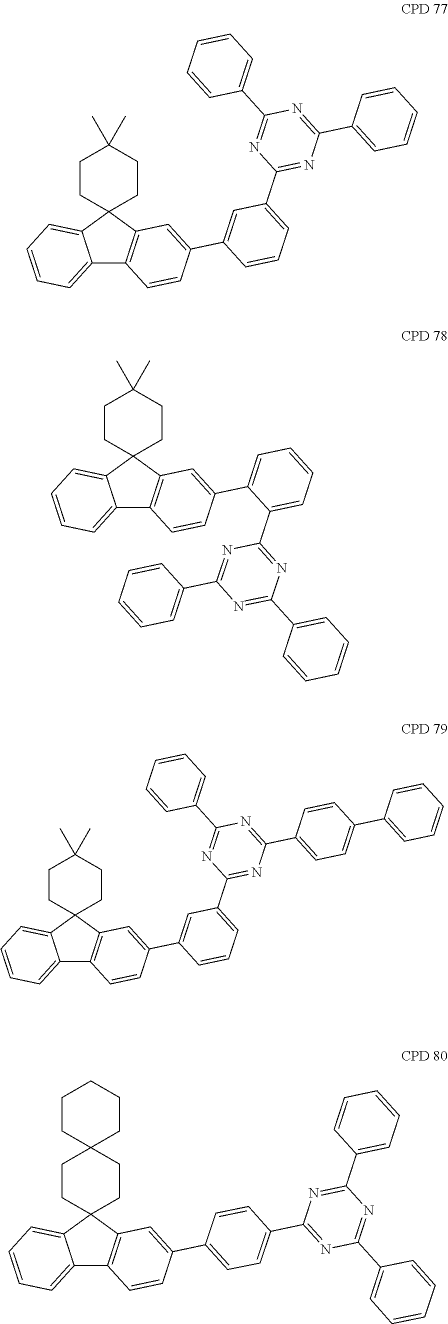 [SMILES: CC1(C)CCC2(CC1)c1ccccc1-c1ccc(-c3cccc(-c4nc(-c5ccccc5)nc(-c5ccccc5)n4)c3)cc12 ; CC1(C)CCC2(CC1)c1ccccc1-c1ccc(-c3ccccc3-c3nc(-c4ccccc4)nc(-c4ccccc4)n3)cc12 ; CC1(C)CCC2(CC1)c1ccccc1-c1ccc(-c3cccc(-c4nc(-c5ccccc5)nc(-c5ccc(-c6ccccc6)cc5)n4)c3)cc12 ; c1ccc(-c2nc(-c3ccccc3)nc(-c3ccc(-c4ccc5c(c4)C4(CCC6(CCCCC6)CC4)c4ccccc4-5)cc3)n2)cc1]

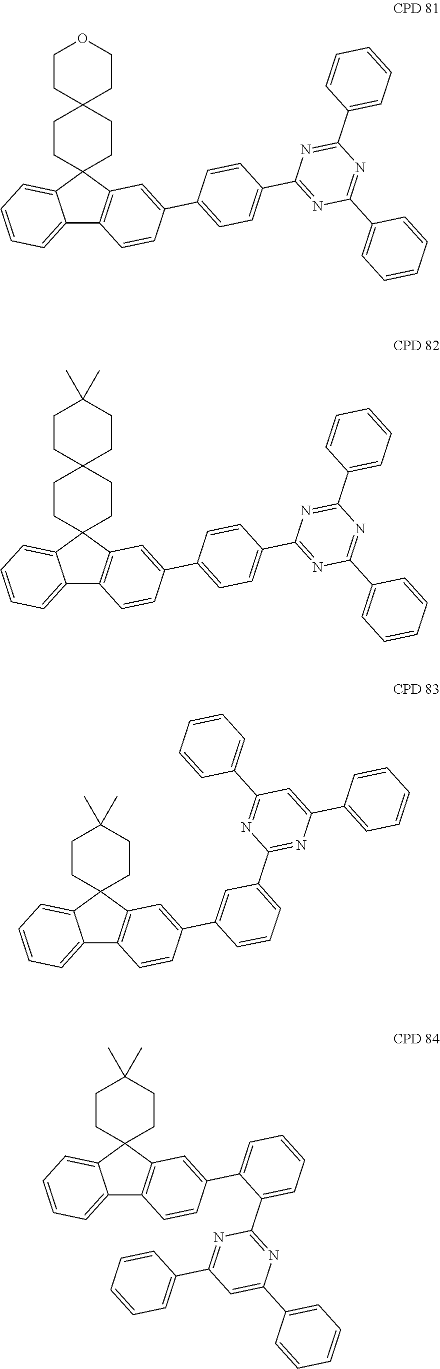 [SMILES: c1ccc(-c2nc(-c3ccccc3)nc(-c3ccc(-c4ccc5c(c4)C4(CCC6(CCOCC6)CC4)c4ccccc4-5)cc3)n2)cc1 ; CC1(C)CCC2(CC1)CCC1(CC2)c2ccccc2-c2ccc(-c3ccc(-c4nc(-c5ccccc5)nc(-c5ccccc5)n4)cc3)cc21 ; CC1(C)CCC2(CC1)c1ccccc1-c1ccc(-c3cccc(-c4nc(-c5ccccc5)cc(-c5ccccc5)n4)c3)cc12 ; CC1(C)CCC2(CC1)c1ccccc1-c1ccc(-c3ccccc3-c3nc(-c4ccccc4)cc(-c4ccccc4)n3)cc12]

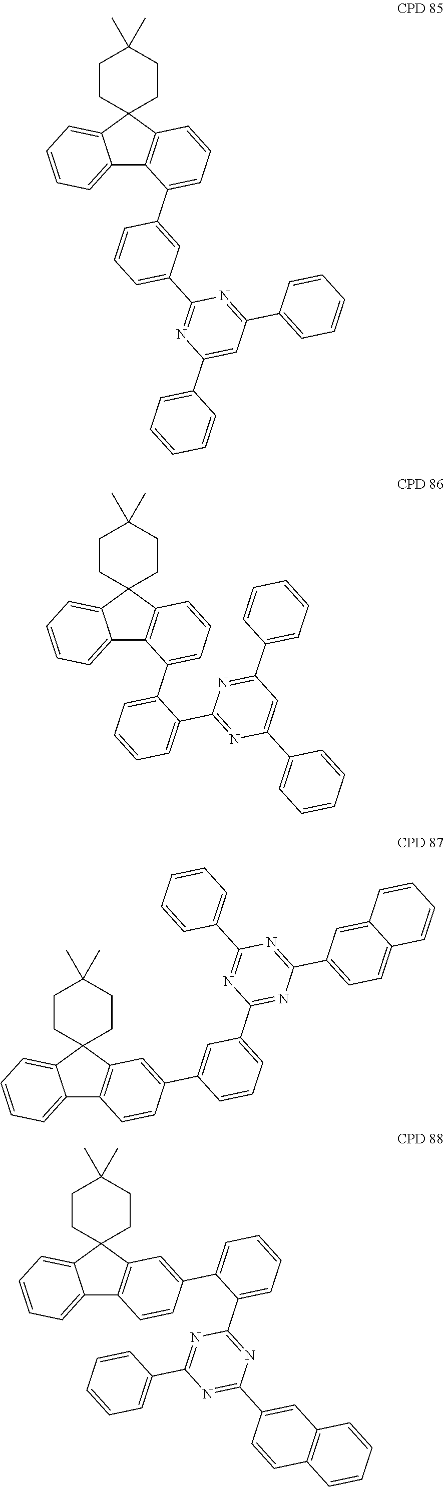

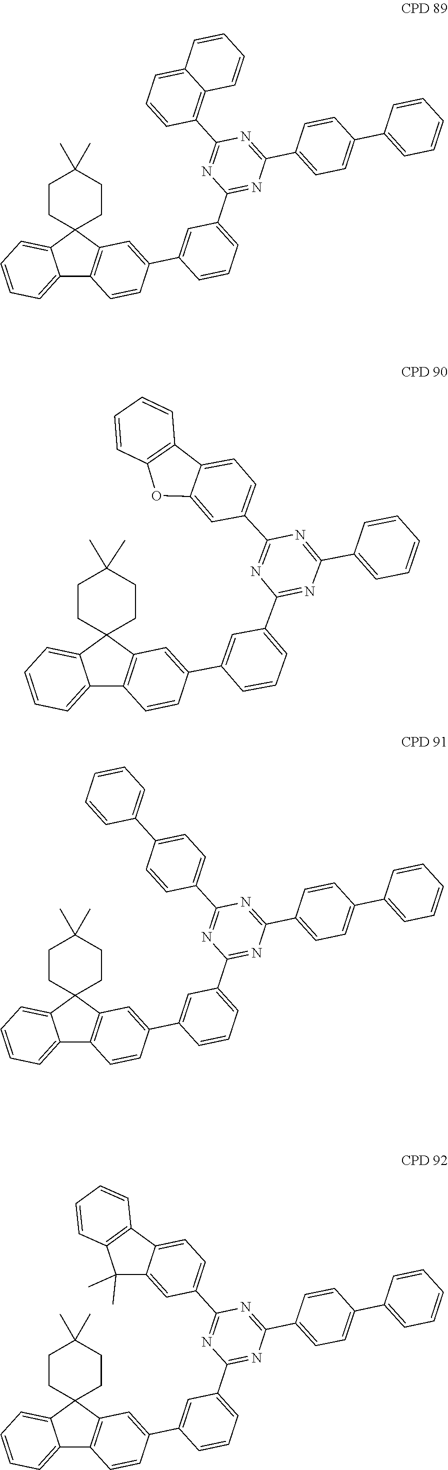 [SMILES: CC1(C)CCC2(CC1)c1ccccc1-c1ccc(-c3cccc(-c4nc(-c5ccc(-c6ccccc6)cc5)nc(-c5cccc6ccccc56)n4)c3)cc12 ; CC1(C)CCC2(CC1)c1ccccc1-c1ccc(-c3cccc(-c4nc(-c5ccccc5)nc(-c5ccc6c(c5)oc5ccccc56)n4)c3)cc12 ; CC1(C)CCC2(CC1)c1ccccc1-c1ccc(-c3cccc(-c4nc(-c5ccc(-c6ccccc6)cc5)nc(-c5ccc(-c6ccccc6)cc5)n4)c3)cc12 ; CC1(C)CCC2(CC1)c1ccccc1-c1ccc(-c3cccc(-c4nc(-c5ccc(-c6ccccc6)cc5)nc(-c5ccc6c(c5)C(C)(C)c5ccccc5-6)n4)c3)cc12]

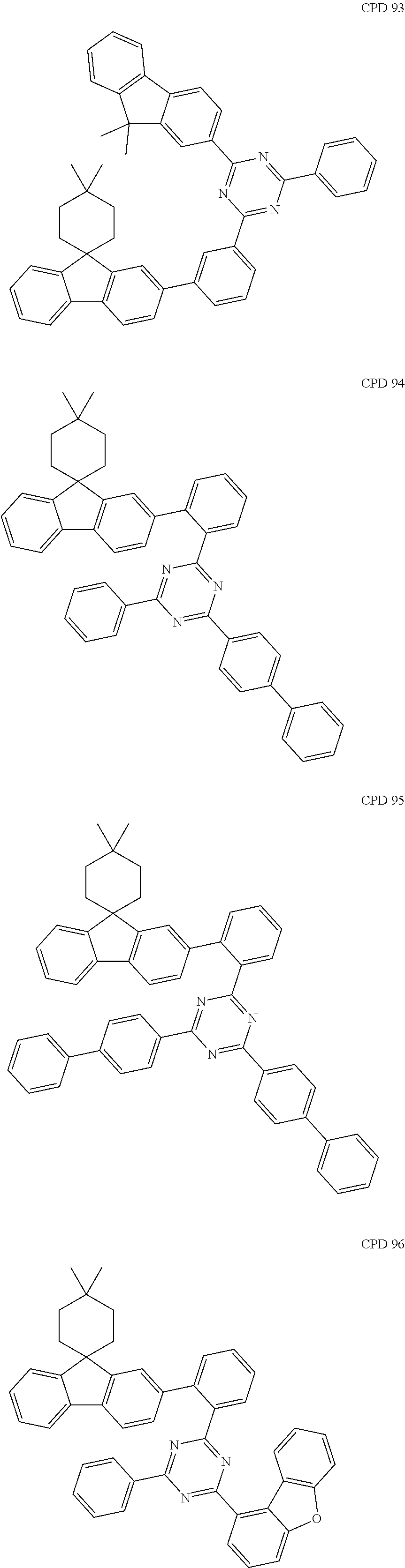 [SMILES: CC1(C)CCC2(CC1)c1ccccc1-c1ccc(-c3cccc(-c4nc(-c5ccccc5)nc(-c5ccc6c(c5)C(C)(C)c5ccccc5-6)n4)c3)cc12 ; CC1(C)CCC2(CC1)c1ccccc1-c1ccc(-c3ccccc3-c3nc(-c4ccccc4)nc(-c4ccc(-c5ccccc5)cc4)n3)cc12 ; CC1(C)CCC2(CC1)c1ccccc1-c1ccc(-c3ccccc3-c3nc(-c4ccc(-c5ccccc5)cc4)nc(-c4ccc(-c5ccccc5)cc4)n3)cc12 ; CC1(C)CCC2(CC1)c1ccccc1-c1ccc(-c3ccccc3-c3nc(-c4ccccc4)nc(-c4cccc5oc6ccccc6c45)n3)cc12]

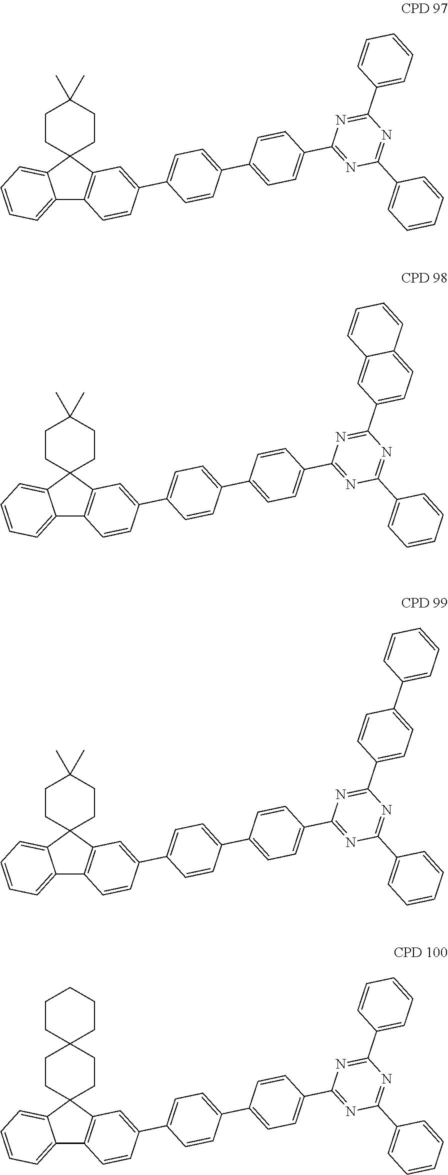 [SMILES: CC1(C)CCC2(CC1)c1ccccc1-c1ccc(-c3ccc(-c4ccc(-c5nc(-c6ccccc6)nc(-c6ccccc6)n5)cc4)cc3)cc12 ; CC1(C)CCC2(CC1)c1ccccc1-c1ccc(-c3ccc(-c4ccc(-c5nc(-c6ccccc6)nc(-c6ccc7ccccc7c6)n5)cc4)cc3)cc12 ; CC1(C)CCC2(CC1)c1ccccc1-c1ccc(-c3ccc(-c4ccc(-c5nc(-c6ccccc6)nc(-c6ccc(-c7ccccc7)cc6)n5)cc4)cc3)cc12 ; c1ccc(-c2nc(-c3ccccc3)nc(-c3ccc(-c4ccc(-c5ccc6c(c5)C5(CCC7(CCCCC7)CC5)c5ccccc5-6)cc4)cc3)n2)cc1]

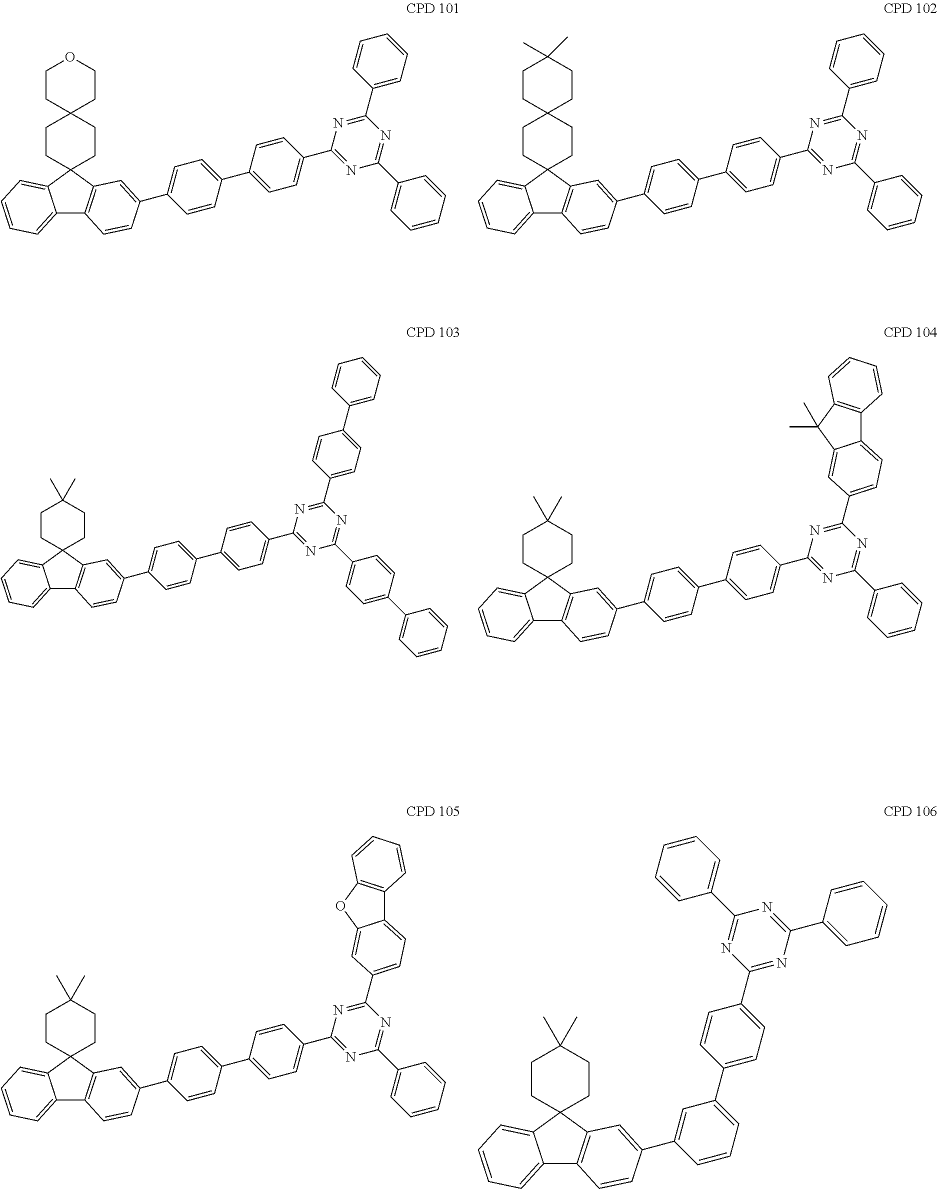

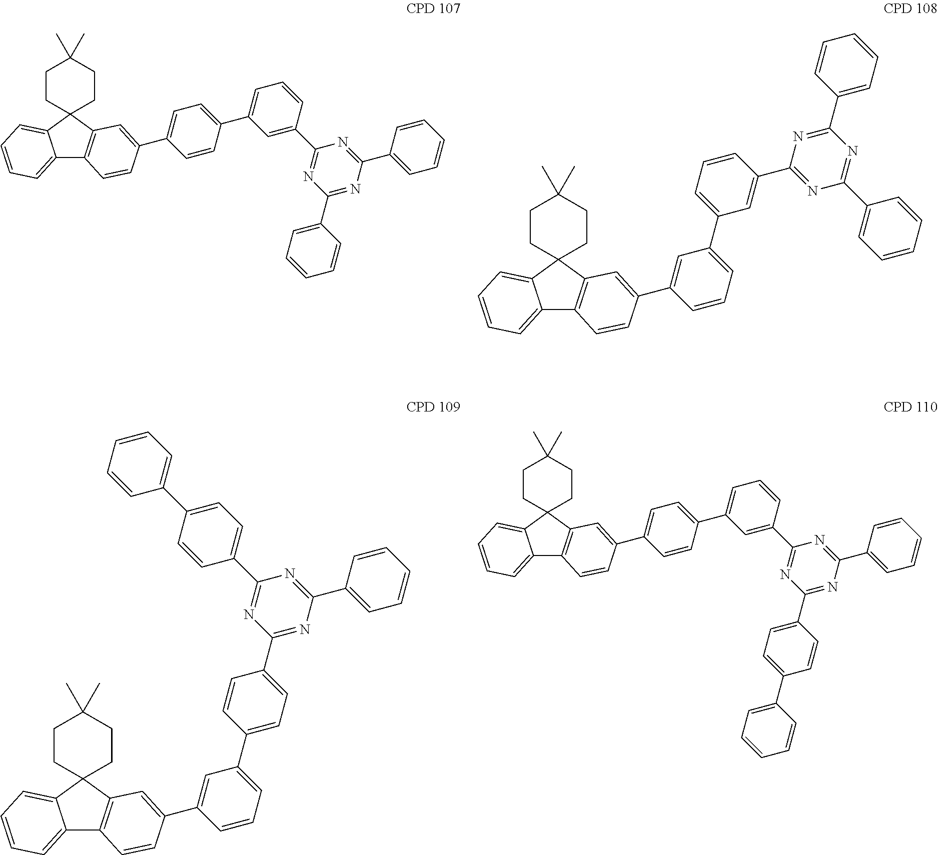 [SMILES: CC1(C)CCC2(CC1)c1ccccc1-c1ccc(-c3ccc(-c4cccc(-c5nc(-c6ccccc6)nc(-c6ccccc6)n5)c4)cc3)cc12 ; CC1(C)CCC2(CC1)c1ccccc1-c1ccc(-c3cccc(-c4cccc(-c5nc(-c6ccccc6)nc(-c6ccccc6)n5)c4)c3)cc12 ; CC1(C)CCC2(CC1)c1ccccc1-c1ccc(-c3cccc(-c4ccc(-c5nc(-c6ccccc6)nc(-c6ccc(-c7ccccc7)cc6)n5)cc4)c3)cc12 ; CC1(C)CCC2(CC1)c1ccccc1-c1ccc(-c3ccc(-c4cccc(-c5nc(-c6ccccc6)nc(-c6ccc(-c7ccccc7)cc6)n5)c4)cc3)cc12]

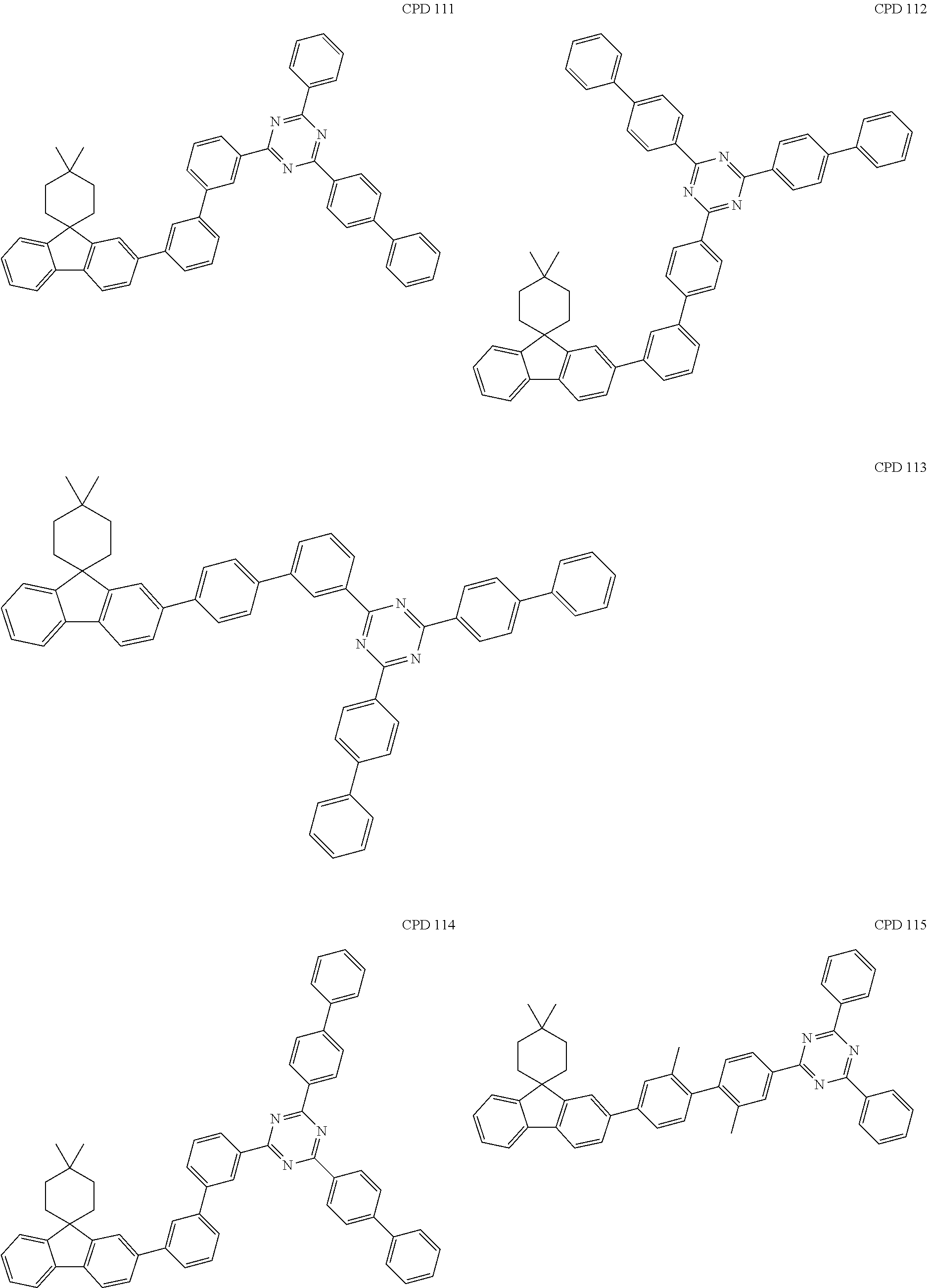

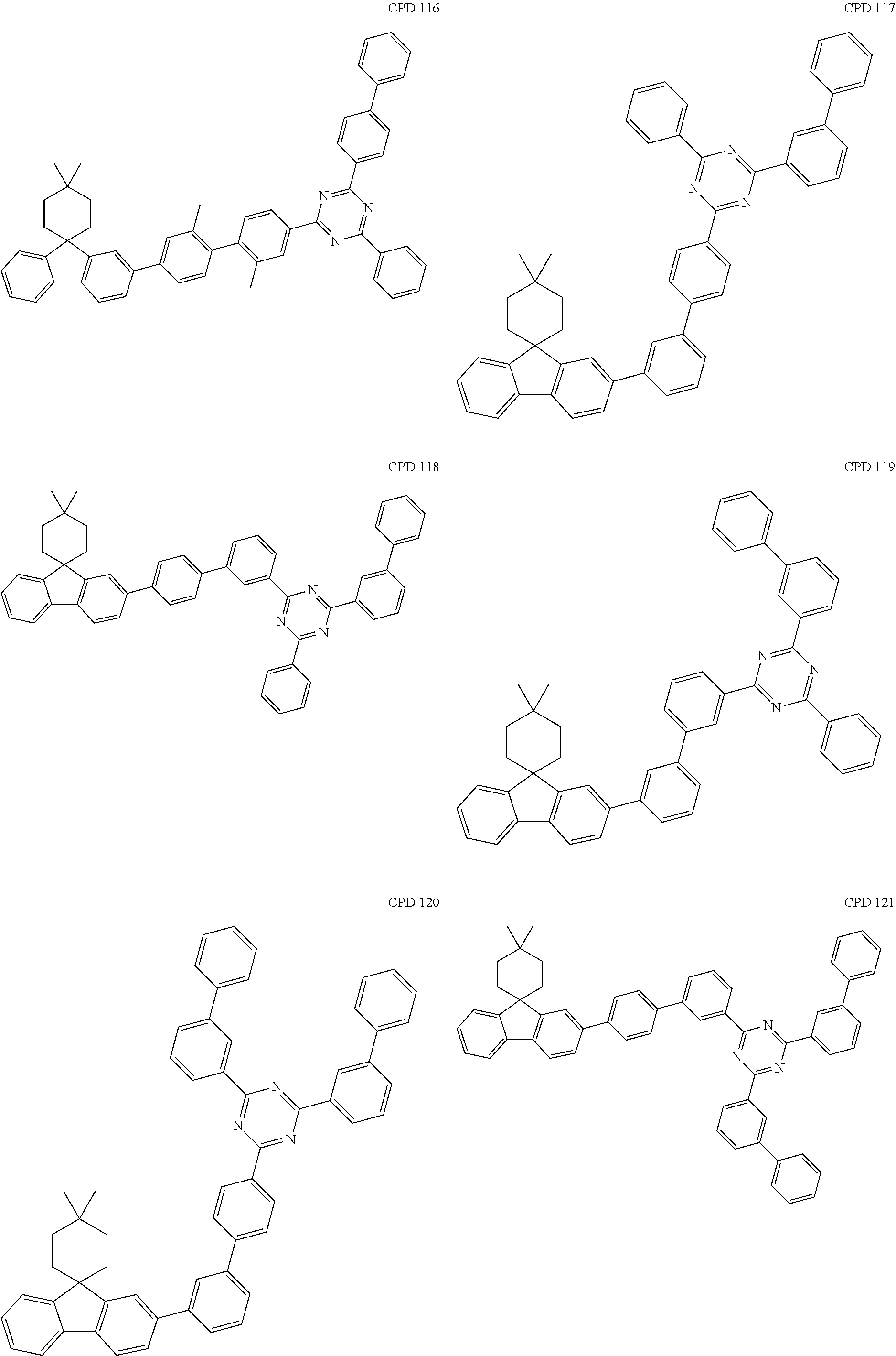

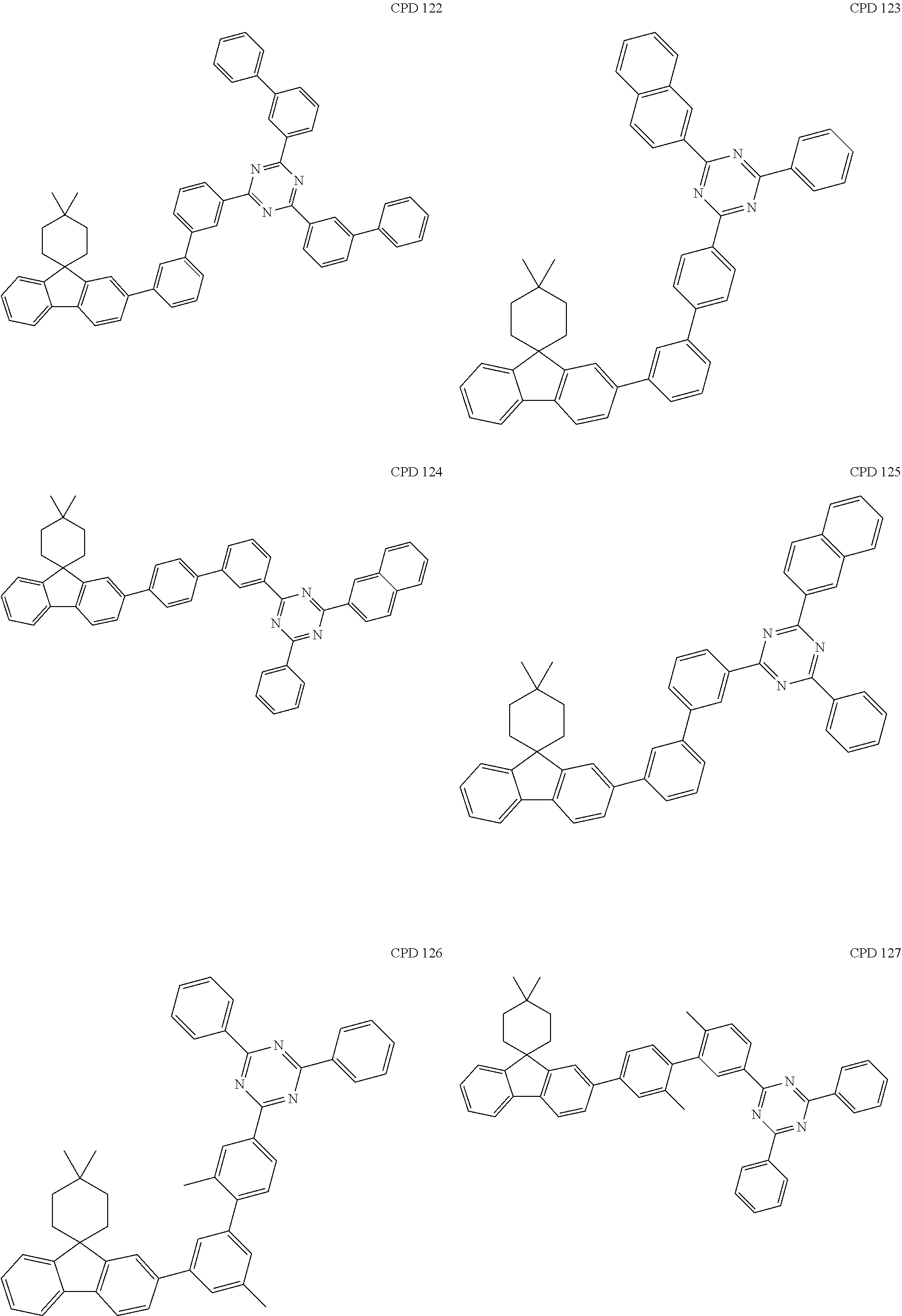

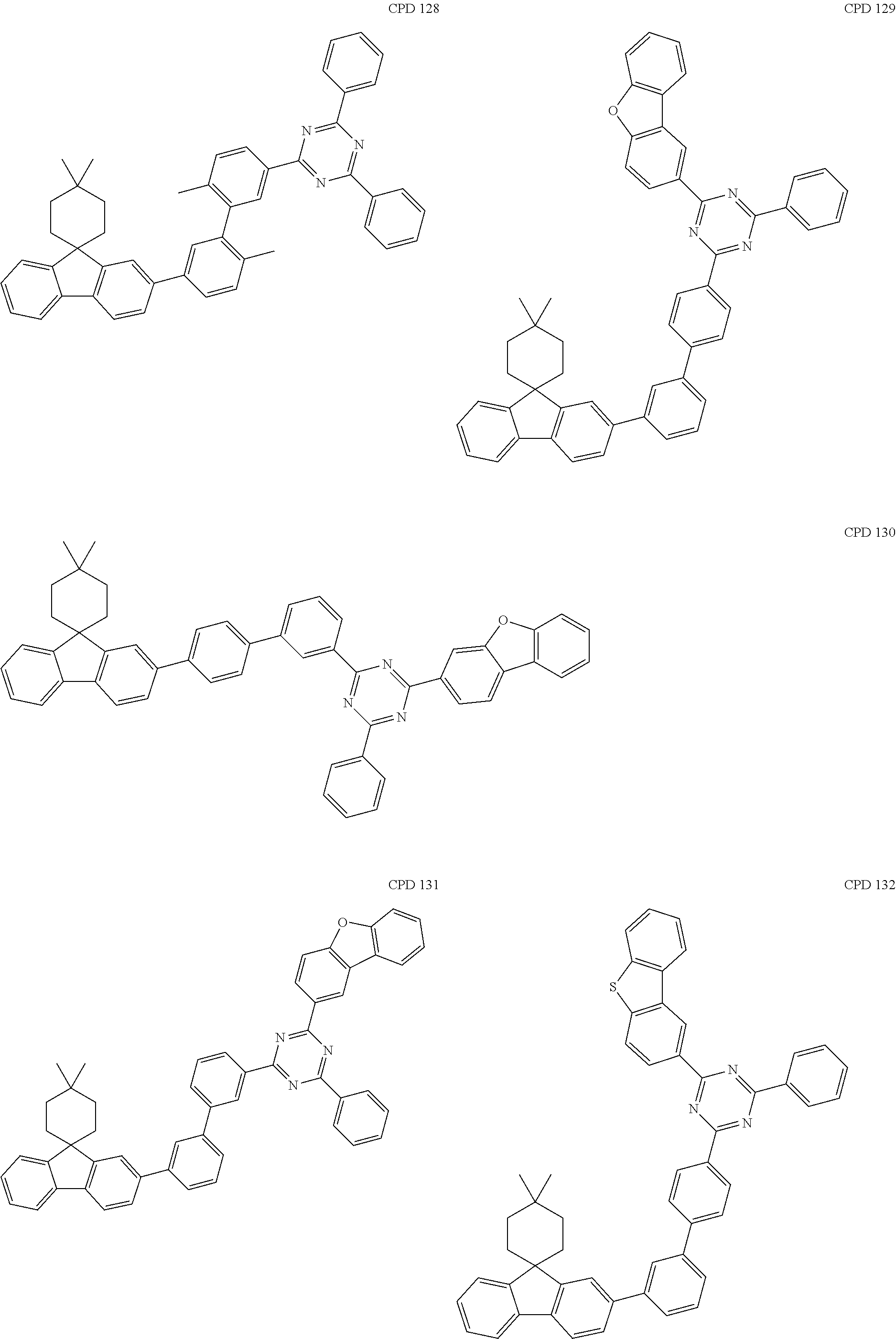

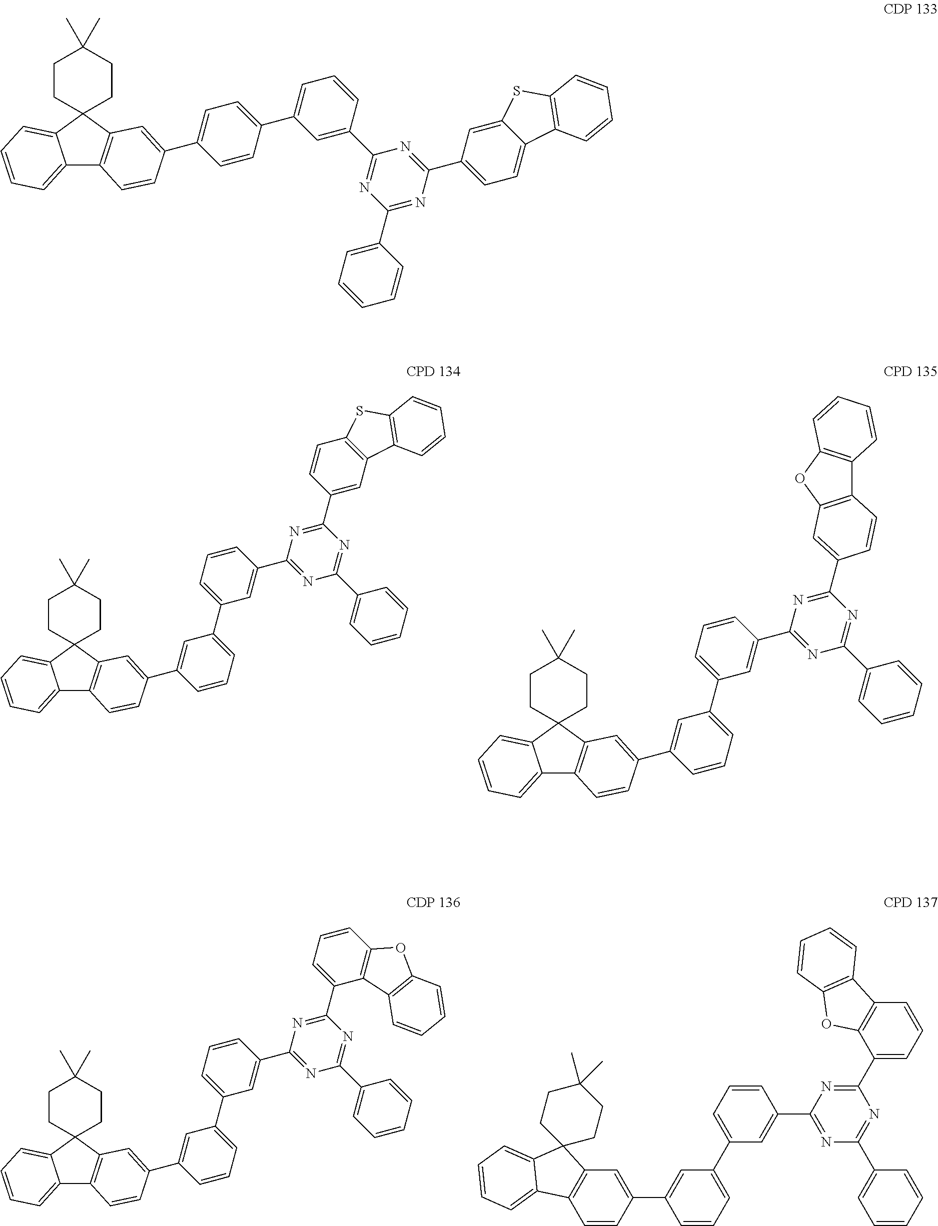

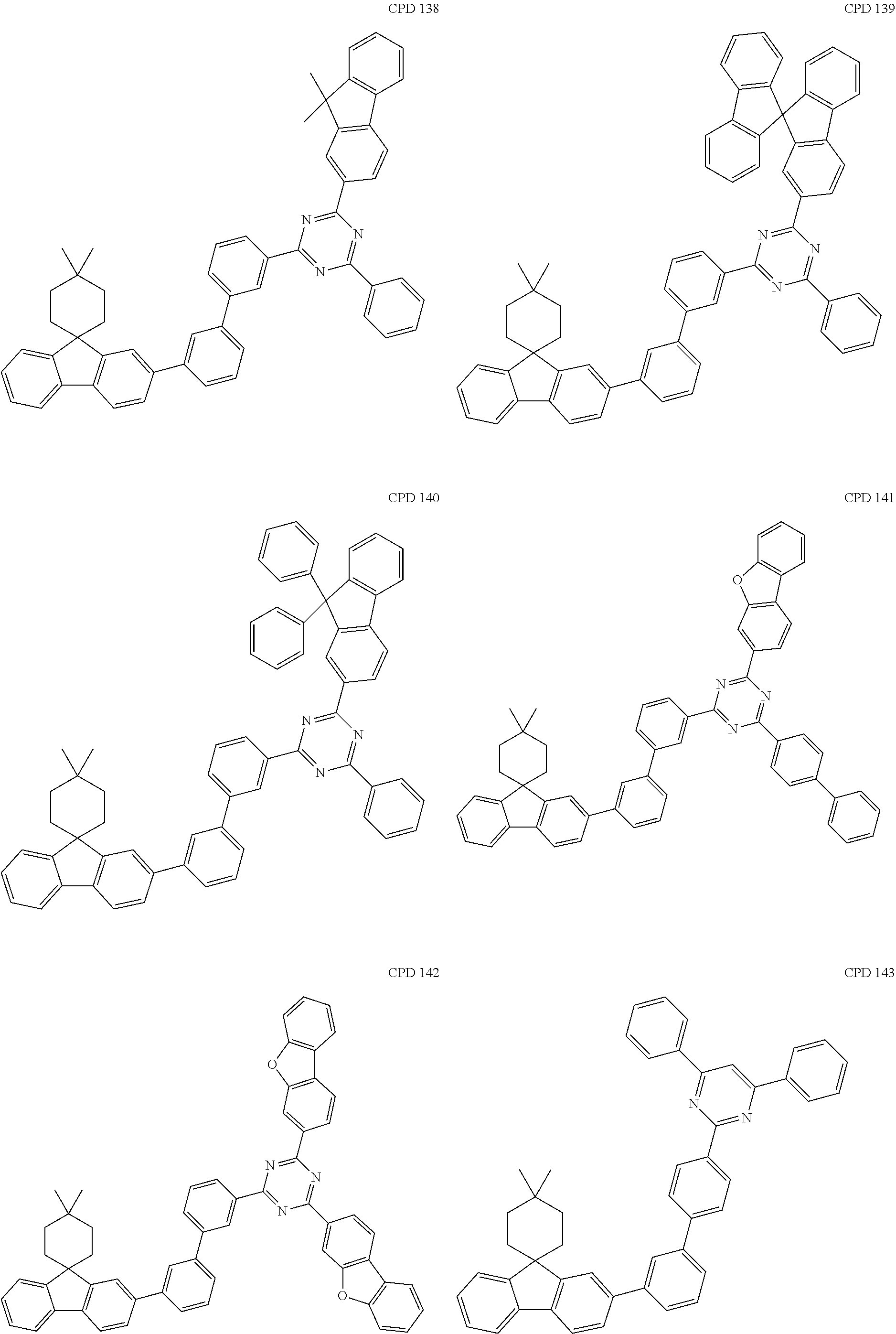

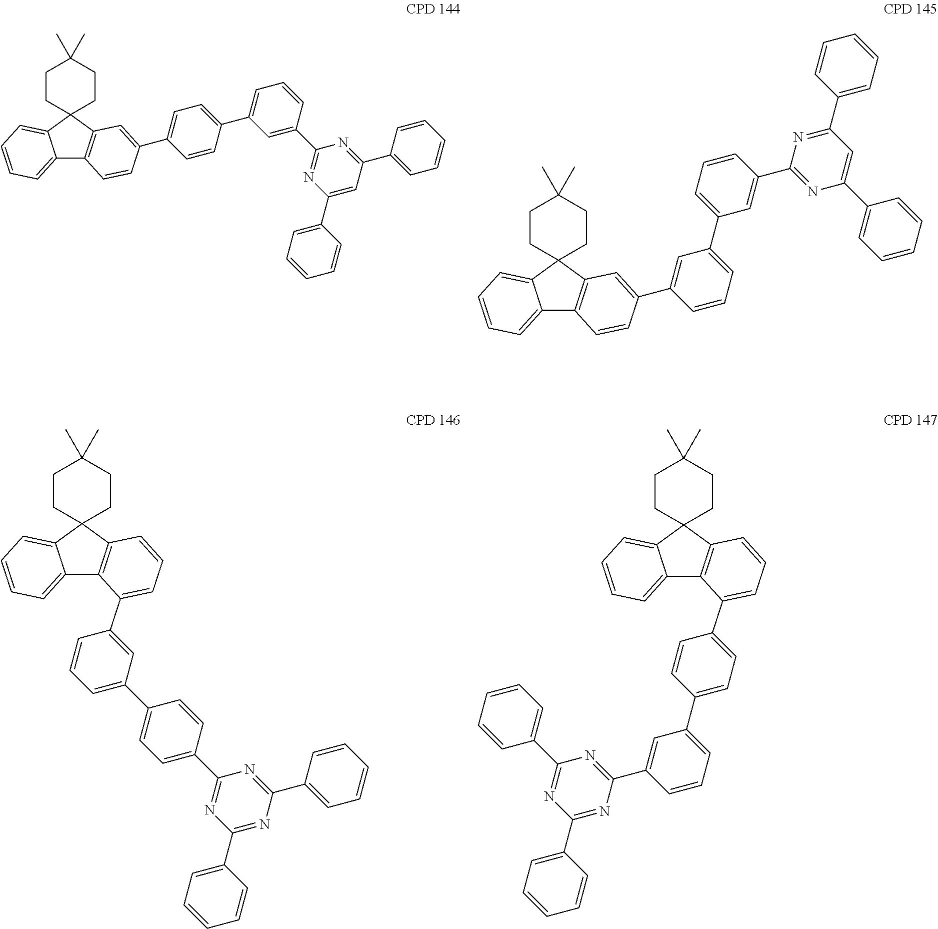 [SMILES: CC1(C)CCC2(CC1)c1ccccc1-c1ccc(-c3ccc(-c4cccc(-c5nc(-c6ccccc6)cc(-c6ccccc6)n5)c4)cc3)cc12 ; CC1(C)CCC2(CC1)c1ccccc1-c1ccc(-c3cccc(-c4cccc(-c5nc(-c6ccccc6)cc(-c6ccccc6)n5)c4)c3)cc12 ; CC1(C)CCC2(CC1)c1ccccc1-c1c(-c3cccc(-c4ccc(-c5nc(-c6ccccc6)nc(-c6ccccc6)n5)cc4)c3)cccc12 ; CC1(C)CCC2(CC1)c1ccccc1-c1c(-c3ccc(-c4cccc(-c5nc(-c6ccccc6)nc(-c6ccccc6)n5)c4)cc3)cccc12]

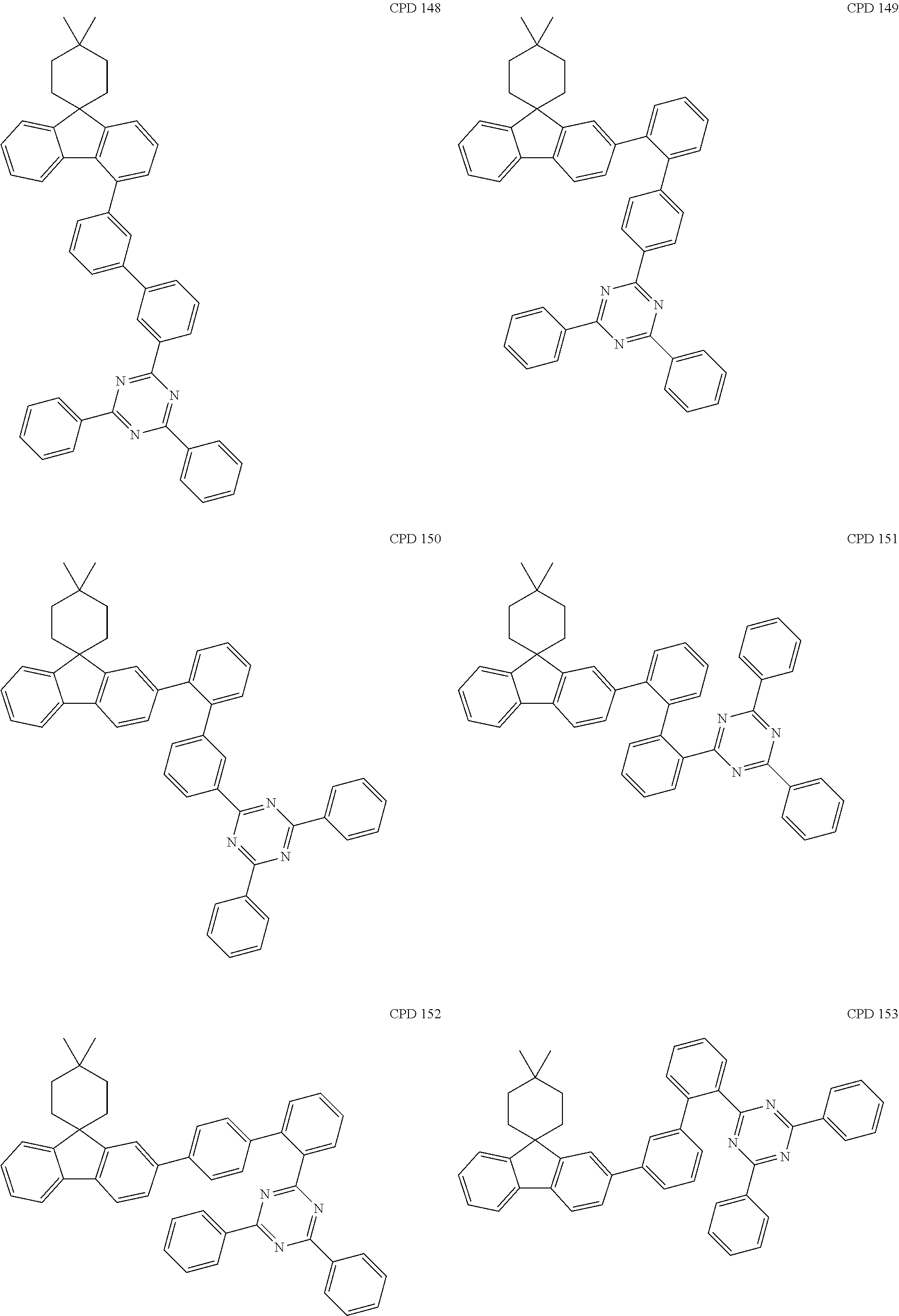

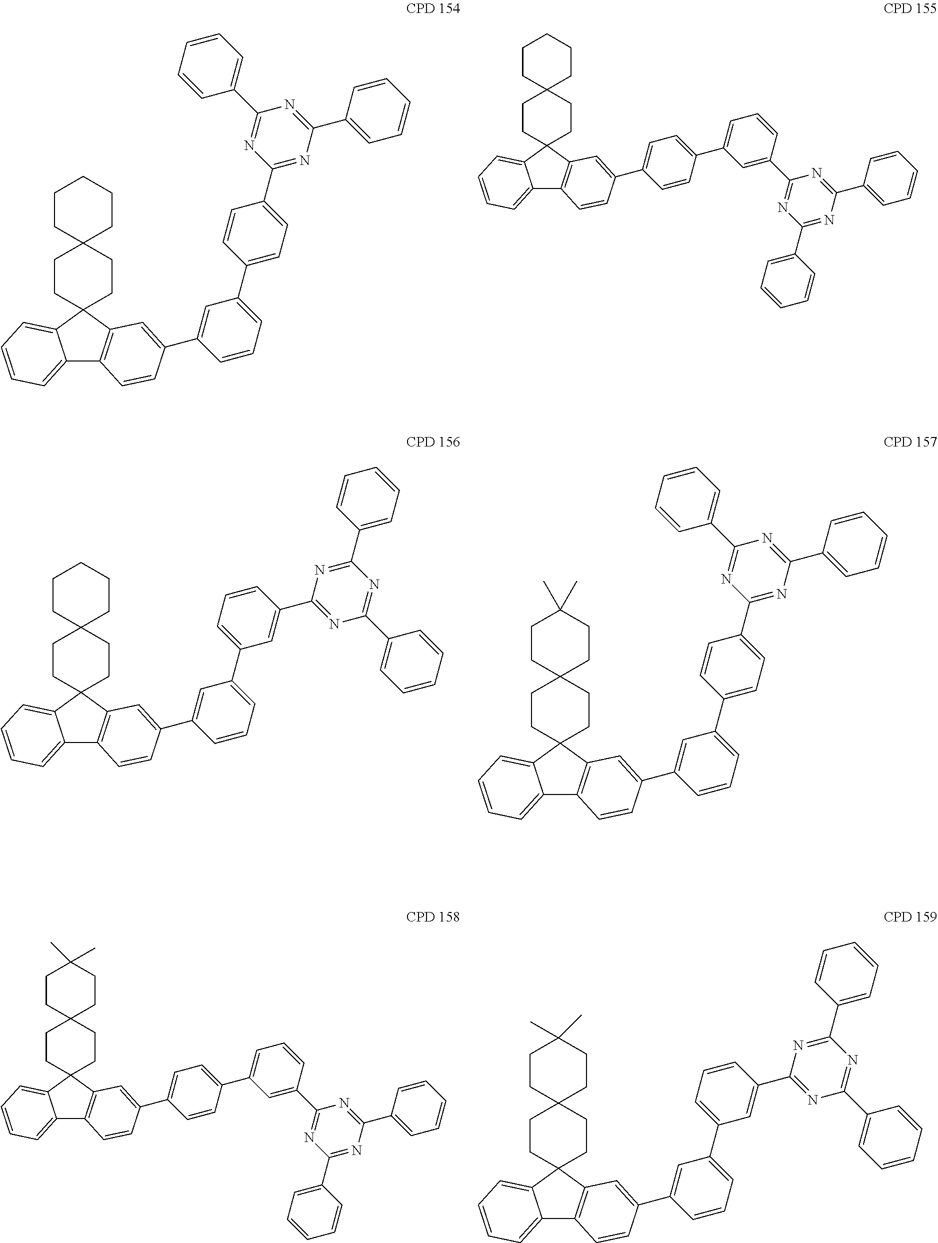

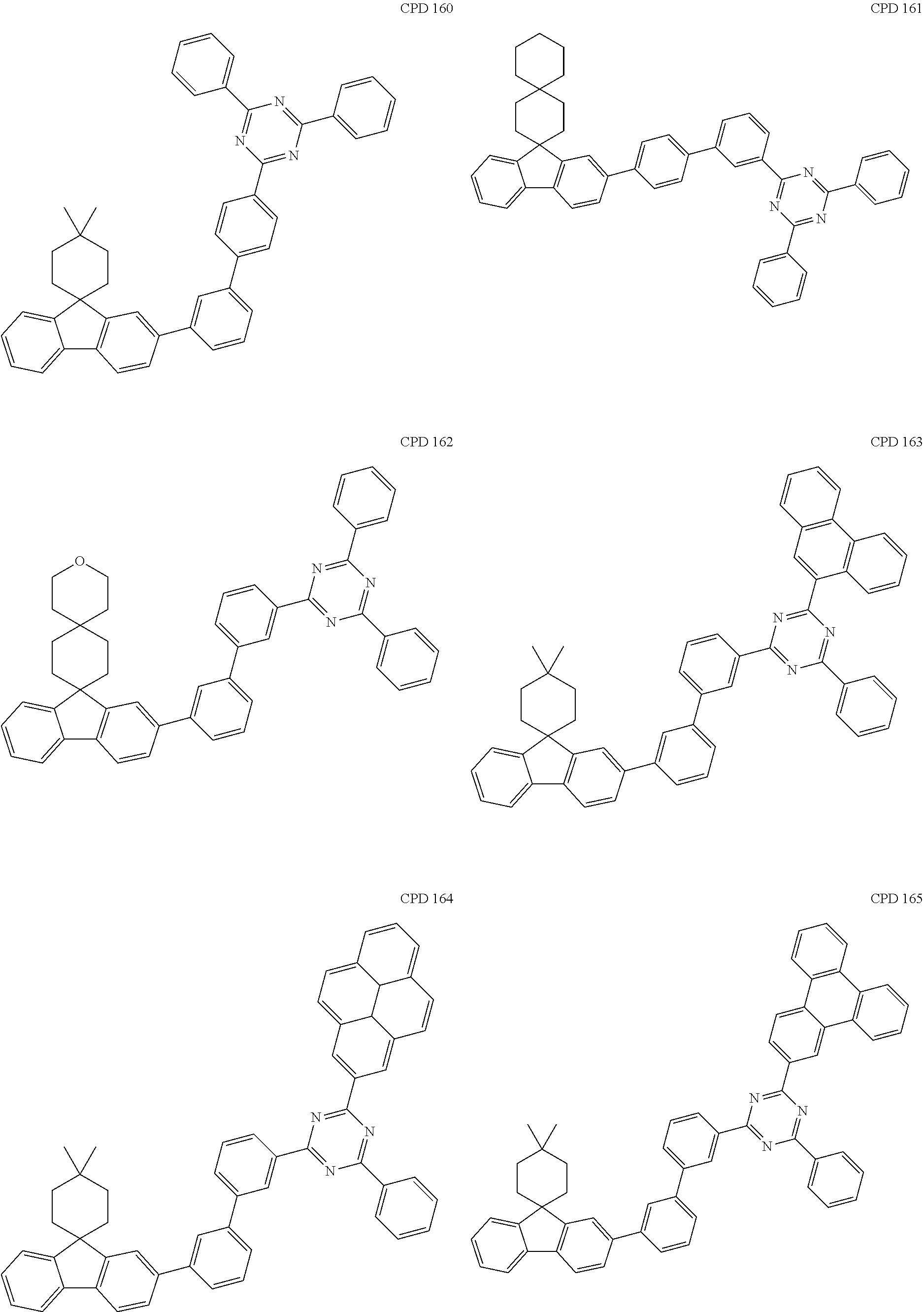

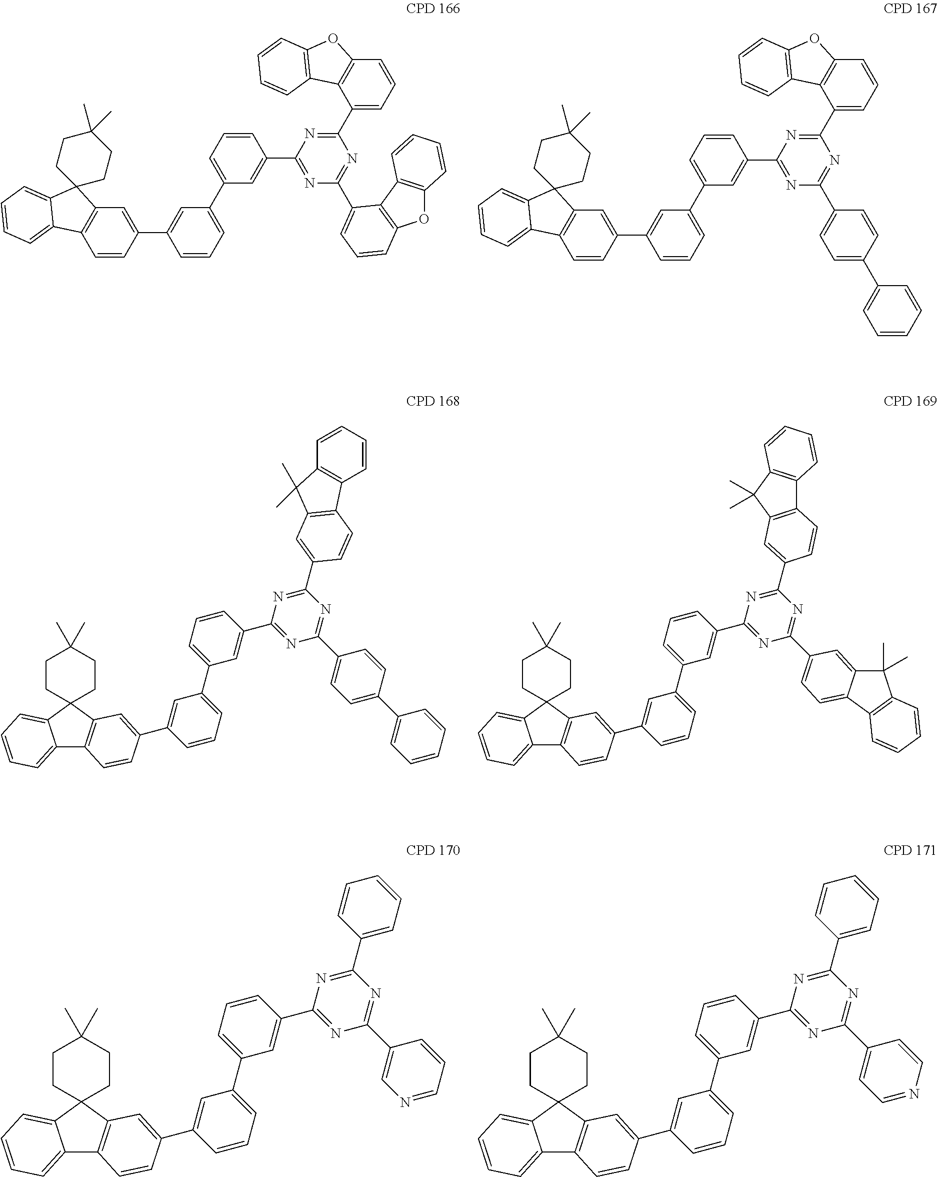

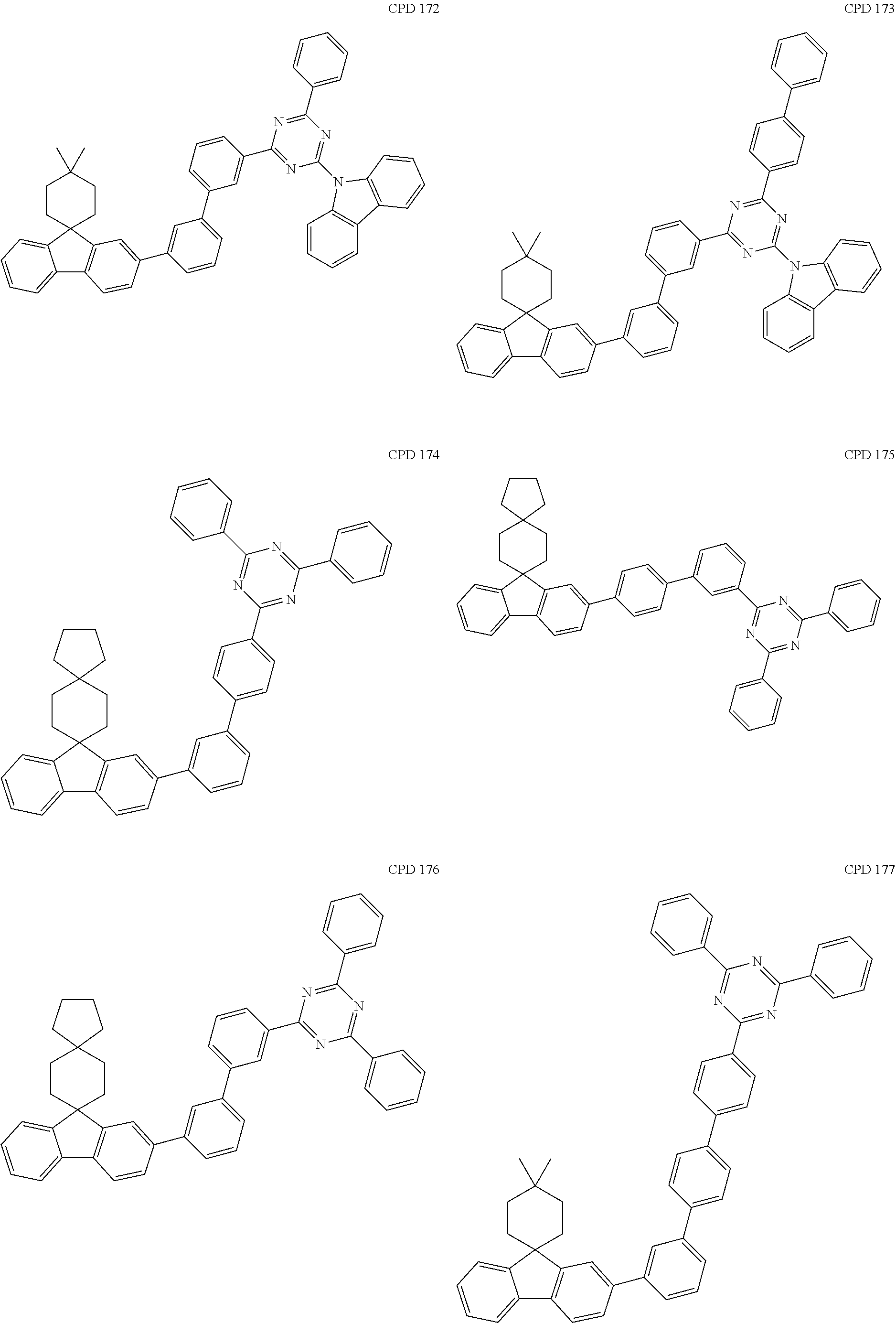

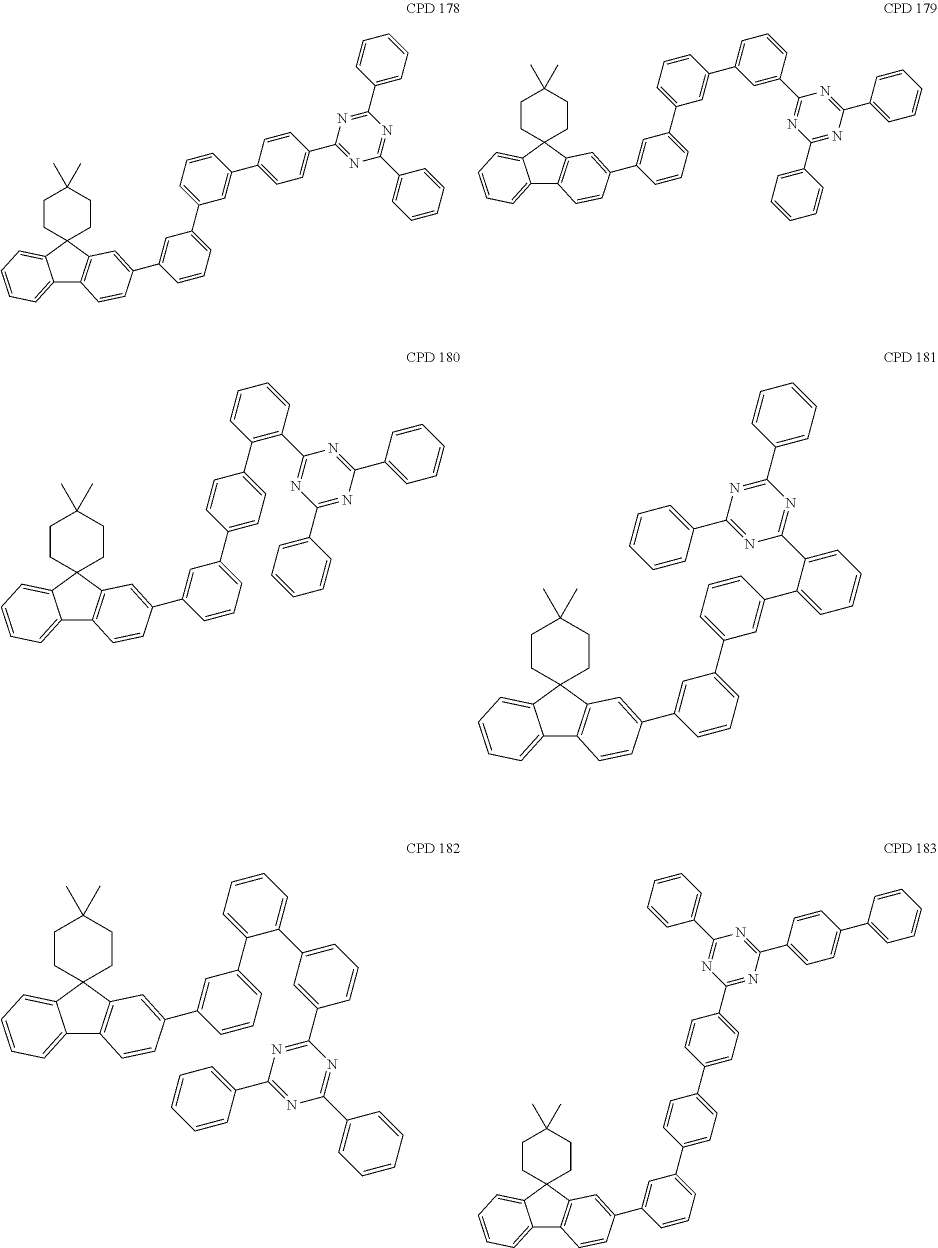

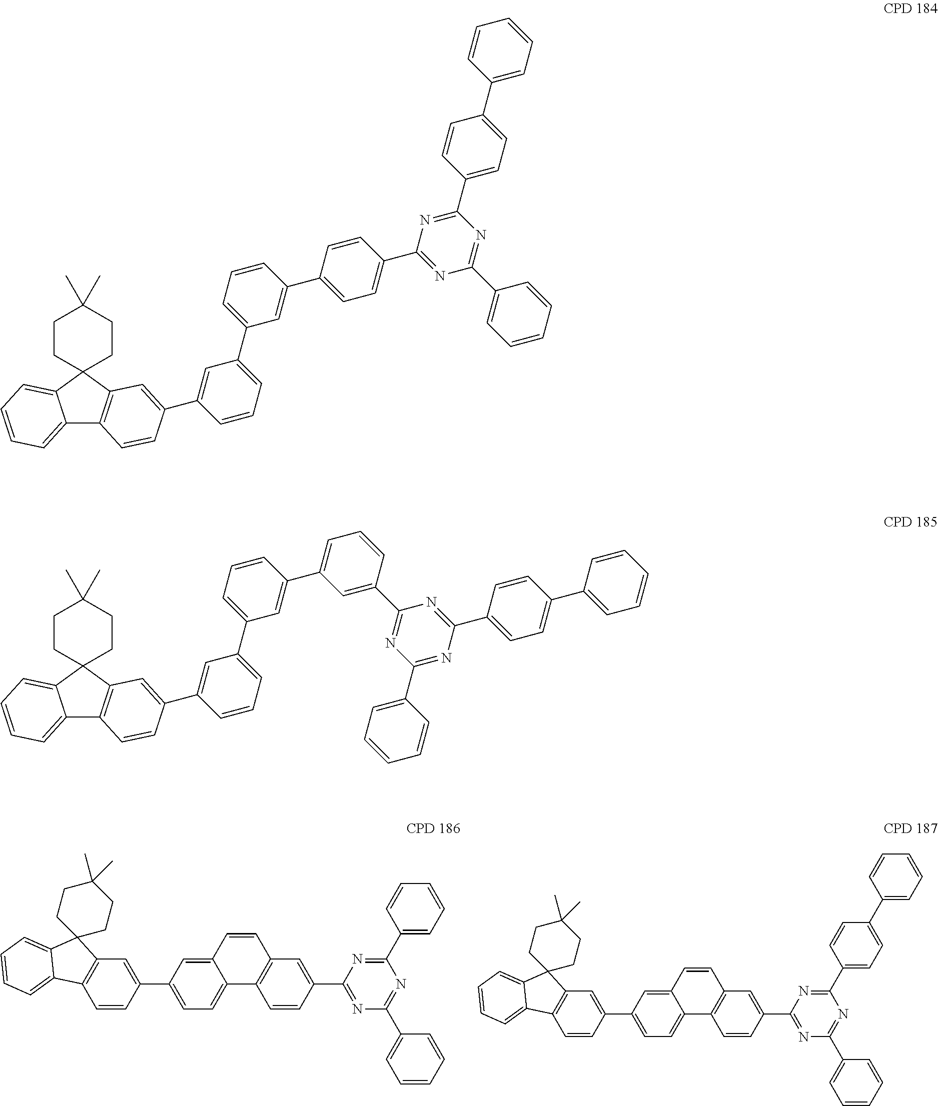 [SMILES: CC1(C)CCC2(CC1)c1ccccc1-c1ccc(-c3cccc(-c4cccc(-c5ccc(-c6nc(-c7ccccc7)nc(-c7ccc(-c8ccccc8)cc7)n6)cc5)c4)c3)cc12 ; CC1(C)CCC2(CC1)c1ccccc1-c1ccc(-c3cccc(-c4cccc(-c5cccc(-c6nc(-c7ccccc7)nc(-c7ccc(-c8ccccc8)cc7)n6)c5)c4)c3)cc12 ; CC1(C)CCC2(CC1)c1ccccc1-c1ccc(-c3ccc4c(ccc5cc(-c6nc(-c7ccccc7)nc(-c7ccccc7)n6)ccc54)c3)cc12 ; CC1(C)CCC2(CC1)c1ccccc1-c1ccc(-c3ccc4c(ccc5cc(-c6nc(-c7ccccc7)nc(-c7ccc(-c8ccccc8)cc7)n6)ccc54)c3)cc12]

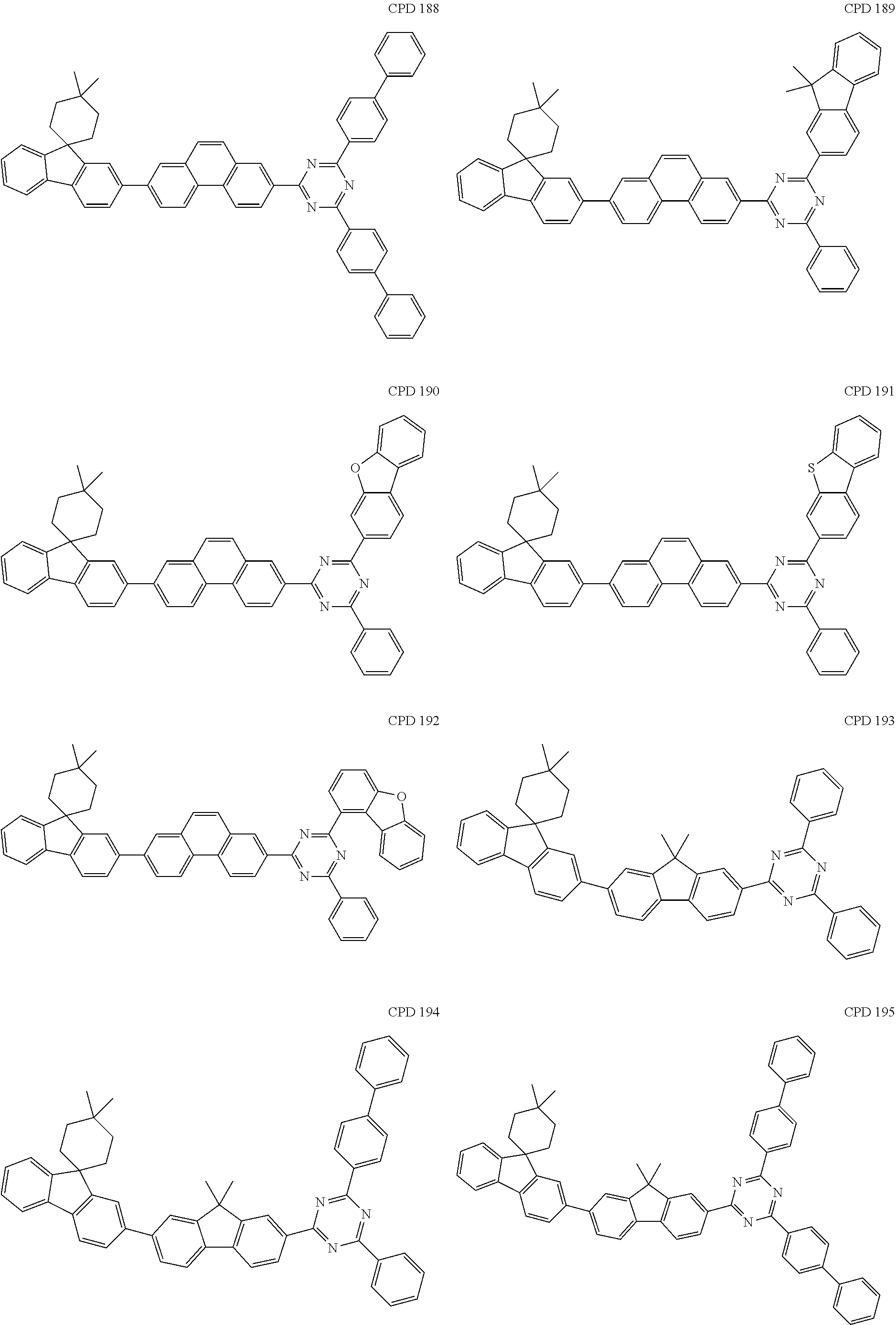

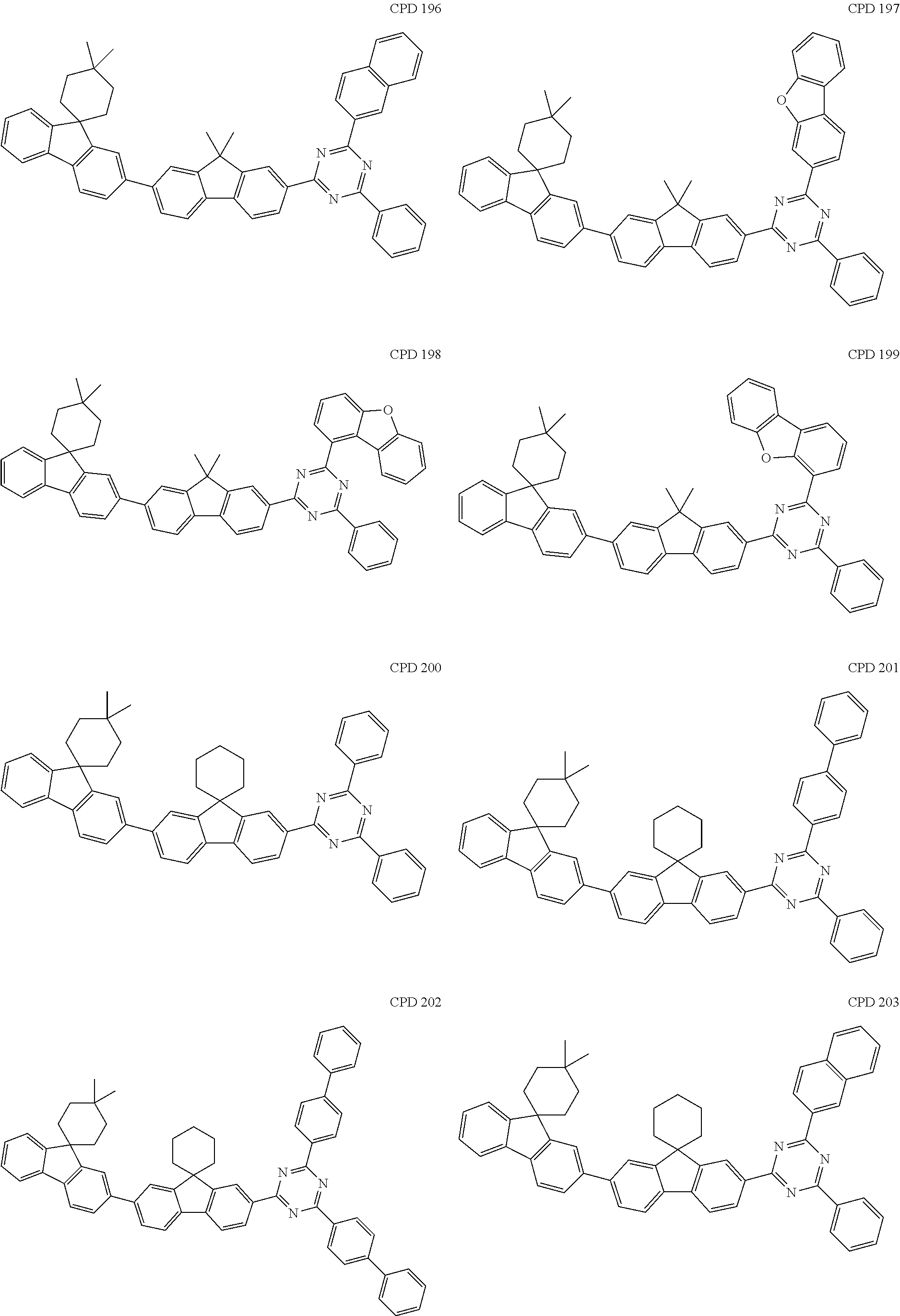

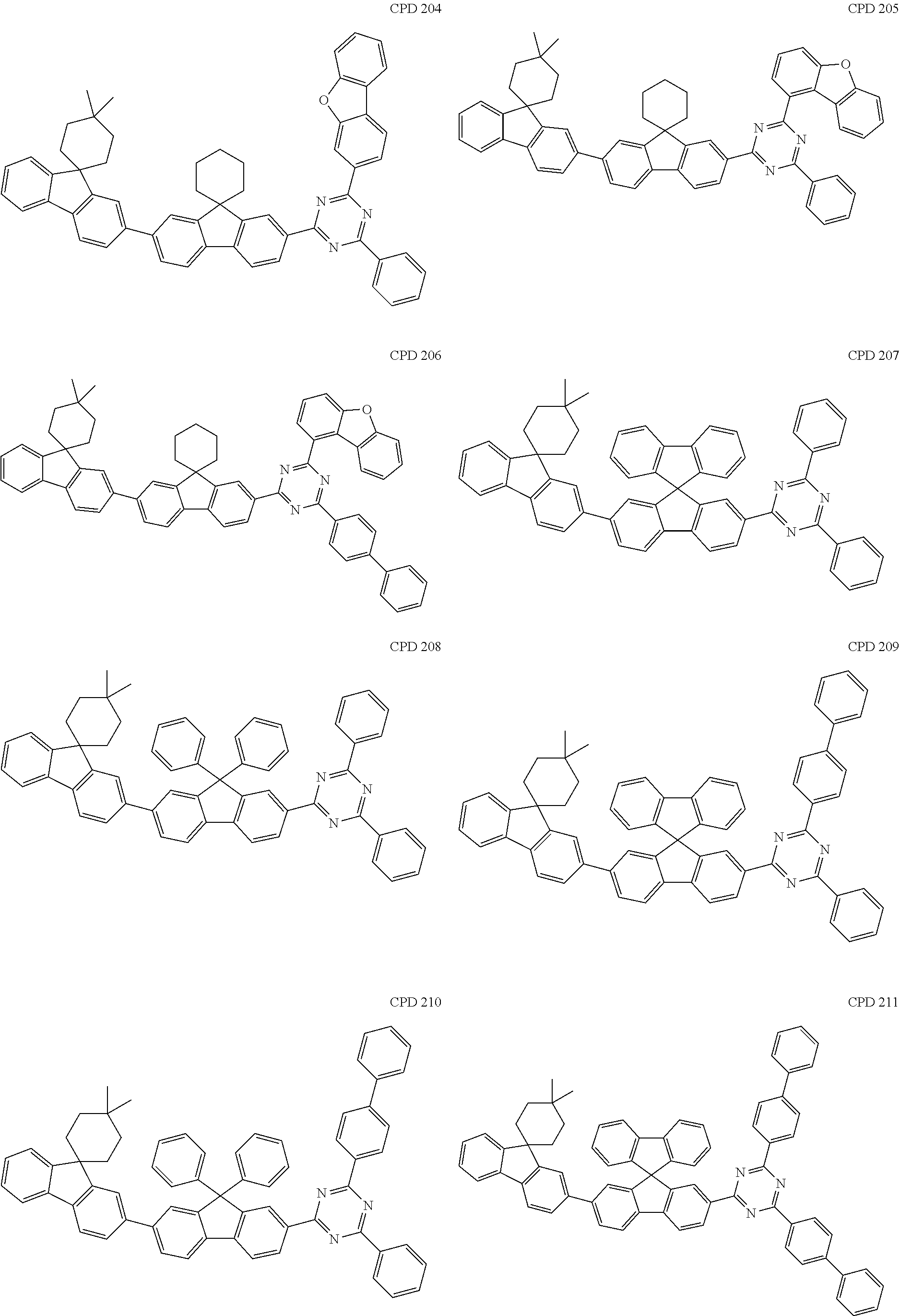

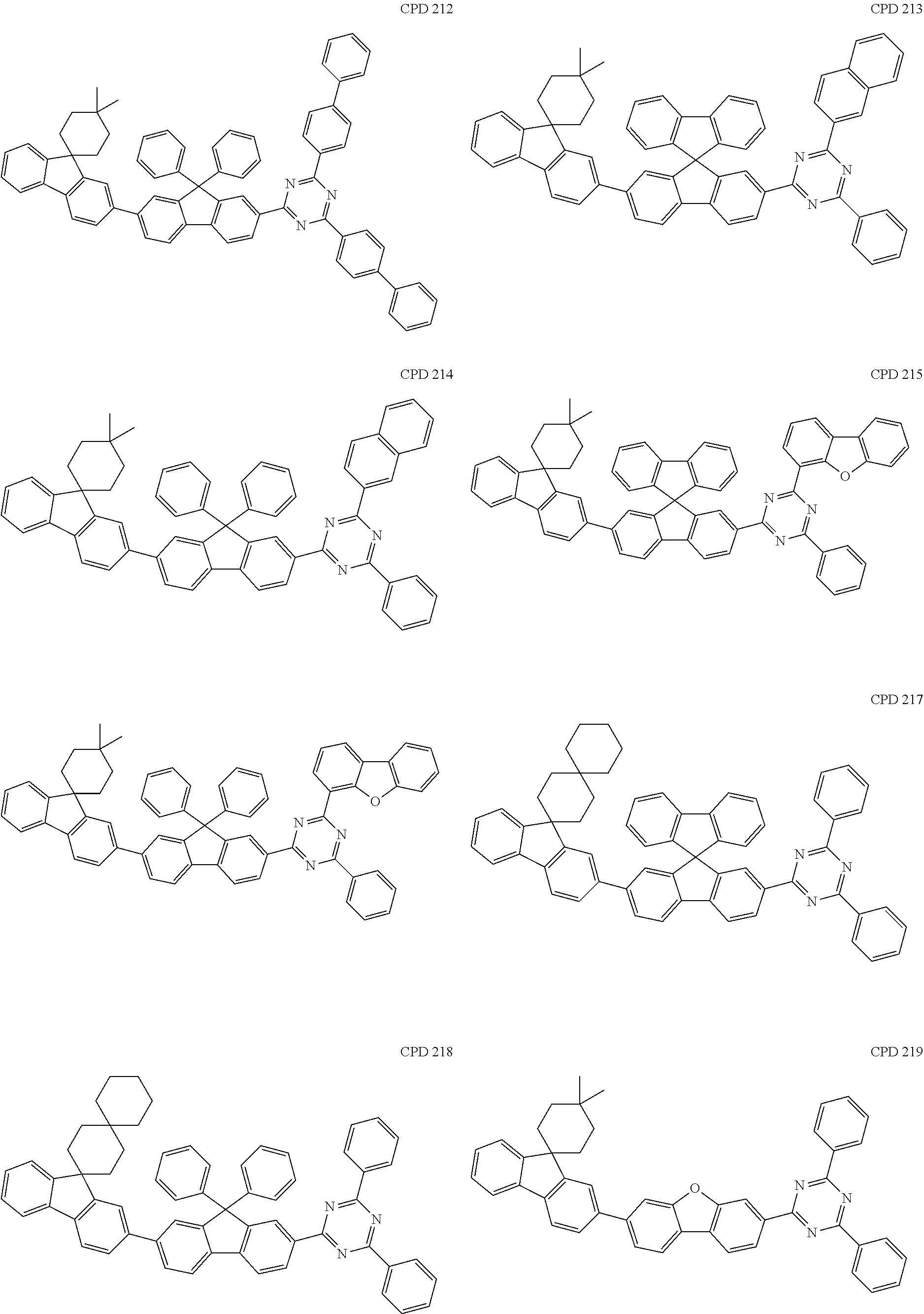

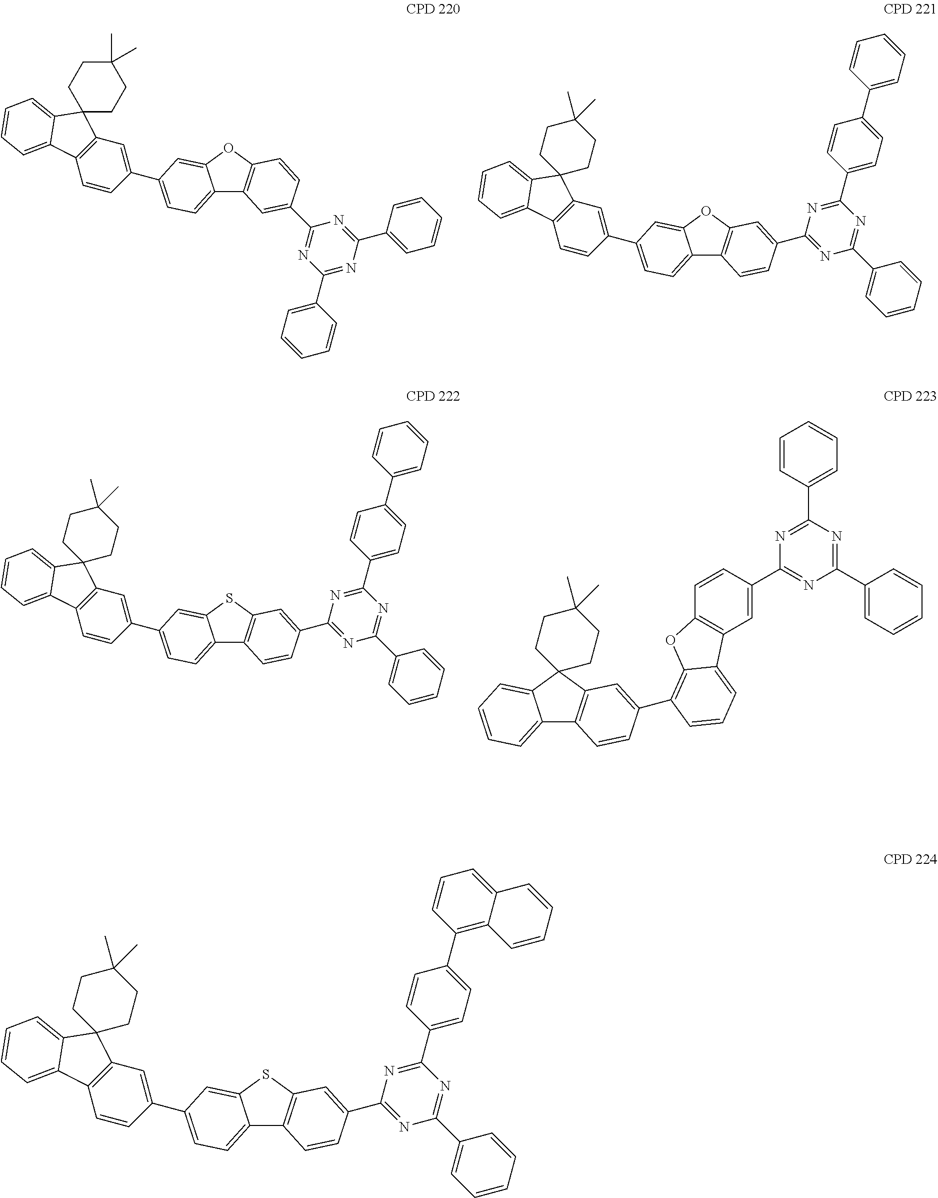 [SMILES: CC1(C)CCC2(CC1)c1ccccc1-c1ccc(-c3ccc4c(c3)oc3ccc(-c5nc(-c6ccccc6)nc(-c6ccccc6)n5)cc34)cc12 ; CC1(C)CCC2(CC1)c1ccccc1-c1ccc(-c3ccc4c(c3)oc3cc(-c5nc(-c6ccccc6)nc(-c6ccc(-c7ccccc7)cc6)n5)ccc34)cc12 ; CC1(C)CCC2(CC1)c1ccccc1-c1ccc(-c3ccc4c(c3)sc3cc(-c5nc(-c6ccccc6)nc(-c6ccc(-c7ccccc7)cc6)n5)ccc34)cc12 ; CC1(C)CCC2(CC1)c1ccccc1-c1ccc(-c3cccc4c3oc3ccc(-c5nc(-c6ccccc6)nc(-c6ccccc6)n5)cc34)cc12 ; CC1(C)CCC2(CC1)c1ccccc1-c1ccc(-c3ccc4c(c3)sc3cc(-c5nc(-c6ccccc6)nc(-c6ccc(-c7cccc8ccccc78)cc6)n5)ccc34)cc12]

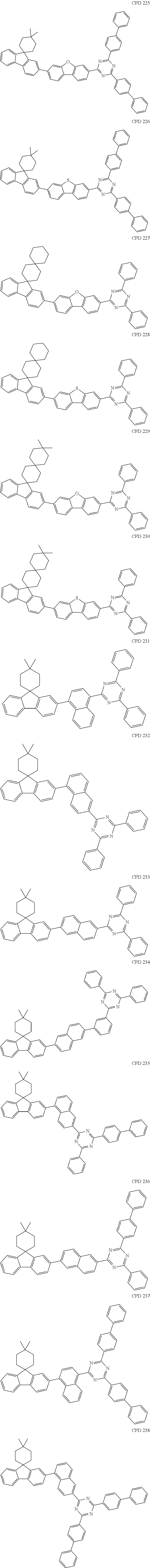

[chemical structure]

[chemical structure]

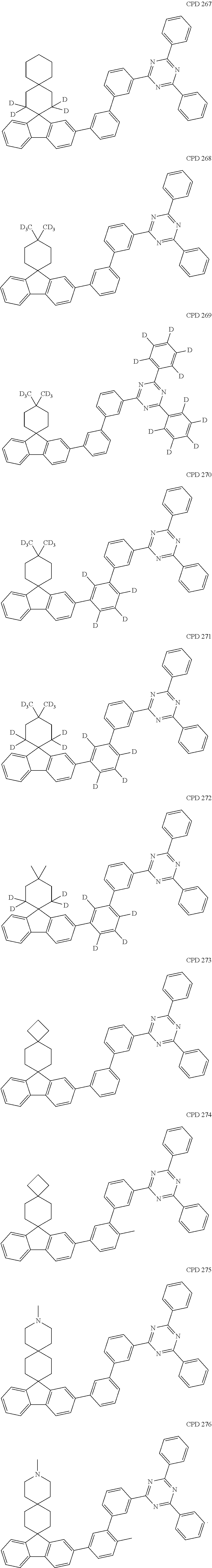

14. A light-emitting device, comprising an anode, a cathode, and one or more organic layers between the anode and the cathode, and at least one of the organic layers comprises the compound according to claim 1.

15. The light-emitting device according to claim 14, wherein the compound severs as a material in any one of a light-emitting layer, a hole blocking layer or an electron transport layer in the organic layer.

16. The light-emitting device according to claim 15, wherein the compound severs as a hole blocking layer material.

17. The light-emitting device according to claim 14, wherein the light-emitting device is an organic electroluminescent device.

18. The compound according to claim 7, wherein R₁, R₂ and R₃ are each independently selected from the group consisting of deuterium, halogen, cyano, nitro, C1-C10 alkyl, C1-C10 heteroalkyl, C2-C10 alkenyl, C2-C10 alkynyl, C3-C10 cycloalkyl, C3-C10 heterocycloalkyl, C6-C20 aryl, C5-C20 heteroaryl, C1-C10 alkoxy, C6-C20 aryloxy, C3-C10 alkylsilyl, C6-C20 arylsilyl, C1-C10 alkylboryl, C6-C20 arylboryl, C6-C20 monoarylphosphino, C6-C20 diarylphosphino, and C6-C30 arylamino.

19. The compound according to claim 8, wherein X₁ and X₂ are N, and X₃ is CH.

20. The compound according to claim 18, wherein R₁, R₂ and R₃ are each independently selected from the group consisting of deuterium, halogen, cyano, nitro, C1-C10 alkyl, C1-C10 heteroalkyl, C3-C10 cycloalkyl, C3-C10 heterocycloalkyl, C6-C12 aryl, C5-C12 heteroaryl, C1-C10 alkoxy, C6-C12 aryloxy, and C6-C12 arylamino.
